(19) United States
(12) Patent Application Publication
(10) Pub. No.: US 2018/0244785 A1
(43) Pub. Date: Aug. 30, 2018

(54) ANTI-FGFR ANTIBODIES AND METHODS OF USE

(71) Applicant: Merrimack Pharmaceuticals, Inc., Cambridge, MA (US)

(72) Inventors: Tamara DAKE, Sharon, MA (US); Gregory J. FINN, Watertown, MA (US); Melissa GEDDIE, Arlington, MA (US); Yasmin HASHAMBHOY-RAMSAY, Watertown, MA (US); Marco MUDA, Somerville, MA (US); Birgit M. SCHOEBERL, Cambridge, MA (US)

(21) Appl. No.: 15/864,764

(22) Filed: Jan. 8, 2018

Related U.S. Application Data
(60) Provisional application No. 62/555,859, filed on Sep. 8, 2017.
(60) Provisional application No. 62/478,943, filed on Mar. 30, 2017.
(60) Provisional application No. 62/444,174, filed on Jan. 9, 2017.

(51) Int. Cl. C07K 16/28; G01N 33/574; A61P 35/00; A61K 47/68

(52) U.S. Cl. CPC C07K 16/2863; G01N 33/57492; A61P 35/00; C07K 2317/31; C07K 2317/92; G01N 2333/705; C07K 2317/21; A61K 47/6849

(57) ABSTRACT

Anti-FGFR antibodies which bind to particular isoforms of FGFR1-4, therapeutic compositions comprising the anti-FGFR antibodies, and methods of using such antibodies and compositions in the treatment of FGFR-related disorders (e.g., cancer) are disclosed.

### RELATED INFORMATION

This patent application claims the benefit of U.S. Provisional Patent Application Ser. No. 62/444174, filed Jan. 9, 2017, U.S. Provisional Patent Application Ser. No. 62/478943, filed Mar. 30, 2017, and U.S. Provisional Patent Application Ser. No. 62/555859, filed Sep. 8, 2017. The entire contents of the above-referenced provisional patent applications are incorporated herein by reference.

### BACKGROUND

In humans, the fibroblast growth factor (FGF) family of ligands comprises 22 genes. FGF family members share amino acid sequence identity ranging from 16 to 65%, and have been shown to regulate a variety of responses ranging from embryo morphogenesis, wound healing, control of nervous system, metabolism, skeletal function, tumor angiogenesis, and tumor proliferation.

The FGF cognate-receptor family includes four genes that, like all tyrosine kinase receptors, are composed of an extracellular ligand binding domain, a single transmembrane α-helix, and a cytoplasmic tyrosine kinase domain. The FGFR extracellular domain is composed of up to three immunoglobulin-like domains (D 1-3), with D2 and D3 comprising the ligand binding portion. In addition, D2 domain also includes a positively charged region that serves as a binding domain for heparan sulfate proteoglycan (HSPG), and one characteristic of the FGF receptor-ligand signaling system is the formation of a ternary complex between ligand, receptor and HSPG. Another critical distinctive feature among members of the FGF receptor (FGFR) family is the extensive use of alternative splicing to generate multiple isoforms. Splicing variants have been identified for all four mammalian FGFRs, and the most characterized variants are the splicing variations generated within the D3 domain. In fact, the alternative usage of two exons within FGF receptor genes 1, 2 and 3 (but not FGFR4) generate two receptor variants named “c” and “b” respectively. The alternative splicing of the D3 domain into the b or c forms confers this system additional complexity.

Among numerous reports describing the role of various FGFRs in cancer, several have identified FGFR1 as a cancer driver in both blood and solid tumors. For example, chromosomal translocation and genetic fusion between intracellular kinase domain of FGFR1 and various genes has been found to cause 8p11 myelo-proliferative syndrome (Knights et al., Pharmacol Ther 2010; 125:105-17), and the FGFR1 gene locus has been found to be amplified in approximately 10% of breast cancer patients and was recently shown to drive proliferation and tamoxifen resistance in various cancer cell lines (Turner et al., Cancer Res 2010; 70:2085-94). Finally, FGFR1 amplification was found in 20% of squamous cell lung cancer patient (Weiss et al., Sci Transl Med 2010; 2:62ra93). However, the development of FGFR1 antagonistic antibodies have stalled due to anorexic side effects associated with anti-FGFR1 monoclonal antibodies in animal models.

Given that FGFR is a potential target for anti-cancer therapy, novel agents and methods of inhibiting FGFR activity that are not associated with debilitating side effects are desirable. The present disclosure addresses this unmet need.

### SUMMARY

Provided herein are isolated antibodies, such as monoclonal antibodies (e.g., human monoclonal antibodies) that specifically bind to particular isoforms of FGFR proteins and have desirable properties, such as high binding affinity to FGFR IIIc isoforms, and the ability to block the binding of FGF ligand to FGFR proteins, inhibit FGFR signaling, and inhibit FGF-mediated cell viability. The antibodies described herein can be used to inhibit tumor growth, treat cancer (e.g., FGFR-expressing cancers), and detect FGFR proteins in a sample.

Accordingly, in one aspect, provided herein are antibodies (e.g., isolated monoclonal antibodies), or antigen-binding portions thereof, which bind to human FGFR1c, FGFR2c, FGFR3c, and/or FGFR4. In some embodiments, the antibodies do not bind to FGFR1b, FGFR2b, and/or FGFR3b.

In another aspect, provided herein are isolated monoclonal antibodies, or antigen-binding portions thereof, which specifically bind to FGFR1c, FGFR2c, FGFR3c, and/or FGFR4, and comprise the three variable heavy chain CDRs and the three light chain CDRs that are in the heavy and light chain variable region pairs selected from the group consisting of SEQ ID NOs: 29 and 30; 40 and 41; 51 and 52; 62 and 63; 73 and 74; 84 and 85; 95 and 96; 106 and 107; 117 and 118; 128 and 129; 139 and 140; 150 and 151; 161 and 162; 172 and 173; 183 and 184; 194 and 195; 204 and 205; 214 and 215; 224 and 225; 234 and 235; 244 and 245; 254 and 255; 264 and 265; 274 and 275; 284 and 285; 294 and 295; 304 and 305; 314 and 315; 324 and 325; 334 and 335; 344 and 345; 354 and 355; 364 and 365; 374 and 375; 384 and 385; 394 and 395; 404 and 405; 414 and 415; 424 and 425; 434 and 435; 444 and 445; 454 and 455; 464 and 465; 474 and 475; 484 and 485; 494 and 495; 504 and 505; 514 and 515; 524 and 525; 534 and 535; 544 and 545; 554 and 555; 564 and 565; 574 and 575; 584 and 585; 594 and 595; 604 and 605; 614 and 615; 624 and 625; 634 and 635; 644 and 645; 654 and 655; 664 and 665; 674 and 675; 684 and 685; 694 and 695; 704 and 705; 714 and 715; 724 and 725; 734 and 735; 744 and 745; 754 and 755; 764 and 765; 774 and 775; 784 and 785; 794 and 795; 804 and 805; 814 and 815; 824 and 825; 834 and 835; 844 and 845; 854 and 855; 864 and 865; 874 and 875; 884 and 885; 894 and 895; 904 and 905; 914 and 915; 924 and 925; 934 and 935; 944 and 945; 954 and 955; 964 and 965; 974 and 975; 984 and 985; 994 and 995; 1004 and 1005; 1014 and 1015; 1024 and 1025; 1034 and 1035; 1044 and 1045; 1054 and 1055; 1064 and 1065; 1074 and 1075; 1084 and 1085; 1094 and 1095; and 1104 and 1105, wherein the antibodies bind to human FGFR1c, FGFR2c, FGFR3c, and/or FGFR4. In some embodiments, the antibodies do not bind to FGFR1b, FGFR2b, and/or FGFR3b. In some embodiments, the CDRs are defined according to the Kabat numbering system. In some embodiments, the CDRs are defined according to the IMGT numbering system.

In another aspect, provided herein are isolated monoclonal antibodies, or antigen-binding portions thereof, which specifically bind to FGFR1c, FGFR2c, FGFR3c, and/or FGFR4, and comprise heavy chain CDR1, CDR2, and CDR3 sequences and light chain CDR1, CDR2, and CDR3 sequences selected from the group consisting of SEQ ID NOs: 23-25 and 26-28; 34-36 and 37-39; 45-47 and 48-50; 56-58 and 59-61, 67-69 and 70-72; 78-80 and 81-83; 89-91 and 92-94; 100-102 and 103-105; 111-113 and 114-116; 122-124 and 125-127; 133-135 and 136-138; 144-146 and 147-149; 155-157 and 158-160, 166-168 and 169-171; 177-179 and 180-182; 188-190 and 191-193; 198-200 and 201-203; 208-210 and 211-213; 218-220 and 221-223; 228-230 and 231-233; 238-240 and 241-243; 248-250 and 251-253; 258-260 and 261-263; 268-270 and 271-273; 278-280 and 281-283; 288-290 and 291-293; 298-300 and 301-303; 308-310 and 311-313; 318-320 and 321-323; 228-330 and 331-333; 338-340 and 341-343; 348-350 and 351-353; 358-360 and 361-363; 368-370 and 371-373; 378-380 and 381-383; 388-390 and 391-393; 398-400 and 401-403; 408-410 and 411-413; 418-420 and 421-423; 428-430 and 431-433; 438-440 and 441-443; 448-450 and 451-453; 458-460 and 461-463; 468-470 and 471-473; 478-480 and 481-483; 488-490 and 491-493; 498-500 and 501-503; 508-510 and 511-513; 518-520 and 521-523; 528-530 and 531-533; 538-540 and 541-543; 548-550 and 551-553; 558-560 and 561-563; 568-570 and 571-573; 578-580 and 581-583; 588-590 and 591-593; 598-600 and 601-603; 608-610 and 611-613; 618-620 and 621-623; 628-630 and 631-633; 638-640 and 641-643; 648-650 and 651-653; 658-660 and 661-663; 668-670 and 671-673; 678-680 and 681-683; 688-690 and 691-693; 698-700 and 701-703; 708-710 and 711-713; 718-720 and 721-723; 728-730 and 731-733; 738-740 and 741-743; 748-750 and 751-753; 758-760 and 761-763; 768-770 and 771-773; 778-780 and 781-783; 788-790 and 791-793; 798-800 and 801-803; 808-810 and 811-813; 818-820 and 821-823; 828-830 and 831-833; 838-840 and 841-843; 848-850 and 851-853; 858-860 and 861-863; 868-870 and 871-873; 878-880 and 881-883; 888-890 and 891-893; 898-900 and 901-903; 908-910 and 911-913; 918-920 and 921-923; 928-930 and 931-933; 938-940 and 941-943; 948-950 and 951-953; 958-960 and 961-963; 968-970 and 971-973; 978-980 and 981-983; 988-990 and 991-993; 998-1000 and 1001-1003; 1008-1010 and 1011-1013; 1018-1020 and 1021-1023; 1028-1030 and 1031-1033; 1038-1040 and 1041-1043; 1048-1050 and 1051-1053; 1058-1060 and 1061-1063; 1068-1070 and 1071-1073; 1078-1080 and 1081-1083; 1088-1090 and 1091-1093; and 1098-1100 and 1101-1103. In some embodiments, the antibodies do not bind to FGFR1b, FGFR2b, and/or FGFR3b.

In another aspect, provided herein are isolated monoclonal antibodies, or antigen-binding portions thereof, which specifically bind to FGFR1c, FGFR2c, FGFR3c, and/or FGFR4, and comprise IMGT heavy chain CDR1, CDR2, and CDR3 sequences and IMGT light chain CDR1, CDR2, and CDR3 sequences selected from the group consisting of SEQ ID NOs: 1544-1546 and 1547-1549; 1550-1552 and 1553-1555; 1556-1558 and 1559-1561; 1562-1564 and 1565-1567; 1568-1570 and 1571-1573; ;1574-1576 and 1577-1579; 1580-1582 and 1583-1585; 1586-1588 and 1589-1591; 1592-1594 and 1595-1597; 1598-1600 and 1601-1603; 1604-1606 and 1607-1609; 1610-1612 and 1613-1615; 1616-1618 and 1619-1621; 1622-1624 and 1625-1627; 1628-1630 and 1631-1633; 1634-1636 and 1637-1639; 1640-1642 and 1643-1645; 1646-1648 and 1649-1651; 1652-1654 and 1655-1657; 1658-1660 and 1661-1663; 1664-1666 and 1667-1669; 1670-1672 and 1673-1675; 1676-1678 and 1679-1681; 1682-1684 and 1685-1687; 1688-1690 and 1691-1693; 1694-1696 and 1697-1699; 1700-1702 and 1703-1705; 1706-1708 and 1709-1711; 1712-1714 and 1715-1717; 1718-1720 and 1721-1723; 1724-1726 and 1727-1729; 1730-1732 and 1733-1735; 1736-1738 and 1739-1741; 1742-1744 and 1745-1747; 1748-1750 and 1751-1753; 1754-1756 and 1757-1759; 1760-1762 and 1763-1765; 1766-1768 and 1769-1771; 1772-1774 and 1775-1777; 1778-1780 and 1781-1783; 1784-1786 and 1787-1789; 1790-1792 and 1793-1795; 1796-1798 and 1799-1801; 1802-1804 and 1805-1807; 1808-1810 and 1811-1813; 1814-1816 and 1817-1819; 1820-1822 and 1823-1825; 1826-1828 and 1829-1831; 1832-1834 and 1835-1837; 1838-1840 and 1841-1843; 1844-1846 and 1847-1849; 1850-1852 and 1853-1855; 1856-1858 and 1859-1861; 1862-1864 and 1865-1867; 1868-1870 and 1871-1873; 1874-1876 and 1877-1879; 1880-1882 and 1883-1885; 1886-1888 and 1889-1891; 1892-1894 and 1895-1897; 1898-1900 and 1901-1903; 1904-1906 and 1907-1909; 1910-1912 and 1913-1915; 1916-1918 and 1919-1921; 1922-1924 and 1925-1927; 1928-1930 and 1931-1933; 1934-1936 and 1937-1939; 1940-1942 and 1943-1945; 1946-1948 and 1949-1951; 1952-1954 and 1955-1957; 1958-1960 and 1961-1963; 1964-1966 and 1967-1969; 1970-1972 and 1973-1975; 1976-1978 and 1979-1981; 1982-1984 and 1985-1987; 1988-1990 and 1991-1993; 1994-1996 and 1997-1999; 2000-2002 and 2003-2005; 2006-2008 and 2009-2011; 2012-2014 and 2015-2017; 2018-2020 and 2021-2023; 2024-2026 and 2027-2029; 2030-2032 and 2033-2035; 2036-2038 and 2039-2041; 2042-2044 and 2045-2047; 2048-2050 and 2051-2053; 2054-2056 and 2057-2059; 2060-2062 and 2063-2065; 2066-2068 and 2069-2071; 2072-2074 and 2075-2077; 2078-2080 and 2081-2083;2084-2086 and 2087-2089; 2090-2092 and 2093-2095; 2096-2098 and 2099-2101; 2102-2104 and 2105-2107; 2108-2110 and 2111-2113; 2114-2116 and 2117-2119; 2120-2122 and 2123-2125; 2126-2128 and 2129-2131; 2132-2134 and 2135-2137; 2138-2140 and 2141-2143; 2144-2146 and 2147-2149; 2150-2152 and 2153-2155; 2156-2158 and 2159-2161; 2162-2164 and 2165-2167; 2168-2170 and 2171-2173; 2174-2176 and 2177-2179; and 2180-2182 and 2183-2185. In some embodiments, the antibodies do not bind to FGFR1b, FGFR2b, and/or FGFR3b.

In another aspect, provided herein are isolated monoclonal antibodies, or antigen-binding portions thereof, which bind to FGFR1c, FGFR2c, FGFR3c, and/or FGFR4, and comprise heavy and light chain variable regions, wherein the heavy chain variable region comprises an amino acid sequence which is at least 90%, 95%, 98%, 99%, or 100% identical to the amino acid sequence selected from the group consisting of SEQ ID NOs: 29; 40; 51; 62; 73; 84; 95; 106; 117; 128; 139; 150; 161; 172; 183; 194; 204; 214; 224; 234; 244; 254; 264; 274; 284; 294; 304; 314; 324; 334; 344; 354; 364; 374; 384; 394; 404; 414; 424; 434; 444; 454; 464; 474; 484; 494; 504; 514; 524; 534; 544; 554; 564; 574; 584; 594; 604; 614; 624; 634; 644; 654; 664; 674; 684; 694; 704; 714; 724; 734; 744; 754; 764; 774; 784; 794; 804; 814; 824; 834; 844; 854; 864; 874; 884 ; 894; 904; 914; 924; 934; 944; 954; 964; 974; 984; 994; 1004; 1014; 1024; 1034; 1044; 1054; 1064; 1074; 1084; 1094; 1104; 1111; and 1115, wherein the antibodies bind to human FGFR1c, FGFR2c, FGFR3c, and/or FGFR4. In some embodiments, the antibodies do not bind to FGFR1b, FGFR2b, and/or FGFR3b. In some embodiments, the heavy chain variable region is paired with a light chain variable region comprising an amino acid sequence selected from the group consisting of SEQ ID NOs: 1119, 1123, 1127, 1131; 1135; 1139; 1143; 1147; 1151; 1155; 1159; 1163; and 1167.

In another aspect, provided herein are isolated monoclonal antibodies, or antigen-binding portions thereof, which bind to FGFR1c, FGFR2c, FGFR3c, and/or FGFR4, and comprise heavy and light chain variable regions, wherein the heavy chain variable region comprises heavy chain CDR1, CDR2, and CDR3 sequences set forth in SEQ ID NOs: 177-179; 1108-1110; or 1112-1114. In some embodiments, the antibodies do not bind to FGFR1b, FGFR2b, and/or FGFR3b. In some embodiments, the heavy chain variable region is paired with a light chain variable region comprising light chain CDR1, CDR2, and CDR3 sequences selected from the group consisting of SEQ ID NOs: 180-182; 1116-1118; 1120-1122; 1124-1126; 1128-1130; 1132-1134; 1136-1138; 1140-1142; 1144-1146; 1148-1150; 1152-1154; 1156-1158; 1160-1162; and 1164-1166.

In another aspect, provided herein are isolated monoclonal antibodies, or antigen-binding portions thereof, which bind to FGFR1c, FGFR2c, FGFR3c, and/or FGFR4, and comprise heavy and light chain variable regions, wherein the heavy chain variable region comprises IMGT heavy chain CDR1, CDR2, and CDR3 sequences set forth in SEQ ID NOs: 1628-1630; 2186-2188; or 2189-2191. In some embodiments, the antibodies do not bind to FGFR1b, FGFR2b, and/or FGFR3b. In some embodiments, the heavy chain variable region is paired with a light chain variable region comprising IMGT light chain CDR1, CDR2, and CDR3 sequences selected from the group consisting of SEQ ID NOs: 1631-1633; 2192-2194; 2195-2197; 2198-2200; 2201-2203; 2204-2206; 2207-2209; 2210-2212; 2213-2215; 2216-2218; 2219-2221; 2222-2224; 2225-2227; and 2228-2230.

In another aspect, provided herein are isolated monoclonal antibodies, or antigen-binding portions thereof, which bind to FGFR1c, FGFR2c, FGFR3c, and/or FGFR4, and comprise heavy and light chain variable regions, wherein the light chain variable region comprises an amino acid sequence which is at least 90%, 95%, 98%, 99%, or 100% identical to the amino acid sequence selected from the group consisting of SEQ ID NOs: 30; 41; 52; 63; 74; 85; 96; 107; 118; 129; 140; 151; 162; 173; 184; 195; 205; 215; 225; 235; 245; 255; 265; 275; 285; 295; 305; 315; 325; 335; 345; 355; 365; 375; 385; 395; 405; 415; 425; 435; 445; 455; 465; 475; 485; 495; 505; 515; 525; 535; 545; 555; 565; 575; 585; 595; 605; 615; 625; 635; 645; 655; 665; 675; 685; 695; 705; 715; 725; 735; 745; 755; 765; 775; 785; 795; 805; 815; 825; 835; 845; 855; 865; 875; 885; 895; 905; 915; 925; 935; 945; 955; 965; 975; 985; 995; 1005; 1015; 1025; 1035; 1045; 1055; 1065; 1075; 1085; 1095; 1105; 1119; 1123; 1127; 1131; 1135; 1139; 1143; 1147; 1151; 1155; 1159; 1163; and 1167, wherein the antibodies bind to human FGFR1c, FGFR2c, FGFR3c, and/or FGFR4. In some embodiments, the antibodies do not bind to FGFR1b, FGFR2b, and/or FGFR3b. In some embodiments, the light chain variable region is paired with a heavy chain variable region comprising the amino acid sequence of SEQ ID NO: 1111 or 1115.

In another aspect, provided herein are isolated monoclonal antibodies, or antigen-binding portions thereof, which bind to FGFR1c, FGFR2c, FGFR3c, and/or FGFR4, and comprise heavy and light chain variable regions, wherein the light chain variable region comprises light chain CDR1, CDR2, and CDR3 sequences set forth in SEQ ID NOs: 180-182; 1116-1118; 1120-1122; 1124-1126; 1128-1130; 1132-1134; 1136-1138; 1140-1142; 1144-1146; 1148-1150; 1152-1154; 1156-1158; 1160-1162; and 1164-1166. In some embodiments, the antibodies do not bind to FGFR1b, FGFR2b, and/or FGFR3b. In some embodiments, the light chain variable region is paired with a heavy chain variable region comprising heavy chain CDR1, CDR2, and CDR3 sequences selected from the group consisting of SEQ ID NOs: 177-179; 1108-1110; or 1112-1114.

In another aspect, provided herein are isolated monoclonal antibodies, or antigen-binding portions thereof, which bind to FGFR1c, FGFR2c, FGFR3c, and/or FGFR4, and comprise heavy and light chain variable regions, wherein the light chain variable region comprises IMGT light chain CDR1, CDR2, and CDR3 sequences set forth in SEQ ID NOs: 1631-1633; 2192-2194; 2195-2197; 2198-2200; 2201-2203; 2204-2206; 2207-2209; 2210-2212; 2213-2215; 2216-2218; 2219-2221; 2222-2224; 2225-2227; and 2228-2230. In some embodiments, the antibodies do not bind to FGFR1b, FGFR2b, and/or FGFR3b. In some embodiments, the light chain variable region is paired with a heavy chain variable region comprising IMGT heavy chain CDR1, CDR2, and CDR3 sequences selected from the group consisting of SEQ ID NOs: 1628-1630; 2186-2188; or 2189-2191.

In another aspect, provided herein are isolated monoclonal antibodies, or antigen-binding portions thereof, which bind to FGFR1c, FGFR2c, FGFR3c, and/or FGFR4, and comprises heavy and light chain variable region sequences which are at least 90%, 95%, 98%, 99%, or 100% identical to the amino acid sequences selected from the group consisting of SEQ ID NOs: 29 and 30; 40 and 41; 51 and 52; 62 and 63; 73 and 74; 84 and 85; 95 and 96; 106 and 107; 117 and 118; 128 and 129; 139 and 140; 150 and 151; 161 and 162; 172 and 173; 183 and 184; 194 and 195; 204 and 205; 214 and 215; 224 and 225; 234 and 235; 244 and 245; 254 and 255; 264 and 265; 274 and 275; 284 and 285; 294 and 295; 304 and 305; 314 and 315; 324 and 325; 334 and 335; 344 and 345; 354 and 355; 364 and 365; 374 and 375; 384 and 385; 394 and 395; 404 and 405; 414 and 415; 424 and 425; 434 and 435; 444 and 445; 454 and 455; 464 and 465; 474 and 475; 484 and 485; 494 and 495; 504 and 505; 514 and 515; 524 and 525; 534 and 535; 544 and 545; 554 and 555; 564 and 565; 574 and 575; 584 and 585; 594 and 595; 604 and 605; 614 and 615; 624 and 625; 634 and 635; 644 and 645; 654 and 655; 664 and 665; 674 and 675; 684 and 685; 694 and 695; 704 and 705; 714 and 715; 724 and 725; 734 and 735; 744 and 745; 754 and 755; 764 and 765; 774 and 775; 784 and 785; 794 and 795; 804 and 805; 814 and 815; 824 and 825; 834 and 835; 844 and 845; 854 and 855; 864 and 865; 874 and 875; 884 and 885; 894 and 895; 904 and 905; 914 and 915; 924 and 925; 934 and 935; 944 and 945; 954 and 955; 964 and 965; 974 and 975; 984 and 985; 994 and 995; 1004 and 1005; 1014 and 1015; 1024 and 1025; 1034 and 1035; 1044 and 1045; 1054 and 1055; 1064 and 1065; 1074 and 1075; 1084 and 1085; 1094 and 1095; and 1104 and 1105, wherein the antibodies bind to human FGFR1c, FGFR2c, FGFR3c, and/or FGFR4. In some embodiments, the antibodies do not bind to FGFR1b, FGFR2b, and/or FGFR3b.

In another aspect, provided herein are isolated monoclonal antibodies, or antigen-binding portions thereof, which bind to FGFR1c, FGFR2c, FGFR3c, and/or FGFR4, comprising heavy and light chains, wherein the heavy chains comprise or consist of an amino acid sequence which is at least 90%, 95%, 98%, 99%, or 100% identical to the amino acid sequences selected from the group consisting of SEQ ID NO: 32; 43; 54; 65; 76; 87; 98; 109; 120; 131; 142; 153; 164; 175; 186; 196; 206; 216; 226; 236; 246; 256; 266; 276; 286; 296; 306; 316; 326; 336; 346; 356; 366; 376; 386; 396; 406; 416; 426; 436; 446; 456; 466; 476; 486; 496; 506 516; 526; 536; 546; 556; 566; 576; 586; 596; 606; 616; 626; 636; 646; 656; 666; 676; 686; 696; 706; 716; 726; 736; 746; 756; 766; 776; 786; 796; 806; 816; 826; 836; 846; 856; 866; 876; 886; 896; 906; 916; 926; 936; 946; 956; 966; 976; 986; 996; 1006; 1016; 1026; 1036; 1046; 1056; 1066; 1076; 1086; 1096; and 1106, wherein the antibodies bind to human FGFR1c, FGFR2c, FGFR3c, and/or FGFR4. In some embodiments, the antibodies do not bind to FGFR1b, FGFR2b, and/or FGFR3b.

In another aspect, provided herein are isolated monoclonal antibodies, or antigen-binding portions thereof, which bind to FGFR1c, FGFR2c, FGFR3c, and/or FGFR4, comprising heavy and light chains, wherein the light chain comprises or consists of an amino acid sequence which is at least 90%, 95%, 98%, 99%, or 100% identical to an amino acid sequence selected from the group consisting of SEQ ID NO: 33; 44; 55; 66; 77; 88; 99; 110; 121; 132; 143; 154; 165; 176; 186; 196; 206; 216; 226; 236; 246; 256; 266; 277; 287; 297; 307; 317; 327; 337; 347; 357; 367; 377; 387; 397; 407; 417; 427; 437; 447; 457; 467; 477; 487; 497; 507; 517; 527; 537; 547; 557; 567; 577; 587; 597; 607; 617; 627; 637; 647; 657; 667; 677; 687; 697; 707; 717; 727; 737; 747 757; 767; 777; 787; 797; 807; 817; 827; 837; 847; 857; 867; 877; 887; 897; 907; 917; 927; 937; 947; 957; 967; 977; 987; 997; 1007; 1017; 1027; 1037; 1047; 1057; 1067; 1077; 1087; 1097; and 1107, wherein the antibodies bind to human FGFR1c, FGFR2c, FGFR3c, and/or FGFR4. In some embodiments, the antibodies do not bind to FGFR1b, FGFR2b, and/or FGFR3b.In another aspect, provided herein are isolated monoclonal antibodies, or antigen-binding portions thereof, which bind to FGFR1c, FGFR2c, FGFR3c, and/or FGFR4, and comprises or consists of heavy and light chain sequences which are at least 90%, 95%, 98%, 99%, or 100% identical to the amino acid sequences selected from the group consisting of SEQ ID NOs: 32 and 33; 43 and 44; 54 and 55; 65 and 66; 76 and 77; 87 and 88; 98 and 99; 109 and 110; 120 and 121; 131 and 132; 142 and 143; 153 and 154; 164 and 165; 175 and 176; 186 and 187; 196 and 197; 206 and 207; 216 and 217; 226 and 227; 236 and 237; 246 and 247; 256 and 257; 266 and 267; 276 and 277; 286 and 287; 296 and 297; 306 and 307; 316 and 317; 326 and 327; 336 and 337; 346 and 347; 356 and 357; 366 and 367; 376 and 377; 386 and 387; 396 and 397; 406 and 407; 416 and 417; 426 and 427; 436 and 437; 446 and 447; 456 and 457; 466 and 467; 476 and 477; 486 and 487; 496 and 497; 506 and 507; 516 and 517; 526 and 527; 536 and 537; 546 and 547; 556 and 557; 566 and 567; 576 and 577; 586 and 587; 596 and 597; 606 and 607; 616 and 617; 626 and 627; 636 and 637; 646 and 647; 656 and 657; 666 and 667; 676 and 677; 686 and 687; 696 and 697; 706 and 707; 716 and 717; 726 and 727; 736 and 737; 746 and 747; 756 and 757; 766 and 767; 776 and 777; 786 and 787; 796 and 797; 806 and 807; 816 and 817; 826 and 827; 836 and 837; 846 and 847; 856 and 857; 866 and 867; 876 and 877; 886 and 887; 896 and 897; 906 and 907; 916 and 917; 926 and 927; 936 and 937; 946 and 947; 956 and 957; 966 and 967; 976 and 977; 986 and 987; 996 and 997; 1006 and 1007; 1016 and 1017; 1026 and 1027; 1036 and 1037; 1046 and 1047; 1056 and 1057; 1066 and 1067; 1076 and 1077; 1086 and 1087; 1096 and 1097; and 1106 and 1107, wherein the antibodies bind to human FGFR1c, FGFR2c, FGFR3c, and/or FGFR4. In some embodiments, the antibodies do not bind to FGFR1b, FGFR2b, and/or FGFR3b, wherein the antibodies bind to human FGFR1c, FGFR2c, FGFR3c, and/or FGFR4. In some embodiments, the antibodies do not bind to FGFR1b, FGFR2b, and/or FGFR3b.

In another aspect, provided herein are antibodies (e.g., isolated monoclonal antibodies), or antigen-binding portions thereof, which bind to FGFR1c, FGFR2c, FGFR3c, and/or FGFR4, and comprise heavy chain CDR1, CDR2, and CDR3 sequences [SGHT][YH]A[MI]H (SEQ ID NO: 2231), [VL]ISYDGS[NE]KYYADS[VA]KG (SEQ ID NO: 2232), and GAG[RTLQHMI]G[YLFW][TPASIM][YNFHLWRK]GPDGDFI (SEQ ID NO: 2233), respectively, and light chain CDR1, CDR2, and CDR3 sequences [TRK]SS[RQE]SL[LVI][HWYF][SRGT]DG[KNI]TY[VL][YSN] (SEQ ID NO: 2234), [EKQ][LVI]S[NS]RFS (SEQ ID NO: 2235), and MQ[YA][IVTK][EQNR][AFL]P[LW]T (SEQ ID NO: 2236), respectively.

In another aspect, provided herein are antibodies (e.g., isolated monoclonal antibodies), or antigen-binding portions thereof, which bind to FGFR1c, FGFR2c, FGFR3c, and/or FGFR4, and comprise heavy chain CDR1, CDR2, and CDR3 sequences [SG]YA[MI]H (SEQ ID NO: 2237), [VL]ISYDGSNKYYADS[VA]KG (SEQ ID NO: 2238), and GAG[RTLQHMI]G[YLFW][TPASIM][YNFHLWRK]GPDGDFI (SEQ ID NO: 2239), respectively, and light chain CDR1, CDR2, and CDR3 sequences [TRK]SS[RQE]SL[LV][HWY][SR]DG[KN]TY[VL][YS] (SEQ ID NO: 2240), [EK][LV]SNRFS (SEQ ID NO: 2241), and MQ[YA][IVT][EQ][AF]P[LW]T (SEQ ID NO: 2242), respectively.

In another aspect, provided herein are antibodies (e.g., isolated monoclonal antibodies), or antigen-binding portions thereof, which bind to FGFR1c, FGFR2c, FGFR3c, and/or FGFR4, and comprise heavy chain CDR1, CDR2, and CDR3 sequences [SG]YAMH (SEQ ID NO: 2243), VISYDGSNKYYADSVKG (SEQ ID NO: 2244), and GAG[RTLQHMI]G[YLFW][TPASIM][YNFHLWRK]GPDGDFI (SEQ ID NO: 2245), respectively, and light chain CDR1, CDR2, and CDR3 sequences [TRK]SS[RQ]SLL[HW]SDGKTY[VL]Y (SEQ ID NO: 2246), ELSNRFS (SEQ ID NO: 2247), and MQY[IV]EAPLT (SEQ ID NO: 2248), respectively.

In another aspect, provided herein are antibodies (e.g., isolated monoclonal antibodies), or antigen-binding portions thereof, which bind to FGFR1c, FGFR2c, FGFR3c, and/or FGFR4, and comprise IMGT heavy chain CDR1, CDR2, and CDR3 sequences GF[TSD]F[SGTA][SGHT][YH]A (SEQ ID NO: 2249), ISYDGS[NE]K (SEQ ID NO: 2250), and VRGAG[RTLQHMI]G[YLFW][TPASIM][YNFHLWRK]GPDGDFI (SEQ ID NO: 2251), respectively, and IMGT light chain CDR1, CDR2, and CDR3 sequences [RQE]SL[LVI][HWYF][SRGT]DG[KNI]TY (SEQ ID NO: 2252), [EKQ][LVI]S (SEQ ID NO: 2253), and MQ[YA][IVTK][EQNR][AFL]P[LW]T (SEQ ID NO: 2254), respectively.

In another aspect, provided herein are antibodies (e.g., isolated monoclonal antibodies), or antigen-binding portions thereof, which bind to FGFR1c, FGFR2c, FGFR3c, and/or FGFR4, and comprise IMGT heavy chain CDR1, CDR2, and CDR3 sequences GF[TD]F[SA][SG]YA (SEQ ID NO: 2255), ISYDGSNK (SEQ ID NO: 2256), and VRGAG[RTLQHMI]G[YLFW][TPASIM][YNFHLWRK]GPDGDFI (SEQ ID NO: 2257), respectively, and IMGT light chain CDR1, CDR2, and CDR3 sequences [RQE]SL[LV][HWY][SR]DG[KN]TY (SEQ ID NO: 2258), [EK][LV]S (SEQ ID NO: 2259), and MQ[YA][IVT][EQ][AF]P[LW]T (SEQ ID NO: 2260), respectively.

In another aspect, provided herein are antibodies (e.g., isolated monoclonal antibodies), or antigen-binding portions thereof, which bind to FGFR1c, FGFR2c, FGFR3c, and/or FGFR4, and comprise IMGT heavy chain CDR1, CDR2, and CDR3 sequences GF[TD]F[SA][SG]YA (SEQ ID NO: 2261), ISYDGSNK (SEQ ID NO: 2262), and VRGAG[RTLQHMI]G[YLFW][TPASIM][YNFHLWRK]GPDGDFI (SEQ ID NO: 2263), respectively, and IMGT light chain CDR1, CDR2, and CDR3 sequences [RQ]SLL[HW]SDGKTY (SEQ ID NO: 2264), ELS (SEQ ID NO: 2265), and MQY[IV]EAPLT (SEQ ID NO: 2266), respectively.

In another aspect, provided herein are antibodies, or antigen-binding portions thereof, which bind to the same epitope on FGFR1c, FGFR2c, FGFR3c, and/or FGFR4 as the anti-FGFR antibodies described herein.

In another aspect, provided herein are antibodies, or antigen-binding portions thereof, which compete for binding to FGFR1c, FGFR2c, FGFR3c, and/or FGFR4 with the anti-FGFR antibodies described herein.

In another aspect, provided herein are modified antibodies, or antigen-binding portions thereof, that bind to FGFR1c, wherein the antibodies exhibit increased tolerability (e.g., measured as a reduction in weight loss) as compared to an antibody comprising identical heavy and light chain variable region sequences and an IgG1 constant region when administered to a mammal (e.g., a mouse or human). In another aspect, provided herein are modified antibodies, or antigen-binding portions thereof, that bind to FGFR1c, wherein administration of the antibodies to mammals does not result in significant weight loss (e.g., weight loss of <15%, ≤10%, or ≤5%). In some embodiments, the antibodies also bind to FGFR2c, FGFR3c, and/or FGFR4. In some embodiments, the antibodies do not bind to FGFR1b, FGFR2b, and FGFR3b. In some embodiments, the reduction in weight loss when the antibody is administered to mice is about 15% or less, e.g., ≤10% or less, ≤5% or less, or lower, relative to the weight loss observed for the same antibody in IgG1 form when the antibody is administered once every week, 2 weeks, or 3 weeks, for 6 weeks at a dose of 0.5 mg/kg, 1 mg/kg or 2 mg/kg.

In another aspect, provided herein are multispecific molecules comprising the anti-FGFR antibodies, or antigen-binding portions thereof, described herein linked to a molecule having a further binding specificity for a target molecule which is not a FGF receptor.

In another aspect, provided herein are immunoconjugates comprising the anti-FGFR antibodies, or antigen-binding portions thereof, described herein linked to a binding moiety, a labeling moiety, a biologically active moiety, or a therapeutic agent.

In another aspect, provided herein are nucleic acids encoding the heavy and/or light chain variable regions of the anti-FGFR antibodies, or antigen-binding portions thereof, and multispecific molecules described herein, expression vectors comprising the nucleic acids, and cells transformed with the expression vectors.

In another aspect, provided herein are compositions comprising the anti-FGFR antibodies (e.g., monoclonal antibodies), or antigen-binding portions thereof, multispecific molecules, or immunoconjugates described herein. In one embodiment, the composition is an antibody composition comprising one or more antibodies, or antigen-binding portions thereof, which collectively bind to FGFR1c, FGFR2c, FGFR3c, and FGFR4, but not FGFR1b, FGFR2b, and/or FGFR3b.

In another aspect, provided herein are kits comprising the anti-FGFR antibodies (e.g., monoclonal antibodies), or antigen-binding portions thereof, multispecific molecules, or immunoconjugates described herein, and instructions for use.

In some embodiments, the antibodies described herein do not bind to FGFR1b, FGFR2b, and/or FGFR3b.

In some embodiments, the antibodies described herein binds to FGFR1c (e.g., human FGFR1c), FGFR2c (e.g., human FGFR2c), FGFR3c (e.g., human FGFR3c), and/or FGFR4 (e.g., human FGFR4) with a K_{D} of 10⁻⁷ M or less, e.g., as assessed by bio-layer interferometry. In some embodiments, the antibodies described herein block the binding of FGF1 and/or FGF2 to FGFR1c, FGFR2c, FGFR3c, and/or FGFR4. In further embodiments, the antibodies inhibit FGF2-mediated phosphorylation of ERK, e.g., with an IC50 of 100 nM or less, e.g., as assessed by ELISA. In some embodiments, the antibodies inhibit FGF2-mediated cell viability, e.g., with an IC50 of 100 nM or less, e.g., as assessed with a cell viability assay (e.g., CellTiterGlo). In some embodiments, the antibodies described herein have a serum half-life of 25 hours or more, 50 hours or more, or 100 hours or more in mice when administered intravenously at a single dose of 40 mg/kg.

In some embodiments, the antibodies described herein are in scFv format. In some embodiments, the antibodies described herein are human antibodies. In some embodiments, the antibodies are monoclonal antibodies. In some embodiments, the antibodies are monoclonal human antibodies. In some embodiments, the antibodies herein are IgG1, IgG2, IgG3, or IgG4 antibodies, or variants thereof. In some embodiments, the antibodies described herein comprise Fc regions with reduced or no effector function. For example, in some embodiments, the antibodies described herein are IgG2 antibodies. In one embodiment, the antibodies described herein comprise a hybrid Ig2/IgG4 constant region, e.g., a constant region comprising the amino acid sequence of SEQ ID NO: 1173 (optionally with the first 3 amino acids “AST” removed).

In another aspect, provided herein is a method of preparing an anti-FGFR antibody, or antigen-binding portion thereof, comprising expressing the antibody, or antigen binding portion thereof, described herein in cells, and isolating the antibody, or antigen binding portion thereof, from the cell.

In another aspect, provided herein are methods of blocking FGF1 or FGF2 binding to FGFR1c, FGFR2c, FGFR3c, and/or FGFR4 in a cell comprising contacting the cell with an effective amount of an anti-FGFR antibody (e.g., a monoclonal antibody), or antigen-binding portion thereof, multispecific molecule, or immunoconjugate, described herein. In another aspect, provided herein is the use of an antibody, or antigen-binding portion thereof, multispecific molecule, or immunoconjugate, described herein for the manufacture of a medicament for blocking FGF (e.g., FGF1, FGF2, or FGF18) binding to FGFR1c, FGFR2c, FGFR3c, and/or FGFR4 in a cell. In another aspect, provided herein is an antibody, or antigen-binding portion thereof, multispecific molecule, or immunoconjugate, described herein for use in blocking FGF (e.g., FGF1, FGF2, or FGF18) binding to FGFR1c, FGFR2c, FGFR3c, and/or FGFR4 in a cell.

In another aspect, provided herein is a method of inhibiting FGF-mediated signaling in a cell comprising contacting the cell with an effective amount of an anti-FGFR antibody (e.g., a monoclonal antibody), or antigen-binding portion thereof, multispecific molecule, or immunoconjugate, described herein. In another aspect, provided herein is the use of an antibody, or antigen-binding portion thereof, multispecific molecule, or immunoconjugate, described herein for the manufacture of a medicament for the inhibition of FGF-mediated signaling in a cell. In another aspect, provided herein is an antibody, or antigen-binding portion thereof, multispecific molecule, or immunoconjugate, described herein for use in the inhibition of FGF-mediated signaling in a cell.

In another aspect, provided herein is a method of inhibiting the growth of tumor cells comprising administering to a subject with a tumor a therapeutically effective amount of an anti-FGFR antibody (e.g., a monoclonal antibody), or antigen-binding portion, multispecific molecule, or immunoconjugate, described herein. In another aspect, provided herein is the use of an antibody, or antigen-binding portion thereof, multispecific molecule, or immunoconjugate, described herein for the manufacture of a medicament for the inhibition of tumor cell growth. In another aspect, provided herein is an antibody, or antigen-binding portion thereof, multispecific molecule, or immunoconjugate, described herein for use in the inhibition of tumor cell growth.

In another aspect, provided herein is a method of treating cancer comprising administering to a subject in need thereof a therapeutically effective amount of an anti-FGFR antibody (e.g., a monoclonal antibody), or antigen-binding portion, multispecific molecule, or immunoconjugate, described herein. In another aspect, provided herein is the use of an antibody, or antigen-binding portion thereof, multispecific molecule, or immunoconjugate, described herein for the manufacture of a medicament for the treatment of cancer. In another aspect, provided herein is an antibody, or antigen-binding portion thereof, multispecific molecule, or immunoconjugate, described herein for use in the treatment of cancer. In some embodiments, the cancer is a mesenchymal-like solid tumor. In some embodiments, the cancer is lung cancer, renal cancer, breast cancer, or ovarian cancer. In some embodiments, one or more additional therapeutics is administered. In some embodiments, administration of the antibody, or antigen-binding portion thereof, multispecific molecule, or immunoconjugate in the methods described above does not induce weight loss in the subject.

In another aspect, provided herein is a method of treating cancer comprising administering to a subject in need thereof a therapeutically effective amount of an IgG2 antibody that binds to FGFR1c. In another aspect, provided herein is the use of an IgG2 antibody that binds to FGFR1c for the manufacture of a medicament for the treatment of cancer. In another aspect, provided herein is an IgG2 antibody that binds to FGFR1c for use in the treatment of cancer.

In another aspect, provided herein is a method of detecting the presence of FGFR (e.g., FGFR1c, FGFR2c, FGFR3c, and/or FGFR4) in a sample comprising contacting the sample with an anti-FGFR antibody or immunoconjugate described herein under conditions that allow for formation of a complex between the antibody and FGFR protein, and detecting the formation of a complex.

Other features and advantages of the instant disclosure will be apparent from the following detailed description and examples, which should not be construed as limiting.

### BRIEF DESCRIPTION OF THE FIGURES

FIGS. 1A-1D are graphs showing the inhibition of cancer cell viability (NCI-H2286, IGROV1, Caki1, Cal51 lines) by antibodies targeting individual FGFRs or multiple FGFRs, as assessed with CellTiterGlo. FIG. 1E is a graph showing relative levels of FGFR1-4 in NCI-H2286, IGROV1, Caki1, and Cal51 cells.

FIG. 2 shows an alignment of the heavy chain variable region (VH and VL, respectively) sequences of a subset of the anti-FGFR antibodies described herein. In the alignment, #5 refers to Ab5, #73 to Ab10, #78 to Ab3, #26 to Ab15, #60 to Ab8, #50 to Ab11, #40 to Ab6, #41 to Ab1, #10 to Ab4, #59 to Ab2, #63 to Ab9, #51 to Ab7, #14 to Ab12, #24 to Ab14, and #51 to Ab7.

FIGS. 3A-3D are graphs showing the binding of a subset of anti-FGFR antibodies to recombinant human FGFR1c, FGFR2c, FGFR3c, and FGFR4, respectively, by ELISA. FIG. 3E is a graph showing that none of the antibodies tested bind to FGFR2b.

FIGS. 4A and 4B are graphs showing the inhibition of pERK activation by FGF1 and FGF2, respectively, in Ca151 cells, as assessed using the AlphaScreen SureFire ERK1/2 assay.

FIG. 5 is a graph showing that A1 (an antagonist FGFR1 antibody) in IgG1 format (A1 IgG1) and A1 in IgG2 format (IgG2m4 with C127S mutation) bind to FGFR1c with similar affinities.

FIG. 6 is a graph showing that A1 (IgG1) and A1M5 (IgG2) inhibited FGF2-induced pERK activation with similar efficacy, albeit with A1M5 showing slightly more activity at the higher concentrations.

FIGS. 7A and 7B are graphs showing that the A1 antibody induces a significant decrease in body weight of mice when administered at 1 mg/kg (FIG. 7A) or 10 mg/kg (FIG. 7B).

FIG. 7C is a graph showing that Ab15 does not significantly reduce the body weight of mice when administered at 2 mg/kg or 20 mg/kg.

FIG. 7D is a graph showing that Ab15 with full (IgG1) or partial (IgG1/4) effector function significantly reduces the body weight of mice when administered at 10 and 20 mg, whereas Ab15 with no effector function (IgG2) did not induce significant weight loss at any of the doses tested.

FIGS. 8A-8F are graphs showing the blocking of ligand (FGF1) binding to FGFR1 (FIGS. 8A-8C) and FGFR4 (FIGS. 8D-8F), as assessed by ELISA.

FIGS. 9A-9D are graphs showing the inhibition of ERK signaling, as assessed by phosphorylation ERK using the AlphaScreen SureFire assay. The grey line corresponds to basal pERK levels.

FIG. 10 is a graph showing the ability of anti-FGFR antibodies (FGFRc targeting antibodies) to inhibit viability of IROV-1 cells, as assessed by CellTiterGlo.

FIG. 11 is a graph showing PK profiles of Ab15, Ab19, and Ab90 in mice.

FIGS. 12A-12C are graphs showing the suppression of tumor growth in vivo by Ab15, Ab19, and Ab90 in the MSTO211H model (FIG. 12A), MFE280 model (FIG. 12B), and SN12C model (FIG. 12C). FIG. 12D is a graph showing that Ab15 had a minimal effect on tumor cell viability in vitro in the MSTO211H model.

### DETAILED DESCRIPTION

### I. Overview

Provided herein are isolated antibodies, particularly monoclonal antibodies, e.g., human monocloncal antibodies, which specifically bind to FGFR1c, FGFR2c, FGFR3c, and/or FGFR4 (e.g., human FGFR1c, FGFR2c, FGFR3c, and/or FGFR4), and inhibit FGFR activity. In some embodiments, the antibodies do not bind to FGFR1b, FGFR2b, and/or FGFR3b (e.g., human FGFR1b, FGFR2b, and/or FGFR3b). In some embodiments, the antibodies described herein are derived from particular heavy and light chain germline sequences and/or comprise particular structural features such as CDR regions comprising particular amino acid sequences. Provided herein are isolated antibodies, methods of making the antibodies, immunoconjugates and multispecific molecules comprising such antibodies, and pharmaceutical compositions comprising the antibodies. Also provided herein are methods of inhibiting tumor growth and methods of treating cancer using the antibodies.

### II. Definitions

In order that the present description may be more readily understood, certain terms are first defined. Additional definitions are set forth throughout the detailed description.

The term “FGF receptor” and “FGFR” and are used interchangeably herein and refer to a family of four fibroblast growth factor genes that, like all tyrosine kinase receptors, are composed of an extracellular ligand binding domain, a single transmembrane α-helix, and a cytoplasmic tyrosine kinase domain. The FGFR extracellular domain is composed of up to three immunoglobulin-like domains (D 1-3), with D2 and D3 comprising the ligand binding portion. In addition, D2 domain also includes a positively charged region that serves as a binding domain for heparan sulfate proteoglycan (HSPG), and one characteristic of the FGF receptor-ligand signaling system is the formation of a ternary complex between ligand, receptor and HSPG. Another critical distinctive feature among members of the FGF receptor (FGFR) family is the extensive use of alternative splicing to generate multiple isoforms. Splicing variants have been identified for all four mammalian FGFRs, but the most characterized variants are the splicing variations generated within the D3 domain. In fact, the alternative usage of two exons within FGF receptor genes 1, 2 and 3 (but not FGFR4) generate two receptor variants named “c” and “b” respectively.

Amino acid sequences of the “b” and “c” isoforms (also referred to as “IIIb” and “IIIc” isoforms, respectively) of the four human FGFR proteins (i.e., FGFR1-4) are provided in Table 9 as follows: Human FGFR1b (SEQ ID NO: 3), Human FGFR1c (SEQ ID NO: 1), Human FGFR2b (SEQ ID NO: 8), Human FGFR2c (SEQ ID NO: 5), Human FGFR3b (SEQ ID NO: 15), Human FGFR3c (SEQ ID NO: 11), Human FGFR4 (SEQ ID NO: 9).

The term “antibody” or “immunoglobulin,” as used interchangeably herein, includes whole antibodies and any antigen binding fragment (antigen-binding portion) or single chain cognates thereof. An “antibody” comprises at least one heavy (H) chain and one light (L) chain. In naturally occurring IgGs, for example, these heavy and light chains are inter-connected by disulfide bonds and there are two paired heavy and light chains, these two also inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR) or Joining (J) regions (JH or JL in heavy and light chains respectively). Each V_{H} and V_{L} is composed of three CDRs, three FRs and a J domain, arranged from amino-terminus to carboxy-terminus in the following order: FR1, CDR1, FR2, CDR2, FR3, CDR3, J. The variable regions of the heavy and light chains bind with an antigen. The constant regions of the antibodies may mediate the binding of the immunoglobulin to host tissues or factors, including various cells of the immune system (e.g., effector cells) or humoral factors such as the first component (Clq) of the classical complement system. It has been shown that fragments of a full-length antibody can perform the antigen-binding function of an antibody. Examples of binding fragments denoted as an antigen-binding portion or fragment of an antibody include (i) a Fab fragment, a monovalent fragment consisting of the V_{L}, V_{H}, CL and CH1 domains; (ii) a F(ab')₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb including VH and VL domains; (vi) a dAb fragment (Ward et al. (1989) Nature 341, 544-546), which consists of a V_{H} domain; (vii) a dAb which consists of a VH or a VL domain; and (viii) an isolated complementarity determining region (CDR) or (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions are paired to form monovalent molecules (such a single chain cognate of an immunoglobulin fragment is known as a single chain Fv (scFv). Such single chain antibodies are also intended to be encompassed within the term “antibody”. Antibody fragments are obtained using conventional techniques known to those with skill in the art, and the fragments are screened for utility in the same general manner as are intact antibodies. Antigen-binding portions can be produced by recombinant DNA techniques, or by enzymatic or chemical cleavage of intact immunoglobulins. Unless otherwise specified, the numbering of amino acid positions in the antibodies described herein (e.g., amino acid residues in the Fc region) and identification of regions of interest, e.g., CDRs, use the Kabat system (Kabat, E. A., et al. (1991) Sequences of Proteins of Immunological Interest, Fifth Edition, U.S. Department of Health and Human Services, NIH Publication No. 91-3242). Certain embodiments described herein define CDRs using the IMGT numbering system (Lefranc et al, Dev. Comp. Immunol, 2005; 29(3):185-203). When the IMGT numbering system is used herein, reference to CDRs will be prefaced with or incorporate the term “IMGT”, for example, “IMGT heavy chain CDR1, CDR2, and CDR3 sequences,” “IMGT VHCDR1,” “IMGT VLCDR1-3,” “VHCDR2 (IMGT).”

The term “monoclonal antibody” as used herein refers to an antibody obtained from a population of substantially homogeneous antibodies, i.e., the individual antibodies comprising the population are identical except for possible naturally occurring mutations that may be present in minor amounts. Antigen binding fragments (including scFvs) of such immunoglobulins are also encompassed by the term “monoclonal antibody” as used herein. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. Monoclonal antibodies can be prepared using any art recognized technique and those described herein such as, for example, a hybridoma method, a transgenic animal, recombinant DNA methods (see, e.g., U.S. Pat. No. 4,816,567), or using phage antibody libraries using the techniques described in, for example, US Pat. No. 7,388,088 and U.S. patent application Ser. No. 09/856,907 (PCT Int. Pub. No. WO 00/31246). Monoclonal antibodies include chimeric antibodies, human antibodies, and humanized antibodies and may occur naturally or be produced recombinantly.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain embodiments, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The term “human antibody,” as used herein, is intended to include antibodies having variable regions in which both the framework and CDR regions are derived from human germline immunoglobulin sequences as described, for example, by Kabat et al. (See Kabat, et al. (1991) Sequences of proteins of Immunological Interest, Fifth Edition, U.S. Department of Health and Human Services, NIH Publication No. 91-3242). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The human antibody can have at least one or more amino acids replaced with an amino acid residue, e.g., an activity enhancing amino acid residue that is not encoded by the human germline immunoglobulin sequence. Typically, the human antibody can have up to twenty positions replaced with amino acid residues that are not part of the human germline immunoglobulin sequence. In a particular embodiment, these replacements are within the CDR regions as described in detail below.

The term “humanized antibody” refers to an antibody that includes at least one humanized antibody chain (i.e., at least one humanized light or heavy chain). The term “humanized antibody chain” (i.e., a “humanized immunoglobulin light chain”) refers to an antibody chain (i.e., a light or heavy chain, respectively) having a variable region that includes a variable framework region substantially from a human antibody and complementarity determining regions (CDRs) (e.g., at least one CDR, two CDRs, or three CDRs) substantially from a non-human antibody, and further includes constant regions (e.g., one constant region or portion thereof, in the case of a light chain, and preferably three constant regions in the case of a heavy chain).

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

“Isolated,” as used herein, is intended to refer to an antibody that is substantially free of other antibodies having different antigenic specificities. In addition, an isolated antibody is typically substantially free of other cellular material and/or chemicals.

An “effector function” refers to the interaction of an antibody Fc region with an Fc receptor or ligand, or a biochemical event that results therefrom. Exemplary “effector functions” include Clq binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor; BCR). Such effector functions generally require the Fc region to be combined with a binding domain (e.g., an antibody variable domain).

An “Fc region,” “Fc domain,” or “Fc” refers to the C-terminal region of the heavy chain of an antibody. Thus, an Fc region comprises the constant region of an antibody excluding the first constant region immunoglobulin domain (e.g., CH1 or CL).

An “antigen” is an entity (e.g., a proteinaceous entity or peptide) to which an antibody binds. In various embodiments, an antigen is an FGF receptor. In a particular embodiment, an antigen is human FGFR1c, FGFR2c, FGFR3c, and/or FGFR4.

The terms “specific binding,” “specifically binds,” “selective binding,” and “selectively binds,” mean that an antibody exhibits appreciable affinity for a particular antigen or epitope and, generally, does not exhibit significant cross-reactivity with other antigens and epitopes. “Appreciable” or preferred binding includes binding with a K_{D} of 10⁻⁷, 10⁻⁸, 10⁻⁹, or 10⁻¹⁰ M or better. The K_{D} of an antibody antigen interaction (the affinity constant) indicates the concentration of antibody at which 50% of antibody and antigen molecules are bound together. Thus, at a suitable fixed antigen concentration, 50% of a higher (i.e., stronger) affinity antibody will bind antigen molecules at a lower antibody concentration than would be required to achieve the same percent binding with a lower affinity antibody. Thus a lower K_{D} value indicates a higher (stronger) affinity. As used herein, “better” affinities are stronger affinities, and are of lower numeric value than their comparators, with a K_{D} of 10⁻⁷M being of lower numeric value and therefore representing a better affinity than a K_{D} of 10⁻⁶M. Affinities better (i.e., with a lower K_{D} value and therefore stronger) than 10⁻⁷M, preferably better than 10⁻⁸M, are generally preferred. Values intermediate to those set forth herein are also contemplated, and a preferred binding affinity can be indicated as a range of affinities, for example preferred binding affinities for anti-FGFR antibodies disclosed herein are, 10⁻⁷ to 10⁻¹²M, more preferably 10⁻⁸ to 10⁻¹² M. An antibody that “does not exhibit significant cross-reactivity” or “does not bind with a physiologically-relevant affinity” is one that will not appreciably bind to an off target antigen (e.g., a non-FGFR protein or an FGFR protein of the IIIb isoform). For example, in one embodiment, an antibody that specifically binds to FGFR1c will exhibit at least a two, and preferably three, or four or more orders of magnitude better binding affinity (i.e., binding exhibiting a two, three, or four or more orders of magnitude lower K_{D} value) for FGFR1c than, e.g., FGFR1b or a protein other than FGFR. Specific or selective binding can be determined according to any art-recognized means for determining such binding, including, for example, according to Scatchard analysis, Biacore analysis, bio-layer interferometry, and/or competitive (competition) binding assays as described herein. In one embodiment, “does not specifically bind to FGFR1b, FGFR2b, and/or FGFR3b” refers to an antibody which does not bind to FGFR1b, FGFR2b, and/or FGFR3b with an affinity significantly (statistically) different from a control antibody (e.g., an antibody that binds to an antigen other than FGFR proteins), as assessed by, e.g., bio-layer interferometry or ELISA.

The term “K_{D},” as used herein, is intended to refer to the dissociation equilibrium constant of a particular antibody-antigen interaction or the affinity of an antibody for an antigen. In other embodiments, an antibody binds an antigen with an affinity (K_{D}) of approximately less than 10⁻⁷ M, such as approximately less than 10⁻⁸ M, 10⁻⁹ M or 10⁻¹⁰ M or even lower when determined by bio-layer interferometery with a Pall ForteBio Octet RED96 Bio-Layer Interferometry system or surface plasmon resonance (SPR) technology in a BIACORE 3000 instrument using recombinant FGFR1c, FGFR2c, FGFR3c, and/or FGFR4 as the analyte and the antibody as the ligand, and binds to the predetermined antigen with an affinity that is at least two-fold greater than its affinity for binding to a non-specific antigen (e.g., BSA, casein) other than the predetermined antigen or a closely-related antigen. Other methods for determining K_{D} include equilibrium binding to live cells expressing FGFR1c, FGFR2c, FGFR3c, and/or FGFR4 via flow cytometry (FACS) or in solution using KinExA® technology.

The term “k_{assoc}” or “kₐ”, as used herein, is intended to refer to the association rate of a particular antibody-antigen interaction, whereas the term “k_{dis}”or “k_{d},” as used herein, is intended to refer to the dissociation rate of a particular antibody-antigen interaction. The term “K_{D}”, as used herein, is intended to refer to the dissociation constant, which is obtained from the ratio of k_{d} to kₐ (i.e,. k_{d}/kₐ) and is expressed as a molar concentration (M). K_{D} values for antibodies can be determined using methods well established in the art.

The terms “IC50” and “IC90,” as used herein, refer to the measure of the effectiveness of a compound (e.g., an anti-FGFR antibody described herein) in inhibiting a biological or biochemical function (e.g., the function or activity of FGFR1c, FGFR2c, FGFR3c, and/or FGFR4) by 50% and 90%, respectively. For example, IC50 indicates how much of an anti-FGFR antibody is needed to inhibit the activity of FGFR1c, FGFR2c, FGFR3c, and/or FGFR4 (e.g., the growth of a cell expressing FGFR1c, FGFR2c, FGFR3c, and/or FGFR4) by half. That is, it is the half maximal (50%) inhibitory concentration (IC) of an anti-FGFR antibody (50% IC, or IC₅₀). According to the FDA, IC50 represents the concentration of a drug that is required for 50% inhibition in vitro. The IC50 and IC90 can be determined by techniques known in the art, for example, by constructing a dose-response curve and examining the effect of different concentrations of the antagonist (i.e., the anti-FGFR antibody) on reversing FGFR activity.

The term “EC50” in the context of an in vitro or in vivo assay using an antibody or antigen binding fragment thereof, refers to the concentration of an antibody or an antigen-binding portion thereof that induces a response that is 50% of the maximal response, i.e., halfway between the maximal response and the baseline.

The term “epitope” or “antigenic determinant” refers to a site on an antigen to which an immunoglobulin or antibody specifically binds. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. Methods for determining what epitopes are bound by a given antibody (i.e., epitope mapping) are well known in the art and include, for example, immunoblotting and immunoprecipitation assays, wherein overlapping or contiguous peptides are tested for reactivity with a given antibody. Methods of determining spatial conformation of epitopes include techniques in the art, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)). The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the “same epitope on FGFR1c, FGFR2c, FGFR3c, and/or FGFR4” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc ; 2006; doi:10.1101/pdb.prot4277 or in Chapter 11 of “Using Antibodies” by Ed Harlow and David Lane, Cold Spring Harbor Laboratory Press, Cold Spring Harbor, N.Y., USA 1999. Competing antibodies bind to the same epitope, an overlapping epitope or to adjacent epitopes (e.g., as evidenced by steric hindrance). Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding antibodies or antibody fragments (e.g., V_{H}, V_{L}, CDR3), is intended to refer to a nucleic acid molecule in which the nucleotide sequences are essentially free of other genomic nucleotide sequences, e.g., those encoding antibodies that bind antigens other than FGFR, which other sequences may naturally flank the nucleic acid in human genomic DNA.

The term “modifying,” or “modification,” as used herein, refers to changing one or more amino acids in an antibody or antigen-binding portion thereof. The change can be produced by adding, substituting or deleting an amino acid at one or more positions. The change can be produced using known techniques, such as PCR mutagenesis. For example, in some embodiments, an antibody or an antigen-binding portion thereof identified using the methods provided herein can be modified, to thereby modify the binding affinity of the antibody or antigen-binding portion thereof to FGFR (e.g., FGFR1c, FGFR2c, FGFR3c, and/or FGFR4).

“Conservative amino acid substitutions” in the sequences of the antibodies refer to nucleotide and amino acid sequence modifications which do not abrogate the binding of the antibody encoded by the nucleotide sequence or containing the amino acid sequence, to the antigen (e.g., FGFR1c, FGFR2c, FGFR3c, and/or FGFR4). Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an anti-FGFR antibody is preferably replaced with another amino acid residue from the same class. Methods of identifying nucleotide and amino acid conservative substitutions which do not eliminate antigen binding are well-known in the art.

The term “non-conservative amino acid substitution” refers to the substitution of an amino acid in one class with an amino acid from another class; for example, substitution of an Ala, a class II residue, with a class III residue such as Asp, Asn, Glu, or Gln.

Alternatively, in another embodiment, mutations (conservative or non-conservative) can be introduced randomly along all or part of an anti-FGFR antibody coding sequence, such as by saturation mutagenesis, and the resulting modified anti-FGFR antibodies can be screened for binding activity.

A “consensus sequence” is a sequence formed from the most frequently occurring amino acids (or nucleotides) in a family of related sequences. In a family of proteins, each position in the consensus sequence is occupied by the amino acid occurring most frequently at that position in the family. If two amino acids occur equally frequently, either can be included in the consensus sequence. A “consensus framework” of an immunoglobulin refers to a framework region in the consensus immunoglobulin sequence. Similarly, the consensus sequence for the CDRs of can be derived by optimal alignment of the CDR amino acid sequences of anti-FGFR antibodies provided herein. For example, a consensus CDR sequence may be presented in the form of AB[CD]EF[GH], wherein the residue at the bracketed position (i.e., positions 3 and 6) is selected from a residue listed within the bracket. Accordingly, AB[CD]EF[GH] would encompass the CDR sequences ABCEFG, ABCEFH, ABDEFG, and ABDEFH.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand. For polypeptides, the term “substantial homology” indicates that two polypeptides, or designated sequences thereof, when optimally aligned and compared, are identical, with appropriate amino acid insertions or deletions, in at least about 80% of the amino acids, usually at least about 90% to 95%, and more preferably at least about 98% to 99.5% of the amino acids.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art.

The nucleic acid compositions, while often comprising a native sequence (except for modified restriction sites and the like), from either cDNA, genomic or mixtures thereof may alternately be mutated, in accordance with standard techniques to provide altered gene sequences. For coding sequences, these mutations, may modify the encoded amino acid sequence as desired. In particular, DNA sequences substantially homologous to native V, D, J, constant, switches and other such sequences described herein are contemplated.

As used herein, “percent (%) identity” with respect to a reference polypeptide or nucleotide sequence is defined as the percentage of amino acid or nucleotide residues in a candidate sequence that are identical with the amino acid or nucleotide residues in the reference polypeptide or nucleotide sequence, after aligning the sequences and introducing gaps, if necessary, to achieve the maximum percent sequence identity, and not considering any conservative substitutions as part of the sequence identity. Alignment for purposes of determining percent identity of amino acid or nucleotide sequences can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

For purposes herein, percent identity values are generated using the BLASTN (nucleotides) or BLASTP (polypeptides) algorithm with default settings. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

Different regions within a single polynucleotide or polypeptide target sequence that aligns with a polynucleotide or polypeptide reference sequence can each have their own percent sequence identity. It will be appreciated that where the length of sequence A is not equal to the length of sequence B, the % identity of A to B will not equal the % identity of B to A. Unless specifically stated otherwise, all % identity values used herein are obtained using the BLASTP (for polypeptides) or BLASTN program.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

The term “operably linked” refers to a nucleic acid sequence placed into a functional relationship with another nucleic acid sequence. For example, DNA for a presequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms, “plasmid” and “vector” may be used interchangeably. However, other forms of expression vectors, such as viral vectors (e.g., replication defective retroviruses, adenoviruses and adeno-associated viruses), which serve equivalent functions are also contemplated.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

The term “inhibition” as used herein, refers to any statistically significant decrease in biological activity, including partial and full blocking of the activity. For example, “inhibition” can refer to a statistically significant decrease of about 10%, 20%, 30%, 40%, 50%, 60%, 70%, 80%, 90%, 95%, 96%, 97%, 98%, 99%, or 100% in biological activity.

Inhibition of phosphorylation, as used herein, refers to the ability of an antibody to statistically significantly decrease the phosphorylation of a substrate protein relative to the signaling in the absence of the antibody (control). As is known in the art, intracellular signaling pathways include, for example, the mitogen-activated protein kinase (MAPK/ERK or “ERK”) pathway. As is also known in the art, FGFR-mediated signaling can be measured by assaying for the level phosphorylation of the substrate (e.g., phosphorylation or no phosphorylation of ERK). Accordingly, in one embodiment, the anti-FGFR antibodies and compositions described herein provide statistically significant inhibition of the level of phosphorylation of ERK by at least 10%, or at least 20%, or at least 30%, or at least 40%, or at least 50%, or at least 60%, or at least 70%, or at least 80%, or at least 90%, or about 100% relative to the level of phosphorylation ERK in the absence of such antibody (control). Such FGFR mediated signaling can be measured using art recognized techniques which measure a protein in a cellular cascade involving FGFR, e.g., ELISA, western, or multiplex methods, such as Luminex®.

The phrase “inhibition of the growth of cells expressing FGFR,” as used herein, refers to the ability of an antibody to statistically significantly decrease the growth of a cell expressing FGFR relative to the growth of the cell in the absence of the antibody (control) either in vivo or in vitro. In one embodiment, the growth of a cell expressing FGFR (e.g., a cancer cell) may be decreased by at least 10%, or at least 20%, or at least 30%, or at least 40%, or at least 50%, or at least 60%, or at least 70%, or at least 80%, or at least 90%, or about 100% when the cells are contacted with an antibody or composition disclosed herein, relative to the growth measured in the absence of the antibody or composition (control). Cellular growth can be assayed using art recognized techniques which measure the rate of cell division, the fraction of cells within a cell population undergoing cell division, and/or the rate of cell loss from a cell population due to terminal differentiation or cell death (e.g., using a cell titer glow assay or thymidine incorporation).

The phrase “inhibition of FGFR ligand binding to FGFR,” as used herein, refers to the ability of an antibody to statistically significantly decrease the binding of an FGFR ligand to its receptor, FGFR (e.g., FGFR1c, FGFR2c, FGFR3c and/or FGFR4), relative to the FGFR ligand binding in the absence of the antibody (control). In other words, in the presence of the antibody, the amount of the FGFR ligand that binds to FGFR relative to a control (no antibody), is statistically significantly decreased. The amount of an FGFR ligand which binds FGFR may be decreased in the presence of an antibody composition or combination disclosed herein by at least 10%, or at least 20%, or at least 30%, or at least 40%, or at least 50%, or at least 60%, or at least 70%, or at least 80%, or at least 90%, or about 100% relative to the amount in the absence of the antibody (control). A decrease in FGFR ligand binding can be measured using art-recognized techniques that measure the level of binding of labeled FGFR ligand (e.g., radiolabelled FGF) to cells expressing FGFR in the presence or absence (control) of the antibody.

As used herein, the term “inhibits growth” of a tumor includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or 100%.

The terms “treat,” “treating,” and “treatment,” as used herein, refer to therapeutic or preventative measures described herein. The methods of “treatment” employ administration to a subject, an antibody or antibody pair or trio disclosed herein, for example, a subject having a disease or disorder associated with FGFR-dependent signaling or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

The term “disease associated with FGFR-dependent signaling,” or “disorder associated with FGFR-dependent signaling,” as used herein, includes disease states and/or symptoms associated with a disease state, where increased levels of FGFR and/or activation of cellular cascades involving FGFR are found. The term “disease associated with FGFR-dependent signaling,” also includes disease states and/or symptoms associated with the activation of alternative FGFR signaling pathways. In general, the term “disease associated with FGFR dependent signaling,” refers to any disorder, the onset, progression or the persistence of the symptoms of which requires the participation of FGFR. Exemplary FGFR-mediated disorders include, but are not limited to, for example, cancer.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, melanoma, colorectal cancer, endometrial carcinoma, salivary gland carcinoma, kidney cancer, renal cancer, prostate cancer, vulval cancer, thyroid cancer, hepatic carcinoma and various types of head and neck cancer.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

The term “therapeutic agent” in intended to encompass any and all compounds that have an ability to decrease or inhibit the severity of the symptoms of a disease or disorder, or increase the frequency and/or duration of symptom-free or symptom-reduced periods in a disease or disorder, or inhibit or prevent impairment or disability due to a disease or disorder affliction, or inhibit or delay progression of a disease or disorder, or inhibit or delay onset of a disease or disorder, or inhibit or prevent infection in an infectious disease or disorder. Non-limiting examples of therapeutic agents include small organic molecules, monoclonal antibodies, bispecific antibodies, recombinantly engineered biologics, RNAi compounds, tyrosine kinase inhibitors (e.g., PI3K inhibitors), and commercial antibodies. In certain embodiments, tyrosine kinase inhibitors include, e.g., one or more of erlotinib, gefitinib, and lapatinib, which are currently marketed pharmaceuticals.

As used herein, “administering” refers to the physical introduction of a composition comprising a therapeutic agent to a subject, using any of the various methods and delivery systems known to those skilled in the art. Exemplary routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “patient” includes human and other mammalian subjects that receive either prophylactic or therapeutic treatment.

The term “subject” includes any mammal. For example, the methods and compositions herein disclosed can be used to treat a subject having cancer. In a particular embodiment, the subject is a human.

The term “sample” refers to tissue, body fluid, or a cell (or a fraction of any of the foregoing) taken from a patient or a subject. Normally, the tissue or cell will be removed from the patient, but in vivo diagnosis is also contemplated. In the case of a solid tumor, a tissue sample can be taken from a surgically removed tumor and prepared for testing by conventional techniques. In the case of lymphomas and leukemias, lymphocytes, leukemic cells, or lymph tissues can be obtained (e.g., leukemic cells from blood) and appropriately prepared. Other samples, including urine, tears, serum, plasma, cerebrospinal fluid, feces, sputum, cell extracts etc. can also be useful for particular cancers.

As used herein, the term “about” means plus or minus 10% of a specified value. As used herein, the term “and/or” includes any and all combinations of one or more of the associated listed items. For example, the phrase “FGFR1c, FGFR2c, FGFR3c, and/or FGFR4” is intended to encompass each of FGFR1c, FGFR2c, FGFR3c, and FGFR4 individually, all four of FGFR1c, FGFR2c, FGFR3c, and FGFR4, as well as in any combination thereof (i.e., all combinations of two of FGFR1c, FGFR2c, FGFR3c, and FGFR4, and all combinations of three of FGFR1c, FGFR2c, FGFR3c, and FGFR4). Accordingly, “FGFR1c, FGFR2c, FGFR3c, and/or FGFR4” is intended to encompass the following: FGFR1c; FGFR2c; FGFR3c; FGFR4; FGFR1c and FGFR2c; FGFR1c and FGFR3c; FGFR1c and FGFR4; FGFR2c and FGFR3c; FGFR2c and FGFR4; FGFR3c and FGFR4; FGFR1c, FGFR2c, and FGFR3c; FGFR1c, FGFR3c, and FGFR4; FGFR2c, FGFR3c, and FGFR4; and FGFR1c, FGFR2c, FGFR3c, and FGFR4. Furthermore, “FGFR1b, FGFR2b, and/or FGFR3b” is intended to encompass the following: FGFR1b; FGFR2b; FGFR3b; FGFR1b and FGFR2b; FGFR1b and FGFR3b; FGFR2b and FGFR3b; and FGFR1b, FGFR2b, and FGFR3b.

As used herein, the phrase “Ab1-Ab107” is interchangeable with “Abl through Ab107” and is shorthand for the 107 anti-FGFR antibodies described in Table 9. Specifically, “Ab1-Ab107” encompasses Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, Ab36, Ab37, Ab38, Ab39, Ab40, Ab41, Ab42, Ab43, Ab44, Ab45, Ab46, Ab47, Ab48, Ab49, Ab50, Ab51, Ab52, Ab53, Ab54, Ab55, Ab56, Ab57, Ab58, Ab59, Ab60, Ab61, Ab62, Ab63, Ab64, Ab65, Ab66, Ab67, Ab68, Ab69, Ab70, Ab71, Ab72, Ab73, Ab74, Ab75, Ab76, Ab77, Ab78, Ab79, Ab80, Ab81, Ab82, Ab83, Ab84, Ab85, Ab86, Ab87, Ab88, Ab89, Ab90, Ab91, Ab92, Ab93, Ab94, Ab95, Ab96, Ab97, Ab98, Ab99, Ab100, Ab101, Ab102, Ab103, Ab104, Ab105, Ab106, and Ab107. The phrase “any of Ab1-Ab107” is intended to encompass any one of the 107 antibodies.

As used in the description of the invention and the appended claims, the singular forms “a,” “an” and “the” are intended to include the plural forms as well, unless the context clearly indicates otherwise.

Various aspects of the disclosure are described in further detail in the following subsections.

### III. Anti-FGFR Antibodies

Provided herein are antibodies which specifically bind to the extracellular domain of particular isoforms of FGFR proteins (e.g., human FGFR proteins). For example, the antibodies described herein bind specifically to the extracellular domains of FGFR1c, FGFR2c, FGFR3c, and/or FGFR4 (e.g., human FGFR1c, FGFR2c, FGFR3c, and/or FGFR4). In some embodiments, the antibodies, or antigen-binding portions thereof, do not bind to FGFR1b, FGFR2b, and/or FGFR3b (e.g., human FGFR1b, FGFR2b, and/or FGFR3b).

Accordingly, the antibodies described herein exhibit one or more of the following properties:

(a) binds to human FGFR1c, FGFR2c, FGFR3c, and/or FGFR4;

(b) does not bind to FGFR1b, FGFR2b, and/or FGFR3b , as assessed by ELISA or bio-layer interferometry (e.g., ForteBio assay);

(c) inhibits the binding of FGF1 or FGF2 to FGFR1c, FGFR2c, FGFR3c, and/or FGFR4;

(d) inhibits FGF2-mediated phosphorylation of ERK; and

(e) inhibits FGF2-mediated cell viability.

In some embodiments, the anti-FGFR antibodies bind to FGFR1c, FGFR2c, FGFR3c, or FGFR4c. In some embodiments, the anti-FGFR4 antibodies described herein bind to two FGFR proteins, i.e., FGFR1c and FGFR2c; FGFR1c and FGFR3c, FGFR1c and FGFR4; FGFR2c and FGFR3c; FGFR2c and FGFR4; or FGFR3c and FGFR4. In some embodiments, the anti-FGFR4 antibodies described herein bind to three FGFR proteins, i.e., FGFR1c, FGFR2c, and FGFR3c; FGFR1c, FGFR2c, and FGFR4; or FGFR2c, FGFR3c, and FGFR4. In some embodiments, the anti-FGFR antibodies described herein bind to FGFR1c, FGFR2c, FGFR3c, and FGFR4.

In some embodiments, the anti-FGFR antibodies described herein bind to the extracellular domain of FGFR1c (e.g., human FGFR1c), for example, with a K_{D} of 10⁻⁷ M or less, 10⁻⁸ M or less, 10⁻⁹ M or less, 10⁻¹⁰ M or less, 10⁻¹¹ M or less, 10⁻¹² M or less, 10⁻¹² M to 10⁻⁷ M, 10⁻¹¹ M to 10⁻⁷ M, 10⁻¹⁰ M to 10⁻⁷ M, or 10⁻⁹ M to 10⁻⁷ M, as assessed by, e.g., the ForteBio assay described in Example 9.

In some embodiments, the anti-FGFR antibodies described herein bind to the extracellular domain of FGFR2c (e.g., human FGFR2c), for example, with a K_{D} of 10⁻⁷ M or less, 10⁻⁸ M or less, 10⁻⁹ M or less, 10⁻¹⁰ M or less, 10⁻¹¹ M or less, 10⁻¹² M or less, 10⁻¹² M to 10⁻⁷ M, 10⁻¹¹ M to 10⁻⁷ M, 10⁻¹⁰ M to 10⁻⁷ M, or 10⁻⁹ M to 10⁻⁷ M, as assessed by, e.g., bio-layer interferometry (e.g., ForteBio assay) as described in Example 9.

In some embodiments, the anti-FGFR antibodies described herein bind to the extracellular domain of FGFR3c (e.g., human FGFR3c), for example, with a K_{D} of 10⁻⁷ M or less, 10⁻⁸ M or less, 10⁻⁹ M or less, 10⁻¹⁰ M or less, 10⁻¹¹ M or less, 10⁻¹² M or less, 10⁻¹² M to 10⁻⁷ M, 10⁻¹¹ M to 10⁻⁷ M, 10⁻¹⁰ M to 10⁻⁷ M, or 10⁻⁹ M to 10⁻⁷ M, as assessed by, e.g., bio-layer interferometry (e.g., ForteBio assay) as described in Example 9.

In some embodiments, the anti-FGFR antibodies described herein bind to the extracellular domain of FGFR4 (e.g., human FGFR4), for example, with a K_{D} of 10⁻⁷ M or less, 10⁻⁸ M or less, 10⁻⁹ M or less, 10⁻¹⁰ M or less, 10⁻¹¹ M or less, 10⁻¹² M or less, 10⁻¹² M to 10⁻⁷ M, 10⁻¹¹ M to 10⁻⁷ M, 10⁻¹⁰ M to 10⁻⁷ M, or 10⁻⁹ M to 10⁻⁷ M, as assessed by, e.g., bio-layer interferometry (e.g., ForteBio assay) as described in Example 9.

In some embodiments, the anti-FGFR antibodies described herein bind to the extracellular domain of FGFR1c (e.g., human FGFR1c), for example, with an EC50 of about 200 nM or less, e.g., 150 nM or less, 100 nM or less, 75 nM or less, 50 nM or less, 25 nM or less, 10 nM or less, 5 nM or less, 2.5 nM or less, 1 nM or less, 0.5 nM or less, 0.25 nM or less, 0.1 nM or less, 0.075 nM or less, 0.05 nM or less, 0.01 nM or less, 0.0075 nM or less, 0.005 nM or less, 0.001 nM or less, 200 nM to 0.001 nM, 150 nM to 0.001 nM, 100 nM to 0.005 nM, 50 nM to 0.005 nM or less, or 25 nM to 0.005 nM, as assessed by, e.g., ELISA as described in Example 3.

In some embodiments, the anti-FGFR antibodies described herein bind to the extracellular domain of FGFR2c (e.g., human FGFR2c), for example, with an EC50 of about 200 nM or less, e.g., 150 nM or less, 100 nM or less, 75 nM or less, 50 nM or less, 25 nM or less, 10 nM or less, 5 nM or less, 2.5 nM or less, 1 nM or less, 0.5 nM or less, 0.25 nM or less, 0.1 nM or less, 0.075 nM or less, 0.05 nM or less, 0.01 nM or less, 0.0075 nM or less, 0.005 nM or less, 0.001 nM or less, 200 nM to 0.001 nM, 150 nM to 0.001 nM, 100 nM to 0.005 nM, 50 nM to 0.005 nM or less, or 25 nM to 0.005 nM, as assessed by, e.g., ELISA as described in Example 3.

In some embodiments, the anti-FGFR antibodies described herein bind to the extracellular domain of FGFR3c (e.g., human FGFR3c), for example, with an EC50 of about 200 nM or less, e.g., 150 nM or less, 100 nM or less, 75 nM or less, 50 nM or less, 25 nM or less, 10 nM or less, 5 nM or less, 2.5 nM or less, 1 nM or less, 0.5 nM or less, 0.25 nM or less, 0.1 nM or less, 0.075 nM or less, 0.05 nM or less, 0.01 nM or less, 0.0075 nM or less, 0.005 nM or less, 0.001 nM or less, 200 nM to 0.001 nM, 150 nM to 0.001 nM, 100 nM to 0.005 nM, 50 nM to 0.005 nM or less, or 25 nM to 0.005 nM, as assessed by, e.g., ELISA as described in Example 3.

In some embodiments, the anti-FGFR antibodies described herein bind to the extracellular domain of FGFR4 (e.g., human FGFR4), for example, with an EC50 of about 200 nM or less, e.g., 150 nM or less, 100 nM or less, 75 nM or less, 50 nM or less, 25 nM or less, 10 nM or less, 5 nM or less, 2.5 nM or less, 1 nM or less, 0.5 nM or less, 0.25 nM or less, 0.1 nM or less, 0.075 nM or less, 0.05 nM or less, 0.01 nM or less, 0.0075 nM or less, 0.005 nM or less, 0.001 nM or less, 200 nM to 0.001 nM, 150 nM to 0.001 nM, 100 nM to 0.005 nM, 50 nM to 0.005 nM or less, or 25 nM to 0.005 nM, as assessed by, e.g., ELISA as described in Example 3.

In some embodiments, the anti-FGFR antibodies described herein do not bind to FGFR1b, FGFR2b, and/or FGFR3b (e.g., human FGFR1b, FGFR2b, and/or FGFR3b), as assessed by ELISA or bio-layer interferometry (e.g., ForteBio assay). Accordingly, in some embodiments, the anti-FGFR antibodies described herein do not bind to FGFR1b, FGFR2b, or FGFR3b. In some embodiments, the anti-FGFR antibodies described herein do not bind to FGFR1b and FGFR2b; FGFR1b and FGFR3b; or FGFR2b and FGFR3b. In some embodiments, the anti-FGFR antibodies described herein do not bind to FGFR1b, FGFR2b, and FGFR3b. In some embodiments, the anti-FGFR antibodies described herein do not bind to human FGFR1b, human FGFR2b, and FGFR3b. Accordingly, in a particular embodiment, the anti-FGFR antibodies described herein bind to human FGFR1c, FGFR2c, FGFR3c, and FGFR4, and do not bind to human FGFR1b, FGFR2b, and FGFR3b.

In some embodiments, the anti-FGFR antibodies described herein may inhibit binding of human FGF1 or FGF2 to human FGFR1c, FGFR2c, FGFR3c, and/or FGFR4 with an IC₅₀ of 500 nM or less, for example, 400 nM or less, 300 nM or less, 200 nM or less, 150 nM or less, 100 nM or less, 75 nM or less, 50 nM or less, 25 nM or less, 20 nM or less, 10 nM or less, 5 nM or less, 1 nM or less, 0.5 nM or less, 0.1 nM or less, 0.05 nM or less, 0.01 nM or less, 0.005 nM or less, 0.001 nM or less, 500 nM to 0.001 nM, 400 nM to 0.001 nM, 300 nM to 0.05 nM, 200 nM to 0.05 nM, or 150 nM to 0.5 nM (see Example 6).

In some embodiments, the anti-FGFR antibodies described herein inhibit signaling downstream of FGFRs, for example, FGF2-mediated phosphorylation of ERK. Accordingly, in some embodiments, the antibodies described herein inhibit FGF2-mediated phosphorylation with an IC50 of 200 nM or less, for example, 150 nM or less, 100 nM or less, 75 nM or less, 50 nM or less, 25 nM or less, 20 nM or less, 15 nM or less, 10 nM or less, 5 nM or less, 1 nM or less, 0.5 nM or less, 0.1 nM or less, 0.05 nM or less, 0.01 nM or less, 100 nM to 0.01 nM, 50 nM to 0.05 nM, 25 nM to 0.05 nM, 10 nM to 0.05 nM, as determined by, e.g., pERK SureFire Assay(see Example 7).

In some embodiments, the anti-FGFR antibodies described herein inhibit viability of tumor cells (e.g., a tumor cell line such as IGROV-1 cells) with an IC50 of 200 nM or less, for example, 150 nM or less, 100 nM or less, 75 nM or less, 50 nM or less, 25 nM or less, 20 nM or less, 15 nM or less, 10 nM or less, 5 nM or less, 1 nM or less, 0.5 nM or less, 0.1 nM or less, 0.05 nM or less, 0.01 nM or less, 200 nM to 0.1 nM, 150 nM to 0.5 nM, 100 nM to 1 nM, 50 nM to 1 nM, as determined by, e.g., the CellTiterGlo (CTG) assay described in Example 8.

In some embodiments, the anti-FGFR antibodies described herein has a serum half-life of 25 hour or more, 50 hours or more, 100 hours or more, 150 hours or more, 200 hours or more, 250 hours or more, 300 hours or more, 350 hours or more, 400 hours or more, or longer in mice when administered intravenously at a single dose of 40 mg/kg.

In one embodiment, provided herein is a modified antibody (e.g., an antibody with an IgG2 constant region or variant thereof) that binds to FGFR1c, wherein the antibody exhibits increased tolerability as compared to an antibody comprising identical heavy and light chain variable region sequences and an IgG1 constant region when administered to a mammal (e.g., a mouse or human). In certain embodiments, the reduction in weight loss when the antibody is administered is about 10% or less, for example, about 9% or less, about 8% or less, about 7% or less, about 6% or less, about 5% or less, about 4% or less, about 3% or less, about 2% or less, or about 1% or less, relative to the weight loss observed for the same antibody in IgG1 form.

In another embodiment, provided herein is a modified antibody that binds to FGFR1c, wherein administration of the antibody to a mammal (e.g., a mouse or human) does not result in significant weight loss.

An antibody that exhibits one or more of the functional properties described above (e.g., biochemical, immunochemical, cellular, physiological or other biological activities, or the like) as determined according to methodologies known to the art and described herein, will be understood to relate to a statistically significant difference in the particular activity relative to that seen in the absence of the antibody (e.g., or when a control antibody of irrelevant specificity is present). Preferably, the anti-FGFR antibody-induced increases in a measured parameter effects a statistically significant increase by at least 10% of the measured parameter, more preferably by at least 20%, 30%, 40%, 50%, 60%, 70%, 80%, 90%, 95%, or 100% (i.e, 2 fold), 3 fold, 5 fold or 10 fold. Conversely, anti-FGFR antibody-induced decreases in a measured parameter (e.g., tumor volume, FGF1 binding to FGFR1c, FGFR2c, FGFR3c, and/or FGFR4) effects a statistically significant decrease by at least 10%, 20%, 30%, 40%, 50%, 60%, 70%, 80%, 90%, 95%, 97%, 98%, 99%, or 100%.

Also provided herein are antibodies which bind to particular isoforms of FGFRs, e.g., FGFR1c, FGFR2c, FGFR3c, and/or FGFR4, and have particular variable region or CDR sequences, as described below. In some embodiments, the antibodies do not bind to FGFR1b, FGFR2b, and/or FGFR3b. In some embodiments, the antibodies described herein bind to FGFR1c, FGFR2c, FGFR3c, and/or FGFR4, and do not bind to FGFR1b, FGFR2b, and/or FGFR3b.

Accordingly, in some embodiments, the antibodies described herein comprise the heavy and light chain variable region sequences or CDR sequences of any of Ab1-Ab107 (as described in Table 9). The VH sequences of Ab1-Ab107 are set forth in SEQ ID NOs: 29; 40; 51; 62; 73; 84; 95; 106; 117; 128; 139; 150; 161; 172; 183; 194; 204; 214; 224; 234; 244; 254; 264; 274; 284; 294; 304; 314; 324; 334; 344; 354; 364; 374; 384; 394; 404; 414; 424; 434; 444; 454; 464; 474; 484; 494; 504; 514; 524; 534; 544; 554; 564; 574; 584; 594; 604; 614; 624; 634; 644; 654; 664; 674; 684; 694; 704; 714; 724; 734; 744; 754; 764; 774; 784; 794; 804; 814; 824; 834; 844; 854; 864; 874; 884 ; 894; 904; 914; 924; 934; 944; 954; 964; 974; 984; 994; 1004; 1014; 1024; 1034; 1044; 1054; 1064; 1074; 1084; 1094; and 1104, respectively. The VL sequences of Ab1-Ab107 are set forth in SEQ ID NOs: 30; 41; 52; 63; 74; 85; 96; 107; 118; 129; 140; 151; 162; 173; 184; 195; 205; 215; 225; 235; 245; 255; 265; 275; 285; 295; 305; 315; 325; 335; 345; 355; 365; 375; 385; 395; 405; 415; 425; 435; 445; 455; 465; 475; 485; 495; 505; 515; 525; 535; 545; 555; 565; 575; 585; 595; 605; 615; 625; 635; 645; 655; 665; 675; 685; 695; 705; 715; 725; 735; 745; 755; 765; 775; 785; 795; 805; 815; 825; 835; 845; 855; 865; 875; 885; 895; 905; 915; 925; 935; 945; 955; 965; 975; 985; 995; 1005; 1015; 1025; 1035; 1045; 1055; 1065; 1075; 1085; 1095; and 1105, respectively.

In one embodiment, provided herein are anti-FGFR antibodies comprising heavy and light chain CDR1, CDR2, and CDR3 sequences, wherein the heavy and light chain CDR1, CDR2, and CDR3 sequences are selected from the group consisting of SEQ ID NOs: 23-25 and 26-28, respectively; 34-36 and 37-39; 45-47 and 48-50; 56-58 and 59-61, 67-69 and 70-72; 78-80 and 81-83; 89-91 and 92-94; 100-102 and 103-105; 111-113 and 114-116; 122-124 and 125-127; 133-135 and 136-138; 144-146 and 147-149; 155-157 and 158-160, 166-168 and 169-171; 177-179 and 180-182; 188-190 and 191-193; 198-200 and 201-203; 208-210 and 211-213; 218-220 and 221-223; 228-230 and 231-233; 238-240 and 241-243; 248-250 and 251-253; 258-260 and 261-263; 268-270 and 271-273; 278-280 and 281-283; 288-290 and 291-293; 298-300 and 301-303; 308-310 and 311-313; 318-320 and 321-323; 228-330 and 331-333; 338-340 and 341-343; 348-350 and 351-353; 358-360 and 361-363; 368-370 and 371-373; 378-380 and 381-383; 388-390 and 391-393; 398-400 and 401-403; 408-410 and 411-413; 418-420 and 421-423; 428-430 and 431-433; 438-440 and 441-443; 448-450 and 451-453; 458-460 and 461-463; 468-470 and 471-473; 478-480 and 481-483; 488-490 and 491-493; 498-500 and 501-503; 508-510 and 511-513; 518-520 and 521-523; 528-530 and 531-533; 538-540 and 541-543; 548-550 and 551-553; 558-560 and 561-563; 568-570 and 571-573; 578-580 and 581-583; 588-590 and 591-593; 598-600 and 601-603; 608-610 and 611-613; 618-620 and 621-623; 628-630 and 631-633; 638-640 and 641-643; 648-650 and 651-653; 658-660 and 661-663; 668-670 and 671-673; 678-680 and 681-683; 688-690 and 691-693; 698-700 and 701-703; 708-710 and 711-713; 718-720 and 721-723; 728-730 and 731-733; 738-740 and 741-743; 748-750 and 751-753; 758-760 and 761-763; 768-770 and 771-773; 778-780 and 781-783; 788-790 and 791-793; 798-800 and 801-803; 808-810 and 811-813; 818-820 and 821-823; 828-830 and 831-833; 838-840 and 841-843; 848-850 and 851-853; 858-860 and 861-863; 868-870 and 871-873; 878-880 and 881-883; 888-890 and 891-893; 898-900 and 901-903; 908-910 and 911-913; 918-920 and 921-923; 928-930 and 931-933; 938-940 and 941-943; 948-950 and 951-953; 958-960 and 961-963; 968-970 and 971-973; 978-980 and 981-983; 988-990 and 991-993; 998-1000 and 1001-1003; 1008-1010 and 1011-1013; 1018-1020 and 1021-1023; 1028-1030 and 1031-1033; 1038-1040 and 1041-1043; 1048-1050 and 1051-1053; 1058-1060 and 1061-1063; 1068-1070 and 1071-1073; 1078-1080 and 1081-1083; 1088-1090 and 1091-1093; and 1098-1100 and 1101-1103, wherein the antibody specifically binds to FGFR1c, FGFR2c, FGFR3c, and/or FGFR4. In another embodiment, provided herein are isolated monoclonal antibodies, or antigen-binding portions thereof, which specifically bind to FGFR1c, FGFR2c, FGFR3c, and/or FGFR4, and comprise IMGT heavy chain CDR1, CDR2, and CDR3 sequences and IMGT light chain CDR1, CDR2, and CDR3 sequences selected from the group consisting of SEQ ID NOs: 1544-1546 and 1547-1549; 1550-1552 and 1553-1555; 1556-1558 and 1559-1561; 1562-1564 and 1565-1567; 1568-1570 and 1571-1573; ;1574-1576 and 1577-1579; 1580-1582 and 1583-1585; 1586-1588 and 1589-1591; 1592-1594 and 1595-1597; 1598-1600 and 1601-1603; 1604-1606 and 1607-1609; 1610-1612 and 1613-1615; 1616-1618 and 1619-1621; 1622-1624 and 1625-1627; 1628-1630 and 1631-1633; 1634-1636 and 1637-1639; 1640-1642 and 1643-1645; 1646-1648 and 1649-1651; 1652-1654 and 1655-1657; 1658-1660 and 1661-1663; 1664-1666 and 1667-1669; 1670-1672 and 1673-1675; 1676-1678 and 1679-1681; 1682-1684 and 1685-1687; 1688-1690 and 1691-1693; 1694-1696 and 1697-1699; 1700-1702 and 1703-1705; 1706-1708 and 1709-1711; 1712-1714 and 1715-1717; 1718-1720 and 1721-1723; 1724-1726 and 1727-1729; 1730-1732 and 1733-1735; 1736-1738 and 1739-1741; 1742-1744 and 1745-1747; 1748-1750 and 1751-1753; 1754-1756 and 1757-1759; 1760-1762 and 1763-1765; 1766-1768 and 1769-1771; 1772-1774 and 1775-1777; 1778-1780 and 1781-1783; 1784-1786 and 1787-1789; 1790-1792 and 1793-1795; 1796-1798 and 1799-1801; 1802-1804 and 1805-1807; 1808-1810 and 1811-1813; 1814-1816 and 1817-1819; 1820-1822 and 1823-1825; 1826-1828 and 1829-1831; 1832-1834 and 1835-1837; 1838-1840 and 1841-1843; 1844-1846 and 1847-1849; 1850-1852 and 1853-1855; 1856-1858 and 1859-1861; 1862-1864 and 1865-1867; 1868-1870 and 1871-1873; 1874-1876 and 1877-1879; 1880-1882 and 1883-1885; 1886-1888 and 1889-1891; 1892-1894 and 1895-1897; 1898-1900 and 1901-1903; 1904-1906 and 1907-1909; 1910-1912 and 1913-1915; 1916-1918 and 1919-1921; 1922-1924 and 1925-1927; 1928-1930 and 1931-1933; 1934-1936 and 1937-1939; 1940-1942 and 1943-1945; 1946-1948 and 1949-1951; 1952-1954 and 1955-1957; 1958-1960 and 1961-1963; 1964-1966 and 1967-1969; 1970-1972 and 1973-1975; 1976-1978 and 1979-1981; 1982-1984 and 1985-1987; 1988-1990 and 1991-1993; 1994-1996 and 1997-1999; 2000-2002 and 2003-2005; 2006-2008 and 2009-2011; 2012-2014 and 2015-2017; 2018-2020 and 2021-2023; 2024-2026 and 2027-2029; 2030-2032 and 2033-2035; 2036-2038 and 2039-2041; 2042-2044 and 2045-2047; 2048-2050 and 2051-2053; 2054-2056 and 2057-2059; 2060-2062 and 2063-2065; 2066-2068 and 2069-2071; 2072-2074 and 2075-2077; 2078-2080 and 2081-2083; 2084-2086 and 2087-2089; 2090-2092 and 2093-2095; 2096-2098 and 2099-2101; 2102-2104 and 2105-2107; 2108-2110 and 2111-2113; 2114-2116 and 2117-2119; 2120-2122 and 2123-2125; 2126-2128 and 2129-2131; 2132-2134 and 2135-2137; 2138-2140 and 2141-2143; 2144-2146 and 2147-2149; 2150-2152 and 2153-2155; 2156-2158 and 2159-2161; 2162-2164 and 2165-2167; 2168-2170 and 2171-2173; 2174-2176 and 2177-2179; and 2180-2182 and 2183-2185. In some embodiments, the antibody does not bind to FGFR1b, FGFR2b, and/or FGFR3b. In these lists, the first set of numbers within a pair of semicolons correspond to the heavy chain CDR1, CDR2, and CDR3 sequences and the second set of numbers following the term “and” correspond to the light chain CDR1, CDR2, and CDR3 sequences. For example, “; 34-36 and 37-39;” in the list above indicates that the antibody comprises heavy chain CDR1, CDR2, and CDR3 sequences set forth in SEQ ID NOs: 34-36, respectively, and light chain CDR1, CDR2, and CDR3 sequences set forth in SEQ ID NOs: 37-39, respectively.

In another embodiment, provided herein are isolated monoclonal antibodies, or antigen-binding portions thereof, which bind to FGFR1c, FGFR2c, FGFR3c, and/or FGFR4, and comprise heavy and light chain variable regions, wherein the heavy chain variable region comprises heavy chain CDR1, CDR2, and CDR3 sequences set forth in SEQ ID NOs: 1108-1110 or 1112-1114, or IMGT heavy chain CDR1, CDR2, and CDR3 sequences set forth in SEQ ID NOs: 2186-2188 or 2189-2191. In some embodiments, the antibodies do not bind to FGFR1b, FGFR2b, and/or FGFR3b. In some embodiments, the heavy chain variable region is paired with a light chain variable region comprising light chain CDR1, CDR2, and CDR3 sequences selected from the group consisting of SEQ ID NOs: 1116-1118; 1120-1122; 1124-1126; 1128-1130; 1132-1134; 1136-1138; 1140-1142; 1144-1146; 1148-1150; 1152-1154; 1156-1158; 1160-1162; and 1164-1166. In some embodiments, the heavy chain variable region is paired with a light chain variable region comprising IMGT light chain CDR1, CDR2, and CDR3 sequences selected from the group consisting of SEQ ID NOs: 2192-2194; 2195-2197; 2198-2200; 2201-2203; 2204-2206; 2207-2209; 2210-2212; 2213-2215; 2216-2218; 2219-2221; 2222-2224; 2225-2227; and 2228-2230.

In another embodiment, provided herein are isolated monoclonal antibodies, or antigen-binding portions thereof, which bind to FGFR1c, FGFR2c, FGFR3c, and/or FGFR4, and comprise heavy and light chain variable regions, wherein the light chain variable region comprises light chain CDR1, CDR2, and CDR3 sequences set forth in SEQ ID NOs: 1116-1118; 1120-1122; 1124-1126; 1128-1130; 1132-1134; 1136-1138; 1140-1142; 1144-1146; 1148-1150; 1152-1154; 1156-1158; 1160-1162; and 1164-1166, or IMGT light chain CDR1, CDR2, and CDR3 sequences set forth in SEQ ID NOs: 2192-2194; 2195-2197; 2198-2200; 2201-2203; 2204-2206; 2207-2209; 2210-2212; 2213-2215; 2216-2218; 2219-2221; 2222-2224; 2225-2227; and 2228-2230. In some embodiments, the antibodies do not bind to FGFR1b, FGFR2b, and/or FGFR3b. In some embodiments, the light chain variable region is paired with a heavy chain variable region comprising heavy chain CDR1, CDR2, and CDR3 sequences set forth in SEQ ID NOs: 1108-1110 or 1112-1114. In some embodiments, the light chain variable region is paired with a heavy chain variable region comprising IMGT heavy chain CDR1, CDR2, and CDR3 sequences set forth in SEQ ID NOs: 2186-2188 or 2189-2191.

In another embodiment, provided herein are anti-FGFR antibodies comprising heavy and light chain variable regions, wherein the heavy chain variable region comprises an amino acid sequence selected from the group consisting of SEQ ID NOs: 29; 40; 51; 62; 73; 84; 95; 106; 117; 128; 139; 150; 161; 172; 183; 194; 204; 214; 224; 234; 244; 254; 264; 274; 284; 294; 304; 314; 324; 334; 344; 354; 364; 374; 384; 394; 404; 414; 424; 434; 444; 454; 464; 474; 484; 494; 504; 514; 524; 534; 544; 554; 564; 574; 584; 594; 604; 614; 624; 634; 644; 654; 664; 674; 684; 694; 704; 714; 724; 734; 744; 754; 764; 774; 784; 794; 804; 814; 824; 834; 844; 854; 864; 874; 884 ; 894; 904; 914; 924; 934; 944; 954; 964; 974; 984; 994; 1004; 1014; 1024; 1034; 1044; 1054; 1064; 1074; 1084; 1094; 1104; 1111; and 1115, wherein the antibody specifically binds to FGFR1c, FGFR2c, FGFR3c, and/or FGFR4. In some embodiments, the antibody does not bind to FGFR1b, FGFR2b, and/or FGFR3b.

In another embodiment, provided herein are anti-FGFR antibodies comprising heavy and light chain variable regions, wherein the light chain variable region comprises an amino acid sequence selected from the group consisting of SEQ ID NOs: 30; 41; 52; 63; 74; 85; 96; 107; 118; 129; 140; 151; 162; 173; 184; 195; 205; 215; 225; 235; 245; 255; 265; 275; 285; 295; 305; 315; 325; 335; 345; 355; 365; 375; 385; 395; 405; 415; 425; 435; 445; 455; 465; 475; 485; 495; 505; 515; 525; 535; 545; 555; 565; 575; 585; 595; 605; 615; 625; 635; 645; 655; 665; 675; 685; 695; 705; 715; 725; 735; 745; 755; 765; 775; 785; 795; 805; 815; 825; 835; 845; 855; 865; 875; 885; 895; 905; 915; 925; 935; 945; 955; 965; 975; 985; 995; 1005; 1015; 1025; 1035; 1045; 1055; 1065; 1075; 1085; 1095; 1105; 1119; 1123; 1127; 1131; 1135; 1139; 1143; 1147; 1151; 1155; 1159; 1163; and 1167, wherein the antibody specifically binds to FGFR1c, FGFR2c, FGFR3c, and/or FGFR4. In some embodiments, the antibody does not bind to FGFR1b, FGFR2b, and/or FGFR3b.

In one embodiment, provided herein are anti-FGFR antibodies comprising heavy and light chain variable region sequences comprising SEQ ID NOs: 29 and 30; 40 and 41; 51 and 52; 62 and 63; 73 and 74; 84 and 85; 95 and 96; 106 and 107; 117 and 118; 128 and 129; 139 and 140; 150 and 151; 161 and 162; 172 and 173; 183 and 184; 194 and 195; 204 and 205; 214 and 215; 224 and 225; 234 and 235; 244 and 245; 254 and 255; 264 and 265; 274 and 275; 284 and 285; 294 and 295; 304 and 305; 314 and 315; 324 and 325; 334 and 335; 344 and 345; 354 and 355; 364 and 365; 374 and 375; 384 and 385; 394 and 395; 404 and 405; 414 and 415; 424 and 425; 434 and 435; 444 and 445; 454 and 455; 464 and 465; 474 and 475; 484 and 485; 494 and 495; 504 and 505; 514 and 515; 524 and 525; 534 and 535; 544 and 545; 554 and 555; 564 and 565; 574 and 575; 584 and 585; 594 and 595; 604 and 605; 614 and 615; 624 and 625; 634 and 635; 644 and 645; 654 and 655; 664 and 665; 674 and 675; 684 and 685; 694 and 695; 704 and 705; 714 and 715; 724 and 725; 734 and 735; 744 and 745; 754 and 755; 764 and 765; 774 and 775; 784 and 785; 794 and 795; 804 and 805; 814 and 815; 824 and 825; 834 and 835; 844 and 845; 854 and 855; 864 and 865; 874 and 875; 884 and 885; 894 and 895; 904 and 905; 914 and 915; 924 and 925; 934 and 935; 944 and 945; 954 and 955; 964 and 965; 974 and 975; 984 and 985; 994 and 995; 1004 and 1005; 1014 and 1015; 1024 and 1025; 1034 and 1035; 1044 and 1045; 1054 and 1055; 1064 and 1065; 1074 and 1075; 1084 and 1085; 1094 and 1095; and 1104 and 1105, wherein the antibody specifically binds to FGFR1c, FGFR2c, FGFR3c, and/or FGFR4. In some embodiments, the antibody does not bind to FGFR1b, FGFR2b, and/or FGFR3b. In this list, the first number within a pair of semicolons correspond to the heavy chain variable region sequence, and the second number following the term “and” corresponds to the light chain variable region sequence. For example, “; 40 and 41;” in the list above indicates that the antibody comprises a heavy chain variable region comprising the amino acid sequence set forth in SEQ ID NO: 40, and a light chain variable region comprising the amino acid sequence set forth in SEQ ID NO: 41.

In one embodiment, provided herein are anti-FGFR antibodies comprising or consisting of heavy and light chain sequences selected from the group consisting of SEQ ID NOs: 32 and 33; 43 and 44; 54 and 55; 65 and 66; 76 and 77; 87 and 88; 98 and 99; 109 and 110; 120 and 121; 131 and 132; 142 and 143; 153 and 154; 164 and 165; 175 and 176; 186 and 187; 196 and 197; 206 and 207; 216 and 217; 226 and 227; 236 and 237; 246 and 247; 256 and 257; 266 and 267; 276 and 277; 286 and 287; 296 and 297; 306 and 307; 316 and 317; 326 and 327; 336 and 337; 346 and 347; 356 and 357; 366 and 367; 376 and 377; 386 and 387; 396 and 397; 406 and 407; 416 and 417; 426 and 427; 436 and 437; 446 and 447; 456 and 457; 466 and 467; 476 and 477; 486 and 487; 496 and 497; 506 and 507; 516 and 517; 526 and 527; 536 and 537; 546 and 547; 556 and 557; 566 and 567; 576 and 577; 586 and 587; 596 and 597; 606 and 607; 616 and 617; 626 and 627; 636 and 637; 646 and 647; 656 and 657; 666 and 667; 676 and 677; 686 and 687; 696 and 697; 706 and 707; 716 and 717; 726 and 727; 736 and 737; 746 and 747; 756 and 757; 766 and 767; 776 and 777; 786 and 787; 796 and 797; 806 and 807; 816 and 817; 826 and 827; 836 and 837; 846 and 847; 856 and 857; 866 and 867; 876 and 877; 886 and 887; 896 and 897; 906 and 907; 916 and 917; 926 and 927; 936 and 937; 946 and 947; 956 and 957; 966 and 967; 976 and 977; 986 and 987; 996 and 997; 1006 and 1007; 1016 and 1017; 1026 and 1027; 1036 and 1037; 1046 and 1047; 1056 and 1057; 1066 and 1067; 1076 and 1077; 1086 and 1087; 1096 and 1097; and 1106 and 1107. In this list, the first number within a pair of semicolons correspond to the heavy chain sequence, and the second number following the term “and” corresponds to the light chain sequence. For example, “; 43 and 44;” in the list above indicates that the antibody comprises a heavy chain comprising or consisting of the amino acid sequence set forth in SEQ ID NO: 40, and a light chain comprising or consisting of the amino acid sequence set forth in SEQ ID NO: 41.

In certain aspects, provided herein are antibodies (e.g., isolated monoclonal antibodies), and antigen-binding portions thereof, which bind to FGFR1c, FGFR2c, FGFR3c, and/or FGFR4, and comprise CDR sequences defined by consensus sequences.

Accordingly, in one embodiment, provided herein are antibodies (e.g., isolated monoclonal antibodies), or antigen-binding portions thereof, which bind to FGFR1c, FGFR2c, FGFR3c, and/or FGFR4, and comprise heavy chain CDR1, CDR2, and CDR3 sequences [SGHT][YH]A[MI]H (SEQ ID NO: 2231), [VL]ISYDGS[NE]KYYADS[VA]KG (SEQ ID NO: 2232), and GAG[RTLQHMI]G[YLFW][TPASIM][YNFHLWRK]GPDGDFI (SEQ ID NO: 2233), respectively, and light chain CDR1, CDR2, and CDR3 sequences [TRK]SS[RQE]SL[LVI][HWYF][SRGT]DG[KNI]TY[VL][YSN] (SEQ ID NO: 2234), [EKQ][LVI]S[NS]RFS (SEQ ID NO: 2235), and MQ[YA][IVTK][EQNR][AFL]P[LW]T (SEQ ID NO: 2236), respectively.

In another embodiment, provided herein are antibodies (e.g., isolated monoclonal antibodies), or antigen-binding portions thereof, which bind to FGFR1c, FGFR2c, FGFR3c, and/or FGFR4, and comprise heavy chain CDR1, CDR2, and CDR3 sequences [SG]YA[MI]H (SEQ ID NO: 2237), [VL]ISYDGSNKYYADS[VA]KG (SEQ ID NO: 2238), and GAG[RTLQHMI]G[YLFW][TPASIM][YNFHLWRK]GPDGDFI (SEQ ID NO: 2239), respectively, and light chain CDR1, CDR2, and CDR3 sequences [TRK]SS[RQE]SL[LV][HWY][SR]DG[KN]TY[VL][YS] (SEQ ID NO: 2240), [EK][LV]SNRFS (SEQ ID NO: 2241), and MQ[YA][IVT][EQ][AF]P[LW]T (SEQ ID NO: 2242), respectively.

In another embodiment, provided herein are antibodies (e.g., isolated monoclonal antibodies), or antigen-binding portions thereof, which bind to FGFR1c, FGFR2c, FGFR3c, and/or FGFR4, and comprise heavy chain CDR1, CDR2, and CDR3 sequences [SG]YAMH (SEQ ID NO: 2243), VISYDGSNKYYADSVKG (SEQ ID NO: 2244), and GAG[RTLQHMI]G[YLFW][TPASIM][YNFHLWRK]GPDGDFI (SEQ ID NO: 2245), respectively, and light chain CDR1, CDR2, and CDR3 sequences [TRK]SS[RQ]SLL[HW]SDGKTY[VL]Y (SEQ ID NO: 2246), ELSNRFS (SEQ ID NO: 2247), and MQY[IV]EAPLT (SEQ ID NO: 2248), respectively.

In another embodiment, provided herein are antibodies (e.g., isolated monoclonal antibodies), or antigen-binding portions thereof, which bind to FGFR1c, FGFR2c, FGFR3c, and/or FGFR4, and comprise IMGT heavy chain CDR1, CDR2, and CDR3 sequences GF[TSD]F[SGTA][SGHT][YH]A (SEQ ID NO: 2249), ISYDGS[NE]K (SEQ ID NO: 2250), and VRGAG[RTLQHMI]G[YLFW][TPASIM][YNFHLWRK]GPDGDFI (SEQ ID NO: 2251), respectively, and IMGT light chain CDR1, CDR2, and CDR3 sequences [RQE]SL[LVI][HWYF][SRGT]DG[KNI]TY (SEQ ID NO: 2252), [EKQ][LVI]S (SEQ ID NO: 2253), and MQ[YA][IVTK][EQNR][AFL]P[LW]T (SEQ ID NO: 2254), respectively.

In another embodiment, provided herein are antibodies (e.g., isolated monoclonal antibodies), or antigen-binding portions thereof, which bind to FGFR1c, FGFR2c, FGFR3c, and/or FGFR4, and comprise IMGT heavy chain CDR1, CDR2, and CDR3 sequences GF[TD]F[SA][SG]YA (SEQ ID NO: 2255), ISYDGSNK (SEQ ID NO: 2256), and VRGAG[RTLQHMI]G[YLFW][TPASIM][YNFHLWRK]GPDGDFI (SEQ ID NO: 2257), respectively, and IMGT light chain CDR1, CDR2, and CDR3 sequences [RQE]SL[LV][HWY][SR]DG[KN]TY (SEQ ID NO: 2258), [EK][LV]S (SEQ ID NO: 2259), and MQ[YA][IVT][EQ][AF]P[LW]T (SEQ ID NO: 2260), respectively.

In another embodiment, provided herein are antibodies (e.g., isolated monoclonal antibodies), or antigen-binding portions thereof, which bind to FGFR1c, FGFR2c, FGFR3c, and/or FGFR4, and comprise IMGT heavy chain CDR1, CDR2, and CDR3 sequences GF[TD]F[SA][SG]YA (SEQ ID NO: 2261), ISYDGSNK (SEQ ID NO: 2262), and VRGAG[RTLQHMI]G[YLFW][TPASIM][YNFHLWRK]GPDGDFI (SEQ ID NO: 2263), respectively, and IMGT light chain CDR1, CDR2, and CDR3 sequences [RQ]SLL[HW]SDGKTY (SEQ ID NO: 2264), ELS (SEQ ID NO: 2265), and MQY[IV]EAPLT (SEQ ID NO: 2266), respectively.

In some embodiments, the VH domain of Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, Ab36, Ab37, Ab38, Ab39, Ab40, Ab41, Ab42, Ab43, Ab44, Ab45, Ab46, Ab47, Ab48, Ab49, Ab50, Ab51, Ab52, Ab53, Ab54, Ab55, Ab56, Ab57, Ab58, Ab59, Ab60, Ab61, Ab62, Ab63, Ab64, Ab65, Ab66, Ab67, Ab68, Ab69, Ab70, Ab71, Ab72, Ab73, Ab74, Ab75, Ab76, Ab77, Ab78, Ab79, Ab80, Ab81, Ab82, Ab83, Ab84, Ab85, Ab86, Ab87, Ab88, Ab89, Ab90, Ab91, Ab92, Ab93, Ab94, Ab95, Ab96, Ab97, Ab98, Ab99, Ab100, Ab101, Ab102, Ab103, Ab104, Ab105, Ab106, or Ab107 (the sequences of which are provided in Table 9) is combined with Vk1 (SEQ ID NO: 1119), Vk2 (SEQ ID NO: 1123), Vk3 (SEQ ID NO: 1127), Vk4 (SEQ ID NO: 1131), Vk5 (SEQ ID NO: 1135), Vk6 (SEQ ID NO: 1139), Vk7 (SEQ ID NO: 1143), Vk8 (SEQ ID NO: 1147), Vk9 (SEQ ID NO: 1151), Vk10 (SEQ ID NO: 1155), Vk11 (SEQ ID NO: 1159), Vk12 (SEQ ID NO: 1163), or Vk13 (SEQ ID NO: 1167) to form an anti-FGFR antibody. All combinations are contemplated and are herein referred to as “VH of any of Ab1-Ab107 and any of Vk1-12” or “VH of any of Ab1-Ab107 combined with any of Vk1-12.”

In some embodiments, the VL domain of any of Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab22, Ab23, Ab24, Ab25, Ab26, Ab27, Ab28, Ab29, Ab30, Ab31, Ab32, Ab33, Ab34, Ab35, Ab36, Ab37, Ab38, Ab39, Ab40, Ab41, Ab42, Ab43, Ab44, Ab45, Ab46, Ab47, Ab48, Ab49, Ab50, Ab51, Ab52, Ab53, Ab54, Ab55, Ab56, Ab57, Ab58, Ab59, Ab60, Ab61, Ab62, Ab63, Ab64, Ab65, Ab66, Ab67, Ab68, Ab69, Ab70, Ab71, Ab72, Ab73, Ab74, Ab75, Ab76, Ab77, Ab78, Ab79, Ab80, Ab81, Ab82, Ab83, Ab84, Ab85, Ab86, Ab87, Ab88, Ab89, Ab90, Ab91, Ab92, Ab93, Ab94, Ab95, Ab96, Ab97, Ab98, Ab99, Ab100, Ab101, Ab102, Ab103, Ab104, Ab105, Ab106, and Ab107 (the sequences of which are provided in Table 9) is combined with Vh1 (SEQ ID NO: 1111) or Vh2 (SEQ ID NO: 1115) to form an anti-FGFR antibody. All combinations of Ab1-Ab107 and Vh1 or Vh2 are contemplated, and are herein referred to as “Vh1 or Vh2 and VL of any of Ab1-Ab107” or “Vh1 or Vh2 combined with VL of any of Ab1-Ab107.”

In some embodiments, a VH domain described herein is linked to a constant domain to form a heavy chain, e.g., a full-length heavy chain. In some embodiments, the VH domain is linked to the constant domain of a human IgG, e.g., IgG1, IgG2, IgG3, or IgG4, or variants thereof. Similarly, a VL domain described herein is linked to a constant domain to form a light chain, e.g., a full-length light chain.

Also provided herein are anti-FGFR antibodies that compete for binding to FGFR proteins, e.g., FGFR1c, FGFR2c, FGFR3c, and/or FGFR4, with anti-FGFR antibodies comprising CDRs or variable regions described herein, e.g., those of any of Ab1-Ab107. In some embodiments, anti-FGFR antibodies inhibit binding of any of Ab1-Ab107 to human FGFR1c, FGFR2c, FGFR3c, and/or FGFR4 by at least 10%, 20%, 30%, 40%, 50%, 60%, 70%, 80%, 90% or by 100%. Competing antibodies can be identified based on their ability to competitively inhibit binding to FGFR proteins using standard binding assays known in the art (e.g., competitive ELISA assay).

Also provided herein are anti-FGFR antibodies which bind to the same epitope on FGFR1c, FGFR2c, FGFR3c, and/or FGFR4 with anti-FGFR antibodies comprising CDRs or variable regions described herein, e.g., those of any of Ab1-Ab107. Methods for determining whether antibodies bind to the same epitope on FGFR1c, FGFR2c, FGFR3c, and/or FGFR4 with the antibodies described herein include, for example, epitope mapping methods, monitoring the binding of the antibody to antigen fragments or mutated variations of the antigen where loss of binding due to a modification of an amino acid residue within the antigen sequence is considered an indication of an epitope component (e.g., alanine scanning); MS-based protein footprinting, and assessing the ability of an antibody of interest to affinity isolate specific short peptides (either in native three dimensional form or in denatured form) from combinatorial phage display peptide libraries.

Antibodies disclosed herein include all known forms of antibodies and other protein scaffolds with antibody-like properties. For example, the antibody can be a human antibody, a humanized antibody, a bispecific antibody, an immunoconjugate, a chimeric antibody, or a protein scaffold with antibody-like properties, such as fibronectin or ankyrin repeats. The antibody also can be a Fab, Fab′2, scFv, affibody®, avimer, nanobody, or a domain antibody. The antibody also can have any isotype, including any of the following isotypes: IgG1, IgG2, IgG3, IgG4, IgM, IgAl, IgA2, IgAsec, IgD, and IgE. IgG antibodies are preferred. Full-length antibodies can be prepared from V_{H} and V_{L} sequences using standard recombinant DNA techniques and nucleic acid encoding the desired constant region sequences to be operatively linked to the variable region sequences.

In various embodiments, the antibodies described above exhibit one or more (e.g., 1, 2, 3, 4, 5, or 6) of the following functional properties:

(a) binds to human FGFR1c, FGFR2c, FGFR3c, and/or FGFR4, e.g., with a K_{D} of 10⁻⁷ or less, 10⁻⁸ or less, or 10⁻⁹ or less for FGFR1c; 10⁻⁷ or less, 10⁻⁸ or less, or 10⁻⁹ or less for FGFR2c; 10⁻⁷ or less, 10⁻⁸ or less, or 10⁻⁹ or less for FGFR3c; and/or 10⁻⁷ or less, 10⁻⁸ or less, or 10⁻⁹ or less for FGFR4 when in IgG2 format (e.g., wild-type IgG2 constant region or a variant such as M7 (SEQ ID NO: 1173), e.g., as measured by bio-layer interferometry (e.g., the ForteBio assay described in Example 9);

(b) binds to human FGFR1c, FGFR2c, FGFR3c, and/or FGFR4, e.g., with an EC50 of, for example, 10 nM or less, 1 nM or less, or 0.05 nM or less for FGFR1c; 10 nM or less, 1 nM or less, or 0.05 nM or less for FGFR2c; 10 nM or less, 1 nM or less, or 0.5 nM or less for FGFR3c; and/or 50 nM or less, 25 nM or less, or 10 nM or less for FGFR4 when in IgG2 format (e.g., wild-type IgG2 constant region or a variant such as M7 (SEQ ID NO: 1173), e.g., as measured by ELISA (see, e.g., Examples 3 and 9);

(c) does not bind to human FGFR1b, FGFR2b, and/or FGFR3b , as measured by ELISA or bio-layer interferometry;

(d) inhibits the binding of FGF1 to FGFR1c, FGFR2c, FGFR3c, and/or FGFR4, e.g., with an EC50 of 200 nM or less, 150 nM or less, 50 nM or less, 25 nM or less, or 10 nM or less, e.g., as measured by ELISA (see, e.g., Example 9);

(e) inhibits FGF2-mediated phosphorylation of ERK, e.g., with an IC₅₀ of 50 or less, 25 nM or less, or 10 nM or less, as measured by SureFire Alpha assay (see, e.g., Example 7); and

(f) inhibits FGF2-mediated cell viability, e.g., with an IC₅₀ of 100 nM or less, 75 nM or less, 50 nM or less, 25 nM or less, or 10 nM or less, as assessed with CellTiterGlo (see Example 8).

Also provided herein is an antibody composition comprising one or more anti-FGFR antibodies which collectively bind to FGFR1c, FGFR2c, FGFR3c, and FGFR4, but not FGFR1b, FGFR2b, and/or FGFR3b. In one embodiment, the antibody composition comprises one or more anti-FGFR antibodies which collectively bind to FGFR1c, FGFR2c, FGFR3c, and FGFR4, but not FGFR1b, FGFR2b, and FGFR3b. In another embodiment, the antibody composition includes four antibodies, each of which bind to one of FGFR1c, FGFR2c, FGFR3c, and FGFR4, wherein none of the four antibodies bind to FGFR1b, FGFR2b, and/or FGFR3b. In another embodiment, the antibody composition includes three antibodies which collectively bind to FGFR1c, FGFR2c, FGFR3c, and FGFR4, wherein none of the three antibodies bind to FGFR1b, FGFR2b, and/or FGFR3b. In another embodiment, the antibody composition includes two antibodies which collectively bind to FGFR1c, FGFR2c, FGFR3c, and FGFR4, wherein neither of the antibodies bind to FGFR1b, FGFR2b, and/or FGFR3b. In another embodiment, the antibody composition includes an antibody which binds to all four of FGFR1c, FGFR2c, FGFR3c, and FGFR4, but does not bind to FGFR1b, FGFR2b, and/or FGFR3b.

Antibodies with Altered Sequence

In some embodiments, the variable region sequences, or portions thereof, of the anti-FGFR antibodies described herein are altered to create structurally-related anti-FGFR antibodies (i.e., altered antibodies) that retain binding and thus are functionally equivalent.

For example, amino acid residues within the V_{H} and/or V_{L} CDR1, CDR2 and/or CDR3 regions can be mutated to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Preferably conservative modifications (as discussed above) are introduced. The mutations may be amino acid substitutions, additions or deletions, but are preferably substitutions. Typically no more than one, two, three, four or five residues within a CDR region are altered.

Accordingly, in some embodiments, provided are anti-FGFR antibodies comprising VHCDR1, VHCDR2, VHCDR3, VLCDR1, VLCDR2, and/or VLCDR3 that differs from the corresponding CDR(s) of any of Ab1-Ab107 by 1, 2, 3, 4, 5, 1-2, 1-3, 1-4, or 1-5 amino acid changes (i.e., amino acid substitutions, additions, or deletions). In one embodiment, an anti-FGFR antibody comprises a total of 1-5 amino acid changes across all CDRs relative to the CDRs of any of Ab1-Ab107. In another embodiment, the anti-FGFR antibody comprises 1-5 amino acid changes in each of 6 CDRs relative to the corresponding CDRs of any of Ab1-Ab107. These altered antibodies can be tested, using the in vitro and in vivo assays described herein and in the Examples, to determine whether they retain one or more (e.g., 1, 2, 3, 4, 5, or 6) of the following properties:

(a) binds to human FGFR1c, FGFR2c, FGFR3c, and/or FGFR4, e.g., with a K_{D} of 10⁻⁷ or less, 10⁻⁸ or less, or 10⁻⁹ or less for FGFR1c; 10⁻⁷ or less, 10⁻⁸ or less, or 10⁻⁹ or less for FGFR2c; 10⁻⁷ or less, 10⁻⁸ or less, or 10⁻⁹ or less for FGFR3c; and/or 10⁻⁷ or less, 10⁻⁸ or less, or 10⁻⁹ or less for FGFR4 when in IgG2 format (e.g., wild-type IgG2 constant region or a variant such as M7 (SEQ ID NO: 1173), e.g., as measured by bio-layer interferometry (e.g., the ForteBio assay described in Example 9);

(b) binds to human FGFR1c, FGFR2c, FGFR3c, and/or FGFR4, e.g., with an EC50 of, for example, 10 nM or less, 1 nM or less, or 0.05 nM or less for FGFR1c; 10 nM or less, 1 nM or less, or 0.05 nM or less for FGFR2c; 10 nM or less, 1 nM or less, or 0.5 nM or less for FGFR3c; and/or 50 nM or less, 25 nM or less, or 10 nM or less for FGFR4 when in IgG2 format (e.g., wild-type IgG2 constant region or a variant such as M7 (SEQ ID NO: 1173), e.g., as measured by ELISA (see, e.g., Examples 3 and 9);

(c) does not bind to human FGFR1b, FGFR2b, and/or FGFR3b, as measured by ELISA or bio-layer interferometry;

(d) inhibits the binding of FGF1 to FGFR1c, FGFR2c, FGFR3c, and/or FGFR4, e.g., with an EC50 of 200 nM or less, 150 nM or less, 50 nM or less, 25 nM or less, or 10 nM or less, e.g., as measured by ELISA (see, e.g., Example 9);

(e) inhibits FGF2-mediated phosphorylation of ERK, e.g., with an IC₅₀ of 50 or less, 25 nM or less, or 10 nM or less, as measured by SureFire Alpha assay (see Example 7); and

(f) inhibits FGF2-mediated cell viability, e.g., with an IC₅₀ of 100 nM or less, 75 nM or less, 50 nM or less, 25 nM or less, or 10 nM or less, as assessed with CellTiterGlo (see, e.g., Example 8).

In some embodiments, provided are anti-FGFR antibodies comprising heavy and light chain variable regions, wherein the heavy chain variable region comprises an amino acid sequence which is at least 80%, 85%, 90%, 95%, 96%, 97%, 98%, or 99% identical to an amino acid sequence selected from the group consisting of SEQ ID NOs: 29; 40; 51; 62; 73; 84; 95; 106; 117; 128; 139; 150; 161; 172; 183; 194; 204; 214; 224; 234; 244; 254; 264; 274; 284; 294; 304; 314; 324; 334; 344; 354; 364; 374; 384; 394; 404; 414; 424; 434; 444; 454; 464; 474; 484; 494; 504; 514; 524; 534; 544; 554; 564; 574; 584; 594; 604; 614; 624; 634; 644; 654; 664; 674; 684; 694; 704; 714; 724; 734; 744; 754; 764; 774; 784; 794; 804; 814; 824; 834; 844; 854; 864; 874; 884 ; 894; 904; 914; 924; 934; 944; 954; 964; 974; 984; 994; 1004; 1014; 1024; 1034; 1044; 1054; 1064; 1074; 1084; 1094; 1104; 1111; and 1115, or comprises 1, 2, 3, 4, 5, 1-2, 1-3, 1-4, 1-5, 1-10, 1-15, 1-20, 1-25, or 1-50 amino acid changes relative to the amino acid sequence selected from the group consisting of SEQ ID NOs: 29; 40; 51; 62; 73; 84; 95; 106; 117; 128; 139; 150; 161; 172; 183; 194; 204; 214; 224; 234; 244; 254; 264; 274; 284; 294; 304; 314; 324; 334; 344; 354; 364; 374; 384; 394; 404; 414; 424; 434; 444; 454; 464; 474; 484; 494; 504; 514; 524; 534; 544; 554; 564; 574; 584; 594; 604; 614; 624; 634; 644; 654; 664; 674; 684; 694; 704; 714; 724; 734; 744; 754; 764; 774; 784; 794; 804; 814; 824; 834; 844; 854; 864; 874; 884 ; 894; 904; 914; 924; 934; 944; 954; 964; 974; 984; 994; 1004; 1014; 1024; 1034; 1044; 1054; 1064; 1074; 1084; 1094; 1104; 1111; and 1115, wherein the antibodies specifically bind to human FGFR1c, FGFR2c, FGFR3c, and/or FGFR4 and retain one or more (e.g., 1, 2, 3, 4, 5, or 6) of the following properties:

(a) binds to human FGFR1c, FGFR2c, FGFR3c, and/or FGFR4, e.g., with a K_{D} of 10⁻⁷ or less, 10⁻⁸ or less, or 10⁻⁹ or less for FGFR1c; 10⁻⁷ or less, 10⁻⁸ or less, or 10⁻⁹ or less for FGFR2c; 10⁻⁷ or less, 10⁻⁸ or less, or 10⁻⁹ or less for FGFR3c; and/or 10⁻⁷ or less, 10⁻⁸ or less, or 10⁻⁹ or less for FGFR4 when in IgG2 format (e.g., wild-type IgG2 constant region or a variant such as M7 (SEQ ID NO: 1173), e.g., as measured by bio-layer interferometry (e.g., the ForteBio assay described in Example 9);

(b) binds to human FGFR1c, FGFR2c, FGFR3c, and/or FGFR4, e.g., with an EC50 of, for example, 10 nM or less, 1 nM or less, or 0.05 nM or less for FGFR1c; 10 nM or less, 1 nM or less, or 0.05 nM or less for FGFR2c; 10 nM or less, 1 nM or less, or 0.5 nM or less for FGFR3c; and/or 50 nM or less, 25 nM or less, or 10 nM or less for FGFR4 when in IgG2 format (e.g., wild-type IgG2 constant region or a variant such as M7 (SEQ ID NO: 1173), e.g., as measured by ELISA (see, e.g., Examples 3 and 9);

(c) does not bind to human FGFR1b, FGFR2b, and/or FGFR3b, as measured by ELISA or bio-layer interferometry;

(d) inhibits the binding of FGF1 to FGFR1c, FGFR2c, FGFR3c, and/or FGFR4, e.g., with an EC50 of 200 nM or less, 150 nM or less, 50 nM or less, 25 nM or less, or 10 nM or less, e.g., as measured by ELISA (see, e.g., Example 9);

(e) inhibits FGF2-mediated phosphorylation of ERK, e.g., with an IC₅₀ of 50 or less, 25 nM or less, or 10 nM or less, as measured by SureFire Alpha assay (see, e.g., Example 7); and

(f) inhibits FGF2-mediated cell viability, e.g., with an IC₅₀ of 100 nM or less, 75 nM or less, 50 nM or less, 25 nM or less, or 10 nM or less, as assessed with CellTiterGlo (see, e.g., Example 8).

In some embodiments, provided are anti-FGFR antibodies comprising heavy and light chain variable regions, wherein the light chain variable region comprises an amino acid sequence which is at least 80%, 85%, 90%, 95%, 96%, 97%, 98%, or 99% identical to an amino acid sequence selected from the group consisting of SEQ ID NOs: 30; 41; 52; 63; 74; 85; 96; 107; 118; 129; 140; 151; 162; 173; 184; 195; 205; 215; 225; 235; 245; 255; 265; 275; 285; 295; 305; 315; 325; 335; 345; 355; 365; 375; 385; 395; 405; 415; 425; 435; 445; 455; 465; 475; 485; 495; 505; 515; 525; 535; 545; 555; 565; 575; 585; 595; 605; 615; 625; 635; 645; 655; 665; 675; 685; 695; 705; 715; 725; 735; 745; 755; 765; 775; 785; 795; 805; 815; 825; 835; 845; 855; 865; 875; 885; 895; 905; 915; 925; 935; 945; 955; 965; 975; 985; 995; 1005; 1015; 1025; 1035; 1045; 1055; 1065; 1075; 1085; 1095; 1105; 1119; 1123; 1127; 1131; 1135; 1139; 1143; 1147; 1151; 1155; 1159; 1163; and 1167, or comprises 1, 2, 3, 4, 5, 1-2, 1-3, 1-4, 1-5, 1-10, 1-15, 1-20, 1-25, or 1-50 amino acid changes relative to the amino acid sequence selected from the group consisting of SEQ ID NOs: 30; 41; 52; 63; 74; 85; 96; 107; 118; 129; 140; 151; 162; 173; 184; 195; 205; 215; 225; 235; 245; 255; 265; 275; 285; 295; 305; 315; 325; 335; 345; 355; 365; 375; 385; 395; 405; 415; 425; 435; 445; 455; 465; 475; 485; 495; 505; 515; 525; 535; 545; 555; 565; 575; 585; 595; 605; 615; 625; 635; 645; 655; 665; 675; 685; 695; 705; 715; 725; 735; 745; 755; 765; 775; 785; 795; 805; 815; 825; 835; 845; 855; 865; 875; 885; 895; 905; 915; 925; 935; 945; 955; 965; 975; 985; 995; 1005; 1015; 1025; 1035; 1045; 1055; 1065; 1075; 1085; 1095; 1105; 1119; 1123; 1127; 1131; 1135; 1139; 1143; 1147; 1151; 1155; 1159; 1163; and 1167, wherein the antibodies specifically bind to FGFR1c, FGFR2c, FGFR3c, and/or FGFR4 and retain one or more (e.g., 1, 2, 3, 4, 5, or 6) of the following properties:

(a) binds to human FGFR1c, FGFR2c, FGFR3c, and/or FGFR4, e.g., with a K_{D} of 10⁻⁷ or less, 10⁻⁸ or less, or 10⁻⁹ or less for FGFR1c; 10⁻⁷ or less, 10⁻⁸ or less, or 10⁻⁹ or less for FGFR2c; 10⁻⁷ or less, 10⁻⁸ or less, or 10⁻⁹ or less for FGFR3c; and/or 10⁻⁷ or less, 10⁻⁸ or less, or 10 or less for FGFR4 when in IgG2 format (e.g., wild-type IgG2 constant region or a variant such as M7 (SEQ ID NO: 1173), e.g., as measured by bio-layer interferometry (e.g., the ForteBio assay described in Example 9);

(b) binds to human FGFR1c, FGFR2c, FGFR3c, and/or FGFR4, e.g., with an EC50 of, for example, 10 nM or less, 1 nM or less, or 0.05 nM or less for FGFR1c; 10 nM or less, 1 nM or less, or 0.05 nM or less for FGFR2c; 10 nM or less, 1 nM or less, or 0.5 nM or less for FGFR3c; and/or 50 nM or less, 25 nM or less, or 10 nM or less for FGFR4 when in IgG2 format (e.g., wild-type IgG2 constant region or a variant such as M7 (SEQ ID NO: 1173), e.g., as measured by ELISA (see, e.g., Examples 3 and 9);

(c) does not bind to human FGFR1b, FGFR2b, and/or FGFR3b, as measured by ELISA or bio-layer interferometry;

(d) inhibits the binding of FGF1 to FGFR1c, FGFR2c, FGFR3c, and/or FGFR4, e.g., with an EC50 of 200 nM or less, 150 nM or less, 50 nM or less, 25 nM or less, or 10 nM or less, e.g., as measured by ELISA (see, e.g., Example 9);

(e) inhibits FGF2-mediated phosphorylation of ERK, e.g., with an IC50 of 50 or less, 25 nM or less, or 10 nM or less, as measured by SureFire Alpha assay (see, e.g., Example 7); and

(f) inhibits FGF2-mediated cell viability, e.g., with an IC₅₀ of 100 nM or less, 75 nM or less, 50 nM or less, 25 nM or less, or 10 nM or less, as assessed with CellTiterGlo (see, e.g., Example 8).

In some embodiments, provided are anti-FGFR antibodies comprising heavy and light chain variable regions, wherein the antibodies comprise heavy and light chain variable region sequences which are at least 80%, 85%, 90%, 95%, 96%, 97%, 98%, or 99% identical to the amino acid sequences selected from the group consisting of SEQ ID NOs: 29 and 30; 40 and 41; 51 and 52; 62 and 63; 73 and 74; 84 and 85; 95 and 96; 106 and 107; 117 and 118; 128 and 129; 139 and 140; 150 and 151; 161 and 162; 172 and 173; 183 and 184; 194 and 195; 204 and 205; 214 and 215; 224 and 225; 234 and 235; 244 and 245; 254 and 255; 264 and 265; 274 and 275; 284 and 285; 294 and 295; 304 and 305; 314 and 315; 324 and 325; 334 and 335; 344 and 345; 354 and 355; 364 and 365; 374 and 375; 384 and 385; 394 and 395; 404 and 405; 414 and 415; 424 and 425; 434 and 435; 444 and 445; 454 and 455; 464 and 465; 474 and 475; 484 and 485; 494 and 495; 504 and 505; 514 and 515; 524 and 525; 534 and 535; 544 and 545; 554 and 555; 564 and 565; 574 and 575; 584 and 585; 594 and 595; 604 and 605; 614 and 615; 624 and 625; 634 and 635; 644 and 645; 654 and 655; 664 and 665; 674 and 675; 684 and 685; 694 and 695; 704 and 705; 714 and 715; 724 and 725; 734 and 735; 744 and 745; 754 and 755; 764 and 765; 774 and 775; 784 and 785; 794 and 795; 804 and 805; 814 and 815; 824 and 825; 834 and 835; 844 and 845; 854 and 855; 864 and 865; 874 and 875; 884 and 885; 894 and 895; 904 and 905; 914 and 915; 924 and 925; 934 and 935; 944 and 945; 954 and 955; 964 and 965; 974 and 975; 984 and 985; 994 and 995; 1004 and 1005; 1014 and 1015; 1024 and 1025; 1034 and 1035; 1044 and 1045; 1054 and 1055; 1064 and 1065; 1074 and 1075; 1084 and 1085; 1094 and 1095; and 1104 and 1105,wherein the antibodies specifically bind to human FGFR1c, FGFR2c, FGFR3c, and/or FGFR4 and retain one or more (e.g., 1, 2, 3, 4, 5, or 6) of the following properties:

(a) binds to human FGFR1c, FGFR2c, FGFR3c, and/or FGFR4, e.g., with a K_{D} of 10⁻⁷ or less, 10⁻⁸ or less, or 10⁻⁹ or less for FGFR1c; 10⁻⁷ or less, 10⁻⁸ or less, or 10⁻⁹ or less for FGFR2c; 10⁻⁷ or less, 10⁻⁸ or less, or 10⁻⁹ or less for FGFR3c; and/or 10⁻⁷ or less, 10⁻⁸ or less, or 10 or less for FGFR4 when in IgG2 format (e.g., wild-type IgG2 constant region or a variant such as M7 (SEQ ID NO: 1173)), e.g., as measured by bio-layer interferometry (e.g., the ForteBio assay described in Example 9);

(b) binds to human FGFR1c, FGFR2c, FGFR3c, and/or FGFR4, e.g., with an EC50 of, for example, 10 nM or less, 1 nM or less, or 0.05 nM or less for FGFR1c; 10 nM or less, 1 nM or less, or 0.05 nM or less for FGFR2c; 10 nM or less, 1 nM or less, or 0.5 nM or less for FGFR3c; and/or 50 nM or less, 25 nM or less, or 10 nM or less for FGFR4 when in IgG2 format (e.g., wild-type IgG2 constant region or a variant such as M7 (SEQ ID NO: 1173), e.g., as measured by ELISA (see, e.g., Examples 3 and 9);

(c) does not bind to human FGFR1b, FGFR2b, and/or FGFR3b, as measured by ELISA or bio-layer interferometry;

(d) inhibits the binding of FGF1 to FGFR1c, FGFR2c, FGFR3c, and/or FGFR4, e.g., with an EC50 of 200 nM or less, 150 nM or less, 50 nM or less, 25 nM or less, or 10 nM or less, e.g., as measured by ELISA (see, e.g., Example 9);

(e) inhibits FGF2-mediated phosphorylation of ERK, e.g., with an IC₅₀ of 50 or less, 25 nM or less, or 10 nM or less, as measured by SureFire Alpha assay (see, e.g., Example 7); and

(f) inhibits FGF2-mediated cell viability, e.g., with an IC₅₀ of 100 nM or less, 75 nM or less, 50 nM or less, 25 nM or less, or 10 nM or less, as assessed with CellTiterGlo (see, e.g., Example 8).

In some embodiments, provided are anti-FGFR antibodies comprising heavy and light chains, wherein the heavy chain comprises an amino acid sequence which is at least 80%, 85%, 90%, 95%, 96%, 97%, 98%, or 99% identical to an amino acid sequence selected from the group consisting of SEQ ID NOs: 32; 43; 54; 65; 76; 87; 98; 109; 120; 131; 142; 153; 164; 175; 186; 196; 206; 216; 226; 236; 246; 256; 266; 276; 286; 296; 306; 316; 326; 336; 346; 356; 366; 376; 386; 396; 406; 416; 426; 436; 446; 456; 466; 476; 486; 496; 506 516; 526; 536; 546; 556; 566; 576; 586; 596; 606; 616; 626; 636; 646; 656; 666; 676; 686; 696; 706; 716; 726; 736; 746; 756; 766; 776; 786; 796; 806; 816; 826; 836; 846; 856; 866; 876; 886; 896; 906; 916; 926; 936; 946; 956; 966; 976; 986; 996; 1006; 1016; 1026; 1036; 1046; 1056; 1066; 1076; 1086; 1096; and 1106, or comprises 1, 2, 3, 4, 5, 1-2, 1-3, 1-4, 1-5, 1-10, 1-15, 1-20, 1-25, or 1-50 amino acid changes relative to the amino acid sequence selected from the group consisting of SEQ ID NOs: 32; 43; 54; 65; 76; 87; 98; 109; 120; 131; 142; 153; 164; 175; 186; 196; 206; 216; 226; 236; 246; 256; 266; 276; 286; 296; 306; 316; 326; 336; 346; 356; 366; 376; 386; 396; 406; 416; 426; 436; 446; 456; 466; 476; 486; 496; 506 516; 526; 536; 546; 556; 566; 576; 586; 596; 606; 616; 626; 636; 646; 656; 666; 676; 686; 696; 706; 716; 726; 736; 746; 756; 766; 776; 786; 796; 806; 816; 826; 836; 846; 856; 866; 876; 886; 896; 906; 916; 926; 936; 946; 956; 966; 976; 986; 996; 1006; 1016; 1026; 1036; 1046; 1056; 1066; 1076; 1086; 1096; and 1106, wherein the antibodies specifically bind to human FGFR1c, FGFR2c, FGFR3c, and/or FGFR4 and retain one or more (e.g., 1, 2, 3, 4, 5, or 6) of the following properties:

(a) binds to human FGFR1c, FGFR2c, FGFR3c, and/or FGFR4, e.g., with a K_{D} of 10⁻⁷ or less, 10⁻⁸ or less, or 10⁻⁹ or less for FGFR1c; 10⁻⁷ or less, 10⁻⁸ or less, or 10⁻⁹ or less for FGFR2c; 10⁻⁷ or less, 10⁻⁸ or less, or 10⁻⁹ or less for FGFR3c; and/or 10⁻⁷ or less, 10⁻⁸ or less, or 10 or less for FGFR4 when in IgG2 format (e.g., wild-type IgG2 constant region or a variant such as M7 (SEQ ID NO: 1173)), e.g., as measured by bio-layer interferometry (e.g., the ForteBio assay described in Example 9);

(b) binds to human FGFR1c, FGFR2c, FGFR3c, and/or FGFR4, e.g., with an EC50 of, for example, 10 nM or less, 1 nM or less, or 0.05 nM or less for FGFR1c; 10 nM or less, 1 nM or less, or 0.05 nM or less for FGFR2c; 10 nM or less, 1 nM or less, or 0.5 nM or less for FGFR3c; and/or 50 nM or less, 25 nM or less, or 10 nM or less for FGFR4 when in IgG2 format (e.g., wild-type IgG2 constant region or a variant such as M7 (SEQ ID NO: 1173)), e.g., as measured by ELISA (see, e.g., Examples 3 and 9);

(c) does not bind to human FGFR1b, FGFR2b, and/or FGFR3b , as measured by ELISA or bio-layer interferometry;

(d) inhibits the binding of FGF1 to FGFR1c, FGFR2c, FGFR3c, and/or FGFR4, e.g., with an EC50 of 200 nM or less, 150 nM or less, 50 nM or less, 25 nM or less, or 10 nM or less, e.g., as measured by ELISA (see, e.g., Example 9);

(e) inhibits FGF2-mediated phosphorylation of ERK, e.g., with an IC₅₀ of 50 or less, 25 nM or less, or 10 nM or less, as measured by SureFire Alpha assay (see, e.g., Example 7); and

(f) inhibits FGF2-mediated cell viability, e.g., with an IC₅₀ of 100 nM or less, 75 nM or less, 50 nM or less, 25 nM or less, or 10 nM or less, as assessed with CellTiterGlo (see, e.g., Example 8).

In some embodiments, provided are anti-FGFR antibodies comprising heavy and light chains, wherein the light chain comprises an amino acid sequence which is at least 80%, 85%, 90%, 95%, 96%, 97%, 98%, or 99% identical to an amino acid sequence selected from the group consisting of SEQ ID NOs: 33; 44; 55; 66; 77; 88; 99; 110; 121; 132; 143; 154; 165; 176; 186; 196; 206; 216; 226; 236; 246; 256; 266; 277; 287; 297; 307; 317; 327; 337; 347; 357; 367; 377; 387; 397; 407; 417; 427; 437; 447; 457; 467; 477; 487; 497; 507; 517; 527; 537; 547; 557; 567; 577; 587; 597; 607; 617; 627; 637; 647; 657; 667; 677; 687; 697; 707; 717; 727; 737; 747 757; 767; 777; 787; 797; 807; 817; 827; 837; 847; 857; 867; 877; 887; 897; 907; 917; 927; 937; 947; 957; 967; 977; 987; 997; 1007; 1017; 1027; 1037; 1047; 1057; 1067; 1077; 1087; 1097; and 1107, or comprises 1, 2, 3, 4, 5, 1-2, 1-3, 1-4, 1-5, 1-10, 1-15, 1-20, 1-25, or 1-50 amino acid changes relative to the amino acid sequence selected from the group consisting of SEQ ID NOs: 33; 44; 55; 66; 77; 88; 99; 110; 121; 132; 143; 154; 165; 176; 186; 196; 206; 216; 226; 236; 246; 256; 266; 277; 287; 297; 307; 317; 327; 337; 347; 357; 367; 377; 387; 397; 407; 417; 427; 437; 447; 457; 467; 477; 487; 497; 507; 517; 527; 537; 547; 557; 567; 577; 587; 597; 607; 617; 627; 637; 647; 657; 667; 677; 687; 697; 707; 717; 727; 737; 747 757; 767; 777; 787; 797; 807; 817; 827; 837; 847; 857; 867; 877; 887; 897; 907; 917; 927; 937; 947; 957; 967; 977; 987; 997; 1007; 1017; 1027; 1037; 1047; 1057; 1067; 1077; 1087; 1097; and 1107, wherein the antibodies specifically bind to FGFR1c, FGFR2c, FGFR3c, and/or FGFR4 and retain one or more (e.g., 1, 2, 3, 4, 5, or 6) of the following properties:

(a) binds to human FGFR1c, FGFR2c, FGFR3c, and/or FGFR4, e.g., with a K_{D} of 10⁻⁷ or less, 10⁻⁸ or less, or 10⁻⁹ or less for FGFR1c; 10⁻⁷ or less, 10⁻⁸ or less, or 10⁻⁹ or less for FGFR2c; 10⁻⁷ or less, 10⁻⁸ or less, or 10⁻⁹ or less for FGFR3c; and/or 10⁻⁷ or less, 10⁻⁸ or less, or 10 or less for FGFR4 when in IgG2 format (e.g., wild-type IgG2 constant region or a variant such as M7 (SEQ ID NO: 1173)), e.g., as measured by bio-layer interferometry (e.g., the ForteBio assay described in Example 9);

(b) binds to human FGFR1c, FGFR2c, FGFR3c, and/or FGFR4, e.g., with an EC50 of, for example, 10 nM or less, 1 nM or less, or 0.05 nM or less for FGFR1c; 10 nM or less, 1 nM or less, or 0.05 nM or less for FGFR2c; 10 nM or less, 1 nM or less, or 0.5 nM or less for FGFR3c; and/or 50 nM or less, 25 nM or less, or 10 nM or less for FGFR4 when in IgG2 format (e.g., wild-type IgG2 constant region or a variant such as M7 (SEQ ID NO: 1173)), e.g., as measured by ELISA (see, e.g., Examples 3 and 9);

(c) does not bind to human FGFR1b, FGFR2b, and/or FGFR3b, as measured by ELISA or bio-layer interferometry;

(d) inhibits the binding of FGF1 to FGFR1c, FGFR2c, FGFR3c, and/or FGFR4, e.g., with an EC50 of 200 nM or less, 150 nM or less, 50 nM or less, 25 nM or less, or 10 nM or less, e.g., as measured by ELISA (see, e.g., Example 9);

(e) inhibits FGF2-mediated phosphorylation of ERK, e.g., with an IC₅₀ of 50 or less, 25 nM or less, or 10 nM or less, as measured by SureFire Alpha assay (see, e.g., Example 7); and

(f) inhibits FGF2-mediated cell viability, e.g., with an IC₅₀ of 100 nM or less, 75 nM or less, 50 nM or less, 25 nM or less, or 10 nM or less, as assessed with CellTiterGlo (see, e.g., Example 8).

In some embodiments, provided are anti-FGFR antibodies comprising heavy and light chains, wherein the antibodies comprise heavy and light chain sequences which are at least 80%, 85%, 90%, 95%, 96%, 97%, 98%, or 99% identical to the amino acid sequences selected from the group consisting of SEQ ID NOs: 32 and 33; 43 and 44; 54 and 55; 65 and 66; 76 and 77; 87 and 88; 98 and 99; 109 and 110; 120 and 121; 131 and 132; 142 and 143; 153 and 154; 164 and 165; 175 and 176; 186 and 187; 196 and 197; 206 and 207; 216 and 217; 226 and 227; 236 and 237; 246 and 247; 256 and 257; 266 and 267; 276 and 277; 286 and 287; 296 and 297; 306 and 307; 316 and 317; 326 and 327; 336 and 337; 346 and 347; 356 and 357; 366 and 367; 376 and 377; 386 and 387; 396 and 397; 406 and 407; 416 and 417; 426 and 427; 436 and 437; 446 and 447; 456 and 457; 466 and 467; 476 and 477; 486 and 487; 496 and 497; 506 and 507; 516 and 517; 526 and 527; 536 and 537; 546 and 547; 556 and 557; 566 and 567; 576 and 577; 586 and 587; 596 and 597; 606 and 607; 616 and 617; 626 and 627; 636 and 637; 646 and 647; 656 and 657; 666 and 667; 676 and 677; 686 and 687; 696 and 697; 706 and 707; 716 and 717; 726 and 727; 736 and 737; 746 and 747; 756 and 757; 766 and 767; 776 and 777; 786 and 787; 796 and 797; 806 and 807; 816 and 817; 826 and 827; 836 and 837; 846 and 847; 856 and 857; 866 and 867; 876 and 877; 886 and 887; 896 and 897; 906 and 907; 916 and 917; 926 and 927; 936 and 937; 946 and 947; 956 and 957; 966 and 967; 976 and 977; 986 and 987; 996 and 997; 1006 and 1007; 1016 and 1017; 1026 and 1027; 1036 and 1037; 1046 and 1047; 1056 and 1057; 1066 and 1067; 1076 and 1077; 1086 and 1087; 1096 and 1097; and 1106 and 1107, wherein the antibodies specifically bind to human FGFR1c, FGFR2c, FGFR3c, and/or FGFR4 and retain one or more (e.g., 1, 2, 3, 4, 5, or 6) of the following properties:

(a) binds to human FGFR1c, FGFR2c, FGFR3c, and/or FGFR4, e.g., with a K_{D} of 10⁻⁷ or less, 10⁻⁸ or less, or 10⁻⁹ or less for FGFR1c; 10⁻⁷ or less, 10⁻⁸ or less, or 10⁻⁹ or less for FGFR2c; 10⁻⁷ or less, 10⁻⁸ or less, or 10⁻⁹ or less for FGFR3c; and/or 10⁻⁷ or less, 10⁻⁸ or less, or 10⁻⁹ or less for FGFR4 when in IgG2 format (e.g., wild-type IgG2 constant region or a variant such as M7 (SEQ ID NO: 1173)), e.g., as measured by bio-layer interferometry (e.g., the ForteBio assay described in Example 9);

(b) binds to human FGFR1c, FGFR2c, FGFR3c, and/or FGFR4, e.g., with an EC50 of, for example, 10 nM or less, 1 nM or less, or 0.05 nM or less for FGFR1c; 10 nM or less, 1 nM or less, or 0.05 nM or less for FGFR2c; 10 nM or less, 1 nM or less, or 0.5 nM or less for FGFR3c; and/or 50 nM or less, 25 nM or less, or 10 nM or less for FGFR4 when in IgG2 format (e.g., wild-type IgG2 constant region or a variant such as M7 (SEQ ID NO: 1173)), e.g., as measured by ELISA (see, e.g., Examples 3 and 9);

(c) does not bind to human FGFR1b, FGFR2b, and/or FGFR3b , as measured by ELISA or bio-layer interferometry;

(d) inhibits the binding of FGF1 to FGFR1c, FGFR2c, FGFR3c, and/or FGFR4, e.g., with an EC50 of 200 nM or less, 150 nM or less, 50 nM or less, 25 nM or less, or 10 nM or less, e.g., as measured by ELISA (see, e.g., Example 9);

(e) inhibits FGF2-mediated phosphorylation of ERK, e.g., with an IC₅₀ of 50 or less, 25 nM or less, or 10 nM or less, as measured by SureFire Alpha assay (see, e.g., Example 7); and

(f) inhibits FGF2-mediated cell viability, e.g., with an IC₅₀ of 100 nM or less, 75 nM or less, 50 nM or less, 25 nM or less, or 10 nM or less, as assessed with CellTiterGlo (see, e.g., Example 8).

In some embodiments, some or all of the amino acid changes made to the CDR(s) or variable regions of the altered anti-FGFR antibodies described above are conservative modifications, wherein the antibodies retain the desired functional properties of the anti-FGFR antibodies described herein.

Certain positions within the VH and/or VL CDR sequences of antibodies are substitutable (variation-tolerant) positions, i.e., a particular position of one or more VH and/or VL CDR sequences in an antibody that may be substituted by different amino acids without significantly decreasing the binding activity of the antibody. Once such a position is identified, the amino acid at that position may be substituted for a different amino acid without significantly decreasing the binding activity of the antibody. In order to identify a substitutable position of an antibody, the amino acid sequence of that antibody is compared to the sequences of other antibodies belonging to the same group as that antibody (e.g., affinity matured and parental antibodies, a group of distinct antibodies generated from immunizing an animal with a particular antigen). If the identity of that amino acid varies between the different related antibodies of a group at any particular position, that position is a substitutable position of the antibody. In other words, a substitutable position is a position in which the identity of the amino acid varies between the related antibodies.

In one embodiment, the above method may be employed to provide a consensus antibody sequence. In such a consensus sequence, a non-substitutable position is indicated by the amino acid present at that position, and a substitutable position is indicated as an “X,” wherein X can be any amino acid, any amino acid present at that position in a related antibody, or a conservatively substituted amino acid present at that position in a related antibody. For example, if unrelated amino acids (e.g., ala, gly, cys, glu and thr) are present at a certain position of a group of related antibodies, then any amino acid could be substituted at that position without significantly reducing binding activity of the antibody. Similarly, if a subset of non-polar amino acids (e.g., val, ile, ala and met) are present at a certain position of a set of related antibodies, then other non-polar amino acids (e.g., leu) could be substituted at that position without significantly reducing binding activity of the antibody. Any antibody having a sequence that is encompassed by the consensus should bind to the same antigen as any of the related antibodies, and this can be tested using binding assays known in the art, such as those described herein. The antibodies can also be tested, using methods disclosed herein, for their ability to bind to human FGFR1c, FGFR2c, FGFR3c, and/or FGFR4; lack of binding to human FGFR1b, FGFR2b, and/or FGFR3b; inhibit the binding of FGF1 to human FGFR1c, FGFR2c, FGFR3c, and/or FGFR4; inhibit FGF2-mediated phosphorylation of ERK; and/or inhibit FGF2-mediated cell viability, as described above.

Detailed methods for identifying substitutable positions are described in US2015/0038370, the contents of which are herein incorporated by reference.

In general, the framework regions of antibodies are usually substantially identical, and more often, identical to the framework regions of the human germline sequences from which they were derived. Many of the amino acids in the framework region make little or no direct contribution to the specificity or affinity of an antibody. Thus, many individual conservative substitutions of framework residues can be tolerated without appreciable change of the specificity or affinity of the resulting immunoglobulin. Thus, in one embodiment, the variable framework region of the antibody shares at least 85% sequence identity to a human germline variable framework region sequence or consensus of such sequences. In another embodiment, the variable framework region of the antibody shares at least 90%, 95%, 96%, 97%, 98% or 99% sequence identity to a human germline variable framework region sequence or consensus of such sequences. In a preferred embodiment made within one or more of the framework regions, FR1, FR2, FR3 and FR4, of the heavy and/or the light chain variable regions of an antibody, do not eliminate the binding of the antibody to its antigen (e.g., FGFR1c, FGFR2c, FGFR3c, and/or FGFR4).

In another aspect, the structural features of an anti-FGFR antibody described herein, e.g. Ab1-Ab107, are used to create structurally-related anti-FGFR antibodies that retain at least one functional property of the antibodies described herein, such as binding to human FGFR1c, FGFR2c, FGFR3c, and/or FGFR4. For example, one or more CDR regions ofany of Ab1-Ab107, or altered sequences thereof, can be combined recombinantly with known framework regions and/or other CDRs to create additional, recombinantly-engineered, anti-FGFR antibodies.

Antibodies having sequences with homology to the variable region or CDR sequences of any of Ab1-Ab107 can be generated by mutagenesis (e.g., site-directed or PCR-mediated mutagenesis) of nucleic acid molecules encoding the respective variable regions, followed by testing to determine whether the altered antibody retains the desired function.

Also provided herein are anti-FGFR antibodies wherein the V_{H} CDR1, 2 and 3 sequences and V_{L} CDR1, 2 and 3 sequences, or VH and VL sequences, are “mixed and matched” (i.e., CDRs from different antibodies, e.g., from any of Ab1-Ab107), although each antibody must contain a V_{H} CDR1, 2 and 3 and a V_{L} CDR1, 2 and 3 to create other anti-FGFR antibodies. Assays to determine whether the resultant antibodies retain the desired features (including binding to FGFR1c, FGFR2c, FGFR3c, and/or FGFR4) can be determined using the methods described in the Examples.

Antibodies with Modified Fc Regions

In some embodiments, provided herein are anti-FGFR antibodies comprising a modified heavy chain Fc region. In one embodiment, the anti-FGFR antibody comprises an IgG2 or variant IgG2 Fc region.

In one embodiment, an anti-FGFR antibody comprises an Fc region comprising the substitutions A330S/P331S, which reduces effector function.

In a particular embodiment, the anti-FGFR antibody comprises a hybrid IgG2/IgG4 Fc region, for example, an IgG2 Fc region with four amino acid residue changes derived from IgG4 (i.e., H268Q, V309L, A330S, and P331S), also referred to as IgG2m4 (An et al. mAbs 2009;1:572-579).

Additional modified Fc regions suitable for use with the anti-FGFR antibodies described herein include, but are not limited to, the Fc regions comprising amino acid sequences set forth in SEQ ID NOs: 1172-1175 (optionally with the first three amino acids “AST” removed).

Accordingly, provided herein are anti-FGFR antibodies comprising the VH and VL sequences of Ab1-Ab107 and a Fc region with an IgG2 constant region or a variant IgG2 constant region (e.g., a hybrid IgG2/IgG4 Fc region). In some embodiments, provided herein are anti-FGFR antibodies comprising the VH and VL sequences of Ab1-Ab107 and an Fc region with an amino acid sequence selected from the group consisting of SEQ ID NOs: 1172-1175 (optionally with the first three amino acids “AST” removed).

Also contemplated are anti-FGFR antibodies comprising an Fc region with reduced or no effector function (e.g., the Fc of IgG2 or IgG4). Generally, the variable regions described herein may be linked to an Fc comprising one or more modification, typically to alter one or more functional properties of the antibody, such as serum half-life, complement fixation, Fc receptor binding, and/or antigen-dependent cellular cytotoxicity. For example, modifications may be made in the Fc region to generate an Fc variant that (a) has decreased antibody-dependent cell-mediated cytotoxicity (ADCC), (b) decreased complement mediated cytotoxicity (CDC), (c) has decreased affinity for C1q and/or (d) has increased or decreased affinity for a Fc receptor relative to the parent Fc. Such Fc variants may comprise one or more amino cid modifications. For example, a variant Fc region may include two, three, four, five, etc. substitutions therein, such as the substitutions described below. The numbering of residues in the Fc region described below is based on the EU index of Kabat.

In some embodiments, sites involved in interaction with complement, such as the C1q binding site, may be removed from the Fc region. For example, the EKK sequence of human IgG1 may be deleted. In some embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1. Specific examples of variant Fc domains are disclosed for example, in WO 97/34631 and WO 96/32478.

In some embodiments, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the Cl component of complement. This approach is described in further detail in U.S. Pat. No. 5,624,821 and U.S. Pat. No. 5,648,260, both by Winter et al.

In further embodiments, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Patent No. 6,194,551 by Idusogie et al.

In some embodiments, one or more amino acid residues within amino acid positions 231 and 239 are altered to thereby alter the ability of the antibody to fix complement. This approach is described further in PCT Publication WO 94/29351 by Bodmer et al.

Other Fc modifications that can be made to Fcs are those for reducing or ablating binding to FcγR and/or complement proteins, thereby reducing or ablating Fc-mediated effector functions such as ADCC, ADCP, and CDC. Exemplary modifications include but are not limited substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcyR and complement interactions include substitutions 297A, 234A, 235A, 237A, 318A, 228P, 236E, 268Q, 309L, 330S, 331S, 220S, 226S, 2295, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

### IV. Nucleic Acid Molecules

Also provided herein are nucleic acid molecules that encode the antibodies described herein. The nucleic acids may be present in whole cells, in a cell lysate, or in a partially purified or substantially pure form. A nucleic acid described herein can be, for example, DNA or RNA and may or may not contain intronic sequences. In a certain embodiments, the nucleic acid is a cDNA molecule. The nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

In some embodiments, provided herein are nucleic acid molecules that encode the VH and/or VL sequences, or heavy and/or light chain sequences, of any of Ab1-Ab107, as well as nucleic acid molecules which are at least 80%, at least 85%, at least 90%, at least 95%, or at least 99% identical to nucleic acid molecules encoding the VH and/or VL sequences, or heavy and/or light chain sequences, of any of Ab1-Ab107. Host cells comprising the nucleotide sequences (e.g., nucleic acid molecules) described herein are encompassed herein.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2 and/or CH3). The sequences of human heavy chain constant region genes are known in the art (see e.g., Kabat, E. A., el al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

Also provided herein are nucleic acid molecules with conservative substitutions that do not alter the resulting amino acid sequence upon translation of the nucleic acid molecule.

### V. Methods for Producing Antibodies

The anti-FGFR antibodies provided herein typically are prepared by standard recombinant DNA techniques based on the amino acid sequences of the V_{H} and V_{L} regions disclosed herein. Additionally or alternatively, monoclonal antibodies can be produced using a variety of known techniques, such as the standard somatic cell hybridization technique, viral or oncogenic transformation of B lymphocytes, or yeast or phage display techniques using libraries of human antibody genes. In particular embodiments, the antibodies are fully human monoclonal antibodies.

In one embodiment, a hybridoma method is used to produce an antibody that binds FGFRs (e.g., human FGFRs such as human FGFR1c, FGFR2c, FGFR3c, and/or FGFR4). In this method, a mouse or other appropriate host animal can be immunized with a suitable antigen in order to elicit lymphocytes that produce or are capable of producing antibodies that will specifically bind to the antigen used for immunization. Alternatively, lymphocytes may be immunized in vitro. Lymphocytes can then be fused with myeloma cells using a suitable fusing agent, such as polyethylene glycol, to form a hybridoma cell. Culture medium in which hybridoma cells are growing is assayed for production of monoclonal antibodies directed against the antigen. After hybridoma cells are identified that produce antibodies of the desired specificity, affinity, and/or activity, the clones may be subcloned by limiting dilution procedures and grown by standard methods. Suitable culture media for this purpose include, for example, D-MEM or RPMI-1640 medium. In addition, the hybridoma cells may be grown in vivo as ascites tumors in an animal. The monoclonal antibodies secreted by the subclones can be separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies can also be produced in a host cell transfectoma using, for example, a combination of recombinant DNA techniques and gene transfection methods known in the art (Morrison, S. (1985) Science 229:1202). For example, to express antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” means that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or both genes are inserted into the same expression vector. The antibody genes are inserted into the expression vector(s) by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector.

For expression of light and heavy chains, the expression vector(s) encoding the heavy and light chains is transfected into a host cell by standard techniques. Although it is possible to express the antibodies described herein in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Preferred mammalian host cells for expressing the recombinant antibodies described herein include Chinese Hamster Ovary (CHO cells) (including dhfr- CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

In another embodiment, antibodies that bind FGFR can be isolated from antibody libraries generated using well know techniques such as those described in, for example, U.S. Pat. Nos. 5,223,409; 5,403,484; and U.S. Pat. No. 5,571,698 to Ladner et al.; U.S. Pat. No. 5,427,908 and U.S. Pat. No. 5,580,717 to Dower et al.; U.S. Pat. No. 5,969,108 and U.S. Pat. No. 6,172,197 to McCafferty et al.; and U.S. Pat. Nos. 5,885,793; 6,521,404; 6,544,731; 6,555,313; 6,582,915 and U.S. Pat. No. 6,593,081 to Griffiths et al.. Additionally, production of high affinity (nM range) human antibodies by chain shuffling, as well as combinatorial infection and in vivo recombination as a strategy for constructing very large phage libraries may also be used. See, e.g., U.S. patent application Ser. No. 09/856,907 (PCT Int. Pub. No. WO 00/31246)

In a particular embodiment, the monoclonal antibody that binds FGFR is produced using phage display. This technique involves the generation of a human Fab library having a unique combination of immunoglobulin sequences isolated from human donors and having synthetic diversity in the heavy-chain CDRs is generated. The library is then screened for Fabs that bind to FGFR.

In yet another embodiment, human monoclonal antibodies directed against FGFR can be generated using transgenic or transchromosomic mice carrying parts of the human immune system rather than the mouse system (see e.g., U.S. Pat. Nos. 5,545,806; 5,569,825; 5,625,126; 5,633,425; 5,789,650; 5,877,397; 5,661,016; 5,814,318; 5,874,299; and U.S. Pat. No. 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.).

In another embodiment, human antibodies can be raised using a mouse that carries human immunoglobulin sequences on transgenes and transchomosomes, such as a mouse that carries a human heavy chain transgene and a human light chain transchromosome (see e.g., PCT Publication WO 02/43478 to Ishida et al.).

Still further, alternative transgenic animal systems expressing human immunoglobulin genes are available in the art and can be used to raise anti-FGFR antibodies. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and U.S. Pat. No. 6,162,963 to Kucherlapati et al. Another suitable transgenic animal system is the HuMAb mouse (Medarex, Inc), which contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368(6474): 856-859). Yet another suitable transgenic animal system is the KM mouse, described in detail in PCT publication W002/43478.

Alternative transchromosomic animal systems expressing human immunoglobulin genes are available in the art and can be used to raise anti-FGFR antibodies. For example, mice carrying both a human heavy chain transchromosome and a human light chain tranchromosome can be used. Furthermore, cows carrying human heavy and light chain transchromosomes have been described in the art and can be used to raise anti-FGFR antibodies.

In yet another embodiment, antibodies can be prepared using a transgenic plant and/or cultured plant cells (such as, for example, tobacco, maize and duckweed) that produce such antibodies. For example, transgenic tobacco leaves expressing antibodies can be used to produce such antibodies by, for example, using an inducible promoter. Also, transgenic maize can be used to express such antibodies and antigen binding portions thereof. Antibodies can also be produced in large amounts from transgenic plant seeds including antibody portions, such as single chain antibodies (scFv's), for example, using tobacco seeds and potato tubers.

The binding specificity of monoclonal antibodies (or portions thereof) that bind FGFR prepared using any technique including those disclosed here, can be determined by immunoprecipitation or by an in vitro binding assay, such as radioimmunoassay (RIA) or enzyme-linked immunoabsorbent assay (ELISA). The binding affinity of a monoclonal antibody or portion thereof also can be determined by Scatchard analysis.

In certain embodiments, an anti-FGFR antibody produced using any of the methods discussed above may be further altered or optimized to achieve a desired binding specificity and/or affinity using art recognized techniques, such as those described herein.

### VI. Multispecific Antibodies

Multispecific antibodies provided herein include at least a binding affinity for one or more FGFR proteins (e.g., human FGFR1c, FGFR2c, FGFR3c, and/or FGFR4), such as an anti-FGFR antibody described herein, and at least one other non-FGFR binding specificity. In some embodiments, the non-FGFR binding specificity is a binding specificity for a cancer antigen. Multispecific antibodies can be prepared as full length antibodies or antibody fragments (e.g. F(ab′)₂ antibodies).

Methods for making multispecific antibodies are well known in the art (see, e.g., WO 05117973 and WO 06091209). For example, production of full length multispecific antibodies can be based on the coexpression of two paired immunoglobulin heavy chain-light chains, where the two chains have different specificities. Various techniques for making and isolating multispecific antibody fragments directly from recombinant cell culture have also been described. For example, multispecific antibodies can be produced using leucine zippers. Another strategy for making multispecific antibody fragments by the use of single-chain Fv (sFv) dimers has also been reported.

In a particular embodiment, the multispecific antibody comprises a first antibody (or binding portion thereof) which binds to an FGFR protein derivatized or linked to another functional molecule, e.g., another peptide or protein (e.g., another antibody or ligand for a receptor) to generate a multispecific molecule that binds to one or more of FGFR1c, FGFR2c, FGFR3c, and/or FGFR4 and a non-FGFR target molecule. An antibody may be derivatized or linked to more than one other functional molecule to generate multispecific molecules that bind to more than two different binding sites and/or target molecules. To create a multispecific molecule, an antibody disclosed herein can be functionally linked (e.g., by chemical coupling, genetic fusion, noncovalent association or otherwise) to one or more other binding molecules, such as another antibody, antibody fragment, peptide or binding mimetic, such that a multispecific molecule results.

Accordingly, multispecific molecules comprising at least one first binding specificity for an FGFR protein (e.g., human FGFR1c, FGFR2c, FGFR3c, and/or FGFR4) and a second binding specificity for a second non-FGFR target epitope are contemplated. In a particular embodiment, the second target epitope is an Fc receptor, e.g., human FcγRI (CD64) or a human Fcα receptor (CD89). Therefore, multispecific molecules capable of binding both to FcγR, FcαR or FcεR expressing effector cells (e.g., monocytes, macrophages or polymorphonuclear cells (PMNs)), and to target cells expressing FGFR are also provided. These multispecific molecules target FGFR-expressing cells to effector cells and trigger Fc receptor-mediated effector cell activities, such as phagocytosis of FGFR-expressing cells, antibody dependent cell-mediated cytotoxicity (ADCC), cytokine release, or generation of superoxide anion.

In one embodiment, the multispecific molecules comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778.

The multispecific molecules can be prepared by conjugating the constituent binding specificities, e.g., the anti-FcR and anti-FGFR binding specificities, using methods known in the art. For example, each binding specificity of the multispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-l-carboxylate (sulfo-SMCC). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the multispecific molecule is a mAb×mAb, mAb×Fab, Fab×F(ab′)₂ or ligand×Fab fusion protein. A multispecific molecule can be a single chain molecule comprising one single chain antibody and a binding determinant, or a single chain bispecific molecule comprising two binding determinants. Multispecific molecules may comprise at least two single chain molecules. Methods for preparing multispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the multispecific molecules to their specific targets can be confirmed by, for example, enzyme-linked immunosorbent assay (ELISA), radioimmunoassay (RIA), FACS analysis, bioassay (e.g., growth inhibition), or western blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA). The radioactive isotope can be detected by such means as the use of a αγ-β counter or a scintillation counter or by autoradiography.

### VII. Immunoconjugates

Immunoconjugates provided herein can be formed by conjugating the antibodies described herein to another therapeutic agent. Suitable agents include, for example, a cytotoxic agent (e.g., a chemotherapeutic agent), a toxin (e.g. an enzymatically active toxin of bacterial, fungal, plant or animal origin, or fragments thereof), and/or a radioactive isotope (i.e., a radioconjugate).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, neomycin, and the tricothecenes. Additional examples of cytotoxins or cytotoxic agents include, e.g., taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

A variety of radionuclides are available for the production of radioconjugated anti-FGFR antibodies. Examples include ²¹²Bi, ¹³¹I, ¹³¹In, ⁹⁰Y and ¹⁸⁶Re.

Immunoconjugates can also be used to modify a given biological response, and the drug moiety is not to be construed as limited to classical chemical therapeutic agents. For example, the drug moiety may be a protein or polypeptide possessing a desired biological activity (e.g., lymphokines, tumor necrosis factor, IFNγ, growth factors).

Immunoconjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). Carbon-14-labeled 1-isothiocyanatobenzyl-3-methyldiethylene triaminepentaacetic acid (MX-DTPA) is an exemplary chelating agent for conjugation of radionucleotide to the antibody (see, e.g., W094/11026).

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### VIII. Assays

Subsequent to producing antibodies, they can be screened for various properties, such as those described herein, using a variety of assays known in the art.

In one embodiment, the antibodies are screened (e.g., by flow cytometry, ELISA, Biacore, or ForteBio assay) for binding to FGFR using, for example, purified FGFR and/or FGFR-expres sing cells. The epitopes bound by the anti-FGFR antibodies can further be identified and compared, for example, to identify non-competing antibodies (e.g., antibodies that bind different epitopes), as well as antibodies which compete for binding and/or bind the same or overlapping epitopes.

Competitive antibodies and non-competitive antibodies can be identified using routine techniques. Such techniques include, for example, an immunoassay, which shows the ability of one antibody to block (or not block) the binding of another antibody to a target antigen, i.e., a competitive binding assay. Competitive binding is determined in an assay in which the immunoglobulin under test inhibits specific binding of a reference antibody to a common antigen, such as FGFR. Numerous types of competitive binding assays are known, for example: solid phase direct or indirect radioimmunoassay (RIA), solid phase direct or indirect enzyme immunoassay (EIA), sandwich competition assay; solid phase direct biotin-avidin EIA; solid phase direct labeled assay, solid phase direct labeled sandwich assay; solid phase direct ¹²⁵I labeled RIA; solid phase direct biotin-avidin EIA; and direct labeled RIA. Surface plasmon resonance can also be used for this purpose. Typically, such an assay involves the use of purified antigen bound to a solid surface or cells bearing either of these, an unlabeled test immunoglobulin, and a labeled reference immunoglobulin. Competitive inhibition is measured by determining the amount of label bound to the solid surface or cells in the presence of the test immunoglobulin. The test immunoglobulin is typically present in excess. Usually, when a competing antibody is present in excess, it will inhibit specific binding of a reference antibody to a common antigen by at least 30%, at least 40%, at least 50%, at least 60%, at least 70%, at least 80%, at least 90%, at least 95%, or more.

Other screening techniques for determining the epitope bound by antibodies disclosed herein include, for example, x-ray analysis of crystals of antigen:antibody complexes, which provides atomic resolution of the epitope. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

In another embodiment, the antibodies (e.g., non-competing anti-FGFR antibodies) are screened for the ability to bind to epitopes exposed upon binding to ligand, e.g., FGF1 (i.e., do not inhibit the binding of FGFR-binding ligands to FGFR). Such antibodies can be identified by, for example, contacting cells which express FGFR with a labeled FGFR ligand (e.g., radiolabeled or biotinylated FGF) in the absence (control) or presence of the anti-FGFR antibody. If the antibody does not inhibit FGF binding to FGFR, then no statistically significantly decrease in the amount of label recovered, relative to the amount in the absence of the antibody, will be observed. Alternatively, if the antibody inhibits FGF binding to FGFR, then a statistically significantly decrease in the amount of label recovered, relative to the amount in the absence of the antibody, will be observed.

Methods for analyzing binding affinity, cross-reactivity, and binding kinetics of various anti-FGFR antibodies include standard assays known in the art, for example, Biacore surface plasmon resonance (SPR) analysis using a Biacore 2000 SPR instrument (Biacore AB, Uppsala, Sweden) or bio-layer interferometry (e.g., ForteBio assay), as described in the Examples.

Antibodies also can be screened for their ability to inhibit signaling through FGFR using signaling assays, such as those described in the Examples. In one embodiment, the ability of an antibody to inhibit FGFR ligand-mediated phosphorylation of ERK can be assessed by treating cells expressing FGFR (e.g., FGFR1c, FGFR2c, FGFR3c, and/or FGFR4) with an FGFR ligand (e.g., FGF1) in the presence and absence of the antibody. The cells can then be lysed, crude lysates centrifuged to remove insoluble material, EGF phosphorylation measured, for example, by western blotting followed by probing with an antibody which specifically recognizes phosphorylated ERK, and IC50 and/or IC90 values determined.

Antibodies can also be tested for their ability to inhibit the proliferation or viability of cells expressing FGFR(s) (either in vivo or in vitro), such as tumor cells, using art recognized techniques, including the Cell Titer-Glo Assay described in the Examples or a tritium-labeled thymidine incorporation assay.

### IX. Compositions

In another aspect, provided herein is a composition, e.g., a pharmaceutical composition, containing an anti-FGFR antibody disclosed herein, formulated together with a pharmaceutically acceptable carrier. Pharmaceutical compositions are prepared using standard methods known in the art by mixing the active ingredient (e.g., anti-FGFR antibodies described herein) having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, Pa.). In one embodiment, the composition includes a combination of multiple (e.g., two, three, or four antibodies) isolated anti-FGFR antibodies which collectively bind to human FGFR1c, FGFR2c, FGFR3c, and FGFR4. In another embodiment, the composition includes an antibody which binds to FGFR1c, FGFR2c, FGFR3c, and FGFR4. In some embodiments, the anti-FGFR antibodies do not bind to FGFR1b, FGFR2b, and/or FGFR3b. Preferred pharmaceutical compositions are sterile compositions, compositions suitable for injection, and sterile compositions suitable for injection by a desired route of administration, such as by intravenous injection.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, may be coated in a material to protect the compound from the action of acids and other natural conditions that may inactivate the compound.

Compositions can be administered alone or in combination therapy, i.e., combined with other agents. For example, the combination therapy can include a composition provided herein with at least one or more additional therapeutic agents, e.g., other compounds, drugs, and/or agents used for the treatment of cancer (e.g., an anti-cancer agent(s). Particular combinations of anti-FGFR antibodies may also be administered separately or sequentially, with or without additional therapeutic agents.

Compositions can be administered by a variety of methods known in the art. As will be appreciated by the skilled artisan, the route and/or mode of administration will vary depending upon the desired results. The antibodies can be prepared with carriers that will protect the antibodies against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art.

To administer compositions by certain routes of administration, it may be necessary to coat the constituents, e.g., antibodies, with, or co-administer the compositions with, a material to prevent its inactivation. For example, the compositions may be administered to a subject in an appropriate carrier, for example, liposomes, or a diluent. Acceptable diluents include saline and aqueous buffer solutions. Liposomes include water-in-oil-in-water CGF emulsions as well as conventional liposomes.

Acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional medium or agent is incompatible with the antibodies, use thereof in compositions provided herein is contemplated. Supplementary active constituents can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Including in the composition an agent that delays absorption, for example, monostearate salts and gelatin can bring about prolonged absorption of the injectable compositions.

Sterile injectable solutions can be prepared by incorporating the monoclonal antibodies in the required amount in an appropriate solvent with one or a combination of ingredients enumerated above, as required, followed by sterilization microfiltration. Generally, dispersions are prepared by incorporating the antibodies into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, human antibodies may be administered once or twice weekly by subcutaneous injection or once or twice monthly by subcutaneous injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of antibodies calculated to produce the desired therapeutic effect in association with the required pharmaceutical carrier. The specification for the dosage unit forms provided herein are dictated by and directly dependent on (a) the unique characteristics of the antibodies and the particular therapeutic effect to be achieved, and (b) the limitations inherent in the art of compounding such antibodies for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations include those suitable for oral, nasal, topical (including buccal and sublingual), rectal, and parenteral administration. Parenteral administration is the most common route of administration for therapeutic compositions comprising antibodies. The formulations may conveniently be presented in unit dosage form and may be prepared by any methods known in the art of pharmacy. The amount of antibodies that can be combined with a carrier material to produce a single dosage form will vary depending upon the subject being treated, and the particular mode of administration. This amount of antibodies will generally be an amount sufficient to produce a therapeutic effect. Generally, out of 100%, this amount will range from about 0.001% to about 90% of antibody by mass, preferably from about 0.005% to about 70%, most preferably from about 0.01% to about 30%.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Particular examples of adjuvants which are well-known in the art include, for example, inorganic adjuvants (such as aluminum salts, e.g., aluminum phosphate and aluminum hydroxide), organic adjuvants (e.g., squalene), oil-based adjuvants, virosomes (e.g., virosomes which contain a membrane-bound heagglutinin and neuraminidase derived from the influenza virus).

Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of one or more agents that delay absorption such as aluminum monostearate or gelatin.

When compositions are administered as pharmaceuticals, to humans and animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

Regardless of the route of administration selected, compositions provided herein, may be used in a suitable hydrated form, and they may be formulated into pharmaceutically acceptable dosage forms by conventional methods known to those of skill in the art.

Actual dosage levels of the antibodies in the pharmaceutical compositions provided herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the composition required. For example, the physician or veterinarian could start doses of the antibodies at levels lower than that required to achieve the desired therapeutic effect and gradually increasing the dosage until the desired effect is achieved. In general, a suitable daily dose of compositions provided herein will be that amount of the antibodies which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic composition may be administered as two, three, four, five, six or more sub-doses administered separately at appropriate intervals throughout the day, optionally, in unit dosage forms. While it is possible for antibodies to be administered alone, it is preferable to administer antibodies as a formulation (composition).

Dosages and frequency of administration may vary according to factors such as the route of administration and the particular antibody used, the nature and severity of the disease to be treated, and the size and general condition of the subject. Appropriate dosages can be determined by procedures known in the pertinent art, e.g. in clinical trials that may involve dose escalation studies. An exemplary treatment regime entails administration once per week, once every two weeks, once every three weeks, once every four weeks, once a month, once every 3 months, or once every three to 6 months. In some embodiments, the antibodies described herein are administered at a flat dose (flat dose regimen).

Therapeutic compositions can be administered with medical devices known in the art, such as, for example, those disclosed in U.S. Pat. Nos. 5,399,163, 5,383,851, 5,312,335, 5,064,413, 4,941,880, 4,790,824, 4,596,556, 4,487,603, 4.,486,194, 4,447,233, 4,447,224, 4,439,196, and U.S. Pat. No. 4,475,196.

The ability of a compound to inhibit cancer can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

Uses of the above-described anti-FGFR antibodies and compositions comprising the same are provided in the manufacture of a medicament for the treatment of a disease associated with FGFR-dependent signaling. The above-described anti-FGFR antibodies and compositions are also provided for the treatment of cancer (or to be used in the manufacture of a medicament for the treatment of cancer), such as an FGFR-expressing cancer or a cancer with altered FGFR signaling. In some embodiments, the cancer is a mesenchymal-like solid tumors. Exemplary cancers include, but are not limited to, lung cancer, renal cancer, breast cancer, and ovarian cancer.

Additionally, contemplated compositions may further include, or be prepared for use as a medicament in combination therapy with, an additional therapeutic agent, e.g., an additional anti-cancer agent. An “anti-cancer agent” is a drug used to treat tumors, cancers, malignancies, and the like. Drug therapy (e.g., with antibody compositions disclosed herein) may be administered without other treatment, or in combination with other treatments.

A “therapeutically effective dosage” of an anti-FGFR antibody or composition described herein preferably results in a decrease in severity of disease symptoms, an increase in frequency and duration of disease symptom-free periods, or a prevention of impairment or disability due to the disease affliction. In the context of cancer, a therapeutically effective dose preferably results in increased survival, and/or prevention of further deterioration of physical symptoms associated with cancer. A therapeutically effective dose may prevent or delay onset of cancer, such as may be desired when early or preliminary signs of the disease are present.

### X. Kits

Also provided are kits comprising the anti-FGFR antibodies, multispecific molecules, or immunoconjugates disclosed herein, optionally contained in a single vial or container, and include, e.g., instructions for use in treating or diagnosing a disease associated with FGFR upregulation and/or FGFR-dependent signaling. The kits may include a label indicating the intended use of the contents of the kit. The term label includes any writing, marketing materials or recorded material supplied on or with the kit, or which otherwise accompanies the kit. Such kits may comprise the antibody, multispecific molecule, or immunoconjugate in unit dosage form, such as in a single dose vial or a single dose pre-loaded syringe.

### XI. Methods of Using Antibodies

Antibodies and compositions disclosed herein can be used in a broad variety of therapeutic and diagnostic applications, particularly oncological applications. Accordingly, in another aspect, provided herein are methods for inhibiting FGFR activity in a subject by administering one or more antibodies or compositions described herein in an amount sufficient to inhibit FGFR-mediated activity. Particular therapeutic indications which can be treated include, for example, cancers of organs or tissues such as lung, kidney, breast, and ovary.

Antibodies disclosed herein also can be used to diagnose or prognose diseases (e.g., cancers) associated with FGFR, for example, by contacting an antibody disclosed herein (e.g., ex vivo or in vivo) with cells from the subject, and measuring the level of binding to FGFR on the cells, wherein abnormally high levels of binding to FGFR indicate that the subject has a cancer associated with FGFR.

Also provided herein is a method of detecting the presence of FGFR (e.g., FGFR1c, FGFR2c, FGFR3c, and/or FGFR4) in a sample. In some embodiments, the method comprises contacting the sample with an anti-FGFR antibody described herein under conditions that allow for formation of a complex between the antibody and FGFR protein, and detecting the formation of a complex. In some embodiments, the anti-FGFR antibodies described herein can be used to detect the presence or expression levels of FGFR proteins on the surface of cells in cell culture or in a cell population. In another embodiment, the anti-FGFR antibodies described herein can be used to detect the amount of FGFR proteins in a biological sample (e.g., a biopsy). In yet another embodiment, the anti-FGFR antibodies described herein can be used in in vitro assays (e.g., immunoassays such as Western blot, radioimmunoassays, ELISA) to detect FGFR proteins. The anti-FGFR antibodies described herein can also be used for fluorescence activated cell sorting (FACS).

Also provided are methods of blocking FGF1 or FGF2 binding to FGFR1c, FGFR2c, FGFR3c, and/or FGFR4 in a cell comprising contacting the cell with an effective amount of an antibody described herein.

In some embodiments, provided herein are methods of inhibiting FGF-mediated signaling in a cell comprising contacting the cell with an effective amount of an antibody described herein.

Also provided are methods of using the anti-FGFR antibodies disclosed herein in a variety of ex vivo and in vivo diagnostic and therapeutic applications involving FGFR-dependent signaling, including a variety of cancers.

Accordingly, in one embodiment, a method is provided for treating a disease associated with FGFR-dependent signaling by administering to a subject an antibody provided herein in an amount effective (e.g., a therapeutically effective amount) to treat the disease. Suitable diseases include, for example, a variety of cancers including, but not limited to, mesenchymal-like solid tumors, such as subsets of lung cancer, renal cancer, breast cancer, and ovarian cancer.

In another embodiment, a method is provided for inhibiting the growth of tumor cells comprising administering to a subject an antibody described herein in a therapeutically effective amount.

In another embodiment, a method is provided for treating cancer comprising administering to a subject in need thereof a therapeutically effective amount of an IgG2 antibody that binds to FGFR1c.

In some embodiments, the antibody binds to FGFR2c, FGFR3c, and/or FGFR4. In some embodiments, the antibody does not bind to FGFR1b, FGFR2b, and/or FGFR3b. In some embodiments, administration of the antibody in the methods described above does not induce weight loss in the subject.

The antibody can be administered alone or with another therapeutic agent that acts in conjunction with or synergistically with the antibody to treat the disease associated with FGFR-mediated signaling.

The present invention is further illustrated by the following examples which should not be construed as further limiting. The contents of Sequence Listing, figures and all references, patents and published patent applications cited throughout this application are expressly incorporated herein by reference.

### EXAMPLES

Commercially available reagents referred to in the Examples below were used according to manufacturer's instructions unless otherwise indicated. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

### Example 1

### Targeting Multiple FGF Receptors Enhances Inhibition of Tumor Cell Viability

This Example demonstrates that targeting multiple FGF receptors (FGFRs) can significantly decrease downstream signaling activity.

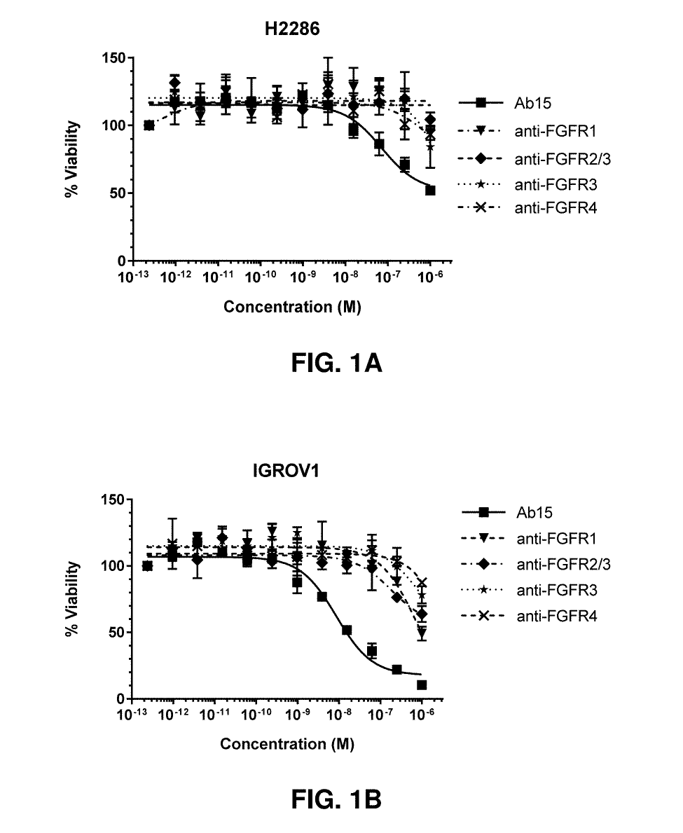

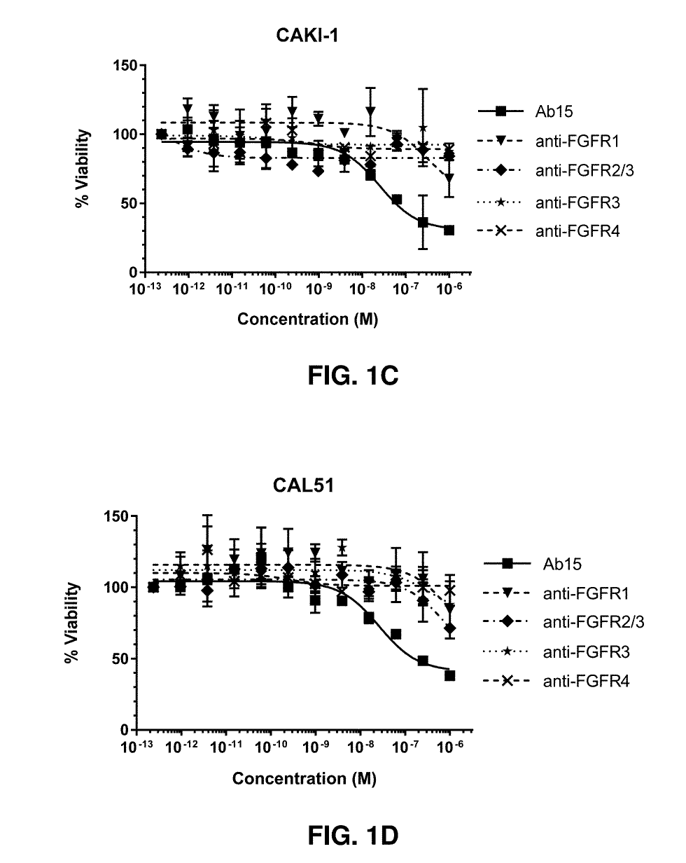

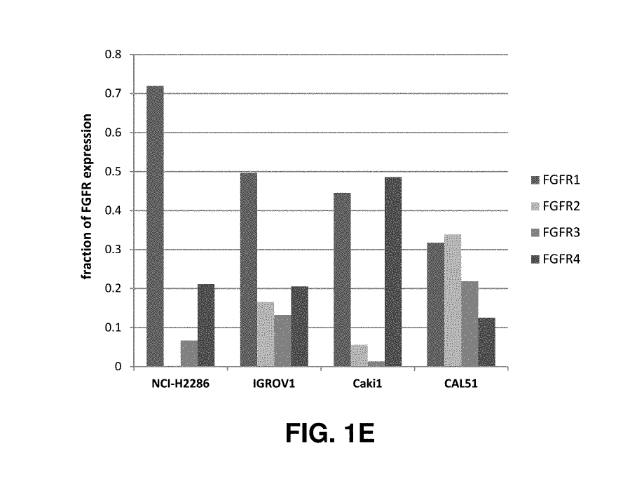

FGFR1 has been reported to be the main driver of FGFR-related signaling in cancer. To test the hypothesis that targeting and inhibiting FGF2-dependent signaling downstream of FGFR1 is sufficient to inhibit cancer cell viability, we used a series of antibodies targeting individual or combinations of FGFRs: FGFR1 only, FGFR2+FGFR3, FGFR3 only, FGFR4 only, and FGFR1+FGFR2+FGFR3+FGFR4 (Ab15), and an FGFR1-driven cancer cell line, NCI-H2286. While the FGFR1-specific antibody reduced cell viability in this cell line, Ab15 showed superior activity (FIG. 1A). The same antibodies were tested in cancer cell lines with various expression levels of the individual FGFRs (IGROV1, Caki1, Ca151; FIGS. 1B-1D, respectively), and again Ab15 demonstrated superior activity. The relative levels of FGFR1-4 in these cell lines are shown in FIG. 1E. The activity of Ab15 in comparison to the other monospecific antibodies tested may be due to the fact that Ab15 binds to all four FGFRs (see Example 2) and reinforces the idea that targeting multiple FGFRs is necessary to substantially inhibit this pathway.

### Example 2

### Generation of Pan-FGFR Specific Binding Antibodies

Given the finding that targeting multiple FGFR receptors can enhance the therapeutic effects of anti-FGFR therapy, antibodies that specifically bind to all 4 FGFRs (FGFR1-4) were generated.

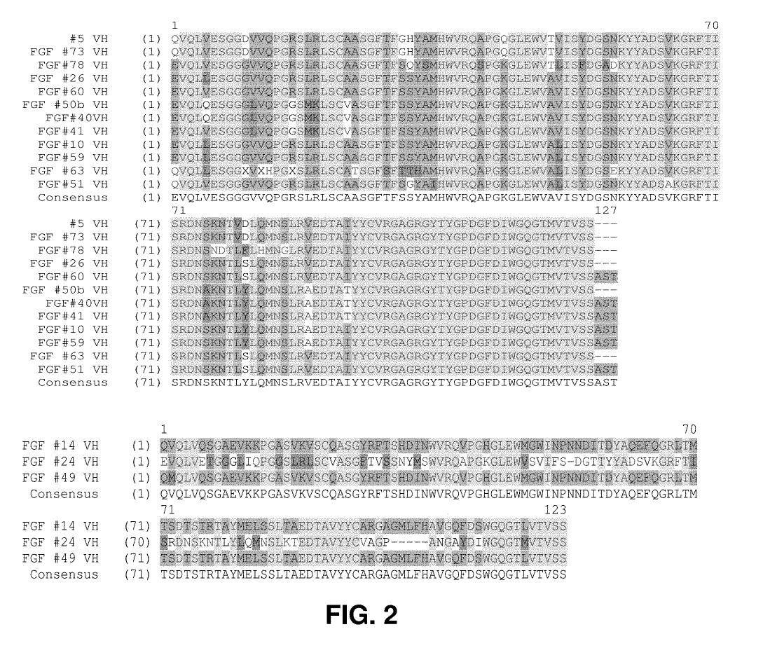

A summary of a subset of the anti-FGFR antibodies generated is provided with an alignment of heavy chain variable region sequences in FIG. 2.

### Example 3

### Cross-Reactivity of Anti-FGFR Antibodies with FGFR1c, FGFR2c, FGFR3c, and FGFR4

This Example describes the characterization of the cross-reactivity of the anti-FGFR antibodies generated in Example 2.

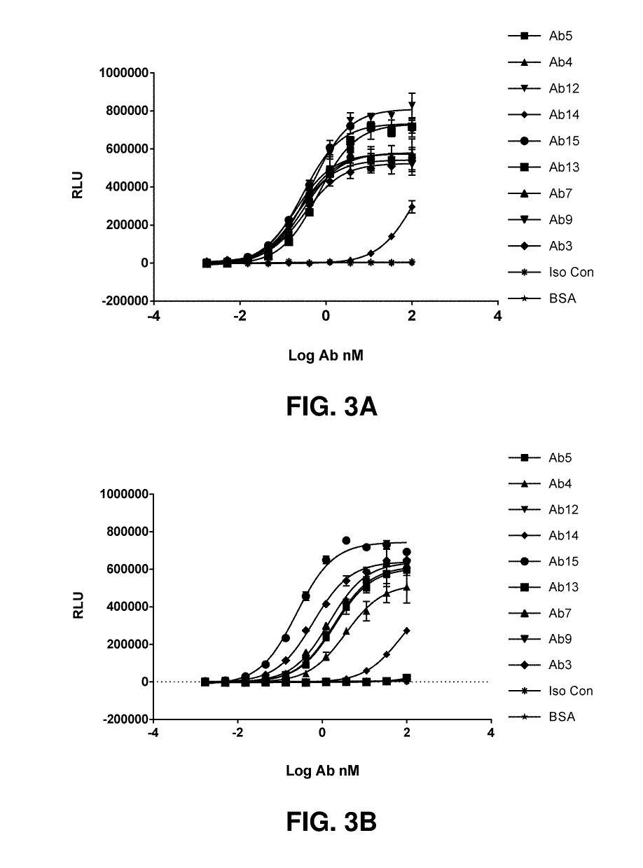

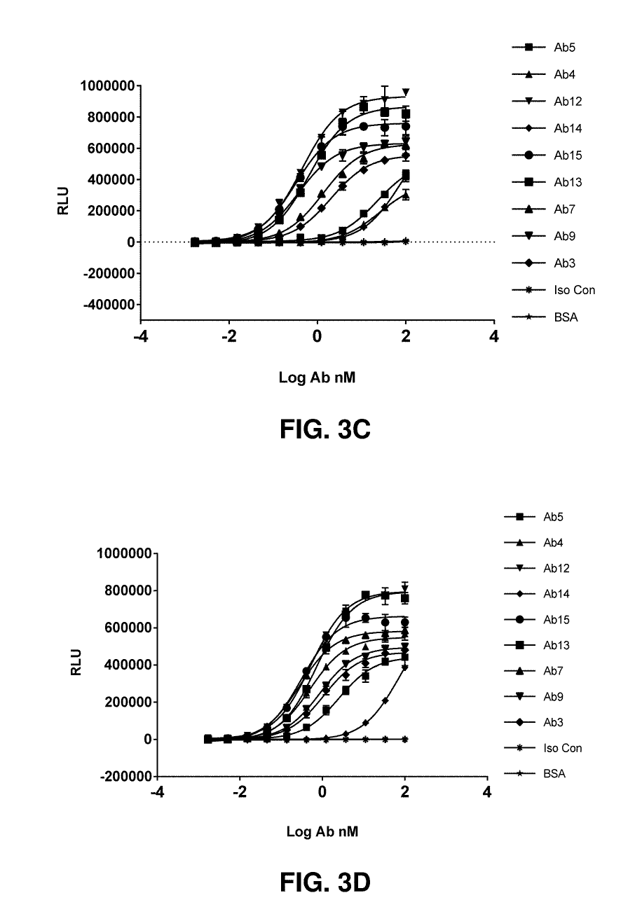

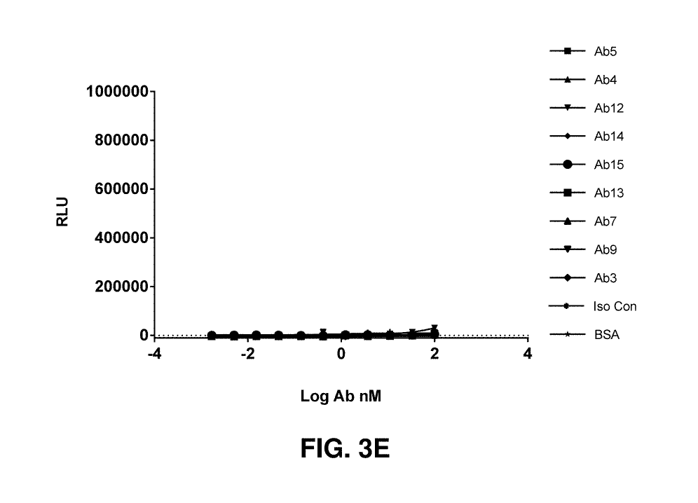

To characterize the novel anti-FGFR antibodies, their relative ability to bind to each individual FGFR in vitro was compared. A solid phase-based ELISA was used to measure the relative binding of the candidate antibodies to immobilized recombinant human FGFR1c, FGFR2c, FGFR3c, and FGFR4. EC50 binding curves were generated for a subset of candidates (FIGS. 3A-3D, respectively; Table 1).

For the ELISA assay, the ability of each mAb to cross react with the Mc isoform FGF receptors was tested across a concentration curve in order to determine the relative binding across FGFR1-4. FGFR2IIIb was used as a negative control. Black 384-well Maxisorp plates were coated overnight with FGFR-Fc fusions in PBS to 1 ug/ml before washing and blocking with 75 ul/well Pierce Protein Free (PBS) Blocking Buffer for 1 hour at RT. Antibody dilutions were prepared freshly in 1% BSA- PBS+0.05% Tween20 and 25 ul of each antibody was added to wells in duplicate. Plates were incubated at RT for 2 hours, followed by addition of detection antibody at 50 ng/ml in 1% BSA PBS, 0.05% Tween20. ECL substrate (Pierce) was added and plates were read on the EnVision plate reader.

As shown in FIG. 3E, none of the mAbs bound to FGFR2IIIb, indicating that these antibodies specifically target the Mc isoform rather than the IIIb isoform of FGFRs. The data in FIG. 3 illustrates that multiple candidates demonstrate binding properties consistent with cross-reactivity to all four FGFRs namely FGFR1c, FGFR2c, FGFR3c and FGFR4.

TABLE 1
EC50 values for anti-FGFR antibodies (in scFv format) binding to FGFR1c, FGFR2c, FGFR3c, and FGFR4

| EC50 [nM] | Ab5 | Ab4 | Ab12 | Ab14 | Ab15 | Ab13 | Ab7 | Ab9 | Ab3 |
|---|---|---|---|---|---|---|---|---|---|
| FGFR2IIIb | 0.06703 | 8.166 | 231.9 | ~556619 | ~79950 | ~103342 | ~96735 | 31.66 | ~108169 |
| FGFR1IIIc | 0.3041 | 0.2472 | 0.4619 | 128.6 | 0.2934 | 0.6607 | 0.2011 | 0.2204 | 0.3194 |
| FGFR2IIIc | 1.998 | 3.599 | ~53764 | 75.71 | 0.2474 | ~886911 | 1.47 | 1.927 | 0.6198 |
| FGFR3IIIc | 22.36 | 25.86 | 0.4475 | 77.19 | 0.3263 | 0.6464 | 1.356 | 0.3645 | 2.017 |
| FGFR4 | 2.539 | 0.5065 | 0.5304 | 71.97 | 0.3118 | 0.7363 | 0.2783 | 0.8632 | 1.075 |

### Example 4

### Anti-FGFR Antibodies Block the Binding of FGF Ligand to FGFR1 and FGFR4

This Example demonstrates the ability of candidate anti-FGFR antibodies to block the binding of ligand to FGF receptors.

To compare the ligand inhibitory potential of candidate anti-FGFR antibodies on FGF2-mediated ERK phosphorylation/activation, the AlphaScreen® SureFire® ERK 1/2 assay (cat # TGRTES500, Perkin Elmer, Waltham, Mass.) was performed. Ca151 cells were seeded at a density of 35,000 cells per well in 96-well plates and allowed to adhere overnight in complete medium consisting of RPMI supplemented with glutamine and 10% bovine serum albumin (BSA). The following day, all wells were washed with PBS and starved overnight in RPMI supplemented with 0.5% BSA (Sigma-Aldrich, St. Louis Mo., USA). Twenty-four hours later, cells were treated with a dilution series of each IgG diluted in RPMI/0.5% BSA for one hour prior to the addition of FGF1 or FGF2 (RnD Systems, Minn. USA) at 4 ng/ml for 10 min. Stimulation was halted by washing cells with ice cold PBS and the addition ice cold lysis buffer and storage at −80° C. AlphaScreen® SureFire® assays were performed according to the manufacturer's instructions. Data were analyzed using GraphPad Prism® software and plotted as a function of inhibition in comparison to ligand-only stimulated cells.

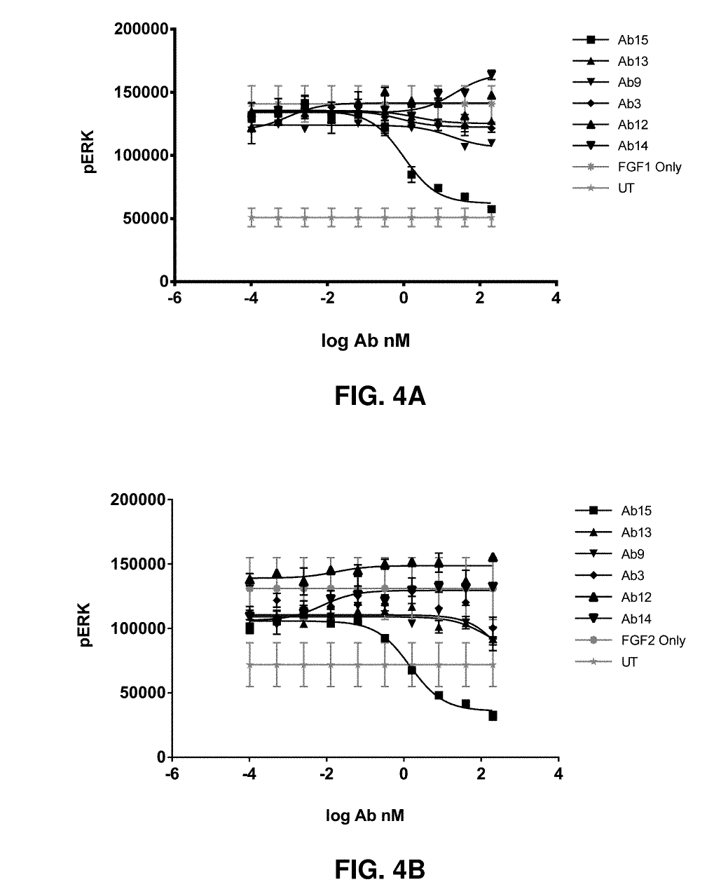

As shown in FIGS. 4A and 4B, a panel of FGFR targeting antibodies blocked pERK activation by FGF1 or FGF2, respectively. The data demonstrates that both FGF1 and FGF2 strongly activate pERK signaling in Ca151 cells and that targeting multiple FGFRs inhibits pERK activation.

### Example 5

### Isotype Switching, Characterization of mAb Formats, and Comparison of Weight Loss Induced by Anti-FGFRl Antibodies A1 and A1M5 in rodents

Sun et al. (Am J Physiol Endocrinol Metab 2007; 292:E964-76) previously reported that systemic delivery of the antagonist FGFR1 antibody, A1, caused potent but reversible hypophagia and weight loss in rodents and monkeys. This example demonstrates that antibody isotype and consequently immune effector functions contribute to the weight loss observed for this phenomenon in animals.

The A1 DNA sequence used in this Example was derived from the disclosure of WO 2005/037235 and modified with sequences to facilitate restriction enzyme digestion and cloning. The IgG2m4 sequence is described in An et al. (MAbs 2009;1:572-9), and was modified to include the C127S mutation. The genes were chemically synthesized using the DNA 2.0 expression system (Life Technologies, Grand Island, N.Y.) and cloned into the pCEP4 mammalian expression vector (Cat # V044-50, Life Technologies). Separate expression vectors were used to produce each protein chain (light and heavy) and co-expression of proteins was accomplished by co-transfection of selected combinations of vectors.

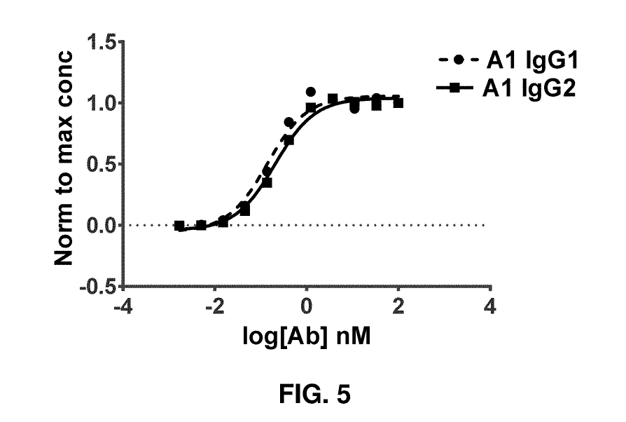

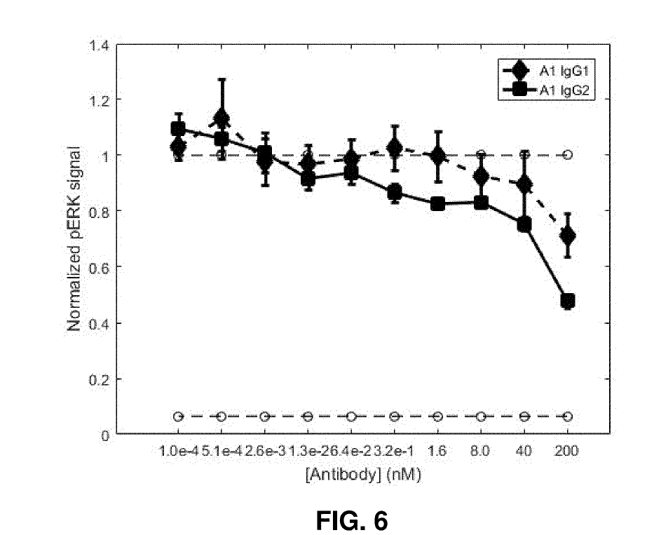

To ensure that isotype switching does not affect targeting of FGFR1, IgG1 and IgG2 formats of A1 (A1 and A1M5, respectively) were tested for solid-phase binding to FGFR1c-Fc by ELISA and in the signaling assay described in Example 4. FIG. 5 demonstrates that both A1 and A1M5 showed similar binding to FGFR1c. FIG. 6 demonstrates that both A1 (IgG1) and A1M5 (IgG2) inhibited FGF2-induced pERK activation with similar efficacy, albeit with A1M5 showing slightly more activity at the higher mAb concentrations. This data is in agreement with the FGFR1 binding ELISA (FIG. 5) and suggests that isotype does not grossly affect the activity of the two antibodies.

Next, the effect of isotype switching on the pharmacokinetic (PK) profile of A1 was tested in order to allow for a valid comparison of A1 and A1M5 antagonist activity (on-target effects) or isotype (off-target effects) on weight loss in vivo. To this end, the clearance rates of A1 and A1M5 from mice injected i.v. with a single dose of A1 and A1M5 were assessed by measuring plasma levels of human antibodies as a function of time. Specifically, normal female C57/BL6 mice were injected with 10 mg/kg of A1 or A1M5 and clearance rate was calculated by analysis of serum plasma levels of circulating human antibodies by ELISA as a function of time over 10 days.

As shown in Table 2, no significant difference was observed between the half-life of A1 and A1M5. Both A1 and A1M5 displayed a terminal half-life of approximately 180 hours, or about 1 week. This clearance rate is reported to be within the normal time range for clearance of human antibodies in mice.

TABLE 2
Pharmacokinetic profiles of A1 and
A1M5 monoclonal antibodies in mice

| | | | |
|---|---|---|---|
| IgG | No. of | Avg. half-life (h) | AUC |

| isotype | animals | α-phase | β-phase | (μg/mL/h) |
|---|---|---|---|---|
| IgG1A1 | 4 | 1.313 | 177.7 | 101.5 |
| IgG2M5 | 4 | 0.7523 | 159.7 | 100.6 |

Note:
AUC was calculated based on a bi-exponential fit of the data over 72 hours.

Taken together, these data indicate that swapping the isotype of A1 from IgG1 to an effector-reduced isotype (IgG2) does not compromise the activity or pharmacokinetic profile of A1. Accordingly, a valid comparison can be made on the relative contribution of antibody isotype to the weight loss observed with systemic administration of A1 to rodents and monkeys, as described by Sun et al. (supra).

To test this hypothesis, normal female C57BL/6 mice were injected with A1, A1M5, or a control monoclonal antibody at 1.0 and 10 mg/kg intraperitoneally (ip) three times per week for 2 weeks. Body weight and general health were recorded before treatment and every day during the treatment regime. To analyze the data, all weight changes were expressed as a percentage of the starting weight (g) and the experimental antibody weights of animal groups were plotted alongside antibody and saline controls to facilitate direct comparisons.

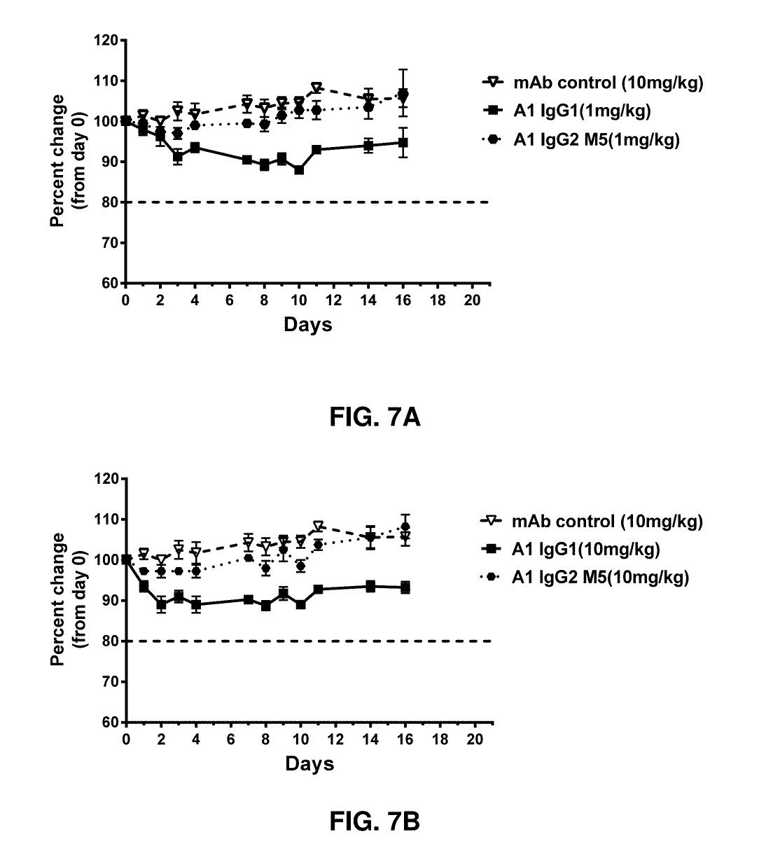

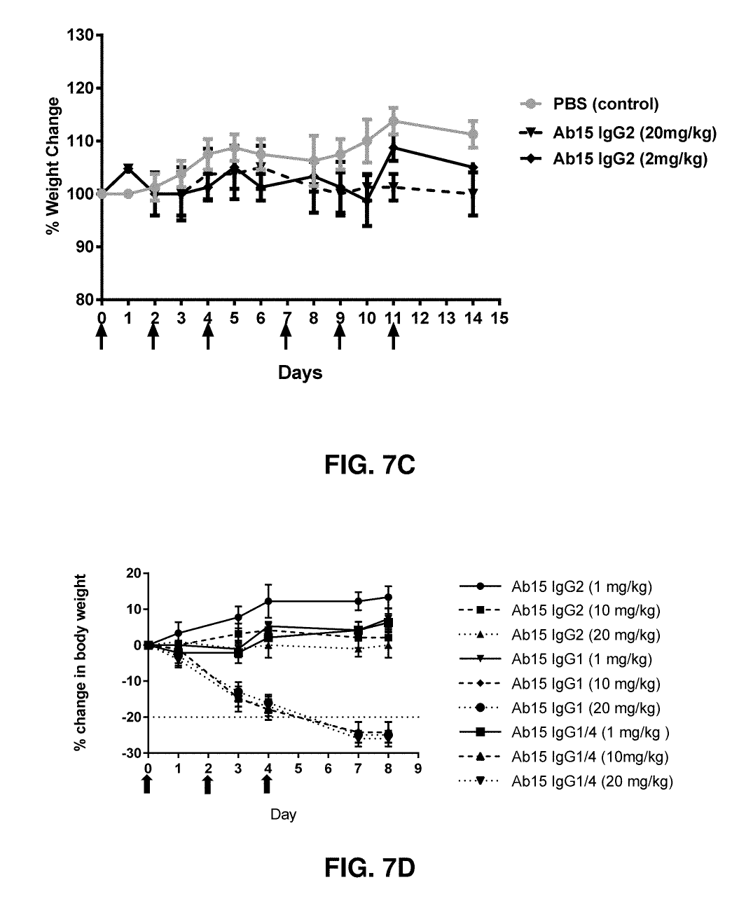

As shown in FIG. 7A, when A1 was dosed at 1 mg/kg, there was a significant decrease in body weight by day 4 that was not observed in the A1M5 or the control mAb. The weight loss observed for A1, but not A1M5 or controls, at 1 mg/kg was sustained during the two week treatment course. Furthermore, when the dose of antibodies was increased to 10 mg/kg, the weight loss observed for A1, but not A1M5 and controls, was more rapid than observed for the 1 mg/kg dose with a 10% loss of body weight observed on day 2 (FIG. 7B). As with the 1 mg/kg dose, the weight loss observed for A1, but not A1M5 or controls, at 10 mg/kg was sustained during the two week treatment course. As shown in FIG. 7C, when Ab15 (which has an M7 constant region) was dosed at 20 or 2 mg/kg, there was no significant decrease in body weight. These results suggest that the weight loss observed in mice with the A1 antibody is associated with the particular isotype used (IgG1), and that use of an antibody with reduced effector function (e.g., IgG2) could avoid the anorexic effects associated with the IgG1 isotype.

In another experiment, normal female C57BL/6 mice were injected with Ab15 variants with either no effector function (IgG2), full effector function (IgG1) or partial (IgG1/4) effector function at 1.0, 10 and 20 mg/kg intraperitoneally (ip) three times per week for 1 week. Body weight and general health were recorded before treatment and every day during the treatment regime. All weight changes were expressed as a percentage of the starting weight (g). As shown in FIG. 7D, when the Ab15 variants with either full or partial effector function were dosed at 10 or 20 mg/kg, there was a significant decrease in body weight by day 4. Ab15 in an IgG2 format was well tolerated and no weight loss was observed at any of the doses tested.

Furthermore, an experiment in which an antibody comprising the VH and VL region of an anti-FGFR1c antibody and the Fc regions of M9 (IgGl/IgG4 hybrid; SEQ ID NO: 1174) and M11 (IgG1 D265A/297Q; SEQ ID NO: 1175) caused weight loss similar to the A1 antibody.

### Example 6

### Anti-FGFR Antibodies Block the Binding of FGF Ligand to FGFR1 and FGFR4

This Example demonstrates the ability of candidate anti-FGFR antibodies to block the binding of ligand to FGFR1 and FGFR4 receptors using an in vitro binding assay. ELISA plates were coated with 50 ug/ml Heparin Sulfate (HSPG) (Sigma Aldrich) and incubated overnight at room temperature. Plates were then washed extensively with PBS containing 0.05% v/v Tween20 (PBST). Plates were then blocked with 2.5% BSA in PBST for one hour and washed extensively as before. 40-60 μL of FGF1 or FGF2 was then added to each well at 40 μg/ml and allowed to bind at room temperature for 2 hours. Plates were then washed extensively with PBST as before. Stock solutions of each 6His-Fc-FGFR (FGFR1-4) were prepared at 500 ng/ml in 2% v/v BSA-PBST and each antibody was prepared as a 2×-dilution series in 2% v/v BSA-PBST. Antibodies dilution series and 6His-Fc-FGFR1-4 were diluted in a 1:1 fashion and immediately added to the relative wells of the plate and allowed to bind for 2 hours at room temperature. Plates were washed extensively as before with PBST and the remaining fraction of bound 6His-Fc-FGFR was detected using a polyclonal-anti-His-6-HRP conjugated antibody (Abcam) for one hour at room temperature. After extensive washing with PBST, 50 μl of Pierce Chemiluminescent substrate (Thermo Fisher Scientific, Rockford, USA) was added and plates were measured on an Envision plate reader (Perkin Elmer). Raw data was background corrected and plotted using Prism software.

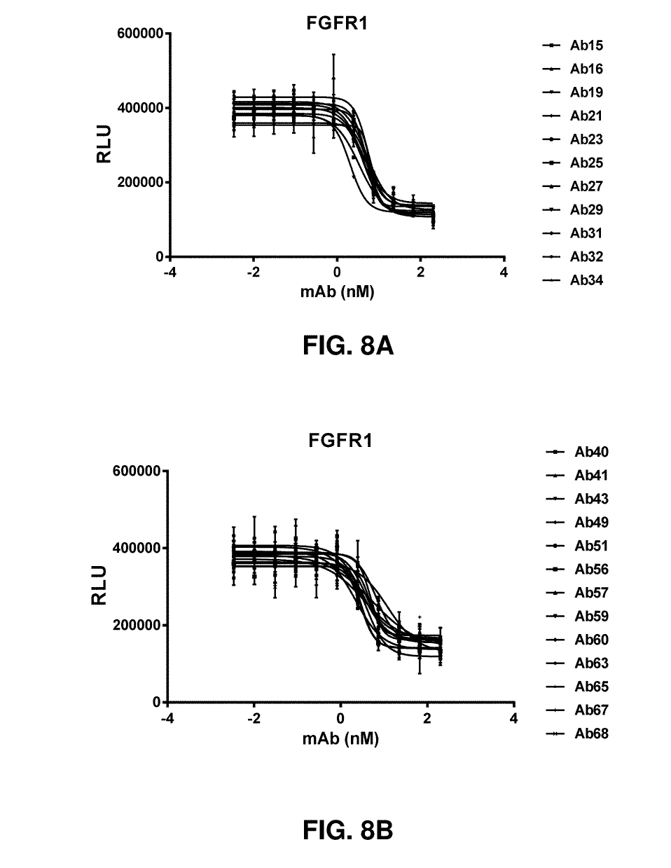

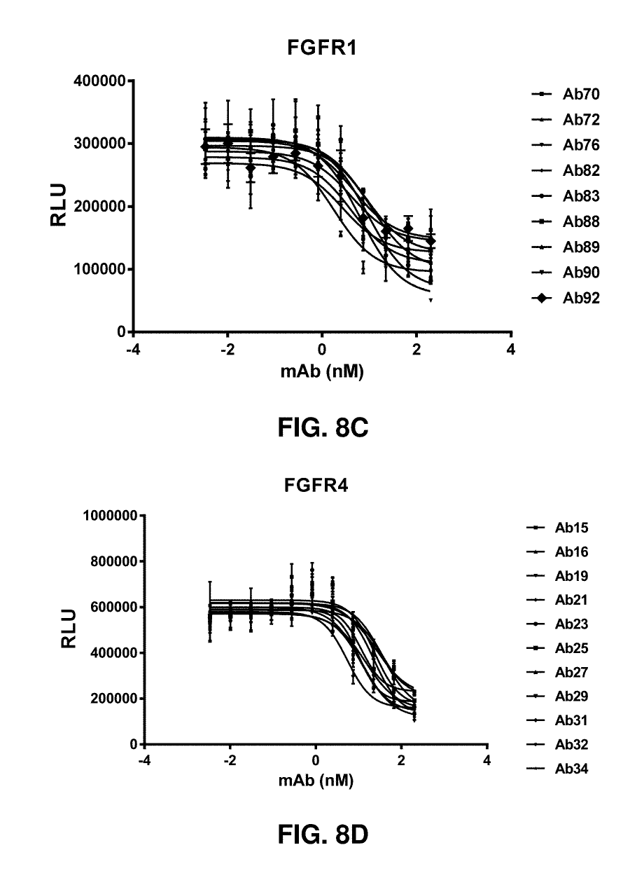

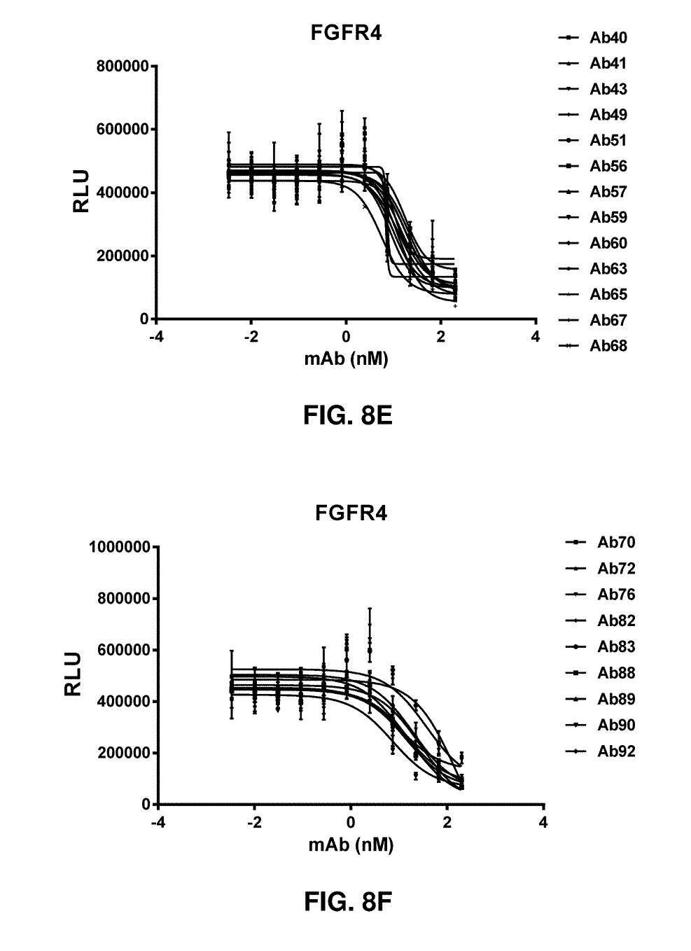

As shown in FIGS. 8A-8F, a panel of FGFR targeting antibodies blocked FGF1 binding to FGFR1 and FGFR4, respectively. The data demonstrates that multiple candidates bound to FGFR1 and FGFR4 and blocked ligand binding. Relative inhibitory capacity is presented in FIGS. 8A-8F). The data for blocking FGF2 binding to FGFR1c and FGFR4 is summarized in Table 3.

TABLE 3

| | IC50-FGFR1c [nM] | IC50-FGFR4 [nM] |
|---|---|---|
| Ab15 | 3.35 | 25.7 |
| Ab16 | 6.281 | 40.1 |
| Ab19 | 2.033 | 11.77 |
| Ab21 | 5.539 | 9.955 |
| Ab23 | 4.517 | 23.79 |
| Ab25 | 4.954 | 11.56 |
| Ab27 | 5.445 | 30.12 |
| Ab29 | 4.192 | 5.405 |
| Ab31 | 5.212 | 22.21 |
| Ab32 | 4.293 | 11.52 |
| Ab34 | 5.232 | 14.35 |
| Ab40 | 4.849 | 17.51 |
| Ab41 | 3.534 | 9.869 |
| Ab43 | 6.62 | 10.02 |
| Ab49 | 4.672 | 18.37 |
| Ab51 | 3.856 | 21.08 |
| Ab56 | 3.028 | 13.58 |
| Ab57 | 2.799 | 8.415 |
| Ab59 | 2.217 | ~6.857 |
| Ab60 | 3.834 | ~7.323 |
| Ab63 | 12.2 | 17.44 |
| Ab65 | 3.18 | 13.41 |
| Ab67 | 5.355 | 12.85 |
| Ab68 | 2.958 | 5.673 |
| Ab70 | 4.591 | 19.82 |
| Ab72 | 1.771 | 11.55 |
| Ab76 | 6.297 | 37.3 |
| Ab82 | 2.702 | 10.43 |
| Ab83 | 9.232 | 154.4 |
| Ab88 | 8.023 | 19 |
| Ab89 | 11.1 | 27.36 |
| Ab90 | 4.148 | 7.385 |
| Ab92 | 4.158 | 14.04 |

### Example 7

### Anti-FGFR Antibodies Block Activation of ERKIMAPK by Autocrine FGFR Ligand

This Example describes the characterization of the antagonist activities of candidate anti-FGFR antibodies by measuring their ability to block the activation of MAPK/ERK, a major signal transduction pathway directly downstream of FGFRs (Turner & Grose, 2010) in cellular systems.

An established assay that measures phosphorylated ERK (pERK) in cellular lysates (AlphaScreen SureFire) was used to assess the antagonist activities of the candidate anti-FGFR antibodies. This assay is an antibody-based assay which captures total ERK and measures the relative level of phosphorylation by means of a phosphorylation-specific antibody (Osmond et al., J Biomol Screen 2005; 10:730-7). The lung cancer cell line, NCI-H1581, has previously been shown to have an activated FGFR1 pathway driven by autocrine FGF18 (Weiss et al., Sci Transl Med 2010;2:62ra93). Consequently this cell line can be used to test FGFR targeting drugs.

Cells were seeded at 35,000 cells per well in 96-well plates. The following day, cells were incubated with a dilution series of candidate anti-FGFR antibodies (starting at 500 nM and with 3-fold dilutions) for 1 hour to allow binding of the antibody to FGFRs. The relative activation of pERK was calculated by comparison of signal strength to cells that were not incubated with the candidate antibodies, thereby allowing the calculation of relative IC50 values.

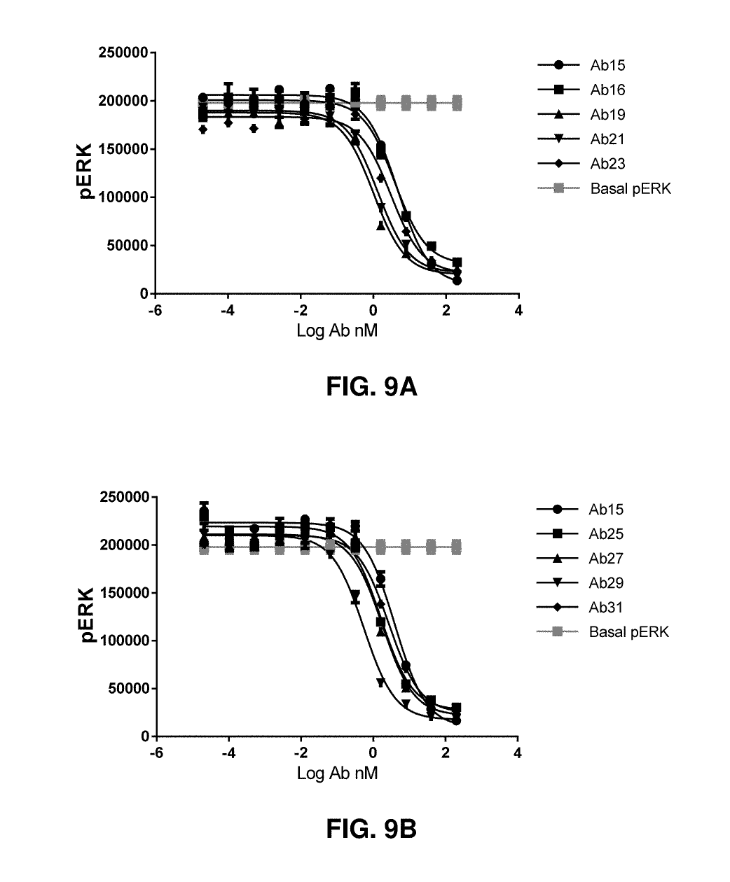

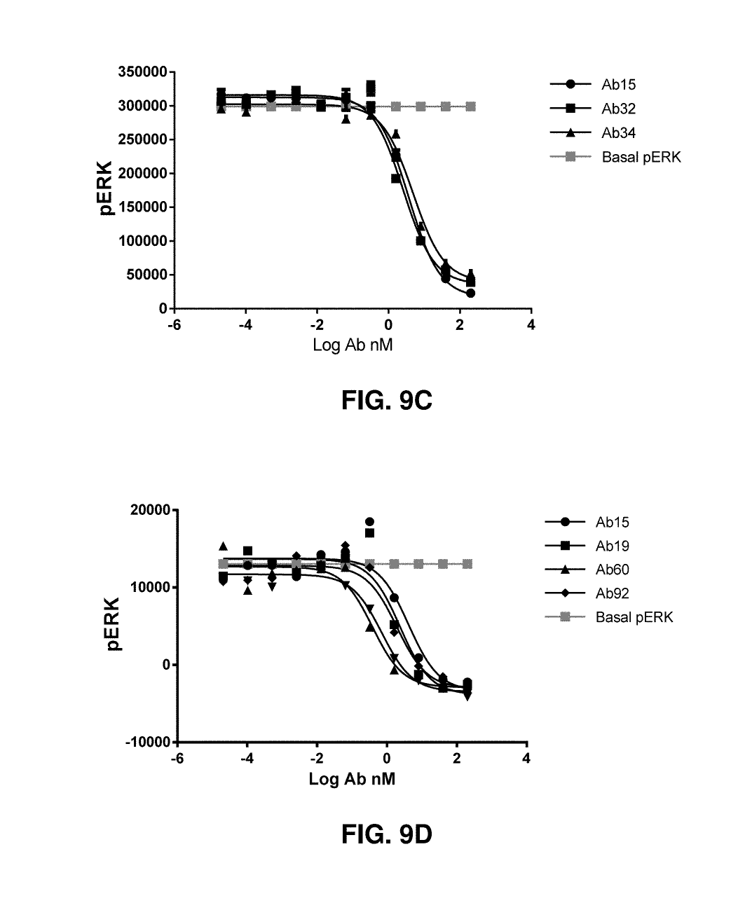

As shown in FIGS. 9A-9D,multiple candidates showed substantial inhibition of downstream signaling. The data is summarized in Table 4.

TABLE 4

| | IC50 [nM] |
|---|---|
| Ab15 | 4.12475 |
| Ab16 | 3.879 |
| Ab19 | 1.015 |
| Ab21 | 1.312 |
| Ab23 | 3.039 |
| Ab25 | 1.635 |
| Ab27 | 1.774 |
| Ab29 | 0.5404 |
| Ab31 | 2.717 |
| Ab32 | 2.55 |
| Ab34 | 4.835 |
| Ab60 | 0.3851 |
| Ab90 | 0.7152 |
| Ab92 | 1.912 |

### Example 8

### Anti-FGFR Antibodies Inhibit Viability of Tumor Cells

This Example demonstrates that anti-FGFR antibodies reduce the viability of tumor cells.

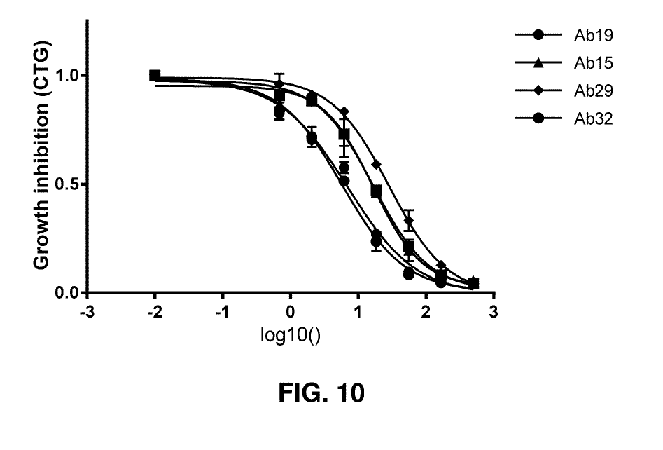

Each antibody was tested at a full concentration range to determine its relative inhibition of cell viability as measured by CellTiterGlo (CTG) in IGROV-1 cells (a human ovarian adenocarcinoma cell line). Cells were grown in complete growth medium (RPMI1640, 10% FBS, 1% Pen/Strep) and plated in 3% FBS on 96-well SCIVAX plates at 5,000 cells/well on Day 0. On Day 2 plates were examined for growth of cellular spheres and inhibitors of interest were added in 3-fold serial dilutions starting at 500 nM. Plates were placed in a TC incubator for three days before adding CTG reagent and reading chemiluminescence on a plate reader. As shown in FIG. 10, multiple FGFRIIIc targeting candidate antibodies reduced the viability of IGROV-1 cells. The data is summarized in Table 5.

TABLE 5

| | IC50 [nM] |
|---|---|
| Ab15 | 16.48 |
| Ab19 | 7.02 |
| Ab21 | 16.7 |
| Ab29 | 28.54 |
| Ab32 | 5.811 |

### Example 9

### Pharmacokinetic Comparison, Assessment of Efficacy, and Two Modes of Action of Pan-FGFR Antibodies in vivo

This Example describes the in vitro and in vivo characterization of Ab15 and Ab15 variants, and demonstrates their anti-tumor effects in mouse tumor models.

Table 6 shows the affinity of Ab15, Ab19, Ab60, Ab90, and Ab92 to FGFR1c, FGFR2c, FGFR3c, and FGFR4 using a solid phase ELISA as described in Example 3.

TABLE 6

| EC₅₀ [nM] | Ab15 | Ab19 | Ab60 | Ab90 | Ab92 |
|---|---|---|---|---|---|
| FGFR1c | 0.01120 | 0.006743 | 0.01165 | 0.01303 | 0.02419 |
| FGFR2c | 0.01470 | 0.01267 | 0.02240 | 0.03595 | 0.03248 |
| FGFR3c | 0.1291 | 0.07447 | 0.06873 | 0.1710 | 0.2642 |
| FGFR4c | 7.557 | 38.24 | 0.3104 | 1.262 | 13.05 |

Tables 7A and 7B show the affinity of Ab15, Ab19, Ab60, Ab90, Ab92, Vk1, Vk2, Vk3, Ab15, Vk5, Vk6, Vk7, Vk9, Vk10, Vk11, Vk12, Vk13, Ab15, Vk8, Vh1, and Vh2 for FGFR1c, FGFR2c, FGFR3c, and FGFR4 using the ForteBio assay, a bio-layer interferometery assay. Briefly, the Pall ForteBio Octet RED96 Bio-Layer Interferometry system was used to determine the specificity of anti-FGFR antibodies for FGFR1c, FGFR2c, FGFR3c, and FGFR4. The antibodies and soluble receptor proteins were prepared in PBS at a concentration of 300 nM. First, the candidate antibodies were immobilized on the surface of anti-human IgG Fc biosensor tips for 120 seconds. After immobilization, the biosensors were dipped in PBS solution for 60 seconds (baseline step) to assess assay drift, and determine loading level of antibodies. The baseline step was followed by an association step of 300 seconds wherein the binding interaction between the immobilized antibodies and soluble receptors was measured. Following the association step, the biosensors were dipped into PBS buffer for 600 seconds to measure the dissociation of the bound receptor proteins from the immobilized antibodies. Each binding response was measured and reported real time on a sensorgram trace. For data analysis, a simple 1:1 binding model was used which measures the rate of complex formation of one immobilized antibody and one soluble receptor protein.

TABLE 7A

| | FGFR1c | | | FGFR2c | | |
|---|---|---|---|---|---|---|
| | KD (M) | kon(1/Ms) | kdis(1/s) | KD (M) | kon(1/Ms) | kdis(1/s) |
| Ab15 | 1.37E−08 | 7.65E+04 | 1.21E−03 | 1.03E−08 | 1.83E+05 | 1.93E−03 |
| Ab19 | 8.85E−09 | 9.16E+04 | 9.67E−04 | 6.61E−09 | 2.52E+05 | 1.67E−03 |
| Ab60 | 1.45E−08 | 1.11E+05 | 1.65E−03 | 6.93E−09 | 2.54E+05 | 1.88E−03 |
| Ab90 | 1.20E−08 | 8.51E+04 | 1.16E−03 | 4.62E−09 | 2.18E+05 | 1.10E−03 |
| Ab92 | 2.00E−08 | 5.70E+04 | 1.29E−03 | 8.75E−09 | 1.47E+05 | 1.42E−03 |
| Vk1 | 4.70E−09 | 5.17E+04 | 2.43E−04 | 1.27E−08 | 1.93E+05 | 2.45E−03 |
| Vk2 | 5.74E−09 | 5.45E+04 | 3.13E−04 | 1.08E−08 | 2.11E+05 | 2.28E−03 |
| Vk3 | 4.79E−09 | 5.46E+04 | 2.62E−04 | 1.03E−08 | 2.14E+05 | 2.20E−03 |
| Ab15 | 4.72E−09 | 4.89E+04 | 2.31E−04 | 1.18E−08 | 1.99E+05 | 2.34E−03 |
| Vk5 | 3.98E−09 | 5.41E+04 | 2.15E−04 | 8.88E−09 | 1.98E+05 | 1.76E−03 |
| Vk6 | 6.45E−09 | 5.34E+04 | 3.44E−04 | 1.43E−08 | 2.19E+05 | 3.13E−03 |
| Vk7 | 5.84E−09 | 5.76E+04 | 3.36E−04 | 1.31E−08 | 2.10E+05 | 2.74E−03 |
| Vk9 | 2.39E−09 | 5.72E+04 | 1.37E−04 | 7.35E−09 | 2.34E+05 | 1.72E−03 |
| Vk10 | 5.33E−09 | 5.52E+04 | 2.94E−04 | 6.49E−09 | 2.20E+05 | 1.42E−03 |
| Vk11 | 7.62E−09 | 4.29E+04 | 3.27E−04 | 1.09E−08 | 2.12E+05 | 2.31E−03 |
| Vk12 | 6.51E−09 | 5.30E+04 | 3.45E−04 | 9.76E−09 | 2.15E+05 | 2.10E−03 |
| Vk13 | 7.33E−06 | 3.49E+04 | 2.56E−01 | 6.13E−06 | 1.04E+05 | 6.39E−01 |
| Ab15 | 7.39E−09 | 4.46E+04 | 3.30E−04 | 1.15E−08 | 2.05E+05 | 2.35E−03 |
| Vk8 | 2.51E−05 | 4.94E+03 | 1.24E−01 | 5.55E−08 | 6.26E+05 | 3.47E−02 |
| Vh1 | 7.49E−09 | 4.75E+04 | 3.56E−04 | 1.09E−08 | 2.21E+05 | 2.40E−03 |
| Vh2 | 5.41E−09 | 4.97E+04 | 2.69E−04 | 7.55E−09 | 2.07E+05 | 1.56E−03 |

*For Tables 7A and 7b, Vhl and Vh2 refer anti-FGFR antibodies in which Vh1 or Vh2 is combined with the VL of Ab15. Vk1, Vk2, Vk3, Vk5, Vk6, Vk7, Vk8, Vk9, Vk10, Vk11, Vk12, and Vk13 refer to anti-FGFR antibodies in which these Vks are combined with the VH of Ab15.

TABLE 7B

| | FGFR3c | | | FGFR4 | | |
|---|---|---|---|---|---|---|
| | KD (M) | kon(1/Ms) | kdis(1/s) | KD (M) | kon(1/Ms) | kdis(1/s) |
| Ab15 | 8.42E−09 | 1.21E+05 | 1.08E−03 | 1.12E−08 | 9.82E+04 | 1.24E−03 |
| Ab19 | 6.29E−09 | 1.51E+05 | 9.83E−04 | 8.63E−09 | 1.25E+05 | 1.20E−03 |
| Ab60 | 1.54E−08 | 1.54E+05 | 2.39E−03 | 1.25E−08 | 1.17E+05 | 1.69E−03 |
| Ab90 | 1.16E−08 | 1.26E+05 | 1.50E−03 | 6.69E−09 | 1.12E+05 | 8.87E−04 |
| Ab92 | 3.76E−08 | 7.56E+04 | 2.86E−03 | 4.03E−08 | 6.06E+04 | 2.48E−03 |
| Vk1 | 1.23E−08 | 1.13E+05 | 1.38E−03 | 8.75E−09 | 1.20E+05 | 1.05E−03 |
| Vk2 | 9.20E−09 | 1.20E+05 | 1.10E−03 | 7.06E−09 | 1.26E+05 | 8.88E−04 |
| Vk3 | 7.97E−09 | 1.26E+05 | 1.01E−03 | 6.79E−09 | 1.26E+05 | 8.55E−04 |
| Ab15 | 8.59E−09 | 1.23E+05 | 1.05E−03 | 7.35E−09 | 1.24E+05 | 9.10E−04 |
| Vk5 | 7.00E−09 | 1.27E+05 | 8.91E−04 | 6.05E−09 | 1.24E+05 | 7.52E−04 |
| Vk6 | 1.29E−08 | 1.21E+05 | 1.57E−03 | 9.31E−09 | 1.20E+05 | 1.12E−03 |
| Vk7 | 8.70E−09 | 1.20E+05 | 1.04E−03 | 8.33E−09 | 1.21E+05 | 1.01E−03 |
| Vk9 | 5.43E−09 | 1.38E+05 | 7.49E−04 | 4.89E−09 | 1.42E+05 | 6.92E−04 |
| Vk10 | 5.23E−09 | 1.34E+05 | 7.01E−04 | 4.43E−09 | 1.32E+05 | 5.85E−04 |
| Vk11 | 1.11E−08 | 1.17E+05 | 1.30E−03 | 8.47E−09 | 1.25E+05 | 1.06E−03 |
| Vk12 | 6.38E−09 | 1.26E+05 | 8.06E−04 | 7.01E−09 | 1.32E+05 | 9.25E−04 |
| Vk13 | 1.09E−06 | 3.53E+04 | 3.86E−02 | 3.64E−11 | 1.15E+05 | 4.19E−06 |
| Ab15 | 8.21E−09 | 1.24E+05 | 1.02E−03 | 7.88E−09 | 1.28E+05 | 1.01E−03 |
| Vk8 | 3.83E−08 | 5.03E+05 | 1.92E−02 | 8.56E−08 | 3.23E+05 | 2.76E−02 |
| Vh1 | 8.56E−09 | 1.17E+05 | 1.00E−03 | 4.01E−09 | 1.70E+05 | 6.82E−04 |
| Vh2 | 6.41E−09 | 1.21E+05 | 7.75E−04 | 2.84E−09 | 1.73E+05 | 4.92E−04 |

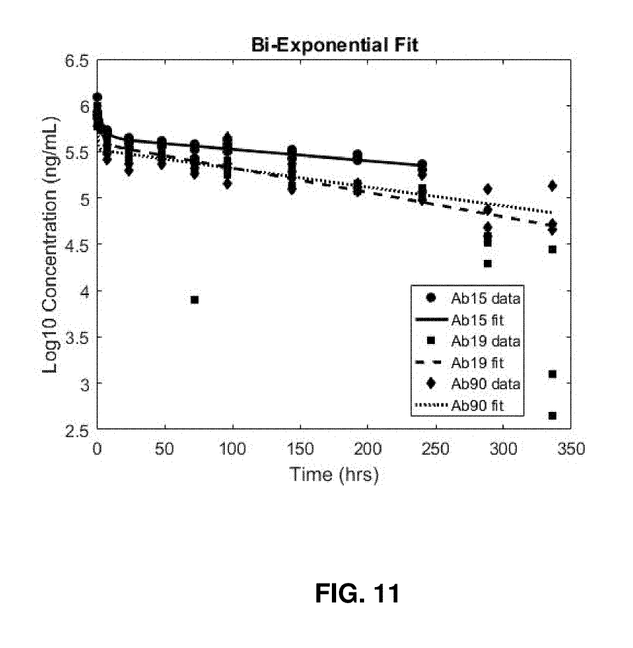

Ab15, Ab19, and Ab90 showed similar PK profiles with respect to their in vivo terminal half-life in vivo (FIG. 11, Table 8). Mice were injected into the tail vein with a single dose of the antibodies (at 5, 10, 20 or 40 mg/kg) and bled at predetermined time points over a 2-week period. Blood was processed to serum before being stored at −80C and analysis by ELISA.

TABLE 8
Pharmacokinetic profiles of pan-FGFR antibodies in mice

| | | |
|---|---|---|
| No. of | Avg. half life (h) | AUC |

| Molecule | animals | α-phase | β-phase | (μg/mL/h) |
|---|---|---|---|---|
| Ab15 | 4 | 3.85 | 239.02 | 428.56 |
| Ab19 | 4 | 2.24 | 113.63 | 332.34 |
| Ab90 | 4 | 0.36 | 147.48 | 286.07 |

Note:
AUC was calculated based on a biexponential fit of the data over 72 hours

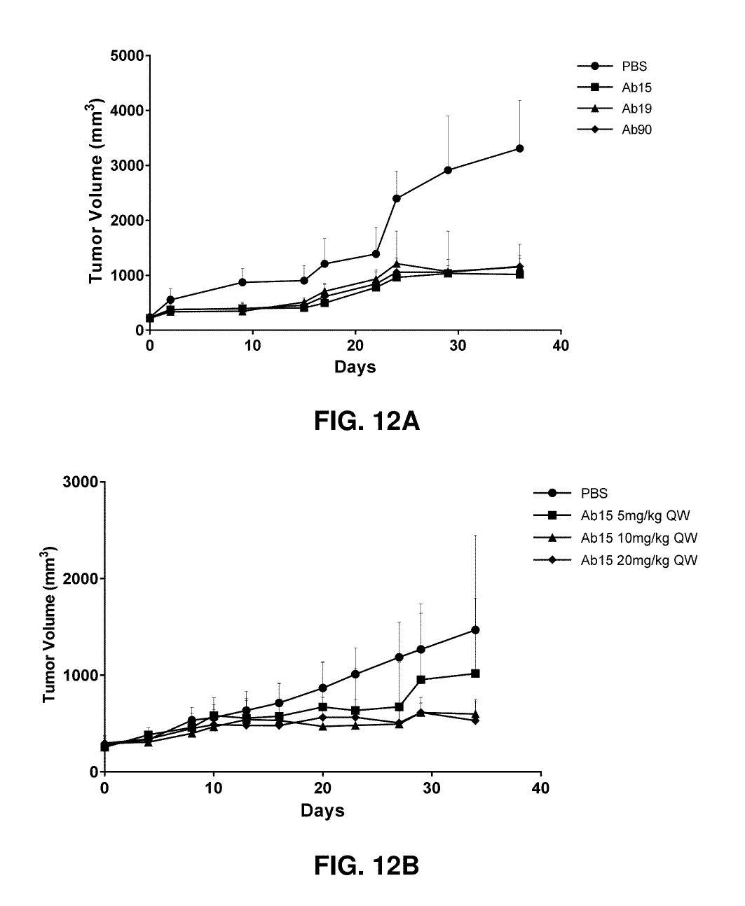

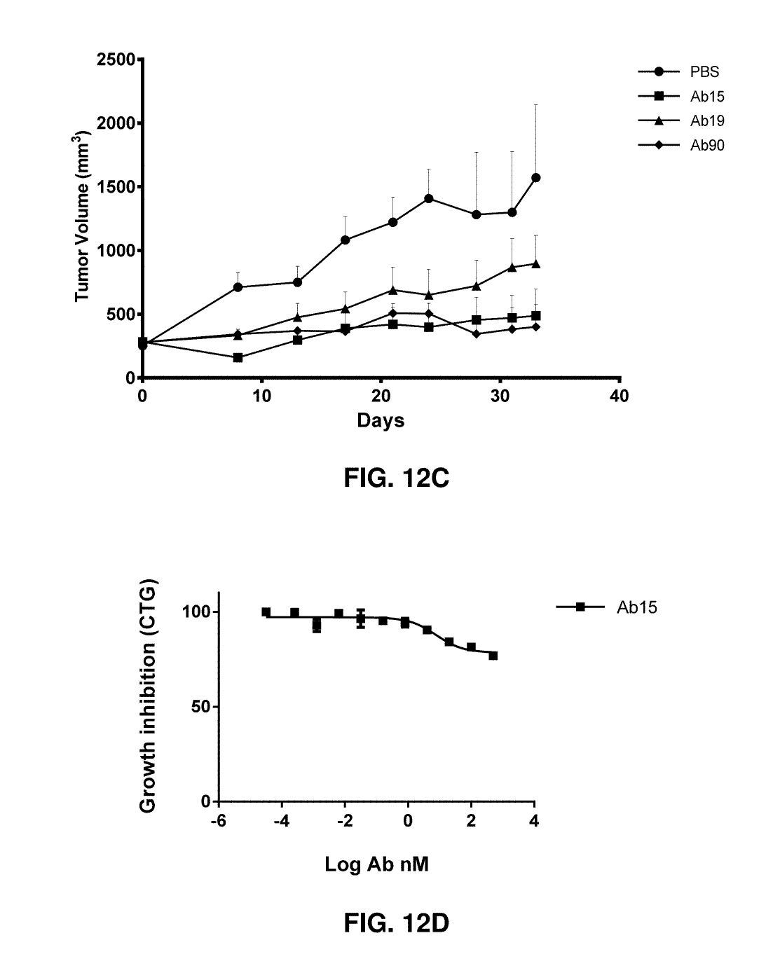

Ab15, Ab19, and Ab90 were assessed with respect to their in vivo efficacy in a mesothelioma model, MFE280 (FIG. 12A). 5×10⁶ cells of the respective cell line were inoculated s.c. into nude mice using 50% GFR matrigel. Anti-FGFR antibodies were dosed Q1W at 8-10 mg/kg by i.p injection. Doses used for the individual antibodies were adjusted based on the PK studies and body weight and tumor volumes were assessed twice weekly. Ab15 was additionally tested in an endometrial xenograft model, MFE280 (FIG. 12B), a renal xenograft model, SN12C (FIG. 12C).

In all models, Ab15 was highly efficacious in suppressing tumor growth in a dose-dependent manner. Ab15 was a non-responder in the mesothelioma model MSTO211H in vitro (FIG. 12D) but Ab15 potently inhibited growth in vivo (FIG. 12A). This suggests that Ab15 not only acts as an anti-proliferative agent, but also as an anti-angiogenic agent.

TABLE 9
SUMMARY OF SEQUENCES

| SEQ | | |
|---|---|---|
| ID | Description | SEQUENCE |
| 1 | FGFR1 IIIc | TDNTKPNPVAPYWTSPEKMEKKLHAVPAAKTVKFKCPSSGTPNPTLRWLKNGKEFKPDH |
| | (human) | RIGGYKVRYATWSIIMDSVVPSDKGNYTCIVENEYGSINHTYQLDVVERSPHRPILQAG |
| | NM_001174066.1 | LPANKTVALGSNVEFMCKVYSDPQPHIQWLKHIEVNGSKIGPDNLPYVQILKTAGVNTT |
| | (mouse) | DKEMEVLHLRNVSFEDAGEYTCLAGNSIGLSHHSAWLTVLEALEERPAVMTSPLYLE |
| | XM_006509014.1, | **For all FGFR sequences (i.e., SEQ ID NOs: 1-22, the sequences |
| | NM_01079908.1, | provided correspond to extracellular fragments of the mature sequence |
| | XP_006509077.1 | after leader peptide cleavage |
| | (cynomolgus) | |
| | AB220417.1 | |
| | (RNA) | |
| 2 | FGFR1 IIIc | TDNTKPNPVAPYWTSPEKMEKKLHAVPAAKTVKFKCPSSGTPSPTLRWLKNGKEFKPDH |
| | (rat) | RIGGYKVRYATWSIIMDSVVPSDKGNYTCIVENEYGSINHTYQLDVVERSPHRPILQAG |
| | XP_006253388, | LPANKTVALGSNVEFMCKVYSDPQPHIQWLKHIEVNGSKIGPDNLPYVQILKTAGVNTT |
| | XM_006253328.1 | DKEMEVLHLRNVSFEDAGEYTCLAGNSIGLSHHSAWLTVLEALEERPAVMTSPLYLE |
| 3 | FGFR1 IIIb | TDNTKPNPVAPYWTSPEKMEKKLHAVPAAKTVKFKCPSSGTPNPTLRWLKNGKEFKPDH |
| | (human) | RIGGYKVRYATWSIIMDSVVPSDKGNYTCIVENEYGSINHTYQLDVVERSPHRPILQAG |
| | FJ809917.1 | LPANKTVALGSNVEFMCKVYSDPQPHIQWLKHIEVNGSKIGPDNLPYVQILKHSGINSS |
| | (mouse) | DAEVLTLFNVTEAQSGEYVCKVSNYIGEANQSAWLTVTRPVAKALEERPAVMTSPLYLE |
| | AF176552.1, | |
| | AF176552.1, | |
| | NW_001030892.1 | |
| | (cynomolgus) | |
| | NW_005092974.1 | |
| 4 | FGFR1 IIIb | TDNTKPNPVAPYWTSPEKMEKKLHAVPAAKTVKFKCPSSGTPSPTLRWLKNGKEFKPDH |
| | (rat) | RIGGYKVRYATWSIIMDSVVPSDKGNYTCIVENEYGSINHTYQLDVVERSPHRPILQAG |
| | AC_000084.1 | LPANKTVALGSNVEFMCKVYSDPQPHIQWLKHIEVNGSKIGPDNLPYVQILKHSGINSS |
| | | DAEVLTLFNVTEAQSGEYVCKVSNYIGEANQSAWLTVTRPVPKALEERPAVMTSPLYLE |
| 5 | FGFR2 IIIc | SNNKRAPYWTNTEKMEKRLHAVPAANTVKFRCPAGGNPMPTMRWLKNGKEFKQEHRIGG |
| | (human) | YKVRNQHWSLIMESVVPSDKGNYTCVVENEYGSINHTYHLDVVERSPHRPILQAGLPAN |
| | XM_006717713.1 | ASTVVGGDVEFVCKVYSDAQPHIQWIKHVEKNGSKYGPDGLPYLKVLKAAGVNTTDKEI |
| | | EVLYIRNVTFEDAGEYTCLAGNSIGISFHSAWLTVLPAPGREKEITASPDYLE |
| 6 | FGFR2 IIIc | RSNQRAPYWTNTEKMEKRLHAVPAANTVKFRCPAGGNPTPTMRWLKNGKEFKQEHRIGG |
| | (mouse) | YKVRNQHWSLIMESVVPSDKGNYTCLVENEYGSINHTYHLDVVERSPHRPILQAGLPAN |
| | XM_06507355.1 | ASTVVGGDVEFVCKVYSDAQPHIQWIKHVEKNGSKYGPDGLPYLKVLKAAGVNTTDKEI |
| | (rat) | EVLYIRNVTFEDAGEYTCLAGNSIGISFHSAWLTVLPAPVREKEITASPDYLE |
| | NM_001109894.1 | |
| 7 | FGFR2 IIIc | GNNKRAPYWTNTEKMEKRLHAVPAANTVKFRCPAGGNPTPTMRWLKNGKEFKQEHRIGG |
| | (cynomolgus) | YKVRNQHWSLIMESVVPSDKGNYTCVVENEYGSINHTYHLDVVERSPHRPILQAGLPAN |
| | XM_005566598.1 | ASTVVGGDVEFVCKVYSDAQPHIQWIKHVEKNGSKYGPDGLPYLKVLKAAGVNTTDKEI |
| | | EVLYIRNVTFEDAGEYTCLAGNSIGISFHSAWLTVLPAPGREKEITASPDYLE |
| 8 | FGFR2 IIIb | SNNKRAPYWTNTEKMEKRLHAVPAANTVKFRCPAGGNPMPTMRWLKNGKEFKQEHRIGG |
| | (human) | YKVRNQHWSLIMESVVPSDKGNYTCVVENEYGSINHTYHLDVVERSPHRPILQAGLPAN |
| | X56191.1 | ASTVVGGDVEFVCKVYSDAQPHIQWIKHVEKNGSKYGPDGLPYLKVLKHSGINSSNAEV |
| | | LALFNVTEADAGEYICKVSNYIGQANQSAWLTVLPKQQAPGREKEITASPDYLE |
| 9 | FGFR2 IIIb | RSNQRAPYWTNTEKMEKRLHAVPAANTVKFRCPAGGNPTPTMRWLKNGKEFKQEHRIGG |
| | (mouse) | YKVRNQHWSLIMESVVPSDKGNYTCLVENEYGSINHTYHLDVVERSPHRPILQAGLPAN |
| | XM_006507356.1, | ASTVVGGDVEFVCKVYSDAQPHIQWIKHVEKNGSKYGPDGLPYLKVLKHSGINSSNAEV |
| | M63503.1 | LALFNVTEMDAGEYICKVSNYIGQANQSAWLTVLPKQQAPVREKEITASPDYLE |
| | (rat) | |
| | XM_006230392.1 | |
| 10 | FGFR2 IIIb | GNNKRAPYWTNTEKMEKRLHAVPAANTVKFRCPAGGNPTPTMRWLKNGKEFKQEHRIGG |
| | (cynomolgus) | YKVRNQHWSLIMESVVPSDKGNYTCVVENEYGSINHTYHLDVVERSPHRPILQAGLPAN |
| | XM_005566597.1 | ASTVVGGDVEFVCKVYSDAQPHIQWIKHVEKNGSKYGPDGLPYLKVLKHSGINSSNAEV |
| | | LALFNVTEADAGEYICKVSNYIGQANQSAWLTVLPKQQAPGREKEITASPDYLE |
| 11 | FGFR3 IIIc | AEDTGVDTGAPYWTRPERMDKKLLAVPAANTVRFRCPAAGNPTPSISWLKNGREFRGEH |
| | (human) | RIGGIKLRHQQWSLVMESVVPSDRGNYTCVVENKFGSIRQTYTLDVLERSPHRPILQAG |
| | XM_06713872.1, | LPANQTAVLGSDVEFHCKVYSDAQPHIQWLKHVEVNGSKVGPDGTPYVTVLKTAGANTT |
| | M58051.1 | DKELEVLSLHNVTFEDAGEYTCLAGNSIGFSHHSAWLVVLPAEEELVEADEAGSV |
| 12 | FGFR3 IIIc | GEDVAEDTGAPYWTRPERMDKKLLAVPAANTVRFRCPAAGNPTPSISWLKNGKEFRGEH |
| | (mouse) | RIGGIKLRHQQWSLVMESVVPSDRGNYTCVVENKFGSIRQTYTLDVLERSPHRPILQAG |
| | NM_001205270.1 | LPANQTAILGSDVEFHCKVYSDAQPHIQWLKHVEVNGSKVGPDGTPYVTVLKTAGANTT |
| | | DKELEVLSLHNVTFEDAGEYTCLAGNSIGFSHHSAWLVVLPAEEELMETDEAGSV |
| 13 | FGFR3 IIIc | GEDVAEDTGAPYWTRPERMDKKLLAVPAANTVRFRCPAAGNPTPSISWLKNGKEFRGEH |
| | (rat) | RIGGIKLRHQQWSLVMESVVPSDRGNYTCVVENKFGSIRQTYTLDVLERSPHRPILQAG |
| | XM_006251394.1 | LPANQTAVLGSDVEFHCKVYSDAQPHIQWLKHVEVNGSKVGPDGTPYVTVLKTAGANTT |
| | | DRELEVLSLHNVTFEDAGEYTCLAGNSIGFSHHSAWLVVLPAEEELMEVDEAGSV |
| 14 | FGFR3 IIIc | AEDTGVDTGAPYWTRPERMDKKLLAVPAANTVRFRCPAAGNPTPSISWLKNGKEFRGEH |
| | (cynomolgus) | RIGGIKLRHQQWSLVMESVVPSDRGNYTCVVENKFGSIRQTYTLDVLERSPHRPILQAG |
| | XM_005554287.1 | LPANQTAVLGSDVEFHCKVYSDAQPHIQWLKHVEVNGSKVGPDGTPYVTVLKTAGANTT |
| | | DKELEVLSLHNVTFEDAGEYTCLAGNSIGFSHHSAWLVVLPAEEELVEADEAGSV |
| 15 | FGFR3 IIIb | AEDTGVDTGAPYWTRPERMDKKLLAVPAANTVRFRCPAAGNPTPSISWLKNGREFRGEH |
| | (human) | RIGGIKLRHQQWSLVMESVVPSDRGNYTCVVENKFGSIRQTYTLDVLERSPHRPILQAG |
| | NM_001163213.1, | LPANQTAVLGSDVEFHCKVYSDAQPHIQWLKHVEVNGSKVGPDGTPYVTVLKSWISESV |
| | XM_006713869.1 | EADVRLRLANVSERDGGEYLCRATNFIGVAEKAFWLSVHGPRAAEEELVEADEAGSV |
| 16 | FGFR3 IIIb | GEDVAEDTGAPYWTRPERMDKKLLAVPAANTVRFRCPAAGNPTPSISWLKNGKEFRGEH |
| | (mouse) | RIGGIKLRHQQWSLVMESVVPSDRGNYTCVVENKFGSIRQTYTLDVLERSPHRPILQAG |
| | NM_001163217.2 | LPANQTAILGSDVEFHCKVYSDAQPHIQWLKHVEVNGSKVGPDGTPYVTVLKSWISENV |
| | | EADARLRLANVSERDGGEYLCRATNFIGVAEKAFWLRVHGPQAAEEELMETDEAGSV |
| 17 | FGFR3 IIIb | GEDVAEDTGAPYWTRPERMDKKLLAVPAANTVRFRCPAAGNPTPSISWLKNGKEFRGEH |
| | (rat) | RIGGIKLRHQQWSLVMESVVPSDRGNYTCVVENKFGSIRQTYTLDVLERSPHRPILQAG |
| | XM_006251392.1 | LPANQTAVLGSDVEFHCKVYSDAQPHIQWLKHVEVNGSKVGPDGTPYVTVLKSWISENV |
| | | EADARLRLANVSERDGGEYLCRATNFIGVAEKAFWLRVHGPQAAEEELMEVDEAGSV |
| 18 | FGFR3 IIIb | AEDTGVDTGAPYWTRPERMDKKLLAVPAANTVRFRCPAAGNPTPSISWLKNGKEFRGEH |
| | (cynomolgus) | RIGGIKLRHQQWSLVMESVVPSDRGNYTCVVENKFGSIRQTYTLDVLERSPHRPILQAG |
| | XM_005554285.1 | LPANQTAVLGSDVEFHCKVYSDAQPHIQWLKHVEVNGSKVGPDGTPYVTVLKSWISESV |
| | | EADVRLRLANVSERDGGEYLCRATNFIGVAEKAFWLSVHRPRAAEEELVEADEAGSV |
| 19 | FGFR4 | QQAPYWTHPQRMEKKLHAVPAGNTVKFRCPAAGNPTPTIRWLKDGQAFHGENRIGGIRL |
| | (human) | RHQHWSLVMESVVPSDRGTYTCLVENAVGSIRYNYLLDVLERSPHRPILQAGLPANTTA |
| | JN007482.1, | VVGSDVELLCKVYSDAQPHIQWLKHIVINGSSFGADGFPYVQVLKTADINSSEVEVLYL |
| | XR_427801.1 | RNVSAEDAGEYTCLAGNSIGLSYQSAWLTVLPEEDPTWTAAAPEARYTD |
| 20 | FGFR4 | QQAPYWTHPQRMEKKLHAVPAGNTVKFRCPAAGNPMPTIHWLKDGQAFHGENRIGGIRL |
| | (mouse) | RHQHWSLVMESVVPSDRGTYTCLVENSLGSIRYSYLLDVLERSPHRPILQAGLPANTTA |
| | AY493377.2, | VVGSDVELLCKVYSDAQPHIQWLKHVVINGSSFGADGFPYVQVLKTTDINSSEVEVLYL |
| | NM_008011.2, | RNVSAEDAGEYTCLAGNSIGLSYQSAWLTVLPEEDLTWTTATPEARYTD |
| | XM_006517099.1 | |
| 21 | FGFR4 | QQAPYWTHPQRMEKKLHAVPAGNTVKFRCPAAGNPMPTIHWLKNGQAFHGENRIGGIRL |
| | (rat) | RHQHWSLVMESVVPSDRGTYTCLVENSLGSIRYSYLLDVLERSPHRPILQAGLPANTTA |
| | NM_001109904.1, | VVGSNVELLCKVYSDAQPHIQWLKHIVINGSSFGADGFPYVQVLKTTDINSSEVEVLYL |
| | BC100260, | RNVSAEDAGEYTCLAGNSIGLSYQSAWLTVLPAEEEDLAWTTATSEARYTD |
| | XM_006253605.1 | |
| 22 | FGFR4 | QQAPYWTHPQRMEKKLHAVPAGNTVKFRCPAAGNPTPTIRWLKDGQAFHGENRIGGIRL |
| | (cynomolgus) | RHQHWSLVMESVVPSDRGTYTCLVENAVGSIRYNYLLDVLERSPHRPILQAGLPANTTA |
| | XM_005558623.1 | VVGSDVELLCKVYSDAQPHIQWLKHIVINGSSFGADGFPYVQVLKTADINSSEVEVLYL |
| | | RNVSAEDAGEYTCLAGNSIGLSYQSAWLTVLPEEDLTWTAATPEARYTD |
| 23 | VHCDR1 Ab1 | SYAMH |
| 24 | VHCDR2 Ab1 | VISYDGSNKYYADSVKG |
| 25 | VHCDR3 Ab1 | GAGRGYTYGPDGFDI |
| 26 | VLCDR1 Ab1 | RSSQSLVYTDGITYLS |
| 27 | VLCDR2 Ab1 | EISNRFS |
| 28 | VLCDR3 Ab1 | MQATQFPWT |
| 29 | VH Ab1 | EVQLVESGGGLVQPGGSMKLSCVASGFTFSSYAMHWVRQAPGKGLEWVAVISYDGSNKY |
| | (referred to as | YADSVKGRFTISRDNAKNTLYLQMNSLRAEDTATYYCVRGAGRGYTYGPDGFDIWGQGT |
| | FGF#41 in FIG. | MVTVSS |
| | 2) | |
| 30 | VL Ab1 | DIVMTQSPRSLPVTLGQPASISCRSSQSLVYTDGITYLSWLQQRPGQPPRLLIYEISNR |
| | | FSGVPDRFSGSGAGTDFTLKISRVEAEDVGVYYCMQATQFPWTFGQGTKVDIK |
| 31 | VHVL Ab1 | EVQLVESGGGLVQPGGSMKLSCVASGFTFSSYAMHWVRQAPGKGLEWVAVISYDGSNKY |
| | (scFv) | YADSVKGRFTISRDNAKNTLYLQMNSLRAEDTATYYCVRGAGRGYTYGPDGFDIWGQGT |
| | | MVTVSSASTGGGGSGGGGGSDIVMTQSPRSLPVTLGQPASISCRSSQSLVYTDGITYLS |
| | | WLQQRPGQPPRLLIYEISNRFSGVPDRFSGSGAGTDFTLKISRVEAEDVGVYYCMQATQ |
| | | FPWTFGQGTKVDIKRTVAAPSHHHHHH |
| 32 | HC Ab1 | EVQLVESGGGLVQPGGSMKLSCVASGFTFSSYAMHWVRQAPGKGLEWVAVISYDGSNKY |
| | | YADSVKGRFTISRDNAKNTLYLQMNSLRAEDTATYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 33 | LC Ab1 | DIVMTQSPRSLPVTLGQPASISCRSSQSLVYTDGITYLSWLQQRPGQPPRLLIYEISNR |
| | | FSGVPDRFSGSGAGTDFTLKISRVEAEDVGVYYCMQATQFPWTFGQGTKVDIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 34 | VHCDR1 Ab2 | SYAMH |
| 35 | VHCDR2 Ab2 | LISYDGSNKYYADSVKG |
| 36 | VHCDR3 Ab2 | GAGRGYTYGPDGFDI |
| 37 | VLCDR1 Ab2 | RVSQSITNYLN |
| 38 | VLCDR2 Ab2 | AASSLQS |
| 39 | VLCDR3 Ab2 | QQSYTTPFT |
| 40 | VH Ab2 | EVQLLESGGGVVQPGRSLRLSCAASGFTFSSYAMHWVRQAPGKGLEWVALISYDGSNKY |
| | (referred to as | YADSVKGRFTISRDNSKNTLYLQMNSLRAEDTAIYYCVRGAGRGYTYGPDGFDIWGQGT |
| | FGF#59 in FIG. | MVTVSS |
| | 2) | |
| 41 | VL Ab2 | DIVMTQSPSSLSASVGDRVSITCRVSQSITNYLNWYQQKPGGAPKLLIYAASSLQSGVP |
| | | SRFSGSGSGSQFTLTISSLQAEDFATYYCQQSYTTPFTFGPGTKVEIK |
| 42 | VHVL Ab2 | EVQLLESGGGVVQPGRSLRLSCAASGFTFSSYAMHWVRQAPGKGLEWVALISYDGSNKY |
| | (scFv) | YADSVKGRFTISRDNSKNTLYLQMNSLRAEDTAIYYCVRGAGRGYTYGPDGFDIWGQGT |
| | | MVTVSSASTGGGGSGGGGSGGGGSGGGGSDIVMTQSPSSLSASVGDRVSITCRVSQSIT |
| | | NYLNWYQQKPGGAPKLLIYAASSLQSGVPSRFSGSGSGSQFTLTISSLQAEDFATYYCQ |
| | | QSYTTPFTFGPGTKVEIKRTVAAPSHHHHHH |
| 43 | HC Ab2 | EVQLLESGGGVVQPGRSLRLSCAASGFTFSSYAMHWVRQAPGKGLEWVALISYDGSNKY |
| | | YADSVKGRFTISRDNSKNTLYLQMNSLRAEDTAIYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 44 | LC Ab2 | DIVMTQSPSSLSASVGDRVSITCRVSQSITNYLNWYQQKPGGAPKLLIYAASSLQSGVP |
| | | SRFSGSGSGSQFTLTISSLQAEDFATYYCQQSYTTPFTFGPGTKVEIKRTVAAPSVFIF |
| | | PPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 45 | VHCDR1 Ab3 | QYSMH |
| 46 | VHCDR2 Ab3 | LISFDGADKYYADSVKG |
| 47 | VHCDR3 Ab3 | GAGRGYTYGPDGFDI |
| 48 | VLCDR1 Ab3 | RTSQTISRYLN |
| 49 | VLCDR2 Ab3 | TATSLQS |
| 50 | VLCDR3 Ab3 | QQTYSAPLT |
| 51 | VH Ab3 | EVQLVESGGGVVQPGRSLRLSCAASGFTFSQYSMHWVRQSPGKGLEWVTLISFDGADKY |
| | (referred to as | YADSVKGRFTISRDNSNDTLFLHMNGLRVEDTAIYYCVRGAGRGYTYGPDGFDIWGQGT |
| | FGF#78 in FIG. | MVTVSS |
| | 2) | |
| 52 | VL Ab3 | DIQLTQSPSSLSASVGDRVTITCRTSQTISRYLNWYQQQPGKAPKLLIYTATSLQSGVP |
| | | SRFSGSGSGTDFTLTIGGLQPEDFAIYFCQQTYSAPLTFGGGTKVEIK |
| 53 | VHVL Ab3 | EVQLVESGGGVVQPGRSLRLSCAASGFTFSQYSMHWVRQSPGKGLEWVTLISFDGADKY |
| | (scFv) | YADSVKGRFTISRDNSNDTLFLHMNGLRVEDTAIYYCVRGAGRGYTYGPDGFDIWGQGT |
| | | MVTVSSASTGGGGSGGGGSGGGGSGGGGSDIQLTQSPSSLSASVGDRVTITCRTSQTIS |
| | | RYLNWYQQQPGKAPKLLIYTATSLQSGVPSRFSGSGSGTDFTLTIGGLQPEDFAIYFCQ |
| | | QTYSAPLTFGGGTKVEIKRTVAAPSHHHHHH |
| 54 | HC Ab3 | EVQLVESGGGVVQPGRSLRLSCAASGFTFSQYSMHWVRQSPGKGLEWVTLISFDGADKY |
| | | YADSVKGRFTISRDNSNDTLFLHMNGLRVEDTAIYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 55 | LC Ab3 | DIQLTQSPSSLSASVGDRVTITCRTSQTISRYLNWYQQQPGKAPKLLIYTATSLQSGVP |
| | | SRFSGSGSGTDFTLTIGGLQPEDFAIYFCQQTYSAPLTFGGGTKVEIKRTVAAPSVFIF |
| | | PPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 56 | VHCDR1 Ab4 | SYAMH |
| 57 | VHCDR2 Ab4 | LISYDGSNKYYADSVKG |
| 58 | VHCDR3 Ab4 | GAGRGYTYGPDGFDI |
| 59 | VLCDR1 Ab4 | KSSQSLIFGDGKTYLY |
| 60 | VLCDR2 Ab4 | QVSNRFS |
| 61 | VLCDR3 Ab4 | MQAKQFPWT |
| 62 | VH Ab4 | EVQLLESGGGVVQPGRSLRLSCAASGFTFSSYAMHWVRQAPGKGLEWVALISYDGSNKY |
| | (referred to as | YADSVKGRFTISRDNSKNTLYLQMNSLRAEDTAIYYCVRGAGRGYTYGPDGFDIWGQGT |
| | FGF#10 in FIG. | MVTVSS |
| | 2) | |
| 63 | VL Ab4 | DIVMTQTPLSLSVTPGQPASIFCKSSQSLIFGDGKTYLYWYLQKPGQPPRLLIYQVSNR |
| | | FSGVPDRFSGSGAGTDFTLKISRVEAEDVGVYYCMQAKQFPWTFGQGTKLEIK |
| 64 | VHVL Ab4 | EVQLLESGGGVVQPGRSLRLSCAASGFTFSSYAMHWVRQAPGKGLEWVALISYDGSNKY |
| | (scFv) | YADSVKGRFTISRDNSKNTLYLQMNSLRAEDTAIYYCVRGAGRGYTYGPDGFDIWGQGT |
| | | MVTVSSASTGGGSGGGGSDIVMTQTPLSLSVTPGQPASIFCKSSQSLIFGDGKTYLYWY |
| | | LQKPGQPPRLLIYQVSNRFSGVPDRFSGSGAGTDFTLKISRVEAEDVGVYYCMQAKQFP |
| | | WTFGQGTKLEIKRTVAAPSHHHHHH |
| 65 | HC Ab4 | EVQLLESGGGVVQPGRSLRLSCAASGFTFSSYAMHWVRQAPGKGLEWVALISYDGSNKY |
| | | YADSVKGRFTISRDNSKNTLYLQMNSLRAEDTAIYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 66 | LC Ab4 | DIVMTQTPLSLSVTPGQPASIFCKSSQSLIFGDGKTYLYWYLQKPGQPPRLLIYQVSNR |
| | | FSGVPDRFSGSGAGTDFTLKISRVEAEDVGVYYCMQAKQFPWTFGQGTKLEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 67 | VHCDR1 Ab5 | HYAMH |
| 68 | VHCDR2 Ab5 | VISYDGSNKYYADSVKG |
| 69 | VHCDR3 Ab5 | GAGRGYTYGPDGFDI |
| 70 | VLCDR1 Ab5 | KSSQSLLYSDGKTYLS |
| 71 | VLCDR2 Ab5 | EVSSRFS |
| 72 | VLCDR3 Ab5 | MQATRFPWT |
| 73 | VH Ab5 | QVQLVESGGDVVQPGRSLRLSCAASGFTFGHYAMHWVRQAPGQGLEWVTVISYDGSNKY |
| | | YADSVKGRFTISRDNSKNTVDLQMNSLRVEDTAIYYCVRGAGRGYTYGPDGFDIWGQGT |
| | | MVTVSS |
| 74 | VL Ab5 | EIVMTQTPLSLSVTPGQPASISCKSSQSLLYSDGKTYLSWYLQKPGQSPQLLIYEVSSR |
| | | FSGVPDRFSGSGAGTDFTLKISRVEAXDVGVYYCMQATRFPWTFGQGTKVEIK |
| 75 | VHVL Ab5 | QVQLVESGGDVVQPGRSLRLSCAASGFTFGHYAMHWVRQAPGQGLEWVTVISYDGSNKY |
| | (scFv) | YADSVKGRFTISRDNSKNTVDLQMNSLRVEDTAIYYCVRGAGRGYTYGPDGFDIWGQGT |
| | | MVTVSSASTGGGGSGGGGSGGGGSGGGGSEIVMTQTPLSLSVTPGQPASISCKSSQSLL |
| | | YSDGKTYLSWYLQKPGQSPQLLIYEVSSRFSGVPDRFSGSGAGTDFTLKISRVEAXDVG |
| | | VYYCMQATRFPWTFGQGTKVEIKRTVAAPSHHHHHH |
| 76 | HC Ab5 | QVQLVESGGDVVQPGRSLRLSCAASGFTFGHYAMHWVRQAPGQGLEWVTVISYDGSNKY |
| | | YADSVKGRFTISRDNSKNTVDLQMNSLRVEDTAIYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 77 | LC Ab5 | EIVMTQTPLSLSVTPGQPASISCKSSQSLLYSDGKTYLSWYLQKPGQSPQLLIYEVSSR |
| | | FSGVPDRFSGSGAGTDFTLKISRVEAXDVGVYYCMQATRFPWTFGQGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 78 | VHCDR1 Ab6 | SYAMH |
| 79 | VHCDR2 Ab6 | VISYDGSNKYYADSVKG |
| 80 | VHCDR3 Ab6 | GAGRGYTYGPDGFDI |
| 81 | VLCDR1 Ab6 | RSSQSLVYTDGITYLS |
| 82 | VLCDR2 Ab6 | EISNRFS |
| 83 | VLCDR3 Ab6 | MQATQFPWT |
| 84 | VH Ab6 | EVQLQESGGGLVQPGGSMKLSCVASGFTFSSYAMHWVRQAPGKGLEWVAVISYDGSNKY |
| | (referred to as | YADSVKGRFTISRDNAKNTLYLQMNSLRAEDTATYYCVRGAGRGYTYGPDGFDIWGQGT |
| | FGF#6 in FIG. 2) | MVTVSS |
| 85 | VL Ab6 | DIVMTQSPRSLPVTLGQPASISCRSSQSLVYTDGITYLSWLQQRPGQPPRLLIYEISNR |
| | | FSGVPDRFSGSGAGTDFTLKISRVEAEDVGVYYCMQATQFPWTFGQGTKVDIK |
| 86 | VHVL Ab6 | EVQLQESGGGLVQPGGSMKLSCVASGFTFSSYAMHWVRQAPGKGLEWVAVISYDGSNKY |
| | (scFv) | YADSVKGRFTISRDNAKNTLYLQMNSLRAEDTATYYCVRGAGRGYTYGPDGFDIWGQGT |
| | | MVTVSSASTGGGGSGGGGGSDIVMTQSPRSLPVTLGQPASISCRSSQSLVYTDGITYLS |
| | | WLQQRPGQPPRLLIYEISNRFSGVPDRFSGSGAGTDFTLKISRVEAEDVGVYYCMQATQ |
| | | FPWTFGQGTKVDIKRTVAAPSHHHHHH |
| 87 | HC Ab6 | EVQLQESGGGLVQPGGSMKLSCVASGFTFSSYAMHWVRQAPGKGLEWVAVISYDGSNKY |
| | | YADSVKGRFTISRDNAKNTLYLQMNSLRAEDTATYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 88 | LC Ab6 | DIVMTQSPRSLPVTLGQPASISCRSSQSLVYTDGITYLSWLQQRPGQPPRLLIYEISNR |
| | | FSGVPDRFSGSGAGTDFTLKISRVEAEDVGVYYCMQATQFPWTFGQGTKVDIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 89 | VHCDR1 Ab7 | GYAIH |
| 90 | VHCDR2 Ab7 | LISYDGSNKYYADSAKG |
| 91 | VHCDR3 Ab7 | GAGRGYTYGPDGFDI |
| 92 | VLCDR1 Ab7 | RSSESLVYRDGNTYLS |
| 93 | VLCDR2 Ab7 | KVSNRFS |
| 94 | VLCDR3 Ab7 | MQATQFPWT |
| 95 | VH Ab7 | QVQLVESGGGVVQPGRSLRLSCAASGFTFSGYAIHWVRQAPGKGLEWVALISYDGSNKY |
| | (referred to as | YADSAKGRFTISRDNSKNTLSLQMNSLRVEDTAIYYCVRGAGRGYTYGPDGFDIWGQGT |
| | FGF#51 in FIG. | MVTVSS |
| | 2) | |
| 96 | VL Ab7 | DIVMTQTPLSSPVTLGQPASISCRSSESLVYRDGNTYLSWLHQRPGQSPRLLIYKVSNR |
| | | FSGVPDRFSGSGAGTDFTLKISRVEAEDVGVYFCMQATQFPWTFGQGTKVEIK |
| 97 | VHVL Ab7 | QVQLVESGGGVVQPGRSLRLSCAASGFTFSGYAIHWVRQAPGKGLEWVALISYDGSNKY |
| | (scFv) | YADSAKGRFTISRDNSKNTLSLQMNSLRVEDTAIYYCVRGAGRGYTYGPDGFDIWGQGT |
| | | MVTVSSASTGGGGSGGGGSGGGGSGGGGSDIVMTQTPLSSPVTLGQPASISCRSSESLV |
| | | YRDGNTYLSWLHQRPGQSPRLLIYKVSNRFSGVPDRFSGSGAGTDFTLKISRVEAEDVG |
| | | VYFCMQATQFPWTFGQGTKVEIKRTVAAPSHHHHHH |
| 98 | HC Ab7 | QVQLVESGGGVVQPGRSLRLSCAASGFTFSGYAIHWVRQAPGKGLEWVALISYDGSNKY |
| | | YADSAKGRFTISRDNSKNTLSLQMNSLRVEDTAIYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 99 | LC Ab7 | DIVMTQTPLSSPVTLGQPASISCRSSESLVYRDGNTYLSWLHQRPGQSPRLLIYKVSNR |
| | | FSGVPDRFSGSGAGTDFTLKISRVEAEDVGVYFCMQATQFPWTFGQGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 100 | VHCDR1 Ab8 | SYAMH |
| 101 | VHCDR2 Ab8 | VISYDGSNKYYADSVKG |
| 102 | VHCDR3 Ab8 | GAGRGYTYGPDGFDI |
| 103 | VLCDR1 Ab8 | RSSQSLVYSDGNTYLN |
| 104 | VLCDR2 Ab8 | KVSNRFS |
| 105 | VLCDR3 Ab8 | MQATRFPWT |
| 106 | VH Ab8 | EVQLVESGGGVVQPGRSLRLSCAASGFTFSSYAMHWVRQAPGKGLEWVAVISYDGSNKY |
| | (referred to as | YADSVKGRFTISRDNSKNTLSLQMNSLRVEDTAIYYCVRGAGRGYTYGPDGFDIWGQGT |
| | FGF#60 in FIG. | MVTVSS |
| | 2) | |
| 107 | VL Ab8 | EIVMTQTPLSSPVTLGQPASISCRSSQSLVYSDGNTYLNWLQQRPGQPPRLLIYKVSNR |
| | | FSGVPDRFSGSGAGTDFTLKISRVEAEDVGVYYCMQATRFPWTFGQGTKVEIK |
| 108 | VHVL Ab8 | EVQLVESGGGVVQPGRSLRLSCAASGFTFSSYAMHWVRQAPGKGLEWVAVISYDGSNKY |
| | (scFv) | YADSVKGRFTISRDNSKNTLSLQMNSLRVEDTAIYYCVRGAGRGYTYGPDGFDIWGQGT |
| | | MVTVSSASTGGGGSGGGGSGGGGSGGGGSEIVMTQTPLSSPVTLGQPASISCRSSQSLV |
| | | YSDGNTYLNWLQQRPGQPPRLLIYKVSNRFSGVPDRFSGSGAGTDFTLKISRVEAEDVG |
| | | VYYCMQATRFPWTFGQGTKVEIKRTVAAPSHHHHHH |
| 109 | HC Ab8 | EVQLVESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 110 | LC Ab8 | EIVMTQTPLSSPVTLGQPASISCRSSQSLVYSDGNTYLNWLQQRPGQPPRLLIYKVSNR |
| | | FSGVPDRFSGSGAGTDFTLKISRVEAEDVGVYYCMQATRFPWTFGQGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 111 | VHCDR1 Ab9 | THAMH |
| 112 | VHCDR2 Ab9 | LISYDGSEKYYADSVKG |
| 113 | VHCDR3 Ab9 | GAGRGYTYGPDGFDI |
| 114 | VLCDR1 Ab9 | KSSQSLLHSDGKTYLY |
| 115 | VLCDR2 Ab9 | EVSSRFS |
| 116 | VLCDR3 Ab9 | MQYINLPLT |
| 117 | VH Ab9 | QVQLLESGGXVXHPGXSLRLSCATSGFSFTTHAMHWVRQAPGKGLEWVALISYDGSEKY |
| | (referred to as | YADSVKGRFTISRDNSKNTLSLQMNSLRVEDTAIYYCVRGAGRGYTYGPDGFDIWGQGT |
| | FGF#63 in FIG. | MVTVSS |
| | 2) | |
| 118 | VL Ab9 | DIVMTQTPLSLSVTPGQPASISCKSSQSLLHSDGKTYLYWYLQKPGQSPQLLMYEVSSR |
| | | FSGVPDRFSGSGSGTDFTLKISRVEAEDVGVYYCMQYINLPLTFGGGTKLEIK |
| 119 | VHVL Ab9 | QVQLLESGGXVXHPGXSLRLSCATSGFSFTTHAMHWVRQAPGKGLEWVALISYDGSEKY |
| | (scFv) | YADSVKGRFTISRDNSKNTLSLQMNSLRVEDTAIYYCVRGAGRGYTYGPDGFDIWGQGT |
| | | MVTVSSASTGGGGSGGGGSGGGGSGGGGSDIVMTQTPLSLSVTPGQPASISCKSSQSLL |
| | | HSDGKTYLYWYLQKPGQSPQLLMYEVSSRFSGVPDRFSGSGSGTDFTLKISRVEAEDVG |
| | | VYYCMQYINLPLTFGGGTKLEIKRTVAAPSHHHHHH |
| 120 | HC Ab9 | QVQLLESGGXVXHPGXSLRLSCATSGFSFTTHAMHWVRQAPGKGLEWVALISYDGSEKY |
| | | YADSVKGRFTISRDNSKNTLSLQMNSLRVEDTAIYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 121 | LC Ab9 | DIVMTQTPLSLSVTPGQPASISCKSSQSLLHSDGKTYLYWYLQKPGQSPQLLMYEVSSR |
| | | FSGVPDRFSGSGSGTDFTLKISRVEAEDVGVYYCMQYINLPLTFGGGTKLEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 122 | VHCDR1 Ab10 | HYAMH |
| 123 | VHCDR2 Ab10 | VISYDGSNKYYADSVKG |
| 124 | VHCDR3 Ab10 | GAGRGYTYGPDGFDI |
| 125 | VLCDR1 Ab10 | RSSQSLVYSDGNTYLN |
| 126 | VLCDR2 Ab10 | KVSNRFS |
| 127 | VLCDR3 Ab10 | |
| 128 | VH Ab10 | QVQLVESGGDVVQPGRSLRLSCAASGFTFGHYAMHWVRQAPGQGLEWVTVISYDGSNKY |
| | (referred to as | YADSVKGRFTISRDNSKNTVDLQMNSLRVEDTAIYYCVRGAGRGYTYGPDGFDIWGQGT |
| | FGF#73 in FIG. | MVTVSS |
| | 2) | |
| 129 | VL Ab10 | EIVLTQTPLSSPVTLGQPASISCRSSQSLVYSDGNTYLNWLQQRPGQPPRLLIYKVSNR |
| | | FSGVPDRFSGSGAGTDFTLKISRVEAED |
| 130 | VHVL Ab10 | QVQLVESGGDVVQPGRSLRLSCAASGFTFGHYAMHWVRQAPGQGLEWVTVISYDGSNKY |
| | (scFv) | YADSVKGRFTISRDNSKNTVDLQMNSLRVEDTAIYYCVRGAGRGYTYGPDGFDIWGQGT |
| | | MVTVSSASTGGGGSGGGGSGGGGSGGGGSEIVLTQTPLSSPVTLGQPASISCRSSQSLV |
| | | YSDGNTYLNWLQQRPGQPPRLLIYKVSNRFSGVPDRFSGSGAGTDFTLKISRVEAED |
| 131 | HC Ab10 | QVQLVESGGDVVQPGRSLRLSCAASGFTFGHYAMHWVRQAPGQGLEWVTVISYDGSNKY |
| | | YADSVKGRFTISRDNSKNTVDLQMNSLRVEDTAIYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 132 | LC Ab10 | EIVLTQTPLSSPVTLGQPASISCRSSQSLVYSDGNTYLNWLQQRPGQPPRLLIYKVSNR |
| | | FSGVPDRFSGSGAGTDFTLKISRVEAED |
| 133 | VHCDR1 Ab11 | SYAMH |
| 134 | VHCDR2 Ab11 | VISYDGSNKYYADSVKG |
| 135 | VHCDR3 Ab11 | GAGRGYTYGPDGFDI |
| 136 | VLCDR1 Ab11 | RSSQSLVYTDGITYLS |
| 137 | VLCDR2 Ab11 | EISNRFS |
| 138 | VLCDR3 Ab11 | MQATQFPWT |
| 139 | VH Ab11 | EVQLQESGGGLVQPGGSMKLSCVASGFTFSSYAMHWVRQAPGKGLEWVAVISYDGSNKY |
| | (referred to as | YADSVKGRFTISRDNAKNTLYLQMNSLRAEDTATYYCVRGAGRGYTYGPDGFDIWGQGT |
| | FGF#50b in FIG. | MVTVSS |
| | 2) | |
| 140 | VL Ab11 | DIVMTQSPRSLPVTLGQPASISCRSSQSLVYTDGITYLSWLQQRPGQPPRLLIYEISNR |
| | | FSGVPDRFSGSGAGTDFTLKISRVEAXDVGVYYCMQATQFPWTFGQGTKVDIK |
| 141 | VHVL Ab11 | EVQLQESGGGLVQPGGSMKLSCVASGFTFSSYAMHWVRQAPGKGLEWVAVISYDGSNKY |
| | (scFv) | YADSVKGRFTISRDNAKNTLYLQMNSLRAEDTATYYCVRGAGRGYTYGPDGFDIWGQGT |
| | | MVTVSSASTGGGGSGGGGGSDIVMTQSPRSLPVTLGQPASISCRSSQSLVYTDGITYLS |
| | | WLQQRPGQPPRLLIYEISNRFSGVPDRFSGSGAGTDFTLKISRVEAXDVGVYYCMQATQ |
| | | FPWTFGQGTKVDIKRTVAAPSHHHHHH |
| 142 | HC Ab11 | EVQLQESGGGLVQPGGSMKLSCVASGFTFSSYAMHWVRQAPGKGLEWVAVISYDGSNKY |
| | | YADSVKGRFTISRDNAKNTLYLQMNSLRAEDTATYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 143 | LC Ab11 | DIVMTQSPRSLPVTLGQPASISCRSSQSLVYTDGITYLSWLQQRPGQPPRLLIYEISNR |
| | | FSGVPDRFSGSGAGTDFTLKISRVEAXDVGVYYCMQATQFPWTFGQGTKVDIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 144 | VHCDR1 Ab12 | SHDIN |
| 145 | VHCDR2 Ab12 | WINPNNDITDYAQEFQG |
| 146 | VHCDR3 Ab12 | GAGMLFHAVGQFDS |
| 147 | VLCDR1 Ab12 | QASQDIRKNLN |
| 148 | VLCDR2 Ab12 | DASNLDT |
| 149 | VLCDR3 Ab12 | LQYGDLPLT |
| 150 | VH Ab12 | QVQLVQSGAEVKKPGASVKVSCQASGYRFTSHDINWVRQVPGHGLEWMGWINPNNDITD |
| | (referred to as | YAQEFQGRLTMTSDTSTRTAYMELSSLTAEDTAVYYCARGAGMLFHAVGQFDSWGQGTL |
| | FGF#14 in FIG. | VTVSS |
| | 2) | |
| 151 | VL Ab12 | AIRMTQSPSSLSASVGDRVTITCQASQDIRKNLNWYQQKPGKAPELLINDASNLDTGVP |
| | | SRFSGGGSGTDFTFTISSLQPEDIATYFCLQYGDLPLTFXGGTKVDIK |
| 152 | VHVL Ab12 | QVQLVQSGAEVKKPGASVKVSCQASGYRFTSHDINWVRQVPGHGLEWMGWINPNNDITD |
| | (scFv) | YAQEFQGRLTMTSDTSTRTAYMELSSLTAEDTAVYYCARGAGMLFHAVGQFDSWGQGTL |
| | | VTVSSASTGGGGSGGGGSGGGGSGGGGSAIRMTQSPSSLSASVGDRVTITCQASQDIRK |
| | | NLNWYQQKPGKAPELLINDASNLDTGVPSRFSGGGSGTDFTFTISSLQPEDIATYFCLQ |
| | | YGDLPLTFXGGTKVDIKRTVAAPSHHHHHH |
| 153 | HC Ab12 | QVQLVQSGAEVKKPGASVKVSCQASGYRFTSHDINWVRQVPGHGLEWMGWINPNNDITD |
| | | YAQEFQGRLTMTSDTSTRTAYMELSSLTAEDTAVYYCARGAGMLFHAVGQFDSWGQGTL |
| | | VTVSSKGPSVFPLAPSSRSTSESTAALGCLVKDYFPEPVTVSWNSGALTSGVHTFPAVL |
| | | QSSGLYSLSSVVTVPSSNFGTQTYTCNVDHKPSNTKVDKTVERKCCVECPPCPAPPVAG |
| | | PSVFLFPPKPKDTLMISRTPEVTCVVVDVSHEDPEVQFNWYVDGVEVHNAKTKPREEQF |
| | | NSTFRVVSVLTVVHQDWLNGKEYKCKVSNKGLPAPIEKTISKTKGQPREPQVYTLPPSR |
| | | EEMTKNQVSLTCLVKGFYPSDIAVEWESNGQPENNYKTTPPMLDSDGSFFLYSKLTVDK |
| | | SRWQQGNVFSCSVMHEALHNHYTQKSLSLSPG |
| 154 | LC Ab12 | AIRMTQSPSSLSASVGDRVTITCQASQDIRKNLNWYQQKPGKAPELLINDASNLDTGVP |
| | | SRFSGGGSGTDFTFTISSLQPEDIATYFCLQYGDLPLTFXGGTKVDIKRTVAAPSVFIF |
| | | PPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 155 | VHCDR1 Ab13 | SHDIN |
| 156 | VHCDR2 Ab13 | WINPNNDITDYAQEFQG |
| 157 | VHCDR3 Ab13 | GAGMLFHAVGQFDS |
| 158 | VLCDR1 Ab13 | RASQSIGRFLN |
| 159 | VLCDR2 Ab13 | TASTLQS |
| 160 | VLCDR3 Ab13 | QQFKNYPT |
| 161 | VH Ab13 | QMQLVQSGAEVKKPGASVKVSCQASGYRFTSHDINWVRQVPGHGLEWMGWINPNNDITD |
| | (referred to as | YAQEFQGRLTMTSDTSTRTAYMELSSLTAEDTAVYYCARGAGMLFHAVGQFDSWGQGTL |
| | FGF#49 in FIG. | VTVSS |
| | 2) | |
| 162 | VL Ab13 | VIWMTQSPSSLSASVGDRVIITCRASQSIGRFLNWYQQTPGKPPKVLIHTASTLQSGVP |
| | | SRFSGSGSGTHFTLTITGLQPEXFATYYCQQFKNYPTFGGGTXVEIK |
| 163 | VHVK Ab13 | QMQLVQSGAEVKKPGASVKVSCQASGYRFTSHDINWVRQVPGHGLEWMGWINPNNDITD |
| | (scFv) | YAQEFQGRLTMTSDTSTRTAYMELSSLTAEDTAVYYCARGAGMLFHAVGQFDSWGQGTL |
| | | VTVSSASTGGGGSGGGGSGGGGSGGGGSVIWMTQSPSSLSASVGDRVIITCRASQSIGR |
| | | FLNWYQQTPGKPPKVLIHTASTLQSGVPSRFSGSGSGTHFTLTITGLQPEXFATYYCQQ |
| | | FKNYPTFGGGTXVEIKRTVAAPSHHHHHH |
| 164 | HC Ab13 | QMQLVQSGAEVKKPGASVKVSCQASGYRFTSHDINWVRQVPGHGLEWMGWINPNNDITD |
| | | YAQEFQGRLTMTSDTSTRTAYMELSSLTAEDTAVYYCARGAGMLFHAVGQFDSWGQGTL |
| | | VTVSSKGPSVFPLAPSSRSTSESTAALGCLVKDYFPEPVTVSWNSGALTSGVHTFPAVL |
| | | QSSGLYSLSSVVTVPSSNFGTQTYTCNVDHKPSNTKVDKTVERKCCVECPPCPAPPVAG |
| | | PSVFLFPPKPKDTLMISRTPEVTCVVVDVSHEDPEVQFNWYVDGVEVHNAKTKPREEQF |
| | | NSTFRVVSVLTVVHQDWLNGKEYKCKVSNKGLPAPIEKTISKTKGQPREPQVYTLPPSR |
| | | EEMTKNQVSLTCLVKGFYPSDIAVEWESNGQPENNYKTTPPMLDSDGSFFLYSKLTVDK |
| | | SRWQQGNVFSCSVMHEALHNHYTQKSLSLSPG |
| 165 | LC Ab13 | VIWMTQSPSSLSASVGDRVIITCRASQSIGRFLNWYQQTPGKPPKVLIHTASTLQSGVP |
| | | SRFSGSGSGTHFTLTITGLQPEXFATYYCQQFKNYPTFGGGTXVEIKRTVAAPSVFIFP |
| | | PSDEQLKSGTASVVCLLNNFYPREAKVQWKVDNALQSGNSQESVTEQDSKDSTYSLSST |
| | | LTLSKADYEKHKVYACEVTHQGLSSPVTKSFNRGEC |
| 166 | VHCDR1 Ab14 | SNYMS |
| 167 | VHCDR2 Ab14 | VIFSDGTTYYADSVKG |
| 168 | VHCDR3 Ab14 | GPANGAYDI |
| 169 | VLCDR1 Ab14 | RASQSISNWLA |
| 170 | VLCDR2 Ab14 | RASTLES |
| 171 | VLCDR3 Ab14 | QQYNVYSGT |
| 172 | VH Ab14 | EVQLVETGGGLIQPGGSLRLSCVASGFTVSSNYMSWVRQAPGKGLEWVSVIFSDGTTYY |
| | (referred to as | ADSVKGRFTISRDNSKNTLYLQMNSLKTEDTAVYYCVAGPANGAYDIWGQGTMVTVSS |
| | FGF#24 in FIG. | |
| | 2) | |
| 173 | VL Ab14 | NIQMTQSPSILSASVGDRVTITCRASQSISNWLAWYQQKPGKAPKLLIFRASTLESGVP |
| | | SRFSGSGSGAEFNLTISSLQPDDFATYYCQQYNVYSGTFGQGTKVEIK |
| 174 | VHVL Ab14 | EVQLVETGGGLIQPGGSLRLSCVASGFTVSSNYMSWVRQAPGKGLEWVSVIFSDGTTYY |
| | (scFv) | ADSVKGRFTISRDNSKNTLYLQMNSLKTEDTAVYYCVAGPANGAYDIWGQGTMVTVSSA |
| | | STGGGSGGGGSNIQMTQSPSILSASVGDRVTITCRASQSISNWLAWYQQKPGKAPKLLI |
| | | FRASTLESGVPSRFSGSGSGAEFNLTISSLQPDDFATYYCQQYNVYSGTFGQGTKVEIK |
| | | RTVAAPSHHHHHH |
| 175 | HC Ab14 | EVQLVETGGGLIQPGGSLRLSCVASGFTVSSNYMSWVRQAPGKGLEWVSVIFSDGTTYY |
| | | ADSVKGRFTISRDNSKNTLYLQMNSLKTEDTAVYYCVAGPANGAYDIWGQGTMVTVSSK |
| | | GPSVFPLAPSSRSTSESTAALGCLVKDYFPEPVTVSWNSGALTSGVHTFPAVLQSSGLY |
| | | SLSSVVTVPSSNFGTQTYTCNVDHKPSNTKVDKTVERKCCVECPPCPAPPVAGPSVFLF |
| | | PPKPKDTLMISRTPEVTCVVVDVSHEDPEVQFNWYVDGVEVHNAKTKPREEQFNSTFRV |
| | | VSVLTVVHQDWLNGKEYKCKVSNKGLPAPIEKTISKTKGQPREPQVYTLPPSREEMTKN |
| | | QVSLTCLVKGFYPSDIAVEWESNGQPENNYKTTPPMLDSDGSFFLYSKLTVDKSRWQQG |
| | | NVFSCSVMHEALHNHYTQKSLSLSPG |
| 176 | LC Ab14 | NIQMTQSPSILSASVGDRVTITCRASQSISNWLAWYQQKPGKAPKLLIFRASTLESGVP |
| | | SRFSGSGSGAEFNLTISSLQPDDFATYYCQQYNVYSGTFGQGTKVEIKRTVAAPSVFIF |
| | | PPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 177 | VHCDR1 Ab15 | SYAMH |
| 178 | VHCDR2 Ab15 | VISYDGSNKYYADSVKG |
| 179 | VHCDR3 Ab15 | GAGRGYTYGPDGFDI |
| 180 | VLCDR1 Ab15 | TSSRSLLHSDGKTYVY |
| 181 | VLCDR2 Ab15 | ELSNRFS |
| 182 | VLCDR3 Ab15 | MQYIEAPLT |
| 183 | VH Ab15 | EVQLLESGGGVVQPGRSLRLSCAASGFTFSSYAMHWVRQAPGKGLEWVAVISYDGSNKY |
| | (referred to as | YADSVKGRFTISRDNSKNTLSLQMNSLRVEDTAIYYCVRGAGRGYTYGPDGFDIWGQGT |
| | FGF#26 in FIG. | MVTVSS |
| | 2) | |
| 184 | VL Ab15 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 185 | VHVK Ab15 | EVQLLESGGGVVQPGRSLRLSCAASGFTFSSYAMHWVRQAPGKGLEWVAVISYDGSNKY |
| | (scFv) | YADSVKGRFTISRDNSKNTLSLQMNSLRVEDTAIYYCVRGAGRGYTYGPDGFDIWGQGT |
| | | MVTVSSASTGGGGSGGGGSGGGGSGGGGSDIVMTQTPLSLSVTPGQPASISCTSSRSLL |
| | | HSDGKTYVYWYVQKSGQPPQLLIYELSNRFSGVPDRFSGSGSRTDFTLKISRVEAEDVG |
| | | VYYCMQYIEAPLTFGGGTKVEIKRTVAAPSHHHHHH |
| 186 | HC Ab15 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 187 | LC Ab15 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 188 | VHCDR1 Ab16 | SYAMH |
| 189 | VHCDR2 Ab16 | VISYDGSNKYYADSVKG |
| 190 | VHCDR3 Ab16 | GAGRGYTRGPDGFDI |
| 191 | VLCDR1 Ab16 | KSSRSLLHSDGKTYVY |
| 192 | VLCDR2 Ab16 | ELSNRFS |
| 193 | VLCDR3 Ab16 | MQYIEAPLT |
| 194 | VH Ab16 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTRGPDGFDIWGQGT |
| | | MVTVSS |
| 195 | VL Ab16 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 196 | HC Ab16 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTRGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 197 | LC Ab16 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 198 | VHCDR1 Ab17 | SYAMH |
| 199 | VHCDR2 Ab17 | VISYDGSNKYYADSVKG |
| 200 | VHCDR3 Ab17 | GAGRGYTNGPDGFDI |
| 201 | VLCDR1 Ab17 | KSSRSLLHSDGKTYVY |
| 202 | VLCDR2 Ab17 | ELSNRFS |
| 203 | VLCDR3 Ab17 | MQYIEAPLT |
| 204 | VH Ab17 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTNGPDGFDIWGQGT |
| | | MVTVSS |
| 205 | VL Ab17 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 206 | HC Ab17 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTNGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 207 | LC Ab17 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 208 | VHCDR1 Ab18 | SYAMH |
| 209 | VHCDR2 Ab18 | VISYDGSNKYYADSVKG |
| 210 | VHCDR3 Ab18 | GAGRGFTWGPDGFDI |
| 211 | VLCDR1 Ab18 | KSSRSLLHSDGKTYVY |
| 212 | VLCDR2 Ab18 | ELSNRFS |
| 213 | VLCDR3 Ab18 | MQYIEAPLT |
| 214 | VH Ab18 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTWGPDGFDIWGQGT |
| | | MVTVSS |
| 215 | VL Ab18 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 216 | HC Ab18 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTWGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 217 | LC Ab18 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 218 | VHCDR1 Ab19 | SYAMH |
| 219 | VHCDR2 Ab19 | VISYDGSNKYYADSVKG |
| 220 | VLCDR3 Ab19 | GAGRGFTLGPDGFDI |
| 221 | VLCDR1 Ab19 | KSSRSLLHSDGKTYVY |
| 222 | VLCDR2 Ab19 | ELSNRFS |
| 223 | VLCDR3 Ab19 | MQYIEAPLT |
| 224 | VH Ab19 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSS |
| 225 | VL Ab19 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 226 | HC Ab19 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 227 | LC Ab19 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 228 | VHCDR1 Ab20 | SYAMH |
| 229 | VHCDR2 Ab20 | VISYDGSNKYYADSVKG |
| 230 | VLCDR3 Ab20 | GAGRGLTFGPDGFDI |
| 231 | VLCDR1 Ab20 | KSSRSLLHSDGKTYVY |
| 232 | VLCDR2 Ab20 | ELSNRFS |
| 233 | VLCDR3 Ab20 | MQYIEAPLT |
| 234 | VH Ab20 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTFGPDGFDIWGQGT |
| | | MVTVSS |
| 235 | VL Ab20 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 236 | HC Ab20 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTFGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 237 | LC Ab20 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 238 | VHCDR1 Ab21 | SYAMH |
| 239 | VHCDR2 Ab21 | VISYDGSNKYYADSVKG |
| 240 | VLCDR3 Ab21 | GAGLGWPYGPDGFDI |
| 241 | VLCDR1 Ab21 | KSSRSLLHSDGKTYVY |
| 242 | VLCDR2 Ab21 | ELSNRFS |
| 243 | VLCDR3 Ab21 | MQYIEAPLT |
| 244 | VH Ab21 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSS |
| 245 | VL Ab21 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 246 | HC Ab21 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 247 | LC Ab21 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 248 | VHCDR1 Ab22 | SYAMH |
| 249 | VHCDR2 Ab22 | VISYDGSNKYYADSVKG |
| 250 | VLCDR3 Ab22 | GAGRGFTKGPDGFDI |
| 251 | VLCDR1 Ab22 | KSSRSLLHSDGKTYVY |
| 252 | VLCDR2 Ab22 | ELSNRFS |
| 253 | VLCDR3 Ab22 | MQYIEAPLT |
| 254 | VH Ab22 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTKGPDGFDIWGQGT |
| | | MVTVSS |
| 255 | VL Ab22 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 256 | HC Ab22 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTKGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 257 | LC Ab22 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 258 | VHCDR1 Ab23 | SYAMH |
| 259 | VHCDR2 Ab23 | VISYDGSNKYYADSVKG |
| 260 | VLCDR3 Ab23 | GAGRGLTYGPDGFDI |
| 261 | VLCDR1 Ab23 | KSSRSLLHSDGKTYVY |
| 262 | VLCDR2 Ab23 | ELSNRFS |
| 263 | VLCDR3 Ab23 | MQYIEAPLT |
| 264 | VH Ab23 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSS |
| 265 | VL Ab23 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 266 | HC Ab23 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 267 | LC Ab23 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 268 | VHCDR1 Ab24 | SYAMH |
| 269 | VHCDR2 Ab24 | VISYDGSNKYYADSVKG |
| 270 | VLCDR3 Ab24 | GAGRGLIYGPDGFDI |
| 271 | VLCDR1 Ab24 | KSSRSLLHSDGKTYVY |
| 272 | VLCDR2 Ab24 | ELSNRFS |
| 273 | VLCDR3 Ab24 | MQYIEAPLT |
| 274 | VH Ab24 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLIYGPDGFDIWGQGT |
| | | MVTVSS |
| 275 | VL Ab24 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 276 | HC Ab24 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLIYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 277 | LC Ab24 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 278 | VHCDR1 Ab25 | SYAMH |
| 279 | VHCDR2 Ab25 | VISYDGSNKYYADSVKG |
| 280 | VLCDR3 Ab25 | GAGLGFTYGPDGFDI |
| 281 | VLCDR1 Ab25 | KSSRSLLHSDGKTYVY |
| 282 | VLCDR2 Ab25 | ELSNRFS |
| 283 | VLCDR3 Ab25 | MQYIEAPLT |
| 284 | VH Ab25 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFTYGPDGFDIWGQGT |
| | | MVTVSS |
| 285 | VL Ab25 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 286 | HC Ab25 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 287 | LC Ab25 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 288 | VHCDR1 Ab26 | SYAMH |
| 289 | VHCDR2 Ab26 | VISYDGSNKYYADSVKG |
| 290 | VLCDR3 Ab26 | GAGQGLSYGPDGFDI |
| 291 | VLCDR1 Ab26 | KSSRSLLHSDGKTYVY |
| 292 | VLCDR2 Ab26 | ELSNRFS |
| 293 | VLCDR3 Ab26 | MQYIEAPLT |
| 294 | VH Ab26 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGLSYGPDGFDIWGQGT |
| | | MVTVSS |
| 295 | VL Ab26 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 296 | HC Ab26 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGLSYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 297 | LC Ab26 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 298 | VHCDR1 Ab27 | SYAMH |
| 299 | VHCDR2 Ab27 | VISYDGSNKYYADSVKG |
| 300 | VLCDR3 Ab27 | GAGRGYTLGPDGFDI |
| 301 | VLCDR1 Ab27 | KSSRSLLHSDGKTYVY |
| 302 | VLCDR2 Ab27 | ELSNRFS |
| 303 | VLCDR3 Ab27 | MQYIEAPLT |
| 304 | VH Ab27 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTLGPDGFDIWGQGT |
| | | MVTVSS |
| 305 | VL Ab27 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 306 | HC Ab27 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTLGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 307 | LC Ab27 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 308 | VHCDR1 Ab28 | SYAMH |
| 309 | VHCDR2 Ab28 | VISYDGSNKYYADSVKG |
| 310 | VLCDR3 Ab28 | GAGRGFTYGPDGFDI |
| 311 | VLCDR1 Ab28 | KSSRSLLHSDGKTYVY |
| 312 | VLCDR2 Ab28 | ELSNRFS |
| 313 | VLCDR3 Ab28 | MQYIEAPLT |
| 314 | VH Ab28 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTYGPDGFDIWGQGT |
| | | MVTVSS |
| 315 | VL Ab28 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 316 | HC Ab28 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 317 | LC Ab28 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 318 | VHCDR1 Ab29 | SYAMH |
| 319 | VHCDR2 Ab29 | VISYDGSNKYYADSVKG |
| 320 | VLCDR3 Ab29 | GAGLGWPYGPDGFDI |
| 321 | VLCDR1 Ab29 | TSSRSLLHSDGKTYVY |
| 322 | VLCDR2 Ab29 | ELSNRFS |
| 323 | VLCDR3 Ab29 | MQYVEAPLT |
| 324 | VH Ab29 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSS |
| 325 | VL Ab29 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 326 | HC Ab29 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 327 | LC Ab29 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 328 | VHCDR1 Ab30 | SYAMH |
| 329 | VHCDR2 Ab30 | VISYDGSNKYYADSVKG |
| 330 | VLCDR3 Ab30 | GAGRGFTWGPDGFDI |
| 331 | VLCDR1 Ab30 | TSSRSLLHSDGKTYVY |
| 332 | VLCDR2 Ab30 | ELSNRFS |
| 333 | VLCDR3 Ab30 | MQYVEAPLT |
| 334 | VH Ab30 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTWGPDGFDIWGQGT |
| | | MVTVSS |
| 335 | VL Ab30 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 336 | HC Ab30 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTWGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 337 | LC Ab30 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 338 | VHCDR1 Ab31 | SYAMH |
| 339 | VHCDR2 Ab31 | VISYDGSNKYYADSVKG |
| 340 | VLCDR3 Ab31 | GAGRGLTYGPDGFDI |
| 341 | VLCDR1 Ab31 | TSSRSLLHSDGKTYVY |
| 342 | VLCDR2 Ab31 | ELSNRFS |
| 343 | VLCDR3 Ab31 | MQYVEAPLT |
| 344 | VH Ab31 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSS |
| 345 | VL Ab31 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 346 | HC Ab31 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 347 | LC Ab31 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 348 | VHCDR1 Ab32 | SYAMH |
| 349 | VHCDR2 Ab32 | VISYDGSNKYYADSVKG |
| 350 | VLCDR3 Ab32 | GAGLGFTYGPDGFDI |
| 351 | VLCDR1 Ab32 | TSSRSLLHSDGKTYVY |
| 352 | VLCDR2 Ab32 | ELSNRFS |
| 353 | VLCDR3 Ab32 | MQYVEAPLT |
| 354 | VH Ab32 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFTYGPDGFDIWGQGT |
| | | MVTVSS |
| 355 | VL Ab32 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 356 | HC Ab32 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 357 | LC Ab32 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 358 | VHCDR1 Ab33 | SYAMH |
| 359 | VHCDR2 Ab33 | VISYDGSNKYYADSVKG |
| 360 | VLCDR3 Ab33 | GAGQGLSYGPDGFDI |
| 361 | VLCDR1 Ab33 | TSSRSLLHSDGKTYVY |
| 362 | VLCDR2 Ab33 | ELSNRFS |
| 363 | VLCDR3 Ab33 | MQYVEAPLT |
| 364 | VH Ab33 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGLSYGPDGFDIWGQGT |
| | | MVTVSS |
| 365 | VL Ab33 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 366 | HC Ab33 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGLSYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 367 | LC Ab33 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 368 | VHCDR1 Ab34 | SYAMH |
| 369 | VHCDR2 Ab34 | VISYDGSNKYYADSVKG |
| 370 | VLCDR3 Ab34 | GAGRGYTRGPDGFDI |
| 371 | VLCDR1 Ab34 | TSSRSLLHSDGKTYVY |
| 372 | VLCDR2 Ab34 | ELSNRFS |
| 373 | VLCDR3 Ab34 | MQYVEAPLT |
| 374 | VH Ab34 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTRGPDGFDIWGQGT |
| | | MVTVSS |
| 375 | VL Ab34 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 376 | HC Ab34 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTRGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 377 | LC Ab34 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 378 | VHCDR1 Ab35 | SYAMH |
| 379 | VHCDR2 Ab35 | VISYDGSNKYYADSVKG |
| 380 | VLCDR3 Ab35 | GAGRGYTNGPDGFDI |
| 381 | VLCDR1 Ab35 | TSSRSLLHSDGKTYVY |
| 382 | VLCDR2 Ab35 | ELSNRFS |
| 383 | VLCDR3 Ab35 | MQYVEAPLT |
| 384 | VH Ab35 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTNGPDGFDIWGQGT |
| | | MVTVSS |
| 385 | VL Ab35 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 386 | HC Ab35 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYTNGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 387 | LC Ab35 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 388 | VHCDR1 Ab36 | SYAMH |
| 389 | VHCDR2 Ab36 | VISYDGSNKYYADSVKG |
| 390 | VLCDR3 Ab36 | GAGRGFTYGPDGFDI |
| 391 | VLCDR1 Ab36 | RSSRSLLHSDGKTYVY |
| 392 | VLCDR2 Ab36 | ELSNRFS |
| 393 | VLCDR3 Ab36 | MQYIEAPLT |
| 394 | VH Ab36 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTYGPDGFDIWGQGT |
| | | MVTVSS |
| 395 | VL Ab36 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 396 | HC Ab36 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 397 | LC Ab36 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 398 | VHCDR1 Ab37 | SYAMH |
| 399 | VHCDR2 Ab37 | VISYDGSNKYYADSVKG |
| 400 | VLCDR3 Ab37 | GAGRGLTYGPDGFDI |
| 401 | VLCDR1 Ab37 | RSSRSLLHSDGKTYVY |
| 402 | VLCDR2 Ab37 | ELSNRFS |
| 403 | VLCDR3 Ab37 | MQYIEAPLT |
| 404 | VH Ab37 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSS |
| 405 | VL Ab37 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 406 | HC Ab37 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 407 | LC Ab37 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 408 | VHCDR1 Ab38 | SYAMH |
| 409 | VHCDR2 Ab38 | VISYDGSNKYYADSVKG |
| 410 | VLCDR3 Ab38 | GAGQGYPYGPDGFDI |
| 411 | VLCDR1 Ab38 | RSSRSLLHSDGKTYVY |
| 412 | VLCDR2 Ab38 | ELSNRFS |
| 413 | VLCDR3 Ab38 | MQYIEAPLT |
| 414 | VH Ab38 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGYPYGPDGFDIWGQGT |
| | | MVTVSS |
| 415 | VL Ab38 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 416 | HC Ab38 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 417 | LC Ab38 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 418 | VHCDR1 Ab39 | SYAMH |
| 419 | VHCDR2 Ab39 | VISYDGSNKYYADSVKG |
| 420 | VLCDR3 Ab39 | GAGTGFTYGPDGFDI |
| 421 | VLCDR1 Ab39 | RSSRSLLHSDGKTYVY |
| 422 | VLCDR2 Ab39 | ELSNRFS |
| 423 | VLCDR3 Ab39 | MQYIEAPLT |
| 424 | VH Ab39 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGTGFTYGPDGFDIWGQGT |
| | | MVTVSS |
| 425 | VL Ab39 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 426 | HC Ab39 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGTGFTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 427 | LC Ab39 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 428 | VHCDR1 Ab40 | SYAMH |
| 429 | VHCDR2 Ab40 | VISYDGSNKYYADSVKG |
| 430 | VLCDR3 Ab40 | GAGMGLTYGPDGFDI |
| 431 | VLCDR1 Ab40 | RSSRSLLHSDGKTYVY |
| 432 | VLCDR2 Ab40 | ELSNRFS |
| 433 | VLCDR3 Ab40 | MQYIEAPLT |
| 434 | VH Ab40 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGMGLTYGPDGFDIWGQGT |
| | | MVTVSS |
| 435 | VL Ab40 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 436 | HC Ab40 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGMGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 437 | LC Ab40 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 438 | VHCDR1 Ab41 | SYAMH |
| 439 | VHCDR2 Ab41 | VISYDGSNKYYADSVKG |
| 440 | VLCDR3 Ab41 | GAGLGFPYGPDGFDI |
| 441 | VLCDR1 Ab41 | RSSRSLLHSDGKTYVY |
| 442 | VLCDR2 Ab41 | ELSNRFS |
| 443 | VLCDR3 Ab41 | MQYIEAPLT |
| 444 | VH Ab41 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPYGPDGFDIWGQGT |
| | | MVTVSS |
| 445 | VL Ab41 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 446 | HC Ab41 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 447 | LC Ab41 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 448 | VHCDR1 Ab42 | SYAMH |
| 449 | VHCDR2 Ab42 | VISYDGSNKYYADSVKG |
| 450 | VLCDR3 Ab42 | GAGLGWAYGPDGFDI |
| 451 | VLCDR1 Ab42 | RSSRSLLHSDGKTYVY |
| 452 | VLCDR2 Ab42 | ELSNRFS |
| 453 | VLCDR3 Ab42 | MQYIEAPLT |
| 454 | VH Ab42 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWAYGPDGFDIWGQGT |
| | | MVTVSS |
| 455 | VL Ab42 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 456 | HC Ab42 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWAYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 457 | LC Ab42 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 458 | VHCDR1 Ab43 | SYAMH |
| 459 | VHCDR2 Ab43 | VISYDGSNKYYADSVKG |
| 460 | VLCDR3 Ab43 | GAGRGYPYGPDGFDI |
| 461 | VLCDR1 Ab43 | RSSRSLLHSDGKTYVY |
| 462 | VLCDR2 Ab43 | ELSNRFS |
| 463 | VLCDR3 Ab43 | MQYIEAPLT |
| 464 | VH Ab43 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSS |
| 465 | VL Ab43 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 466 | HC Ab43 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 467 | LC Ab43 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 468 | VHCDR1 Ab44 | GYAMH |
| 469 | VHCDR2 Ab44 | VISYDGSNKYYADSVKG |
| 470 | VLCDR3 Ab44 | GAGRGLTYGPDGFDI |
| 471 | VLCDR1 Ab44 | RSSRSLLHSDGKTYVY |
| 472 | VLCDR2 Ab44 | ELSNRFS |
| 473 | VLCDR3 Ab44 | MQYIEAPLT |
| 474 | VH Ab44 | EVQLLESGGGVVQPGRSLRLSCAASGFDFAGYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSS |
| 475 | VL Ab44 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 476 | HC Ab44 | EVQLLESGGGVVQPGRSLRLSCAASGFDFAGYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 477 | LC Ab44 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 478 | VHCDR1 Ab45 | SYAMH |
| 479 | VHCDR2 Ab45 | VISYDGSNKYYADSVKG |
| 480 | VLCDR3 Ab45 | GAGLGLTYGPDGFDI |
| 481 | VLCDR1 Ab45 | RSSRSLLHSDGKTYVY |
| 482 | VLCDR2 Ab45 | ELSNRFS |
| 483 | VLCDR3 Ab45 | MQYIEAPLT |
| 484 | VH Ab45 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGLTYGPDGFDIWGQGT |
| | | MVTVSS |
| 485 | VL Ab45 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 486 | HC Ab45 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 487 | LC Ab45 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 488 | VHCDR1 Ab46 | SYAMH |
| 489 | VHCDR2 Ab46 | VISYDGSNKYYADSVKG |
| 490 | VLCDR3 Ab46 | GAGLGYPYGPDGFDI |
| 491 | VLCDR1 Ab46 | RSSRSLLHSDGKTYVY |
| 492 | VLCDR2 Ab46 | ELSNRFS |
| 493 | VLCDR3 Ab46 | MQYIEAPLT |
| 494 | VH Ab46 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPYGPDGFDIWGQGT |
| | | MVTVSS |
| 495 | VL Ab46 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 496 | HC Ab46 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 497 | LC Ab46 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 498 | VHCDR1 Ab47 | SYAMH |
| 499 | VHCDR2 Ab47 | VISYDGSNKYYADSVKG |
| 500 | VLCDR3 Ab47 | GAGRGYPHGPDGFDI |
| 501 | VLCDR1 Ab47 | RSSRSLLHSDGKTYVY |
| 502 | VLCDR2 Ab47 | ELSNRFS |
| 503 | VLCDR3 Ab47 | MQYIEAPLT |
| 504 | VH Ab47 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPHGPDGFDIWGQGT |
| | | MVTVSS |
| 505 | VL Ab47 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 506 | HC Ab47 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPHGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 507 | LC Ab47 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 508 | VHCDR1 Ab48 | SYAMH |
| 509 | VHCDR2 Ab48 | VISYDGSNKYYADSVKG |
| 510 | VLCDR3 Ab48 | GAGIGYPHGPDGFDI |
| 511 | VLCDR1 Ab48 | RSSRSLLHSDGKTYVY |
| 512 | VLCDR2 Ab48 | ELSNRFS |
| 513 | VLCDR3 Ab48 | MQYIEAPLT |
| 514 | VH Ab48 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGIGYPHGPDGFDIWGQGT |
| | | MVTVSS |
| 515 | VL Ab48 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 516 | HC Ab48 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGIGYPHGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 517 | LC Ab48 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 518 | VHCDR1 Ab49 | SYAMH |
| 519 | VHCDR2 Ab49 | VISYDGSNKYYADSVKG |
| 520 | VLCDR3 Ab49 | GAGRGYPYGPDGFDI |
| 521 | VLCDR1 Ab49 | RSSRSLLHSDGKTYVY |
| 522 | VLCDR2 Ab49 | ELSNRFS |
| 523 | VLCDR3 Ab49 | MQYIEAPLT |
| 524 | VH Ab49 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSS |
| 525 | VL Ab49 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 526 | HC Ab49 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 527 | LC Ab49 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 528 | VHCDR1 Ab50 | SYAMH |
| 529 | VHCDR2 Ab50 | VISYDGSNKYYADSVKG |
| 530 | VLCDR3 Ab50 | GAGLGFPFGPDGFDI |
| 531 | VLCDR1 Ab50 | RSSRSLLHSDGKTYVY |
| 532 | VLCDR2 Ab50 | ELSNRFS |
| 533 | VLCDR3 Ab50 | MQYIEAPLT |
| 534 | VH Ab50 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPFGPDGFDIWGQGT |
| | | MVTVSS |
| 535 | VL Ab50 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 536 | HC Ab50 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPFGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 537 | LC Ab50 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 538 | VHCDR1 Ab51 | SYAMH |
| 539 | VHCDR2 Ab51 | VISYDGSNKYYADSVKG |
| 540 | VLCDR3 Ab51 | GAGRGFPYGPDGFDI |
| 541 | VLCDR1 Ab51 | RSSRSLLHSDGKTYVY |
| 542 | VLCDR2 Ab51 | ELSNRFS |
| 543 | VLCDR3 Ab51 | MQYIEAPLT |
| 544 | VH Ab51 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFPYGPDGFDIWGQGT |
| | | MVTVSS |
| 545 | VL Ab51 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 546 | HC Ab51 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 547 | LC Ab51 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 548 | VHCDR1 Ab52 | SYAMH |
| 549 | VHCDR2 Ab52 | VISYDGSNKYYADSVKG |
| 550 | VLCDR3 Ab52 | GAGLGYPNGPDGFDI |
| 551 | VLCDR1 Ab52 | RSSRSLLHSDGKTYVY |
| 552 | VLCDR2 Ab52 | ELSNRFS |
| 553 | VLCDR3 Ab52 | MQYIEAPLT |
| 554 | VH Ab52 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPNGPDGFDIWGQGT |
| | | MVTVSS |
| 555 | VL Ab52 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 556 | HC Ab52 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPNGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 557 | LC Ab52 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 558 | VHCDR1 Ab53 | SYAMH |
| 559 | VHCDR2 Ab53 | VISYDGSNKYYADSVKG |
| 560 | VLCDR3 Ab53 | GAGQGFPYGPDGFDI |
| 561 | VLCDR1 Ab53 | RSSRSLLHSDGKTYVY |
| 562 | VLCDR2 Ab53 | ELSNRFS |
| 563 | VLCDR3 Ab53 | MQYIEAPLT |
| 564 | VH Ab53 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGFPYGPDGFDIWGQGT |
| | | MVTVSS |
| 565 | VL Ab53 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 566 | HC Ab53 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 567 | LC Ab53 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 568 | VHCDR1 Ab54 | SYAMH |
| 569 | VHCDR2 Ab54 | VISYDGSNKYYADSVKG |
| 570 | VLCDR3 Ab54 | GAGQGYPYGPDGFDI |
| 571 | VLCDR1 Ab54 | TSSRSLLHSDGKTYVY |
| 572 | VLCDR2 Ab54 | ELSNRFS |
| 573 | VLCDR3 Ab54 | MQYIEAPLT |
| 574 | VH Ab54 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGYPYGPDGFDIWGQGT |
| | | MVTVSS |
| 575 | VL Ab54 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 576 | HC Ab54 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 577 | LC Ab54 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 578 | VHCDR1 Ab55 | SYAMH |
| 579 | VHCDR2 Ab55 | VISYDGSNKYYADSVKG |
| 580 | VLCDR3 Ab55 | GAGTGFTYGPDGFDI |
| 581 | VLCDR1 Ab55 | TSSRSLLHSDGKTYVY |
| 582 | VLCDR2 Ab55 | ELSNRFS |
| 583 | VLCDR3 Ab55 | MQYIEAPLT |
| 584 | VH Ab55 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGTGFTYGPDGFDIWGQGT |
| | | MVTVSS |
| 585 | VL Ab55 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 586 | HC Ab55 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGTGFTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 587 | LC Ab55 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 588 | VHCDR1 Ab56 | SYAMH |
| 589 | VHCDR2 Ab56 | VISYDGSNKYYADSVKG |
| 590 | VLCDR3 Ab56 | GAGMGLTYGPDGFDI |
| 591 | VLCDR1 Ab56 | TSSRSLLHSDGKTYVY |
| 592 | VLCDR2 Ab56 | ELSNRFS |
| 593 | VLCDR3 Ab56 | MQYIEAPLT |
| 594 | VH Ab56 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGMGLTYGPDGFDIWGQGT |
| | | MVTVSS |
| 595 | VL Ab56 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 596 | HC Ab56 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGMGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 597 | LC Ab56 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 598 | VHCDR1 Ab57 | SYAMH |
| 599 | VHCDR2 Ab57 | VISYDGSNKYYADSVKG |
| 600 | VLCDR3 Ab57 | GAGLGFPYGPDGFDI |
| 601 | VLCDR1 Ab57 | TSSRSLLHSDGKTYVY |
| 602 | VLCDR2 Ab57 | ELSNRFS |
| 603 | VLCDR3 Ab57 | MQYIEAPLT |
| 604 | VH Ab57 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPYGPDGFDIWGQGT |
| | | MVTVSS |
| 605 | VL Ab57 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 606 | HC Ab57 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 607 | LC Ab57 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 608 | VHCDR1 Ab58 | SYAMH |
| 609 | VHCDR2 Ab58 | VISYDGSNKYYADSVKG |
| 610 | VLCDR3 Ab58 | GAGLGWAYGPDGFDI |
| 611 | VLCDR1 Ab58 | TSSRSLLHSDGKTYVY |
| 612 | VLCDR2 Ab58 | ELSNRFS |
| 613 | VLCDR3 Ab58 | MQYIEAPLT |
| 614 | VH Ab58 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWAYGPDGFDIWGQGT |
| | | MVTVSS |
| 615 | VL Ab58 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 616 | HC Ab58 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWAYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 617 | LC Ab58 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 618 | VHCDR1 Ab59 | SYAMH |
| 619 | VHCDR2 Ab59 | VISYDGSNKYYADSVKG |
| 620 | VLCDR3 Ab59 | GAGRGYPYGPDGFDI |
| 621 | VLCDR1 Ab59 | TSSRSLLHSDGKTYVY |
| 622 | VLCDR2 Ab59 | ELSNRFS |
| 623 | VLCDR3 Ab59 | MQYIEAPLT |
| 624 | VH Ab59 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSS |
| 625 | VL Ab59 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 626 | HC Ab59 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 627 | LC Ab59 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 628 | VHCDR1 Ab60 | GYAMH |
| 629 | VHCDR2 Ab60 | VISYDGSNKYYADSVKG |
| 630 | VLCDR3 Ab60 | GAGRGLTYGPDGFDI |
| 631 | VLCDR1 Ab60 | TSSRSLLHSDGKTYVY |
| 632 | VLCDR2 Ab60 | ELSNRFS |
| 633 | VLCDR3 Ab60 | MQYIEAPLT |
| 634 | VH Ab60 | EVQLLESGGGVVQPGRSLRLSCAASGFDFAGYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSS |
| 635 | VL Ab60 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 636 | HC Ab60 | EVQLLESGGGVVQPGRSLRLSCAASGFDFAGYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 637 | LC Ab60 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 638 | VHCDR1 Ab61 | SYAMH |
| 639 | VHCDR2 Ab61 | VISYDGSNKYYADSVKG |
| 640 | VLCDR3 Ab61 | GAGLGLTYGPDGFDI |
| 641 | VLCDR1 Ab61 | TSSRSLLHSDGKTYVY |
| 642 | VLCDR2 Ab61 | ELSNRFS |
| 643 | VLCDR3 Ab61 | MQYIEAPLT |
| 644 | VH Ab61 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGLTYGPDGFDIWGQGT |
| | | MVTVSS |
| 645 | VL Ab61 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 646 | HC Ab61 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 647 | LC Ab61 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 648 | VHCDR1 Ab62 | SYAMH |
| 649 | VHCDR2 Ab62 | VISYDGSNKYYADSVKG |
| 650 | VLCDR3 Ab62 | GAGLGYPYGPDGFDI |
| 651 | VLCDR1 Ab62 | TSSRSLLHSDGKTYVY |
| 652 | VLCDR2 Ab62 | ELSNRFS |
| 653 | VLCDR3 Ab62 | MQYIEAPLT |
| 654 | VH Ab62 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPYGPDGFDIWGQGT |
| | | MVTVSS |
| 655 | VL Ab62 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 656 | HC Ab62 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 657 | LC Ab62 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 658 | VHCDR1 Ab63 | SYAMH |
| 659 | VHCDR2 Ab63 | VISYDGSNKYYADSVKG |
| 660 | VLCDR3 Ab63 | GAGRGYPHGPDGFDI |
| 661 | VLCDR1 Ab63 | TSSRSLLHSDGKTYVY |
| 662 | VLCDR2 Ab63 | ELSNRFS |
| 663 | VLCDR3 Ab63 | MQYIEAPLT |
| 664 | VH Ab63 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPHGPDGFDIWGQGT |
| | | MVTVSS |
| 665 | VL Ab63 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 666 | HC Ab63 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPHGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 667 | LC Ab63 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 668 | VHCDR1 Ab64 | SYAMH |
| 669 | VHCDR2 Ab64 | VISYDGSNKYYADSVKG |
| 670 | VLCDR3 Ab64 | GAGIGYPHGPDGFDI |
| 671 | VLCDR1 Ab64 | TSSRSLLHSDGKTYVY |
| 672 | VLCDR2 Ab64 | ELSNRFS |
| 673 | VLCDR3 Ab64 | MQYIEAPLT |
| 674 | VH Ab64 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGIGYPHGPDGFDIWGQGT |
| | | MVTVSS |
| 675 | VL Ab64 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 676 | HC Ab64 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGIGYPHGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 677 | LC Ab64 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 678 | VHCDR1 Ab65 | SYAMH |
| 679 | VHCDR2 Ab65 | VISYDGSNKYYADSVKG |
| 680 | VLCDR3 Ab65 | GAGRGYPYGPDGFDI |
| 681 | VLCDR1 Ab65 | TSSRSLLHSDGKTYVY |
| 682 | VLCDR2 Ab65 | ELSNRFS |
| 683 | VLCDR3 Ab65 | MQYIEAPLT |
| 684 | VH Ab65 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSS |
| 685 | VL Ab65 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 686 | HC Ab65 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 687 | LC Ab65 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 688 | VHCDR1 Ab66 | SYAMH |
| 689 | VHCDR2 Ab66 | VISYDGSNKYYADSVKG |
| 690 | VLCDR3 Ab66 | GAGLGFPFGPDGFDI |
| 691 | VLCDR1 Ab66 | TSSRSLLHSDGKTYVY |
| 692 | VLCDR2 Ab66 | ELSNRFS |
| 693 | VLCDR3 Ab66 | MQYIEAPLT |
| 694 | VH Ab66 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPFGPDGFDIWGQGT |
| | | MVTVSS |
| 695 | VL Ab66 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 696 | HC Ab66 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPFGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 697 | LC Ab66 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 698 | VHCDR1 Ab67 | SYAMH |
| 699 | VHCDR2 Ab67 | VISYDGSNKYYADSVKG |
| 700 | VLCDR3 Ab67 | GAGRGFPYGPDGFDI |
| 701 | VLCDR1 Ab67 | TSSRSLLHSDGKTYVY |
| 702 | VLCDR2 Ab67 | ELSNRFS |
| 703 | VLCDR3 Ab67 | MQYIEAPLT |
| 704 | VH Ab67 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFPYGPDGFDIWGQGT |
| | | MVTVSS |
| 705 | VL Ab67 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 706 | HC Ab67 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 707 | LC Ab67 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 708 | VHCDR1 Ab68 | SYAMH |
| 709 | VHCDR2 Ab68 | VISYDGSNKYYADSVKG |
| 710 | VLCDR3 Ab68 | GAGLGYPNGPDGFDI |
| 711 | VLCDR1 Ab68 | TSSRSLLHSDGKTYVY |
| 712 | VLCDR2 Ab68 | ELSNRFS |
| 713 | VLCDR3 Ab68 | MQYIEAPLT |
| 714 | VH Ab68 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPNGPDGFDIWGQGT |
| | | MVTVSS |
| 715 | VL Ab68 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 716 | HC Ab68 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPNGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 717 | LC Ab68 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 718 | VHCDR1 Ab69 | SYAMH |
| 719 | VHCDR2 Ab69 | VISYDGSNKYYADSVKG |
| 720 | VLCDR3 Ab69 | GAGQGFPYGPDGFDI |
| 721 | VLCDR1 Ab69 | TSSRSLLHSDGKTYVY |
| 722 | VLCDR2 Ab69 | ELSNRFS |
| 723 | VLCDR3 Ab69 | MQYIEAPLT |
| 724 | VH Ab69 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGFPYGPDGFDIWGQGT |
| | | MVTVSS |
| 725 | VL Ab69 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 726 | HC Ab69 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 727 | LC Ab69 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 728 | VHCDR1 Ab70 | SYAMH |
| 729 | VHCDR2 Ab70 | VISYDGSNKYYADSVKG |
| 730 | VLCDR3 Ab70 | GAGQGYPYGPDGFDI |
| 731 | VLCDR1 Ab70 | KSSRSLLHSDGKTYVY |
| 732 | VLCDR2 Ab70 | ELSNRFS |
| 733 | VLCDR3 Ab70 | MQYIEAPLT |
| 734 | VH Ab70 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGYPYGPDGFDIWGQGT |
| | | MVTVSS |
| 735 | VL Ab70 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 736 | HC Ab70 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 737 | LC Ab70 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 738 | VHCDR1 Ab71 | SYAMH |
| 739 | VHCDR2 Ab71 | VISYDGSNKYYADSVKG |
| 740 | VLCDR3 Ab71 | GAGTGFTYGPDGFDI |
| 741 | VLCDR1 Ab71 | KSSRSLLHSDGKTYVY |
| 742 | VLCDR2 Ab71 | ELSNRFS |
| 743 | VLCDR3 Ab71 | MQYIEAPLT |
| 744 | VH Ab71 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGTGFTYGPDGFDIWGQGT |
| | | MVTVSS |
| 745 | VL Ab71 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 746 | HC Ab71 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGTGFTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 747 | LC Ab71 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 748 | VHCDR1 Ab72 | SYAMH |
| 749 | VHCDR2 Ab72 | VISYDGSNKYYADSVKG |
| 750 | VLCDR3 Ab72 | GAGMGLTYGPDGFDI |
| 751 | VLCDR1 Ab72 | KSSRSLLHSDGKTYVY |
| 752 | VLCDR2 Ab72 | ELSNRFS |
| 753 | VLCDR3 Ab72 | MQYIEAPLT |
| 754 | VH Ab72 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGMGLTYGPDGFDIWGQGT |
| | | MVTVSS |
| 755 | VL Ab72 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 756 | HC Ab72 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGMGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 757 | LC Ab72 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 758 | VHCDR1 Ab73 | SYAMH |
| 759 | VHCDR2 Ab73 | VISYDGSNKYYADSVKG |
| 760 | VLCDR3 Ab73 | GAGLGFPYGPDGFDI |
| 761 | VLCDR1 Ab73 | KSSRSLLHSDGKTYVY |
| 762 | VLCDR2 Ab73 | ELSNRFS |
| 763 | VLCDR3 Ab73 | MQYIEAPLT |
| 764 | VH Ab73 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPYGPDGFDIWGQGT |
| | | MVTVSS |
| 765 | VL Ab73 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 766 | HC Ab73 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 767 | LC Ab73 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 768 | VHCDR1 Ab74 | SYAMH |
| 769 | VHCDR2 Ab74 | VISYDGSNKYYADSVKG |
| 770 | VLCDR3 Ab74 | GAGLGWAYGPDGFDI |
| 771 | VLCDR1 Ab74 | KSSRSLLHSDGKTYVY |
| 772 | VLCDR2 Ab74 | ELSNRFS |
| 773 | VLCDR3 Ab74 | MQYIEAPLT |
| 774 | VH Ab74 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWAYGPDGFDIWGQGT |
| | | MVTVSS |
| 775 | VL Ab74 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 776 | HC Ab74 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWAYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 777 | LC Ab74 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 778 | VHCDR1 Ab75 | SYAMH |
| 779 | VHCDR2 Ab75 | VISYDGSNKYYADSVKG |
| 780 | VLCDR3 Ab75 | GAGRGYPYGPDGFDI |
| 781 | VLCDR1 Ab75 | KSSRSLLHSDGKTYVY |
| 782 | VLCDR2 Ab75 | ELSNRFS |
| 783 | VLCDR3 Ab75 | MQYIEAPLT |
| 784 | VH Ab75 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSS |
| 785 | VL Ab75 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 786 | HC Ab75 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 787 | LC Ab75 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 788 | VHCDR1 Ab76 | GYAMH |
| 789 | VHCDR2 Ab76 | VISYDGSNKYYADSVKG |
| 790 | VLCDR3 Ab76 | GAGRGLTYGPDGFDI |
| 791 | VLCDR1 Ab76 | KSSRSLLHSDGKTYVY |
| 792 | VLCDR2 Ab76 | ELSNRFS |
| 793 | VLCDR3 Ab76 | MQYIEAPLT |
| 794 | VH Ab76 | EVQLLESGGGVVQPGRSLRLSCAASGFDFAGYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSS |
| 795 | VL Ab76 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 796 | HC Ab76 | EVQLLESGGGVVQPGRSLRLSCAASGFDFAGYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 797 | LC Ab76 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 798 | VHCDR1 Ab77 | SYAMH |
| 799 | VHCDR2 Ab77 | VISYDGSNKYYADSVKG |
| 800 | VLCDR3 Ab77 | GAGLGLTYGPDGFDI |
| 801 | VLCDR1 Ab77 | KSSRSLLHSDGKTYVY |
| 802 | VLCDR2 Ab77 | ELSNRFS |
| 803 | VLCDR3 Ab77 | MQYIEAPLT |
| 804 | VH Ab77 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGLTYGPDGFDIWGQGT |
| | | MVTVSS |
| 805 | VL Ab77 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 806 | HC Ab77 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 807 | LC Ab77 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 808 | VHCDR1 Ab78 | SYAMH |
| 809 | VHCDR2 Ab78 | VISYDGSNKYYADSVKG |
| 810 | VLCDR3 Ab78 | GAGLGYPYGPDGFDI |
| 811 | VLCDR1 Ab78 | KSSRSLLHSDGKTYVY |
| 812 | VLCDR2 Ab78 | ELSNRFS |
| 813 | VLCDR3 Ab78 | MQYIEAPLT |
| 814 | VH Ab78 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPYGPDGFDIWGQGT |
| | | MVTVSS |
| 815 | VL Ab78 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 816 | HC Ab78 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 817 | LC Ab78 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 818 | VHCDR1 Ab79 | SYAMH |
| 819 | VHCDR2 Ab79 | VISYDGSNKYYADSVKG |
| 820 | VLCDR3 Ab79 | GAGRGYPHGPDGFDI |
| 821 | VLCDR1 Ab79 | KSSRSLLHSDGKTYVY |
| 822 | VLCDR2 Ab79 | ELSNRFS |
| 823 | VLCDR3 Ab79 | MQYIEAPLT |
| 824 | VH Ab79 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPHGPDGFDIWGQGT |
| | | MVTVSS |
| 825 | VL Ab79 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 826 | HC Ab79 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPHGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 827 | LC Ab79 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 828 | VHCDR1 Ab80 | SYAMH |
| 829 | VHCDR2 Ab80 | VISYDGSNKYYADSVKG |
| 830 | VLCDR3 Ab80 | GAGIGYPHGPDGFDI |
| 831 | VLCDR1 Ab80 | KSSRSLLHSDGKTYVY |
| 832 | VLCDR2 Ab80 | ELSNRFS |
| 833 | VLCDR3 Ab80 | MQYIEAPLT |
| 834 | VH Ab80 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGIGYPHGPDGFDIWGQGT |
| | | MVTVSS |
| 835 | VL Ab80 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 836 | HC Ab80 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGIGYPHGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 837 | LC Ab80 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 838 | VHCDR1 Ab81 | SYAMH |
| 839 | VHCDR2 Ab81 | VISYDGSNKYYADSVKG |
| 840 | VLCDR3 Ab81 | GAGRGYPYGPDGFDI |
| 841 | VLCDR1 Ab81 | KSSRSLLHSDGKTYVY |
| 842 | VLCDR2 Ab81 | ELSNRFS |
| 843 | VLCDR3 Ab81 | MQYIEAPLT |
| 844 | VH Ab81 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSS |
| 845 | VL Ab81 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 846 | HC Ab81 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 847 | LC Ab81 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 848 | VHCDR1 Ab82 | SYAMH |
| 849 | VHCDR2 Ab82 | VISYDGSNKYYADSVKG |
| 850 | VLCDR3 Ab82 | GAGLGFPFGPDGFDI |
| 851 | VLCDR1 Ab82 | KSSRSLLHSDGKTYVY |
| 852 | VLCDR2 Ab82 | ELSNRFS |
| 853 | VLCDR3 Ab82 | MQYIEAPLT |
| 854 | VH Ab82 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPFGPDGFDIWGQGT |
| | | MVTVSS |
| 855 | VL Ab82 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 856 | HC Ab82 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPFGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 857 | LC Ab82 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 858 | VHCDR1 Ab83 | SYAMH |
| 859 | VHCDR2 Ab83 | VISYDGSNKYYADSVKG |
| 860 | VLCDR3 Ab83 | GAGRGFPYGPDGFDI |
| 861 | VLCDR1 Ab83 | KSSRSLLHSDGKTYVY |
| 862 | VLCDR2 Ab83 | ELSNRFS |
| 863 | VLCDR3 Ab83 | MQYIEAPLT |
| 864 | VH Ab83 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFPYGPDGFDIWGQGT |
| | | MVTVSS |
| 865 | VL Ab83 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 866 | HC Ab83 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 867 | LC Ab83 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 868 | VHCDR1 Ab84 | SYAMH |
| 869 | VHCDR2 Ab84 | VISYDGSNKYYADSVKG |
| 870 | VLCDR3 Ab84 | GAGLGYPNGPDGFDI |
| 871 | VLCDR1 Ab84 | KSSRSLLHSDGKTYVY |
| 872 | VLCDR2 Ab84 | ELSNRFS |
| 873 | VLCDR3 Ab84 | MQYIEAPLT |
| 874 | VH Ab84 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPNGPDGFDIWGQGT |
| | | MVTVSS |
| 875 | VL Ab84 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 876 | HC Ab84 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGYPNGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 877 | LC Ab84 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 878 | VHCDR1 Ab85 | SYAMH |
| 879 | VHCDR2 Ab85 | VISYDGSNKYYADSVKG |
| 880 | VLCDR3 Ab85 | GAGQGFPYGPDGFDI |
| 881 | VLCDR1 Ab85 | KSSRSLLHSDGKTYVY |
| 882 | VLCDR2 Ab85 | ELSNRFS |
| 883 | VLCDR3 Ab85 | MQYIEAPLT |
| 884 | VH Ab85 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGFPYGPDGFDIWGQGT |
| | | MVTVSS |
| 885 | VL Ab85 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 886 | HC Ab85 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 887 | LC Ab85 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 888 | VHCDR1 Ab86 | SYAMH |
| 889 | VHCDR2 Ab86 | VISYDGSNKYYADSVKG |
| 890 | VLCDR3 Ab86 | GAGRGLTYGPDGFDI |
| 891 | VLCDR1 Ab86 | KSSRSLLWSDGKTYVY |
| 892 | VLCDR2 Ab86 | ELSNRFS |
| 893 | VLCDR3 Ab86 | MQYIEAPLT |
| 894 | VH Ab86 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSS |
| 895 | VL Ab86 | DIVMTQTPLSLSVTPGQPASISCKSSRSLLWSDGKTYVYWYVQKSGQPPQLLIYELSNR |
| | | FSGVPDRFSGSGSRTDFTLKISRVEAEDVGVYYCMQYIEAPLTFGGGTKVEIK |
| 896 | HC Ab86 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 897 | LC Ab86 | DIVMTQTPLSLSVTPGQPASISCKSSRSLLWSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 898 | VHCDR1 Ab87 | SYAMH |
| 899 | VHCDR2 Ab87 | VISYDGSNKYYADSVKG |
| 900 | VLCDR3 Ab87 | GAGRGFTWGPDGFDI |
| 901 | VLCDR1 Ab87 | KSSRSLLHSDGKTYVY |
| 902 | VLCDR2 Ab87 | ELSNRFS |
| 903 | VLCDR3 Ab87 | MQYVEAPLT |
| 904 | VH Ab87 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTWGPDGFDIWGQGT |
| | | MVTVSS |
| 905 | VL Ab87 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 906 | HC Ab87 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTWGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 907 | LC Ab87 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 908 | VHCDR1 Ab88 | SYAMH |
| 909 | VHCDR2 Ab88 | VISYDGSNKYYADSVKG |
| 910 | VLCDR3 Ab88 | GAGRGLTYGPDGFDI |
| 911 | VLCDR1 Ab88 | KSSRSLLHSDGKTYLY |
| 912 | VLCDR2 Ab88 | ELSNRFS |
| 913 | VLCDR3 Ab88 | MQYIEAPLT |
| 914 | VH Ab88 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSS |
| 915 | VL Ab88 | DIVMTQTPLSLSVTPGQPASISCKSSRSLLHSDGKTYLYWYVQKSGQPPQLLIYELSNR |
| | | FSGVPDRFSGSGSRTDFTLKISRVEAEDVGVYYCMQYIEAPLTFGGGTKVEIK |
| 916 | HC Ab88 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGLTYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 917 | LC Ab88 | DIVMTQTPLSLSVTPGQPASISCKSSRSLLHSDGKTYLYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 918 | VHCDR1 Ab89 | SYAMH |
| 919 | VHCDR2 Ab89 | VISYDGSNKYYADSVKG |
| 920 | VLCDR3 Ab89 | GAGRGFPYGPDGFDI |
| 921 | VLCDR1 Ab89 | RSSRSLLHSDGKTYVY |
| 922 | VLCDR2 Ab89 | ELSNRFS |
| 923 | VLCDR3 Ab89 | MQYIEAPLT |
| 924 | VH Ab89 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFPYGPDGFDIWGQGT |
| | | MVTVSS |
| 925 | VL Ab89 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEVPLTFGGGTKVEIK |
| 926 | HC Ab89 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 927 | LC Ab89 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEVPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 928 | VHCDR1 Ab90 | SYAMH |
| 929 | VHCDR2 Ab90 | VISYDGSNKYYADSVKG |
| 930 | VLCDR3 Ab90 | GAGRGYPYGPDGFDI |
| 931 | VLCDR1 Ab90 | TSSRSLLHSDGKTYVY |
| 932 | VLCDR2 Ab90 | ELSNRFS |
| 933 | VLCDR3 Ab90 | MQYVEAPLT |
| 934 | VH Ab90 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSS |
| 935 | VL Ab90 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEVPLTFGGGTKVEIK |
| 936 | HC Ab90 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGYPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 937 | LC Ab90 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEVPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 938 | VHCDR1 Ab91 | SYAMH |
| 939 | VHCDR2 Ab91 | VISYDGSNKYYADSVKG |
| 940 | VLCDR3 Ab91 | GAGQGYMHGPDGFDI |
| 941 | VLCDR1 Ab91 | RSSRSLLWSDGKTYVY |
| 942 | VLCDR2 Ab91 | ELSNRFS |
| 943 | VLCDR3 Ab91 | MQYIEAPLT |
| 944 | VH Ab91 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGYMHGPDGFDIWGQGT |
| | | MVTVSS |
| 945 | VL Ab91 | DIVMTQTPLSLSVTPGQPASISCRSSRSLLWSDGKTYVYWYVQKSGQPPQLLIYELSNR |
| | | FSGVPDRFSGSGSRTDFTLKISRVEAEDVGVYYCMQYIEAPLTFGGGTKVEIK |
| 946 | HC Ab91 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGQGYMHGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 947 | LC Ab91 | DIVMTQTPLSLSVTPGQPASISCRSSRSLLWSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 948 | VHCDR1 Ab92 | SYAMH |
| 949 | VHCDR2 Ab92 | VISYDGSNKYYADSVKG |
| 950 | VLCDR3 Ab92 | GAGHGFPTGPDGFDI |
| 951 | VLCDR1 Ab92 | RSSRSLLHSDGKTYVY |
| 952 | VLCDR2 Ab92 | ELSNRFS |
| 953 | VLCDR3 Ab92 | MQYVEAPLT |
| 954 | VH Ab92 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGHGFPTGPDGFDIWGQGT |
| | | MVTVSS |
| 955 | VL Ab92 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 956 | HC Ab92 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGHGFPTGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 957 | LC Ab92 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 958 | VHCDR1 Ab93 | SYAMH |
| 959 | VHCDR2 Ab93 | VISYDGSNKYYADSVKG |
| 960 | VLCDR3 Ab93 | GAGLGFPYGPDGFDI |
| 961 | VLCDR1 Ab93 | KSSRSLLHSDGKTYLY |
| 962 | VLCDR2 Ab93 | ELSNRFS |
| 963 | VLCDR3 Ab93 | MQYIEAPLT |
| 964 | VH Ab93 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPYGPDGFDIWGQGT |
| | | MVTVSS |
| 965 | VL Ab93 | DIVMTQTPLSLSVTPGQPASISCKSSRSLLHSDGKTYLYWYVQKSGQPPQLLIYELSNR |
| | | FSGVPDRFSGSGSRTDFTLKISRVEAEDVGVYYCMQYIEAPLTFGGGTKVEIK |
| 966 | HC Ab93 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGFPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 967 | LC Ab93 | DIVMTQTPLSLSVTPGQPASISCKSSRSLLHSDGKTYLYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 968 | VHCDR1 Ab94 | SYAMH |
| 969 | VHCDR2 Ab94 | VISYDGSNKYYADSVKG |
| 970 | VLCDR3 Ab94 | GAGLGWPYGPDGFDI |
| 971 | VLCDR1 Ab94 | RSSRSLLHSDGKTYVY |
| 972 | VLCDR2 Ab94 | ELSNRFS |
| 973 | VLCDR3 Ab94 | MQYIEAPLT |
| 974 | VH Ab94 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSS |
| 975 | VL Ab94 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 976 | HC Ab94 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 977 | LC Ab94 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 978 | VHCDR1 Ab95 | SYAMH |
| 979 | VHCDR2 Ab95 | VISYDGSNKYYADSVKG |
| 980 | VLCDR3 Ab95 | GAGLGWPYGPDGFDI |
| 981 | VLCDR1 Ab95 | TSSRSLLHSDGKTYVY |
| 982 | VLCDR2 Ab95 | ELSNRFS |
| 983 | VLCDR3 Ab95 | MQYIEAPLT |
| 984 | VH Ab95 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSS |
| 985 | VL Ab95 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 986 | HC Ab95 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 987 | LC Ab95 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 988 | VHCDR1 Ab96 | SYAMH |
| 989 | VHCDR2 Ab96 | VISYDGSNKYYADSVKG |
| 990 | VLCDR3 Ab96 | GAGLGWPYGPDGFDI |
| 991 | VLCDR1 Ab96 | KSSRSLLHSDGKTYVY |
| 992 | VLCDR2 Ab96 | ELSNRFS |
| 993 | VLCDR3 Ab96 | MQYIEAPLT |
| 994 | VH Ab96 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSS |
| 995 | VL Ab96 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 996 | HC Ab96 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 997 | LC Ab96 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 998 | VHCDR1 Ab97 | SYAMH |
| 999 | VHCDR2 Ab97 | VISYDGSNKYYADSVKG |
| 1000 | VLCDR3 Ab97 | GAGLGWPYGPDGFDI |
| 1001 | VLCDR1 Ab97 | KSSRSLLHSDGKTYVY |
| 1002 | VLCDR2 Ab97 | ELSNRFS |
| 1003 | VLCDR3 Ab97 | MQYVEAPLT |
| 1004 | VH Ab97 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSS |
| 1005 | VL Ab97 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 1006 | HC Ab97 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1007 | LC Ab97 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1008 | VHCDR1 Ab98 | SYAMH |
| 1009 | VHCDR2 Ab98 | VISYDGSNKYYADSVKG |
| 1010 | VLCDR3 Ab98 | GAGLGWPYGPDGFDI |
| 1011 | VLCDR1 Ab98 | TSSRSLLHSDGKTYVY |
| 1012 | VLCDR2 Ab98 | ELSNRFS |
| 1013 | VLCDR3 Ab98 | MQYVEAPLT |
| 1014 | VH Ab98 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSS |
| 1015 | VL Ab98 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEVPLTFGGGTKVEIK |
| 1016 | HC Ab98 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1017 | LC Ab98 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEVPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1018 | VHCDR1 Ab99 | SYAMH |
| 1019 | VHCDR2 Ab99 | VISYDGSNKYYADSVKG |
| 1020 | VLCDR3 Ab99 | GAGLGWPYGPDGFDI |
| 1021 | VLCDR1 Ab99 | RSSRSLLWSDGKTYVY |
| 1022 | VLCDR2 Ab99 | ELSNRFS |
| 1023 | VLCDR3 Ab99 | MQYIEAPLT |
| 1024 | VH Ab99 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSS |
| 1025 | VL Ab99 | DIVMTQTPLSLSVTPGQPASISCRSSRSLLWSDGKTYVYWYVQKSGQPPQLLIYELSNR |
| | | FSGVPDRFSGSGSRTDFTLKISRVEAEDVGVYYCMQYIEAPLTFGGGTKVEIK |
| 1026 | HC Ab99 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1027 | LC Ab99 | DIVMTQTPLSLSVTPGQPASISCRSSRSLLWSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1028 | VHCDR1 | SYAMH |
| | Ab100 | |
| 1029 | VHCDR2 | VISYDGSNKYYADSVKG |
| | Ab100 | |
| 1030 | VLCDR3 | GAGLGWPYGPDGFDI |
| | Ab100 | |
| 1031 | VLCDR1 | KSSRSLLWSDGKTYVY |
| | Ab100 | |
| 1032 | VLCDR2 | ELSNRFS |
| | Ab100 | |
| 1033 | VLCDR3 | MQYVEAPLT |
| | Ab100 | |
| 1034 | VH Ab100 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSS |
| 1035 | VL Ab100 | DIVMTQTPLSLSVTPGQPASISCKSSRSLLWSDGKTYVYWYVQKSGQPPQLLIYELSNR |
| | | FSGVPDRFSGSGSRTDFTLKISRVEAEDVGVYYCMQYVEAPLTFGGGTKVEIK |
| 1036 | HC Ab100 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGLGWPYGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1037 | LC Ab100 | DIVMTQTPLSLSVTPGQPASISCKSSRSLLWSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1038 | VHCDR1 | SYAMH |
| | Ab101 | |
| 1039 | VHCDR2 | VISYDGSNKYYADSVKG |
| | Ab101 | |
| 1040 | VLCDR3 | GAGRGFTLGPDGFDI |
| | Ab101 | |
| 1041 | VLCDR1 | RSSRSLLHSDGKTYVY |
| | Ab101 | |
| 1042 | VLCDR2 | ELSNRFS |
| | Ab101 | |
| 1043 | VLCDR3 | MQYIEAPLT |
| | Ab101 | |
| 1044 | VH Ab101 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSS |
| 1045 | VL Ab101 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 1046 | HC Ab101 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1047 | LC Ab101 | DIVMTQTPLSLSVTPGQPASISCRSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1048 | VHCDR1 | SYAMH |
| | Ab102 | |
| 1049 | VHCDR2 | VISYDGSNKYYADSVKG |
| | Ab102 | |
| 1050 | VLCDR3 | GAGRGFTLGPDGFDI |
| | Ab102 | |
| 1051 | VLCDR1 | TSSRSLLHSDGKTYVY |
| | Ab102 | |
| 1052 | VLCDR2 | ELSNRFS |
| | Ab102 | |
| 1053 | VLCDR3 | MQYIEAPLT |
| | Ab102 | |
| 1054 | VH Ab102 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSS |
| 1055 | VL Ab102 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 1056 | HC Ab102 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1057 | LC Ab102 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1058 | VHCDR1 | SYAMH |
| | Ab103 | |
| 1059 | VHCDR2 | VISYDGSNKYYADSVKG |
| | Ab103 | |
| 1060 | VLCDR3 | GAGRGFTLGPDGFDI |
| | Ab103 | |
| 1061 | VLCDR1 | KSSRSLLHSDGKTYVY |
| | Ab103 | |
| 1062 | VLCDR2 | ELSNRFS |
| | Ab103 | |
| 1063 | VLCDR3 | MQYIEAPLT |
| | Ab103 | |
| 1064 | VH Ab103 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSS |
| 1065 | VL Ab103 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIK |
| 1066 | HC Ab103 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1067 | LC Ab103 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1068 | VHCDR1 | SYAMH |
| | Ab104 | |
| 1069 | VHCDR2 | VISYDGSNKYYADSVKG |
| | Ab104 | |
| 1070 | VLCDR3 | GAGRGFTLGPDGFDI |
| | Ab104 | |
| 1071 | VLCDR1 | KSSRSLLHSDGKTYVY |
| | Ab104 | |
| 1072 | VLCDR2 | ELSNRFS |
| | Ab104 | |
| 1073 | VLCDR3 | MQYVEAPLT |
| | Ab104 | |
| 1074 | VH Ab104 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSS |
| 1075 | VL Ab104 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIK |
| 1076 | HC Ab104 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1077 | LC Ab104 | DIVMTQTPLSLSVTPGQPASISCKSSRSLLHSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1078 | VHCDR1 | SYAMH |
| | Ab105 | |
| 1079 | VHCDR2 | VISYDGSNKYYADSVKG |
| | Ab105 | |
| 1080 | VLCDR3 | GAGRGFTLGPDGFDI |
| | Ab105 | |
| 1081 | VLCDR1 | TSSRSLLHSDGKTYVY |
| | Ab105 | |
| 1082 | VLCDR2 | ELSNRFS |
| | Ab105 | |
| 1083 | VLCDR3 | MQYVEAPLT |
| | Ab105 | |
| 1084 | VH Ab105 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSS |
| 1085 | VL Ab105 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEVPLTFGGGTKVEIK |
| 1086 | HC Ab105 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1087 | LC Ab105 | DIVMTQTPLSLSVTPGQPASISCTSSRSLLHSDGKTYVYWYVQKSGQPPQLLIYELSNR |
| | | FSGVPDRFSGSGSRTDFTLKISRVEAEDVGVYYCMQYVEVPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1088 | VHCDR1 | SYAMH |
| | Ab106 | |
| 1089 | VHCDR2 | VISYDGSNKYYADSVKG |
| | Ab106 | |
| 1090 | VLCDR3 | GAGRGFTLGPDGFDI |
| | Ab106 | |
| 1091 | VLCDR1 | RSSRSLLWSDGKTYVY |
| | Ab106 | |
| 1092 | VLCDR2 | ELSNRFS |
| | Ab106 | |
| 1093 | VLCDR3 | MQYIEAPLT |
| | Ab106 | |
| 1094 | VH Ab106 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSS |
| 1095 | VL Ab106 | DIVMTQTPLSLSVTPGQPASISCRSSRSLLWSDGKTYVYWYVQKSGQPPQLLIYELSNR |
| | | FSGVPDRFSGSGSRTDFTLKISRVEAEDVGVYYCMQYIEAPLTFGGGTKVEIK |
| 1096 | HC Ab106 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1097 | LC Ab106 | DIVMTQTPLSLSVTPGQPASISCRSSRSLLWSDGKTYVYWYVQKSGQPPQLLIYELSNR |
| | | FSGVPDRFSGSGSRTDFTLKISRVEAEDVGVYYCMQYIEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1098 | VHCDR1 | SYAMH |
| | Ab107 | |
| 1099 | VHCDR2 | VISYDGSNKYYADSVKG |
| | Ab107 | |
| 1100 | VLCDR3 | GAGRGFTLGPDGFDI |
| | Ab107 | |
| 1101 | VLCDR1 | KSSRSLLWSDGKTYVY |
| | Ab107 | |
| 1102 | VLCDR2 | ELSNRFS |
| | Ab107 | |
| 1103 | VLCDR3 | MQYVEAPLT |
| | Ab107 | |
| 1104 | VH Ab107 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSS |
| 1105 | VL Ab107 | DIVMTQTPLSLSVTPGQPASISCKSSRSLLWSDGKTYVYWYVQKSGQPPQLLIYELSNR |
| | | FSGVPDRFSGSGSRTDFTLKISRVEAEDVGVYYCMQYVEAPLTFGGGTKVEIK |
| 1106 | HC Ab107 | EVQLLESGGGVVQPGRSLRLSCAASGFTFSSYAMHWVRQAPGKGLEWVAVISYDGSNKY |
| | | YADSVKGRFTISRDNSKNTLSLQMNSLRVEDTAIYYCVRGAGRGFTLGPDGFDIWGQGT |
| | | MVTVSSKGPSVFPLAPSSRSTSESTAALGCLVKDYFPEPVTVSWNSGALTSGVHTFPAV |
| | | LQSSGLYSLSSVVTVPSSNFGTQTYTCNVDHKPSNTKVDKTVERKCCVECPPCPAPPVA |
| | | GPSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPPS |
| | | REEMTKNQVSLTCLVKGFYPSDIAVEWESNGQPENNYKTTPPMLDSDGSFFLYSKLTVD |
| | | KSRWQQGNVFSCSVMHEALHNHYTQKSLSLSPG |
| 1107 | LC Ab107 | DIVMTQTPLSLSVTPGQPASISCKSSRSLLWSDGKTYVYWYVQKSGQPPQLLIYELSNR |
| | | FSGVPDRFSGSGSRTDFTLKISRVEAEDVGVYYCMQYVEAPLTFGGGTKVEIKRTVAAP |
| | | SVFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 1108 | Vh1 CDR1 | SYAMH |
| 1109 | Vh1 CDR2 | VISYDGSNKYYADSVKG |
| 1110 | Vh1 CDR3 | GAGRGYTYGPDGFDI |
| 1111 | Vh1 | EVQLVESGGGVVQPGRSLRLSCAASGFTFSSYAMHWVRQAPGKGLEWVAVISYDGSNKY |
| | | YADSVKGRFTISRDNSKNTLYLQMNSLRAEDTAIYYCVRGAGRGYTYGPDGFDIWGQGT |
| | | MVTVSS |
| 1112 | Vh2 CDR1 | SYAMH |
| 1113 | Vh2 CDR2 | VISYDGSNKYYADSVKG |
| 1114 | Vh2 CDR3 | GAGRGYTYGPDGFDI |
| 1115 | Vh2 | EVQLVESGGGVVQPGRSLRLSCAASGFTFSSYAMHWVRQAPGKGLEWVAVISYDGSNKY |
| | | YADSVKGRFTISRDNSKNTLYLQMNSLRAEDTAVYYCARGAGRGYTYGPDGFDIWGQGT |
| | | MVTVSS |
| 1116 | Vk1 CDR1 | TSSRSLLHSDGKTYLY |
| 1117 | Vk1 CDR2 | ELSNRFS |
| 1118 | Vk1 CDR3 | MQYIEAPLT |
| 1119 | Vk1 | DIVMTQTPLSLSVTPGQPASISCTSSRSLLHSDGKTYLYWYLQKPGQPPQLLIYELSNR |
| | | FSGVPDRFSGSGSRTDFTLKISRVEAEDVGVYYCMQYIEAPLTFGGGTKVEIK |
| 1120 | Vk2 CDR1 | TSSRSLLHSDGKTYVY |
| 1121 | Vk2 CDR2 | ELSNRFS |
| 1122 | Vk2 CDR3 | MQYIEAPLT |
| 1123 | Vk2 | DIVMTQTPLSLSVTPGQPASISCTSSRSLLHSDGKTYVYWYLQKPGQPPQLLIYELSNR |
| | | FSGVPDRFSGSGSRTDFTLKISRVEAEDVGVYYCMQYIEAPLTFGGGTKVEIK |
| 1124 | Vk3 CDR1 | TSSRSLLHSDGKTYVY |
| 1125 | Vk3 CDR2 | ELSNRFS |
| 1126 | Vk3 CDR3 | MQYIEAPLT |
| 1127 | Vk3 | DIVMTQTPLSLSVTPGQPASISCTSSRSLLHSDGKTYVYWYLQKSGQPPQLLIYELSNR |
| | | FSGVPDRFSGSGSRTDFTLKISRVEAEDVGVYYCMQYIEAPLTFGGGTKVEIK |
| 1128 | Vk4 CDR1 | TSSRSLLHSDGKTYVY |
| 1129 | Vk4 CDR2 | ELSNRFS |
| 1130 | Vk4 CDR3 | MQYIEAPLT |
| 1131 | Vk4 | DIVMTQTPLSLSVTPGQPASISCTSSRSLLHSDGKTYVYWYVQKPGQPPQLLIYELSNR |
| | | FSGVPDRFSGSGSRTDFTLKISRVEAEDVGVYYCMQYIEAPLTFGGGTKVEIK |
| 1132 | Vk5 CDR1 | RSSRSLLHSDGKTYVY |
| 1133 | Vk5 CDR2 | ELSNRFS |
| 1134 | Vk5 CDR3 | MQYIEAPLT |
| 1135 | Vk5 | DIVMTQTPLSLSVTPGQPASISCRSSRSLLHSDGKTYVYWYLQKPGQPPQLLIYELSNR |
| | | FSGVPDRFSGSGSGTDFTLKISRVEAEDVGVYYCMQYIEAPLTFGGGTKVEIK |
| 1136 | Vk6 CDR1 | KSSQSLLHSDGKTYLY |
| 1137 | Vk6 CDR2 | ELSNRFS |
| 1138 | Vk6 CDR3 | MQYIEAPLT |
| 1139 | Vk6 | DIVMTQTPLSLSVTPGQPASISCKSSQSLLHSDGKTYLYWYLQKPGQPPQLLIYELSNR |
| | | FSGVPDRFSGSGSGTDFTLKISRVEAEDVGVYYCMQYIEAPLTFGGGTKVEIK |
| 1140 | Vk7 CDR1 | KSSQSLLHSDGKTYLY |
| 1141 | Vk7 CDR2 | ELSNRFS |
| 1142 | Vk7 CDR3 | MQYIEAPLT |
| 1143 | Vk7 | DIVMTQSPLSLPVTPGEPASISCKSSQSLLHSDGKTYLYWYLQKPGQPPQLLIYELSNR |
| | | FSGVPDRFSGSGSGTDFTLKISRVEAEDVGVYYCMQYIEAPLTFGGGTKVEIK |
| 1144 | Vk8 CDR1 | TSSRSLLHSDGKTYVY |
| 1145 | Vk8 CDR2 | YLGNRFS |
| 1146 | Vk8 CDR3 | MQYIEAPLT |
| 1147 | Vk8 | DIVMTQTPLSLSVTPGQPASISCTSSRSLLHSDGKTYVYWYLQKPGQPPQLLIIYLGNR |
| | | FSGVPDRFSGSGSRTDFTLKISRVEAEDVGVYYCMQYIEAPLTFGGGTKVEIK |
| 1148 | Vk9 CDR1 | TSSRSLLHSDGKTYVY |
| 1149 | Vk9 CDR2 | ELSNRFS |
| 1150 | Vk9 CDR3 | MQYIEAPLT |
| 1151 | Vk9 | DIVMTQSPDSLAVSLGERATINCKSSQSLLHSDGKTYVYWYQQKPGQPPKLLIYELSNR |
| | | FSGVPDRFSGSGSGTDFTLTISSLQAEDVAVYYCMQYIEAPLTFGGGTKVEIK |
| 1152 | Vk10 CDR1 | TSSRSLLHSDGKTYVY |
| 1153 | Vk10 CDR2 | ELSNRFS |
| 1154 | Vk10 CDR3 | MQYIEAPLT |
| 1155 | Vk10 | DIVMTQSPDSLAVSLGERATISCTSSRSLLHSDGKTYVYWYQQKPGQPPKLLIYELSNR |
| | | FSGVPDRFSGSGSRTDFTLTISSLQAEDVAVYYCMQYIEAPLTFGGGTKVEIK |
| 1156 | Vk11 CDR1 | TSSRSLLHSDGKTYVY |
| 1157 | Vk11 CDR2 | ELSNRFS |
| 1158 | Vk11 CDR3 | MQYIEAPLT |
| 1159 | Vk11 | DIVMTQSPLSLPVTPGEPASISCTSSRSLLHSDGKTYVYWYLQKPGQSPQVLIYELSNR |
| | | FSGVPDRFSGSGSGTDFTLKISRVEAEDVGVYYCMQYIEAPLTFGGGTKVEIK |
| 1160 | Vk12 CDR1 | TSSRSLLHSDGKTYVY |
| 1161 | Vk12 CDR2 | ELSNRFS |
| 1162 | Vk12 CDR3 | MQYIEAPLT |
| 1163 | Vk12 | DIVMTQSPLSLPVTPGEPASISCTSSRSLLHSDGKTYVYWYLQKPGQSPQLLIYELSNR |
| | | FSGVPDRFSGSGSGTDFTLKISRVEAEDVGVYYCMQYIEAPLTFGGGTKVEIK |
| 1164 | Vk13 CDR1 | TSSRSLLHSDGKTYVY |
| 1165 | Vk13 CDR2 | ELSNRFS |
| 1166 | Vk13 CDR3 | MQYIEAPLT |
| 1167 | Vk13 | DVVMTQSPLSLPVTLGQPASISCTSSRSLLHSDGKTYVYWFQQRPGQSPRRLIYELSNR |
| | | FSGVPDRFSGSGSGTDFTLKISRVEAEDVGVYYCMQYIEAPLTFGGGTKVEIK |

Constant regions

| | | |
|---|---|---|
| 1168 | human IgG1 | ASTKGPSVFPLAPSSKSTSGGTAALGCLVKDYFPEPVTVSWNSGALTSGVHTFPAVLQ |
| | constant | SSGLYSLSSVVTVPSSSLGTQTYICNVNHKPSNTKVDKKVEPKSCDKTHTCPPCPAPE |
| | region | LLGGPSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| 1169 | human IgG2 | ASTKGPSVFPLAPCSRSTSESTAALGCLVKDYFPEPVTVSWNSGALTSGVHTFPAVLQ |
| | constant | SSGLYSLSSVVTVPSSNFGTQTYTCNVDHKPSNTKVDKTVERKCCVECPPCPAPPVAG |
| | region | PSVFLFPPKPKDTLMISRTPEVTCVVVDVSHEDPEVQFNWYVDGVEVHNAKTKPREEQ |
| | | FNSTFRVVSVLTVVHQDWLNGKEYKCKVSNKGLPAPIEKTISKTKGQPREPQVYTLPP |
| | | SREEMTKNQVSLTCLVKGFYPSDIAVEWESNGQPENNYKTTPPMLDSDGSFFLYSKLT |
| | | VDKSRWQQGNVFSCSVMHEALHNHYTQKSLSLSPG |
| 1170 | human IgG3 | ASTKGPSVFPLAPCSRSTSGGTAALGCLVKDYFPEPVTVSWNSGALTSGVHTFPAVLQ |
| | constant | SSGLYSLSSVVTVPSSSLGTQTYTCNVNHKPSNTKVDKRVELKTPLGDTTHTCPRCPE |
| | region | PKSCDTPPPCPRCPEPKSCDTPPPCPRCPEPKSCDTPPPCPRCPAPELLGGPSVFLFP |
| | | PKPKDTLMISRTPEVTCVVVDVSHEDPEVQFKWYVDGVEVHNAKTKPREEQYNSTFRV |
| | | VSVLTVLHQDWLNGKEYKCKVSNKALPAPIEKTISKTKGQPREPQVYTLPPSREEMTK |
| | | NQVSLTCLVKGFYPSDIAVEWESSGQPENNYNTTPPMLDSDGSFFLYSKLTVDKSRWQ |
| | | QGNIFSCSVMHEALHNRFTQKSLSLSPG |
| 1171 | human IgG4 | ASTKGPSVFPLAPCSRSTSESTAALGCLVKDYFPEPVTVSWNSGALTSGVHTFPAVLQ |
| | constant | SSGLYSLSSVVTVPSSSLGTKTYTCNVDHKPSNTKVDKRVESKYGPPCPSCPAPEFLG |
| | region | GPSVFLFPPKPKDTLMISRTPEVTCVVVDVSQEDPEVQFNWYVDGVEVHNAKTKPREE |
| | | QFNSTYRVVSVLTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVLDSDGSFFLYSRL |
| | | TVDKSRWQEGNVFSCSVMHEALHNHYTQKSLSLSLG |
| 1172 | M5 | ASTKGPSVFPLAPSSRSTSESTAALGCLVKDYFPEPVTVSWNSGALTSGVHTFPAVLQ |
| | | SSGLYSLSSVVTVPSSNFGTQTYTCNVDHKPSNTKVDKTVERKCCVECPPCPAPPVAG |
| | | PSVFLFPPKPKDTLMISRTPEVTCVVVDVSQEDPEVQFNWYVDGVEVHNAKTKPREEQ |
| | | FNSTFRVVSVLTVLHQDWLNGKEYKCKVSNKGLPSSIEKTISKTKGQPREPQVYTLPP |
| | | SREEMTKNQVSLTCLVKGFYPSDIAVEWESNGQPENNYKTTPPMLDSDGSFFLYSKLT |
| | | VDKSRWQQGNVFSCSVMHEALHNHYTQKSLSLSPG |
| 1173 | IgG2 | ASTKGPSVFPLAPSSRSTSESTAALGCLVKDYFPEPVTVSWNSGALTSGVHTFPAVLQS |
| | (C127S) | SGLYSLSSVVTVPSSNFGTQTYTCNVDHKPSNTKVDKTVERKCCVECPPCPAPPVAGPS |
| | (M7) | VFLFPPKPKDTLMISRTPEVTCVVVDVSHEDPEVQFNWYVDGVEVHNAKTKPREEQFNS |
| | | TFRVVSVLTVVHQDWLNGKEYKCKVSNKGLPAPIEKTISKTKGQPREPQVYTLPPSREE |
| | | MTKNQVSLTCLVKGFYPSDIAVEWESNGQPENNYKTTPPMLDSDGSFFLYSKLTVDKSR |
| | | WQQGNVFSCSVMHEALHNHYTQKSLSLSPG |
| 1174 | IgG1_IgG4 | ASTKGPSVFPLAPSSKSTSGGTAALGCLVKDYFPEPVTVSWNSGALTSGVHTFPAVLQS |
| | Fc region | SGLYSLSSVVTVPSSSLGTQTYICNVNHKPSNTKVDKKVEPKSCDKTHTCPSCPAPEAA |
| | (M9) | GGPSVFLFPPKPKDTLMISRTPEVTCVVVDVSQEDPEVQFNWYVDGVEVHNAKTKPREE |
| | | QFNSTYRVVSVLTVLHQDWLNGKEYKCKVSNKGLPSSIEKTISKAKGQPREPQVYTLPP |
| | | SREEMTKNQVSLTCLVKGFYPSDIAVEWESNGQP |
| | | ENNYKTTPPVLDSDGSFFLYSKLTVDKSRWQQGNVFSCSVMHEALHNHYTQKSLSLSPG |
| 1175 | IgG1 | ASTKGPSVFPLAPSSKSTSGGTAALGCLVKDYFPEPVTVSWNSGALTSGVHTFPAVLQS |
| | (D265A/N297Q) | SGLYSLSSVVTVPSSSLGTQTYICNVNHKPSNTKVDKKVEPKSCDKTHTCPPCPAPELL |
| | (M11) | GGPSVFLFPPKPKDTLMISRTPEVTCVVVAVSHEDPEVKFNWYVDGVEVHNAKTKPREE |
| | | QYQSTYRVVSVLTVLHQDWLNGKEYKCKVSNKALPAPIEKTISKAKGQPREPQVYTLPP |
| | | SREEMTKNQVSLTCLVKGFYPSDIAVEWESNGQPENNYKTTPPVLDSDGSFFLYSKLTV |
| | | DKSRWQQGNVFSCSVMHEALHNHYTQKSLSLSPG |

Nucleic acids

| | | |
|---|---|---|
| 1176 | VH Ab16 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATACCCGTGGCCCGGATGGCTTTGATATTTGGGGCCAGGGCACCatgGTGACCGTGAGCAGC |
| 1177 | VH Ab17 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATACCAACGGCCCGGATGGCTTTGATATTTGGGGCCAGGGCACCatgGTGACCGTGAGCAGC |
| 1178 | VH Ab18 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCTGGGGCCCGGATGGCTTTGATATTTGGGGCCAGGGCACCatgGTGACCGTGAGCAGC |
| 1179 | VH Ab19 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCCTGGGCCCGGATGGCTTTGATATTTGGGGCCAGGGCACCatgGTGACCGTGAGCAGC |
| 1180 | VH Ab20 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCC |
| | | TGACCTTTGGCCCGGATGGCTTTGATATTTGGGGCCAGGGCACCatgGTGACCGTGAGCAGC |
| 1181 | VH Ab21 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| 1190 | VH Ab30 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGNNNAAANNNNNNATTTATTATTGCGTGCGTGGCGCGGGCCGTGGCT |
| | | TTACCTGGGGCCCGGATGGCTTTGATATTTGGGGCCAGGGCACCatgGTGACCGTGAGCAGC |
| 1191 | VH Ab31 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCC |
| | | TGACCTATGGCCCGGATGGCTTTGATATTTGGGGCCAGGGCACCatgGTGACCGTGAGCAGC |
| 1192 | VH Ab32 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | TTACCTATGGCCCGGATGGCTTTGATATTTGGGGCCAGGGCACCatgGTGACCGTGAGCAGC |
| 1193 | VH Ab33 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCAGGGCC |
| | | TGAGCTATGGCCCGGATGGCTTTGATATTTGGGGCCAGGGCACCatgGTGACCGTGAGCAGC |
| 1194 | VH Ab34 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATACCCGTGGCCCGGATGGCTTTGATATTTGGGGCCAGGGCACCatgGTGACCGTGAGCAGC |
| 1195 | VH Ab35 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATACCAACGGCCCGGATGGCTTTGATATTTGGGGCCAGGGCACCatgGTGACCGTGAGCAGC |
| 1196 | VH Ab36 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCTATGGCCCGGATGGCTTTGATATTTGGGGCCAGGGCACCatgGTGACCGTGAGCAGC |
| 1197 | VH Ab37 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCC |
| | | TGACCTATGGCCCGGATGGCTTTGATATTTGGGGCCAGGGCACCatgGTGACCGTGAGCAGC |
| 1198 | VH Ab38 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCAGGGCT |
| | | ATCCGTATGGCCCGGATGGCTTTGATATTTGGGGCCAGGGCACCatgGTGACCGTGAGCAGC |
| 1199 | VH Ab39 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCACCGGCT |
| | | TTACCTATGGCCCGGATGGCTTTGATATTTGGGGCCAGGGCACCatgGTGACCGTGAGCAGC |
| 1200 | VH Ab40 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCatgGGCC |
| | | TGACCTATGGCCCGGATGGCTTTGATATTTGGGGCCAGGGCACCatgGTGACCGTGAGCAGC |
| 1201 | VH Ab41 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | TTCCGTATGGCCCGGATGGCTTTGATATTTGGGGCCAGGGCACCatgGTGACCGTGAGCAGC |
| 1202 | VH Ab42 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | GGGCGTATGGCCCGGATGGCTTTGATATTTGGGGCCAGGGCACCatgGTGACCGTGAGCAGC |
| 1203 | VH Ab43 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATCCGTATGGCCCGGATGGCTTTGATATTTGGGGCCAGGGCACCatgGTGACCGTGAGCAGC |
| 1204 | VH Ab44 | GAAGTGCAGCTGCTGGAAAGCGGCGGCGGCGTGGTGCAGCCGGGCCGTAGCCTGCGTCTGAG |
| | | CTGCGCGGCGAGCGGCTTTGATTTTGCGGGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCC |
| | | TGACCTATGGCCCGGATGGCTTTGATATTTGGGGCCAGGGCACCatgGTGACCGTGAGCAGC |
| 1205 | VH Ab45 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCC |
| | | TGACCTATGGCCCGGATGGCTTTGATATTTGGGGCCAGGGCACCatgGTGACCGTGAGCAGC |
| 1206 | VH Ab46 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | ATCCGTATGGCCCGGATGGCTTTGATATTTGGGGCCAGGGCACCatgGTGACCGTGAGCAGC |
| 1207 | VH Ab47 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATCCGCATGGCCCGGATGGCTTTGATATTTGGGGCCAGGGCACCatgGTGACCGTGAGCAGC |
| 1208 | VH Ab48 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCATTGGCT |
| | | ATCCGCATGGCCCGGATGGCTTTGATATTTGGGGCCAGGGCACCatgGTGACCGTGAGCAGC |
| 1209 | VH Ab49 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATCCGTATGGCCCGGATGGCTTTGATATTTGGGGCCAGGGCACCatgGTGACCGTGAGCAGC |
| 1210 | VH Ab50 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | TTCCGTTTGGCCCGGATGGCTTTGATATTTGGGGCCAGGGCACCatgGTGACCGTGAGCAGC |
| 1211 | VH Ab51 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTCCGTATGGCCCGGATGGCTTTGATATTTGGGGCCAGGGCACCatgGTGACCGTGAGCAGC |
| 1212 | VH Ab52 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | ATCCGAACGGCCCGGATGGCTTTGATATTTGGGGCCAGGGCACCatgGTGACCGTGAGCAGC |
| 1213 | VH Ab53 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCAGGGCT |
| | | TTCCGTATGGCCCGGATGGCTTTGATATTTGGGGCCAGGGCACCatgGTGACCGTGAGCAGC |
| 1214 | VH Ab54 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCAGGGCT |
| | | ATCCGTATGGCCCGGATGGCTTTGATATTTGGGGCCAGGGCACCatgGTGACCGTGAGCAGC |
| 1215 | VH Ab55 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCACCGGCT |
| | | TTACCTATGGCCCGGATGGCTTTGATATTTGGGGCCAGGGCACCatgGTGACCGTGAGCAGC |
| 1216 | VH Ab56 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCatgGGCC |
| | | TGACCTATGGCCCGGATGGCTTTGATATTTGGGGCCAGGGCACCatgGTGACCGTGAGCAGC |
| 1217 | VH Ab57 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | TTCCGTATGGCCCGGATGGCTTTGATATTTGGGGCCAGGGCACCatgGTGACCGTGAGCAGC |
| 1218 | VH Ab58 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | GGGCGTATGGCCCGGATGGCTTTGATATTTGGGGCCAGGGCACCatgGTGACCGTGAGCAGC |
| 1219 | VH Ab59 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATCCGTATGGCCCGGATGGCTTTGATATTTGGGGCCAGGGCACCatgGTGACCGTGAGCAGC |
| 1220 | VH Ab60 | GAAGTGCAGCTGCTGGAAAGCGGCGGCGGCGTGGTGCAGCCGGGCCGTAGCCTGCGTCTGAG |
| | | CTGCGCGGCGAGCGGCTTTGATTTTGCGGGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCC |
| | | TGACCTATGGCCCGGATGGCTTTGATATTTGGGGCCAGGGCACCatgGTGACCGTGAGCAGC |
| 1221 | VH Ab61 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCC |
| | | TGACCTATGGCCCGGATGGCTTTGATATTTGGGGCCAGGGCACCatgGTGACCGTGAGCAGC |
| 1222 | VH Ab62 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | ATCCGTATGGCCCGGATGGCTTTGATATTTGGGGCCAGGGCACCatgGTGACCGTGAGCAGC |
| 1223 | VH Ab63 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATCCGCATGGCCCGGATGGCTTTGATATTTGGGGCCAGGGCACCatgGTGACCGTGAGCAGC |
| 1224 | VH Ab64 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCATTGGCT |
| | | ATCCGCATGGCCCGGATGGCTTTGATATTTGGGGCCAGGGCACCatgGTGACCGTGAGCAGC |
| 1225 | VH Ab65 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATCCGTATGGCCCGGATGGCTTTGATATTTGGGGCCAGGGCACCatgGTGACCGTGAGCAGC |
| 1226 | VH Ab66 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | TTCCGTTTGGCCCGGATGGCTTTGATATTTGGGGCCAGGGCACCatgGTGACCGTGAGCAGC |
| 1227 | VH Ab67 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTCCGTATGGCCCGGATGGCTTTGATATTTGGGGCCAGGGCACCatgGTGACCGTGAGCAGC |
| 1228 | VH Ab68 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | ATCCGAACGGCCCGGATGGCTTTGATATTTGGGGCCAGGGCACCatgGTGACCGTGAGCAGC |
| 1229 | VH Ab69 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCAGGGCT |
| | | TTCCGTATGGCCCGGATGGCTTTGATATTTGGGGCCAGGGCACCatgGTGACCGTGAGCAGC |
| 1230 | VH Ab70 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCAGGGCT |
| | | ATCCGTATGGCCCGGATGGCTTTGATATTTGGGGCCAGGGCACCatgGTGACCGTGAGCAGC |
| 1231 | VH Ab71 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCACCGGCT |
| | | TTACCTATGGCCCGGATGGCTTTGATATTTGGGGCCAGGGCACCatgGTGACCGTGAGCAGC |
| 1232 | VH Ab72 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCatgGGCC |
| | | TGACCTATGGCCCGGATGGCTTTGATATTTGGGGCCAGGGCACCatgGTGACCGTGAGCAGC |
| 1233 | VH Ab73 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | TTCCGTATGGCCCGGATGGCTTTGATATTTGGGGCCAGGGCACCatgGTGACCGTGAGCAGC |
| 1234 | VH Ab74 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | GGGCGTATGGCCCGGATGGCTTTGATATTTGGGGCCAGGGCACCatgGTGACCGTGAGCAGC |
| 1235 | VH Ab75 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATCCGTATGGCCCGGATGGCTTTGATATTTGGGGCCAGGGCACCatgGTGACCGTGAGCAGC |
| 1236 | VH Ab76 | GAAGTGCAGCTGCTGGAAAGCGGCGGCGGCGTGGTGCAGCCGGGCCGTAGCCTGCGTCTGAG |
| | | CTGCGCGGCGAGCGGCTTTGATTTTGCGGGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCC |
| | | TGACCTATGGCCCGGATGGCTTTGATATTTGGGGCCAGGGCACCatgGTGACCGTGAGCAGC |
| 1237 | VH Ab77 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCC |
| | | TGACCTATGGCCCGGATGGCTTTGATATTTGGGGCCAGGGCACCatgGTGACCGTGAGCAGC |
| 1238 | VH Ab78 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | ATCCGTATGGCCCGGATGGCTTTGATATTTGGGGCCAGGGCACCatgGTGACCGTGAGCAGC |
| 1239 | VH Ab79 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATCCGCATGGCCCGGATGGCTTTGATATTTGGGGCCAGGGCACCatgGTGACCGTGAGCAGC |
| 1240 | VH Ab80 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCATTGGCT |
| | | ATCCGCATGGCCCGGATGGCTTTGATATTTGGGGCCAGGGCACCatgGTGACCGTGAGCAGC |
| 1241 | VH Ab81 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATCCGTATGGCCCGGATGGCTTTGATATTTGGGGCCAGGGCACCatgGTGACCGTGAGCAGC |
| 1242 | VH Ab82 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | TTCCGTTTGGCCCGGATGGCTTTGATATTTGGGGCCAGGGCACCatgGTGACCGTGAGCAGC |
| 1243 | VH Ab83 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTCCGTATGGCCCGGATGGCTTTGATATTTGGGGCCAGGGCACCatgGTGACCGTGAGCAGC |
| 1244 | VH Ab84 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | ATCCGAACGGCCCGGATGGCTTTGATATTTGGGGCCAGGGCACCatgGTGACCGTGAGCAGC |
| 1245 | VH Ab85 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCAGGGCT |
| | | TTCCGTATGGCCCGGATGGCTTTGATATTTGGGGCCAGGGCACCatgGTGACCGTGAGCAGC |
| 1246 | VH Ab86 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCC |
| | | TGACCTATGGCCCGGATGGCTTTGATATTTGGGGCCAGGGCACCatgGTGACCGTGAGCAGC |
| 1247 | VH Ab87 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCTGGGGCCCGGATGGCTTTGATATTTGGGGCCAGGGCACCatgGTGACCGTGAGCAGC |
| 1248 | VH Ab88 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCC |
| | | TGACCTATGGCCCGGATGGCTTTGATATTTGGGGCCAGGGCACCatgGTGACCGTGAGCAGC |
| 1249 | VH Ab89 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTCCGTATGGCCCGGATGGCTTTGATATTTGGGGCCAGGGCACCatgGTGACCGTGAGCAGC |
| 1250 | VH Ab90 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | ATCCGTATGGCCCGGATGGCTTTGATATTTGGGGCCAGGGCACCatgGTGACCGTGAGCAGC |
| 1251 | VH Ab91 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCAGGGCT |
| | | ATatgCATGGCCCGGATGGCTTTGATATTTGGGGCCAGGGCACCatgGTGACCGTGAGCAGC |
| 1252 | VH Ab92 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCATGGCT |
| | | TTCCGACCGGCCCGGATGGCTTTGATATTTGGGGCCAGGGCACCatgGTGACCGTGAGCAGC |
| 1253 | VH Ab93 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | TTCCGTATGGCCCGGATGGCTTTGATATTTGGGGCCAGGGCACCatgGTGACCGTGAGCAGC |
| 1254 | VH Ab94 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | GGCCGTATGGCCCGGATGGCTTTGATATTTGGGGCCAGGGCACCatgGTGACCGTGAGCAGC |
| 1255 | VH Ab95 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | GGCCGTATGGCCCGGATGGCTTTGATATTTGGGGCCAGGGCACCatgGTGACCGTGAGCAGC |
| 1256 | VH Ab96 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | GGCCGTATGGCCCGGATGGCTTTGATATTTGGGGCCAGGGCACCatgGTGACCGTGAGCAGC |
| 1257 | VH Ab97 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | GGCCGTATGGCCCGGATGGCTTTGATATTTGGGGCCAGGGCACCatgGTGACCGTGAGCAGC |
| 1258 | VH Ab98 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | GGCCGTATGGCCCGGATGGCTTTGATATTTGGGGCCAGGGCACCatgGTGACCGTGAGCAGC |
| 1259 | VH Ab99 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | GGCCGTATGGCCCGGATGGCTTTGATATTTGGGGCCAGGGCACCatgGTGACCGTGAGCAGC |
| 1260 | VH Ab100 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCTGGGCT |
| | | GGCCGTATGGCCCGGATGGCTTTGATATTTGGGGCCAGGGCACCatgGTGACCGTGAGCAGC |
| 1261 | VH Ab101 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCCTGGGCCCGGATGGCTTTGATATTTGGGGCCAGGGCACCatgGTGACCGTGAGCAGC |
| 1262 | VH Ab102 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCCTGGGCCCGGATGGCTTTGATATTTGGGGCCAGGGCACCatgGTGACCGTGAGCAGC |
| 1263 | VH Ab103 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCCTGGGCCCGGATGGCTTTGATATTTGGGGCCAGGGCACCatgGTGACCGTGAGCAGC |
| 1264 | VH Ab104 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCCTGGGCCCGGATGGCTTTGATATTTGGGGCCAGGGCACCatgGTGACCGTGAGCAGC |
| 1265 | VH Ab105 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCCTGGGCCCGGATGGCTTTGATATTTGGGGCCAGGGCACCatgGTGACCGTGAGCAGC |
| 1266 | VH Ab106 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCCTGGGCCCGGATGGCTTTGATATTTGGGGCCAGGGCACCatgGTGACCGTGAGCAGC |
| 1267 | VH Ab107 | GAAGTGCAGCTGCTGGAAAGCGGCGGCGGCGTGGTGCAGCCGGGCCGTAGCCTGCGTCTGAG |
| | | CTGCGCGGCGAGCGGCTTTACCTTTAGCAGCTATGCGatgCATTGGGTGCGTCAGGCGCCGG |
| | | GCAAAGGCCTGGAATGGGTGGCGGTGATTAGCTATGATGGCAGCAACAAATATTATGCGGAT |
| | | AGCGTGAAAGGCCGTTTTACCATTAGCCGTGATAACAGCAAAAACACCCTGAGCCTGCAGat |
| | | gAACAGCCTGCGTGTGGAAGATACCGCGATTTATTATTGCGTGCGTGGCGCGGGCCGTGGCT |
| | | TTACCCTGGGCCCGGATGGCTTTGATATTTGGGGCCAGGGCACCatgGTGACCGTGAGCAGC |
| 1268 | VL Ab16 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1269 | VL Ab17 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1270 | VL Ab18 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1271 | VL Ab19 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1272 | VL Ab20 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1273 | VL Ab21 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1274 | VL Ab22 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1275 | VL Ab23 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1276 | VL Ab24 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1277 | VL Ab25 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1278 | VL Ab26 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1279 | VL Ab27 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1280 | VL Ab28 | GATATTGTGatgACCCAGACCCCGCTGAGCCTGAGCGTGACCCCGGGCCAGCCGGCGAGCAT |
| | | TAGCTGCAAAAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1281 | VL Ab29 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATGTGGAAGCGCCGCTGACCTTTG |
| | | GCGGCGGCACCAAAGTGGAAATTAAA |
| 1282 | VL Ab30 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATGTGGAAGCGCCGCTGACCTTTG |
| | | GCGGCGGCACCAAAGTGGAAATTAAA |
| 1283 | VL Ab31 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATGTGGAAGCGCCGCTGACCTTTG |
| | | GCGGCGGCACCAAAGTGGAAATTAAA |
| 1284 | VL Ab32 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATGTGGAAGCGCCGCTGACCTTTG |
| | | GCGGCGGCACCAAAGTGGAAATTAAA |
| 1285 | VL Ab33 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATGTGGAAGCGCCGCTGACCTTTG |
| | | GCGGCGGCACCAAAGTGGAAATTAAA |
| 1286 | VL Ab34 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATGTGGAAGCGCCGCTGACCTTTG |
| | | GCGGCGGCACCAAAGTGGAAATTAAA |
| 1287 | VL Ab35 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATGTGGAAGCGCCGCTGACCTTTG |
| | | GCGGCGGCACCAAAGTGGAAATTAAA |
| 1288 | VL Ab36 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1289 | VL Ab37 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1290 | VL Ab38 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1291 | VL Ab39 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1292 | VL Ab40 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1293 | VL Ab41 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1294 | VL Ab42 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1295 | VL Ab43 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1296 | VL Ab44 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1297 | VL Ab45 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1298 | VL Ab46 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1299 | VL Ab47 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1300 | VL Ab48 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1301 | VL Ab49 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1302 | VL Ab50 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1303 | VL Ab51 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1304 | VL Ab52 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1305 | VL Ab53 | GATATTGTGatgACCCAGACCCCGCTGAGCCTGAGCGTGACCCCGGGCCAGCCGGCGAGCAT |
| | | TAGCTGCCGTAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1306 | VL Ab54 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1307 | VL Ab55 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1308 | VL Ab56 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1309 | VL Ab57 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1310 | VL Ab58 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1311 | VL Ab59 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1312 | VL Ab60 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1313 | VL Ab61 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1314 | VL Ab62 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1315 | VL Ab63 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1316 | VL Ab64 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1317 | VL Ab65 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1318 | VL Ab66 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1319 | VL Ab67 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1320 | VL Ab68 | GATATTGTGatgACCCAGACCCCGCTGAGCCTGAGCGTGACCCCGGGCCAGCCGGCGAGCAT |
| | | TAGCTGCACCAGCAGCCGTAGCCTGCTGCATAGCGATGGCAAAACCTATGTGTATTGGTATG |
| | | TGCAGAAAAGCGGCCAGCCGCCGCAGCTGCTGATTTATGAACTGAGCAACCGTTTTAGCGGC |
| | | GTGCCGGATCGTTTTAGCGGCAGCGGCAGCCGTACCGATTTTACCCTGAAAATTAGCCGTGT |
| | | GGAAGCGGAAGATGTGGGCGTGTATTATTGCatgCAGTATATTGAAGCGCCGCTGACCTTTG |
| | | GCGGCGGCACCAAAGTGGAAATTAAA |
| 1321 | VL Ab69 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1322 | VL Ab70 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1323 | VL Ab71 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1324 | VL Ab72 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1325 | VL Ab73 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1326 | VL Ab74 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1327 | VL Ab75 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1328 | VL Ab76 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1329 | VL Ab77 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1330 | VL Ab78 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1331 | VL Ab79 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1332 | VL Ab80 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1333 | VL Ab81 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1334 | VL Ab82 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1335 | VL Ab83 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1336 | VL Ab84 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1337 | VL Ab85 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1338 | VL Ab86 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1339 | VL Ab87 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAA |
| 1340 | VL Ab88 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATCTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1341 | VL Ab89 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGTGCCGCTGACCTTTGGCGGC |
| | | GGCACCAAAGTGGAAATTAAA |
| 1342 | VL Ab90 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGTGCCGCTGACCTTTGGCGGC |
| | | GGCACCAAAGTGGAAATTAAA |
| 1343 | VL Ab91 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1344 | VL Ab92 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAA |
| 1345 | VL Ab93 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATCTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1346 | VL Ab94 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1347 | VL Ab95 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1348 | VL Ab96 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1349 | VL Ab97 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAA |
| 1350 | VL Ab98 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGTGCCGCTGACCTTTGGCGGC |
| | | GGCACCAAAGTGGAAATTAAA |
| 1351 | VL Ab99 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 13$$ | VL Ab100 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAA |
| 1353 | VL Ab101 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1354 | VL Ab102 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1355 | VL Ab103 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1356 | VL Ab104 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAA |
| 1357 | VL Ab105 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGTGCCGCTGACCTTTGGCGGC |
| | | GGCACCAAAGTGGAAATTAAA |
| 1358 | VL Ab106 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAA |
| 1359 | VL Ab107 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAA |
| 1360 | HC Ab16 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATACC |
| | | CGTGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1361 | HC Ab17 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATACC |
| | | AACGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1362 | HC Ab18 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | TGGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1363 | HC Ab19 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | CTGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1364 | HC Ab20 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCCTGACC |
| | | TTTGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1365 | HC Ab21 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1366 | HC Ab22 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | AAAGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1367 | HC Ab23 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1368 | HC Ab24 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCCTGATT |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1369 | HC Ab25 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTTTACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1370 | HC Ab26 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCAGGGCCTGAGC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1371 | HC Ab27 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATACC |
| | | CTGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1372 | HC Ab28 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1373 | HC Ab29 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1374 | HC Ab30 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGNNNAAANNNNNNATTTATTATTGCGTGCGTGGCGCGGGCCGTGGCTTTACC |
| | | TGGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1375 | HC Ab31 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1376 | HC Ab32 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTTTACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1377 | HC Ab33 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCAGGGCCTGAGC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1378 | HC Ab34 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATACC |
| | | CGTGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1379 | HC Ab35 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATACC |
| | | AACGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1380 | HC Ab36 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1381 | HC Ab37 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1382 | HC Ab38 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCAGGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1383 | HC Ab39 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCACCGGCTTTACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1384 | HC Ab40 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCatgGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1385 | HC Ab41 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1386 | HC Ab42 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGGCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1387 | HC Ab43 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1388 | HC Ab44 | GAAGTGCAGCTGCTGGAAAGCGGCGGCGGCGTGGTGCAGCCGGGCCGTAGCCTGCGTCTGAGC |
| | | TGCGCGGCGAGCGGCTTTGATTTTGCGGGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1389 | HC Ab45 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1390 | HC Ab46 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1391 | HC Ab47 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATCCG |
| | | CATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1392 | HC Ab48 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCATTGGCTATCCG |
| | | CATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1393 | HC Ab49 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1394 | HC Ab50 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTTTCCG |
| | | TTTGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1395 | HC Ab51 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1396 | HC Ab52 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTATCCG |
| | | AACGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1397 | HC Ab53 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCAGGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1398 | HC Ab54 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCAGGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1399 | HC Ab55 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCACCGGCTTTACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1400 | HC Ab56 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCatgGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1401 | HC Ab57 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1402 | HC Ab58 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGGCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1403 | HC Ab59 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1404 | HC Ab60 | GAAGTGCAGCTGCTGGAAAGCGGCGGCGGCGTGGTGCAGCCGGGCCGTAGCCTGCGTCTGAGC |
| | | TGCGCGGCGAGCGGCTTTGATTTTGCGGGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1405 | HC Ab61 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1406 | HC Ab62 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1407 | HC Ab63 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATCCG |
| | | CATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1408 | HC Ab64 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCATTGGCTATCCG |
| | | CATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1409 | HC Ab65 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1410 | HC Ab66 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTTTCCG |
| | | TTTGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1411 | HC Ab67 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1412 | HC Ab68 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTATCCG |
| | | AACGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1413 | HC Ab69 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCAGGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1414 | HC Ab70 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCAGGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1415 | HC Ab71 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCACCGGCTTTACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1416 | HC Ab72 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCatgGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1417 | HC Ab73 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1418 | HC Ab74 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGGCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1419 | HC Ab75 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1420 | HC Ab76 | GAAGTGCAGCTGCTGGAAAGCGGCGGCGGCGTGGTGCAGCCGGGCCGTAGCCTGCGTCTGAGC |
| | | TGCGCGGCGAGCGGCTTTGATTTTGCGGGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1421 | HC Ab77 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1422 | HC Ab78 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1423 | HC Ab79 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATCCG |
| | | CATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1424 | HC Ab80 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCATTGGCTATCCG |
| | | CATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1425 | HC Ab81 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1426 | HC Ab82 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTTTCCG |
| | | TTTGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1427 | HC Ab83 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1428 | HC Ab84 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTATCCG |
| | | AACGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1429 | HC Ab85 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCAGGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1430 | HC Ab86 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1431 | HC Ab87 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | TGGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1432 | HC Ab88 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCCTGACC |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1433 | HC Ab89 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1434 | HC Ab90 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTATCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1435 | HC Ab91 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCAGGGCTATatg |
| | | CATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1436 | HC Ab92 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCATGGCTTTCCG |
| | | ACCGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1437 | HC Ab93 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTTTCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1438 | HC Ab94 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1439 | HC Ab95 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1440 | HC Ab96 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1441 | HC Ab97 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1442 | HC Ab98 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1443 | HC Ab99 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1444 | HC Ab100 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCTGGGCTGGCCG |
| | | TATGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1445 | HC Ab101 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | CTGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1446 | HC Ab102 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | CTGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1447 | HC Ab103 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | CTGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1448 | HC Ab104 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | CTGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1449 | HC Ab105 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | CTGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1450 | HC Ab106 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | CTGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1451 | HC Ab107 | GAAGTGCAGCTGCTGGAAAGCGGCGGCGGCGTGGTGCAGCCGGGCCGTAGCCTGCGTCTGAGC |
| | | TGCGCGGCGAGCGGCTTTACCTTTAGCAGCTATGCGatgCATTGGGTGCGTCAGGCGCCGGGC |
| | | AAAGGCCTGGAATGGGTGGCGGTGATTAGCTATGATGGCAGCAACAAATATTATGCGGATAGC |
| | | GTGAAAGGCCGTTTTACCATTAGCCGTGATAACAGCAAAAACACCCTGAGCCTGCAGatgAAC |
| | | AGCCTGCGTGTGGAAGATACCGCGATTTATTATTGCGTGCGTGGCGCGGGCCGTGGCTTTACC |
| | | CTGGGCCCGGATGGCTTTGATATTTGGGGCCAGGGCACCatgGTGACCGTGAGCAGCAAGGGA |
| | | CCCTCGGTGTTCCCTCTCGCCCCCTCATCGAGGAGCACGTCCGAATCGACTGCGGCGCTGGGA |
| | | TGTCTCGTGAAGGACTACTTCCCCGAGCCCGTGACCGTGTCATGGAACTCAGGCGCATTGACC |
| | | TCGGGAGTGCACACGTTCCCGGCAGTGTTGCAGTCGTCGGGCCTTTACTCGCTGTCGTCCGTG |
| | | GTCACGGTGCCTTCGTCGAATTTCGGGACGCAGACTTACACATGTAATGTGGATCACAAACCG |
| | | TCCAATACCAAGGTCGATAAGACGGTCGAAAGGAAATGCTGCGTGGAATGTCCTCCCTGCCCA |
| | | GCACCGCCAGTCGCGGGTCCCAGCGTCTTTTTGTTCCCACCGAAGCCCAAGGACACACTGATG |
| | | ATCAGCCGTACGCCGGAGGTAACATGCGTAGTGGTAGATGTCTCGCATGAGGACCCTGAAGTG |
| | | CAGTTCAATTGGTATGTAGACGGCGTGGAGGTGCACAACGCCAAGACCAAGCCACGAGAAGAA |
| | | CAGTTCAACTCGACTTTTAGAGTGGTATCCGTCCTGACCGTCGTGCACCAGGACTGGCTCAAT |
| | | GGAAAGGAGTACAAATGCAAGGTATCCAACAAGGGGTTGCCAGCTCCAATCGAAAAGACCATC |
| | | TCAAAGACAAAGGGGCAGCCCAGAGAGCCCCAAGTGTATACGCTTCCACCCTCAAGGGAGGAG |
| | | ATGACAAAGAATCAGGTATCACTCACATGTTTGGTGAAAGGGTTTTATCCGAGCGATATTGCG |
| | | GTCGAGTGGGAAAGCAACGGTCAACCGGAGAACAACTATAAGACGACTCCCCCTATGCTGGAC |
| | | AGCGACGGGTCGTTCTTTCTGTATTCCAAACTCACGGTGGACAAATCGAGGTGGCAGCAAGGA |
| | | AACGTATTCTCATGCTCAGTAATGCACGAGGCCCTCCATAATCATTACACGCAGAAATCATTA |
| | | AGCTTATCGCCGGGT |
| 1452 | LC Ab16 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1453 | LC Ab17 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1454 | LC Ab18 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1455 | LC Ab19 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1456 | LC Ab20 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1457 | LC Ab21 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1458 | LC Ab22 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1459 | LC Ab23 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1460 | LC Ab24 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1461 | LC Ab25 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1462 | LC Ab26 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1463 | LC Ab27 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1464 | LC Ab28 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1465 | LC Ab29 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1466 | LC Ab30 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1467 | LC Ab31 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1468 | LC Ab32 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1469 | LC Ab33 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1470 | LC Ab34 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1471 | LC Ab35 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1472 | LC Ab36 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1473 | LC Ab37 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1474 | LC Ab38 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1475 | LC Ab39 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1476 | LC Ab40 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1477 | LC Ab41 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1478 | LC Ab42 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1479 | LC Ab43 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1480 | LC Ab44 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1481 | LC Ab45 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1482 | LC Ab46 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1483 | LC Ab47 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1484 | LC Ab48 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1485 | LC Ab49 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1486 | LC Ab50 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1487 | LC Ab51 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1488 | LC Ab52 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1489 | LC Ab53 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1490 | LC Ab54 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1491 | LC Ab55 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1492 | LC Ab56 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1493 | LC Ab57 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1494 | LC Ab58 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1495 | LC Ab59 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1496 | LC Ab60 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1497 | LC Ab61 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1498 | LC Ab62 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1499 | LC Ab63 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1500 | LC Ab64 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1501 | LC Ab65 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1502 | LC Ab66 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1503 | LC Ab67 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1504 | LC Ab68 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1505 | LC Ab69 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1506 | LC Ab70 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1507 | LC Ab71 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1508 | LC Ab72 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1509 | LC Ab73 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1510 | LC Ab74 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1511 | LC Ab75 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1512 | LC Ab76 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1513 | LC Ab77 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1514 | LC Ab78 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1515 | LC Ab79 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1516 | LC Ab80 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1517 | LC Ab81 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1518 | LC Ab82 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1519 | LC Ab83 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1520 | LC Ab84 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1521 | LC Ab85 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1522 | LC Ab86 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1523 | LC Ab87 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1524 | LC Ab88 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATCTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1525 | LC Ab89 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGTGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1526 | LC Ab90 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGTGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1527 | LC Ab91 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1528 | LC Ab92 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1529 | LC Ab93 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATCTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1530 | LC Ab94 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1531 | LC Ab95 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1532 | LC Ab96 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1533 | LC Ab97 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1534 | LC Ab98 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGTGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1535 | LC Ab99 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1536 | LC Ab100 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1537 | LC Ab101 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1538 | LC Ab102 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1539 | LC Ab103 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1540 | LC Ab104 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1541 | LC Ab105 | GATATTGTGatgACCCAGACCCCGCTGAGCCTGAGCGTGACCCCGGGCCAGCCGGCGAGCATT |
| | | AGCTGCACCAGCAGCCGTAGCCTGCTGCATAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGTGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1542 | LC Ab106 | GATATTGTGatgACCCAGACCCCGCTGAGCCTGAGCGTGACCCCGGGCCAGCCGGCGAGCATT |
| | | AGCTGCCGTAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATATTGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1543 | LC Ab107 | GATATTGTGatgACCCAGACCCCGCTGAGCCTGAGCGTGACCCCGGGCCAGCCGGCGAGCATT |
| | | AGCTGCAAAAGCAGCCGTAGCCTGCTGTGGAGCGATGGCAAAACCTATGTGTATTGGTATGTG |
| | | CAGAAAAGCGGCCAGCCGCCGCAGCTGCTGATTTATGAACTGAGCAACCGTTTTAGCGGCGTG |
| | | CCGGATCGTTTTAGCGGCAGCGGCAGCCGTACCGATTTTACCCTGAAAATTAGCCGTGTGGAA |
| | | GCGGAAGATGTGGGCGTGTATTATTGCatgCAGTATGTGGAAGCGCCGCTGACCTTTGGCGGC |
| | | GGCACCAAAGTGGAAATTAAACGTACGGTGGCGGCGCCCAGTGTATTCATCTTCCCTCCCTCC |
| | | GACGAGCAGTTGAAGTCGGGGACCGCGTCAGTCGTGTGCCTGCTCAATAACTTTTACCCGCGA |
| | | GAGGCTAAGGTCCAGTGGAAAGTGGATAATGCGCTGCAAAGCGGAAACTCCCAAGAATCAGTG |
| | | ACGGAACAAGACTCAAAAGACTCGACGTATTCGCTCTCATCGACGCTCACGCTTTCAAAAGCA |
| | | GATTACGAGAAGCACAAGGTGTATGCATGTGAAGTGACACACCAGGGTTTGTCGTCGCCAGTC |
| | | ACCAAGTCATTCAACCGCGGAGAGTGC |
| 1544 | VHCDR1 (IMGT) | GFTFSSYA |
| | Ab1 | |
| 1545 | VHCDR2 (IMGT) | ISYDGSNK |
| | Ab1 | |
| 1546 | VHCDR3 (IMGT) | VRGAGRGYTYGPDGFDI |
| | Ab1 | |
| 1547 | VLCDR1 (IMGT) | QSLVYTDGITY |
| | Ab1 | |
| 1548 | VLCDR2 (IMGT) | EIS |
| | Ab1 | |
| 1549 | VLCDR3 (IMGT) | MQATQFPWT |
| | Ab1 | |
| 1550 | VHCDR1 (IMGT) | GFTFSSYA |
| | Ab2 | |
| 1551 | VHCDR2 (IMGT) | ISYDGSNK |
| | Ab2 | |
| 1552 | VHCDR3 (IMGT) | VRGAGRGYTYGPDGFDI |
| | Ab2 | |
| 1553 | VLCDR1 (IMGT) | QSITNY |
| | Ab2 | |
| 1554 | VLCDR2 (IMGT) | AAS |
| | Ab2 | |
| 1555 | VLCDR3 (IMGT) | QQSYTTPFT |
| | Ab2 | |
| 1556 | VHCDR1 (IMGT) | GFTFSQYS |
| | Ab3 | |
| 1557 | VHCDR2 (IMGT) | ISFDGADK |
| | Ab3 | |
| 1558 | VHCDR3 (IMGT) | VRGAGRGYTYGPDGFDI |
| | Ab3 | |
| 1559 | VLCDR1 (IMGT) | QTISRY |
| | Ab3 | |
| 1560 | VLCDR2 (IMGT) | TAT |
| | Ab3 | |
| 1561 | VLCDR3 (IMGT) | QQTYSAPLT |
| | Ab3 | |
| 1562 | VHCDR1 (IMGT) | GFTFSSYA |
| | Ab4 | |
| 1563 | VHCDR2 (IMGT) | ISYDGSNK |
| | Ab4 | |
| 1564 | VHCDR3 (IMGT) | VRGAGRGYTYGPDGFDI |
| | Ab4 | |
| 1565 | VLCDR1 (IMGT) | QSLIFGDGKTY |
| | Ab4 | |
| 1566 | VLCDR2 (IMGT) | QVS |
| | Ab4 | |
| 1567 | VLCDR3 (IMGT) | MQAKQFPWT |
| | Ab4 | |
| 1568 | VHCDR1 (IMGT) | GFTFGHYA |
| | Ab5 | |
| 1569 | VHCDR2 (IMGT) | ISYDGSNK |
| | Ab5 | |
| 1570 | VHCDR3 (IMGT) | VRGAGRGYTYGPDGFDI |
| | Ab5 | |
| 1571 | VLCDR1 (IMGT) | QSLLYSDGKTY |
| | Ab5 | |
| 1572 | VLCDR2 (IMGT) | EVS |
| | Ab5 | |
| 1573 | VLCDR3 (IMGT) | MQATRFPWT |
| | Ab5 | |
| 1574 | VHCDR1 (IMGT) | GFTFSSYA |
| | Ab6 | |
| 1575 | VHCDR2 (IMGT) | ISYDGSNK |
| | Ab6 | |
| 1576 | VHCDR3 (IMGT) | VRGAGRGYTYGPDGFDI |
| | Ab6 | |
| 1577 | VLCDR1 (IMGT) | QSLVYTDGITY |
| | Ab6 | |
| 1578 | VLCDR2 (IMGT) | EIS |
| | Ab6 | |
| 1579 | VLCDR3 (IMGT) | MQATQFPWT |
| | Ab6 | |
| 1580 | VHCDR1 (IMGT) | GFTFSGYA |
| | Ab7 | |
| 1581 | VHCDR2 (IMGT) | ISYDGSNK |
| | Ab7 | |
| 1582 | VHCDR3 (IMGT) | VRGAGRGYTYGPDGFDI |
| | Ab7 | |
| 1583 | VLCDR1 (IMGT) | ESLVYRDGNTY |
| | Ab7 | |
| 1584 | VLCDR2 (IMGT) | KVS |
| | Ab7 | |
| 1585 | VLCDR3 (IMGT) | MQATQFPWT |
| | Ab7 | |
| 1586 | VHCDR1 (IMGT) | GFTFSSYA |
| | Ab8 | |
| 1587 | VHCDR2 (IMGT) | ISYDGSNK |
| | Ab8 | |
| 1588 | VHCDR3 (IMGT) | VRGAGRGYTYGPDGFDI |
| | Ab8 | |
| 1589 | VLCDR1 (IMGT) | QSLVYSDGNTY |
| | Ab8 | |
| 1590 | VLCDR2 (IMGT) | KVS |
| | Ab8 | |
| 1591 | VLCDR3 (IMGT) | MQATRFPWT |
| | Ab8 | |
| 1592 | VHCDR1 (IMGT) | GFSFTTHA |
| | Ab9 | |
| 1593 | VHCDR2 (IMGT) | ISYDGSEK |
| | Ab9 | |
| 1594 | VHCDR3 (IMGT) | VRGAGRGYTYGPDGFDI |
| | Ab9 | |
| 1595 | VLCDR1 (IMGT) | QSLLHSDGKTY |
| | Ab9 | |
| 1596 | VLCDR2 (IMGT) | EVS |
| | Ab9 | |
| 1597 | VLCDR3 (IMGT) | MQYINLPLT |
| | Ab9 | |
| 1598 | VHCDR1 (IMGT) | GFTFGHYA |
| | Ab10 | |
| 1599 | VHCDR2 (IMGT) | ISYDGSNK |
| | Ab10 | |
| 1600 | VHCDR3 (IMGT) | VRGAGRGYTYGPDGFDI |
| | Ab10 | |
| 1601 | VLCDR1 (IMGT) | QSLVYSDGNTY |
| | Ab10 | |
| 1602 | VLCDR2 (IMGT) | KVS |
| | Ab10 | |
| 1603 | VLCDR3 (IMGT) | ?? |
| | Ab10 | |
| 1604 | VHCDR1 (IMGT) | GFTFSSYA |
| | Ab11 | |
| 1605 | VHCDR2 (IMGT) | ISYDGSNK |
| | Ab11 | |
| 1606 | VHCDR3 (IMGT) | VRGAGRGYTYGPDGFDI |
| | Ab11 | |
| 1607 | VLCDR1 (IMGT) | QSLVYTDGITY |
| | Ab11 | |
| 1608 | VLCDR2 (IMGT) | EIS |
| | Ab11 | |
| 1609 | VLCDR3 (IMGT) | MQATQFPWT |
| | Ab11 | |
| 1610 | VHCDR1 (IMGT) | GYRFTSHD |
| | Ab12 | |
| 1611 | VHCDR2 (IMGT) | INPNNDIT |
| | Ab12 | |
| 1612 | VHCDR3 (IMGT) | ARGAGMLFHAVGQFDS |
| | Ab12 | |
| 1613 | VLCDR1 (IMGT) | QDIRKN |
| | Ab12 | |
| 1614 | VLCDR2 (IMGT) | DAS |
| | Ab12 | |
| 1615 | VLCDR3 (IMGT) | LQYGDLPLT |
| | Ab12 | |
| 1616 | VHCDR1 (IMGT) | GYRFTSHD |
| | Ab13 | |
| 1617 | VHCDR2 (IMGT) | INPNNDIT |
| | Ab13 | |
| 1618 | VHCDR3 (IMGT) | ARGAGMLFHAVGQFDS |
| | Ab13 | |
| 1619 | VLCDR1 (IMGT) | QSIGRF |
| | Ab13 | |
| 1620 | VLCDR2 (IMGT) | TAS |
| | Ab13 | |
| 1621 | VLCDR3 (IMGT) | QQFKNYPT |
| | Ab13 | |
| 1622 | VHCDR1 (IMGT) | GFTVSSNY |
| | Ab14 | |
| 1623 | VHCDR2 (IMGT) | IFSDGTT |
| | Ab14 | |
| 1624 | VHCDR3 (IMGT) | VAGPANGAYDI |
| | Ab14 | |
| 1625 | VLCDR1 (IMGT) | QSISNW |
| | Ab14 | |
| 1626 | VLCDR2 (IMGT) | RAS |
| | Ab14 | |
| 1627 | VLCDR3 (IMGT) | QQYNVYSGT |
| | Ab14 | |
| 1628 | VHCDR1 (IMGT) | GFTFSSYA |
| | Ab15 | |
| 1629 | VHCDR2 (IMGT) | ISYDGSNK |
| | Ab15 | |
| 1630 | VHCDR3 (IMGT) | VRGAGRGYTYGPDGFDI |
| | Ab15 | |
| 1631 | VLCDR1 (IMGT) | RSLLHSDGKTY |
| | Ab15 | |
| 1632 | VLCDR2 (IMGT) | ELS |
| | Ab15 | |
| 1633 | VLCDR3 (IMGT) | MQYIEAPLT |
| | Ab15 | |
| 1634 | VHCDR1 (IMGT) | GFTFSSYA |
| | Ab16 | |
| 1635 | VHCDR2 (IMGT) | ISYDGSNK |
| | Ab16 | |
| 1636 | VHCDR3 (IMGT) | VRGAGRGYTRGPDGFDI |
| | Ab16 | |
| 1637 | VLCDR1 (IMGT) | RSLLHSDGKTY |
| | Ab16 | |
| 1638 | VLCDR2 (IMGT) | ELS |
| | Ab16 | |
| 1639 | VLCDR3 (IMGT) | MQYIEAPLT |
| | Ab16 | |
| 1640 | VHCDR1 (IMGT) | GFTFSSYA |
| | Ab17 | |
| 1641 | VHCDR2 (IMGT) | ISYDGSNK |
| | Ab17 | |
| 1642 | VHCDR3 (IMGT) | VRGAGRGYTNGPDGFDI |
| | Ab17 | |
| 1643 | VLCDR1 (IMGT) | RSLLHSDGKTY |
| | Ab17 | |
| 1644 | VLCDR2 (IMGT) | ELS |
| | Ab17 | |
| 1645 | VLCDR3 (IMGT) | MQYIEAPLT |
| | Ab17 | |
| 1646 | VHCDR1 (IMGT) | GFTFSSYA |
| | Ab18 | |
| 1647 | VHCDR2 (IMGT) | ISYDGSNK |
| | Ab18 | |
| 1648 | VHCDR3 (IMGT) | VRGAGRGFTWGPDGFDI |
| | Ab18 | |
| 1649 | VLCDR1 (IMGT) | RSLLHSDGKTY |
| | Ab18 | |
| 1650 | VLCDR2 (IMGT) | ELS |
| | Ab18 | |
| 1651 | VLCDR3 (IMGT) | MQYIEAPLT |
| | Ab18 | |
| 1652 | VHCDR1 (IMGT) | GFTFSSYA |
| | Ab19 | |
| 1653 | VHCDR2 (IMGT) | ISYDGSNK |
| | Ab19 | |
| 1654 | VHCDR3 (IMGT) | VRGAGRGFTLGPDGFDI |
| | Ab19 | |
| 1655 | VLCDR1 (IMGT) | RSLLHSDGKTY |
| | Ab19 | |
| 1656 | VLCDR2 (IMGT) | ELS |
| | Ab19 | |
| 1657 | VLCDR3 (IMGT) | MQYIEAPLT |
| | Ab19 | |
| 1658 | VHCDR1 (IMGT) | GFTFSSYA |
| | Ab20 | |
| 1659 | VHCDR2 (IMGT) | ISYDGSNK |
| | Ab20 | |
| 1660 | VHCDR3 (IMGT) | VRGAGRGLTFGPDGFDI |
| | Ab20 | |
| 1661 | VLCDR1 (IMGT) | RSLLHSDGKTY |
| | Ab20 | |
| 1662 | VLCDR2 (IMGT) | ELS |
| | Ab20 | |
| 1663 | VLCDR3 (IMGT) | MQYIEAPLT |
| | Ab20 | |
| 1664 | VHCDR1 (IMGT) | GFTFSSYA |
| | Ab21 | |
| 1665 | VHCDR2 (IMGT) | ISYDGSNK |
| | Ab21 | |
| 1666 | VHCDR3 (IMGT) | VRGAGLGWPYGPDGFDI |
| | Ab21 | |
| 1667 | VLCDR1 (IMGT) | RSLLHSDGKTY |
| | Ab21 | |
| 1668 | VLCDR2 (IMGT) | ELS |
| | Ab21 | |
| 1669 | VLCDR3 (IMGT) | MQYIEAPLT |
| | Ab21 | |
| 1670 | VHCDR1 (IMGT) | GFTFSSYA |
| | Ab22 | |
| 1671 | VHCDR2 (IMGT) | ISYDGSNK |
| | Ab22 | |
| 1672 | VHCDR3 (IMGT) | VRGAGRGFTKGPDGFDI |
| | Ab22 | |
| 1673 | VLCDR1 (IMGT) | RSLLHSDGKTY |
| | Ab22 | |
| 1674 | VLCDR2 (IMGT) | ELS |
| | Ab22 | |
| 1675 | VLCDR3 (IMGT) | MQYIEAPLT |
| | Ab22 | |
| 1676 | VHCDR1 (IMGT) | GFTFSSYA |
| | Ab23 | |
| 1677 | VHCDR2 (IMGT) | ISYDGSNK |
| | Ab23 | |
| 1678 | VHCDR3 (IMGT) | VRGAGRGLTYGPDGFDI |
| | Ab23 | |
| 1679 | VLCDR1 (IMGT) | RSLLHSDGKTY |
| | Ab23 | |
| 1680 | VLCDR2 (IMGT) | ELS |
| | Ab23 | |
| 1681 | VLCDR3 (IMGT) | MQYIEAPLT |
| | Ab23 | |
| 1682 | VHCDR1 (IMGT) | GFTFSSYA |
| | Ab24 | |
| 1683 | VHCDR2 (IMGT) | ISYDGSNK |
| | Ab24 | |
| 1684 | VHCDR3 (IMGT) | VRGAGRGLIYGPDGFDI |
| | Ab24 | |
| 1685 | VLCDR1 (IMGT) | RSLLHSDGKTY |
| | Ab24 | |
| 1686 | VLCDR2 (IMGT) | ELS |
| | Ab24 | |
| 1687 | VLCDR3 (IMGT) | MQYIEAPLT |
| | Ab24 | |
| 1688 | VHCDR1 (IMGT) | GFTFSSYA |
| | Ab25 | |
| 1689 | VHCDR2 (IMGT) | ISYDGSNK |
| | Ab25 | |
| 1690 | VHCDR3 (IMGT) | VRGAGLGFTYGPDGFDI |
| | Ab25 | |
| 1691 | VLCDR1 (IMGT) | RSLLHSDGKTY |
| | Ab25 | |
| 1692 | VLCDR2 (IMGT) | ELS |
| | Ab25 | |
| 1693 | VLCDR3 (IMGT) | MQYIEAPLT |
| | Ab25 | |
| 1694 | VHCDR1 (IMGT) | GFTFSSYA |
| | Ab26 | |
| 1695 | VHCDR2 (IMGT) | ISYDGSNK |
| | Ab26 | |
| 1696 | VHCDR3 (IMGT) | VRGAGQGLSYGPDGFDI |
| | Ab26 | |
| 1697 | VLCDR1 (IMGT) | RSLLHSDGKTY |
| | Ab26 | |
| 1698 | VLCDR2 (IMGT) | ELS |
| | Ab26 | |
| 1699 | VLCDR3 (IMGT) | MQYIEAPLT |
| | Ab26 | |
| 1700 | VHCDR1 (IMGT) | GFTFSSYA |
| | Ab27 | |
| 1701 | VHCDR2 (IMGT) | ISYDGSNK |
| | Ab27 | |
| 1702 | VHCDR3 (IMGT) | VRGAGRGYTLGPDGFDI |
| | Ab27 | |
| 1703 | VLCDR1 (IMGT) | RSLLHSDGKTY |
| | Ab27 | |
| 1704 | VLCDR2 (IMGT) | ELS |
| | Ab27 | |
| 1705 | VLCDR3 (IMGT) | MQYIEAPLT |
| | Ab27 | |
| 1706 | VHCDR1 (IMGT) | GFTFSSYA |
| | Ab28 | |
| 1707 | VHCDR2 (IMGT) | ISYDGSNK |
| | Ab28 | |
| 1708 | VHCDR3 (IMGT) | VRGAGRGFTYGPDGFDI |
| | Ab28 | |
| 1709 | VLCDR1 (IMGT) | RSLLHSDGKTY |
| | Ab28 | |
| 1710 | VLCDR2 (IMGT) | ELS |
| | Ab28 | |
| 1711 | VLCDR3 (IMGT) | MQYIEAPLT |
| | Ab28 | |
| 1712 | VHCDR1 (IMGT) | GFTFSSYA |
| | Ab29 | |
| 1713 | VHCDR2 (IMGT) | ISYDGSNK |
| | Ab29 | |
| 1714 | VHCDR3 (IMGT) | VRGAGLGWPYGPDGFDI |
| | Ab29 | |
| 1715 | VLCDR1 (IMGT) | RSLLHSDGKTY |
| | Ab29 | |
| 1716 | VLCDR2 (IMGT) | ELS |
| | Ab29 | |
| 1717 | VLCDR3 (IMGT) | MQYVEAPLT |
| | Ab29 | |
| 1718 | VHCDR1 (IMGT) | GFTFSSYA |
| | Ab30 | |
| 1719 | VHCDR2 (IMGT) | ISYDGSNK |
| | Ab30 | |
| 1720 | VHCDR3 (IMGT) | VRGAGRGFTWGPDGFDI |
| | Ab30 | |
| 1721 | VLCDR1 (IMGT) | RSLLHSDGKTY |
| | Ab30 | |
| 1722 | VLCDR2 (IMGT) | ELS |
| | Ab30 | |
| 1723 | VLCDR3 (IMGT) | MQYVEAPLT |
| | Ab30 | |
| 1724 | VHCDR1 (IMGT) | GFTFSSYA |
| | Ab31 | |
| 1725 | VHCDR2 (IMGT) | ISYDGSNK |
| | Ab31 | |
| 1726 | VHCDR3 (IMGT) | VRGAGRGLTYGPDGFDI |
| | Ab31 | |
| 1727 | VLCDR1 (IMGT) | RSLLHSDGKTY |
| | Ab31 | |
| 1728 | VLCDR2 (IMGT) | ELS |
| | Ab31 | |
| 1729 | VLCDR3 (IMGT) | MQYVEAPLT |
| | Ab31 | |
| 1730 | VHCDR1 (IMGT) | GFTFSSYA |
| | Ab32 | |
| 1731 | VHCDR2 (IMGT) | ISYDGSNK |
| | Ab32 | |
| 1732 | VHCDR3 (IMGT) | VRGAGLGFTYGPDGFDI |
| | Ab32 | |
| 1733 | VLCDR1 (IMGT) | RSLLHSDGKTY |
| | Ab32 | |
| 1734 | VLCDR2 (IMGT) | ELS |
| | Ab32 | |
| 1735 | VLCDR3 (IMGT) | MQYVEAPLT |
| | Ab32 | |
| 1736 | VHCDR1 (IMGT) | GFTFSSYA |
| | Ab33 | |
| 1737 | VHCDR2 (IMGT) | ISYDGSNK |
| | Ab33 | |
| 1738 | VHCDR3 (IMGT) | VRGAGQGLSYGPDGFDI |
| | Ab33 | |
| 1739 | VLCDR1 (IMGT) | RSLLHSDGKTY |
| | Ab33 | |
| 1740 | VLCDR2 (IMGT) | ELS |
| | Ab33 | |
| 1741 | VLCDR3 (IMGT) | MQYVEAPLT |
| | Ab33 | |
| 1742 | VHCDR1 (IMGT) | GFTFSSYA |
| | Ab34 | |
| 1743 | VHCDR2 (IMGT) | ISYDGSNK |
| | Ab34 | |
| 1744 | VHCDR3 (IMGT) | VRGAGRGYTRGPDGFDI |
| | Ab34 | |
| 1745 | VLCDR1 (IMGT) | RSLLHSDGKTY |
| | Ab34 | |
| 1746 | VLCDR2 (IMGT) | ELS |
| | Ab34 | |
| 1747 | VLCDR3 (IMGT) | MQYVEAPLT |
| | Ab34 | |
| 1748 | VHCDR1 (IMGT) | GFTFSSYA |
| | Ab35 | |
| 1749 | VHCDR2 (IMGT) | ISYDGSNK |
| | Ab35 | |
| 1750 | VHCDR3 (IMGT) | VRGAGRGYTNGPDGFDI |
| | Ab35 | |
| 1751 | VLCDR1 (IMGT) | RSLLHSDGKTY |
| | Ab35 | |
| 1752 | VLCDR2 (IMGT) | ELS |
| | Ab35 | |
| 1753 | VLCDR3 (IMGT) | MQYVEAPLT |
| | Ab35 | |
| 1754 | VHCDR1 (IMGT) | GFTFSSYA |
| | Ab36 | |
| 1755 | VHCDR2 (IMGT) | ISYDGSNK |
| | Ab36 | |
| 1756 | VHCDR3 (IMGT) | VRGAGRGFTYGPDGFDI |
| | Ab36 | |
| 1757 | VLCDR1 (IMGT) | RSLLHSDGKTY |
| | Ab36 | |
| 1758 | VLCDR2 (IMGT) | ELS |
| | Ab36 | |
| 1759 | VLCDR3 (IMGT) | MQYIEAPLT |
| | Ab36 | |
| 1760 | VHCDR1 (IMGT) | GFTFSSYA |
| | Ab37 | |
| 1761 | VHCDR2 (IMGT) | ISYDGSNK |
| | Ab37 | |
| 1762 | VHCDR3 (IMGT) | VRGAGRGLTYGPDGFDI |
| | Ab37 | |
| 1763 | VLCDR1 (IMGT) | RSLLHSDGKTY |
| | Ab37 | |
| 1764 | VLCDR2 (IMGT) | ELS |
| | Ab37 | |
| 1765 | VLCDR3 (IMGT) | MQYIEAPLT |
| | Ab37 | |
| 1766 | VHCDR1 (IMGT) | GFTFSSYA |
| | Ab38 | |
| 1767 | VHCDR2 (IMGT) | ISYDGSNK |
| | Ab38 | |
| 1768 | VHCDR3 (IMGT) | VRGAGQGYPYGPDGFDI |
| | Ab38 | |
| 1769 | VLCDR1 (IMGT) | RSLLHSDGKTY |
| | Ab38 | |
| 1770 | VLCDR2 (IMGT) | ELS |
| | Ab38 | |
| 1771 | VLCDR3 (IMGT) | MQYIEAPLT |
| | Ab38 | |
| 1772 | VHCDR1 (IMGT) | GFTFSSYA |
| | Ab39 | |
| 1773 | VHCDR2 (IMGT) | ISYDGSNK |
| | Ab39 | |
| 1774 | VHCDR3 (IMGT) | VRGAGTGFTYGPDGFDI |
| | Ab39 | |
| 1775 | VLCDR1 (IMGT) | RSLLHSDGKTY |
| | Ab39 | |
| 1776 | VLCDR2 (IMGT) | ELS |
| | Ab39 | |
| 1777 | VLCDR3 (IMGT) | MQYIEAPLT |
| | Ab39 | |
| 1778 | VHCDR1 (IMGT) | GFTFSSYA |
| | Ab40 | |
| 1779 | VHCDR2 (IMGT) | ISYDGSNK |
| | Ab40 | |
| 1780 | VHCDR3 (IMGT) | VRGAGMGLTYGPDGFDI |
| | Ab40 | |
| 1781 | VLCDR1 (IMGT) | RSLLHSDGKTY |
| | Ab40 | |
| 1782 | VLCDR2 (IMGT) | ELS |
| | Ab40 | |
| 1783 | VLCDR3 (IMGT) | MQYIEAPLT |
| | Ab40 | |
| 1784 | VHCDR1 (IMGT) | GFTFSSYA |
| | Ab41 | |
| 1785 | VHCDR2 (IMGT) | ISYDGSNK |
| | Ab41 | |
| 1786 | VHCDR3 (IMGT) | VRGAGLGFPYGPDGFDI |
| | Ab41 | |
| 1787 | VLCDR1 (IMGT) | RSLLHSDGKTY |
| | Ab41 | |
| 1788 | VLCDR2 (IMGT) | ELS |
| | Ab41 | |
| 1789 | VLCDR3 (IMGT) | MQYIEAPLT |
| | Ab41 | |
| 1790 | VHCDR1 (IMGT) | GFTFSSYA |
| | Ab42 | |
| 1791 | VHCDR2 (IMGT) | ISYDGSNK |
| | Ab42 | |
| 1792 | VHCDR3 (IMGT) | VRGAGLGWAYGPDGFDI |
| | Ab42 | |
| 1793 | VLCDR1 (IMGT) | RSLLHSDGKTY |
| | Ab42 | |
| 1794 | VLCDR2 (IMGT) | ELS |
| | Ab42 | |
| 1795 | VLCDR3 (IMGT) | MQYIEAPLT |
| | Ab42 | |
| 1796 | VHCDR1 (IMGT) | GFTFSSYA |
| | Ab43 | |
| 1797 | VHCDR2 (IMGT) | ISYDGSNK |
| | Ab43 | |
| 1798 | VHCDR3 (IMGT) | VRGAGRGYPYGPDGFDI |
| | Ab43 | |
| 1799 | VLCDR1 (IMGT) | RSLLHSDGKTY |
| | Ab43 | |
| 1800 | VLCDR2 (IMGT) | ELS |
| | Ab43 | |
| 1801 | VLCDR3 (IMGT) | MQYIEAPLT |
| | Ab43 | |
| 1802 | VHCDR1 (IMGT) | GFDFAGYA |
| | Ab44 | |
| 1803 | VHCDR2 (IMGT) | ISYDGSNK |
| | Ab44 | |
| 1804 | VHCDR3 (IMGT) | VRGAGRGLTYGPDGFDI |
| | Ab44 | |
| 1805 | VLCDR1 (IMGT) | RSLLHSDGKTY |
| | Ab44 | |
| 1806 | VLCDR2 (IMGT) | ELS |
| | Ab44 | |
| 1807 | VLCDR3 (IMGT) | MQYIEAPLT |
| | Ab44 | |
| 1808 | VHCDR1 (IMGT) | GFTFSSYA |
| | Ab45 | |
| 1809 | VHCDR2 (IMGT) | ISYDGSNK |
| | Ab45 | |
| 1810 | VHCDR3 (IMGT) | VRGAGLGLTYGPDGFDI |
| | Ab45 | |
| 1811 | VLCDR1 (IMGT) | RSLLHSDGKTY |
| | Ab45 | |
| 1812 | VLCDR2 (IMGT) | ELS |
| | Ab45 | |
| 1813 | VLCDR3 (IMGT) | MQYIEAPLT |
| | Ab45 | |
| 1814 | VHCDR1 (IMGT) | GFTFSSYA |
| | Ab46 | |
| 1815 | VHCDR2 (IMGT) | ISYDGSNK |
| | Ab46 | |
| 1816 | VHCDR3 (IMGT) | VRGAGLGYPYGPDGFDI |
| | Ab46 | |
| 1817 | VLCDR1 (IMGT) | RSLLHSDGKTY |
| | Ab46 | |
| 1818 | VLCDR2 (IMGT) | ELS |
| | Ab46 | |
| 1819 | VLCDR3 (IMGT) | MQYIEAPLT |
| | Ab46 | |
| 1820 | VHCDR1 (IMGT) | GFTFSSYA |
| | Ab47 | |
| 1821 | VHCDR2 (IMGT) | ISYDGSNK |
| | Ab47 | |
| 1822 | VHCDR3 (IMGT) | VRGAGRGYPHGPDGFDI |
| | Ab47 | |
| 1823 | VLCDR1 (IMGT) | RSLLHSDGKTY |
| | Ab47 | |
| 1824 | VLCDR2 (IMGT) | ELS |
| | Ab47 | |
| 1825 | VLCDR3 (IMGT) | MQYIEAPLT |
| | Ab47 | |
| 1826 | VHCDR1 (IMGT) | GFTFSSYA |
| | Ab48 | |
| 1827 | VHCDR2 (IMGT) | ISYDGSNK |
| | Ab48 | |
| 1828 | VHCDR3 (IMGT) | VRGAGIGYPHGPDGFDI |
| | Ab48 | |
| 1829 | VLCDR1 (IMGT) | RSLLHSDGKTY |
| | Ab48 | |
| 1830 | VLCDR2 (IMGT) | ELS |
| | Ab48 | |
| 1831 | VLCDR3 (IMGT) | MQYIEAPLT |
| | Ab48 | |
| 1832 | VHCDR1 (IMGT) | GFTFSSYA |
| | Ab49 | |
| 1833 | VHCDR2 (IMGT) | ISYDGSNK |
| | Ab49 | |
| 1834 | VHCDR3 (IMGT) | VRGAGRGYPYGPDGFDI |
| | Ab49 | |
| 1835 | VLCDR1 (IMGT) | RSLLHSDGKTY |
| | Ab49 | |
| 1836 | VLCDR2 (IMGT) | ELS |
| | Ab49 | |
| 1837 | VLCDR3 (IMGT) | MQYIEAPLT |
| | Ab49 | |
| 1838 | VHCDR1 (IMGT) | GFTFSSYA |
| | Ab50 | |
| 1839 | VHCDR2 (IMGT) | ISYDGSNK |
| | Ab50 | |
| 1840 | VHCDR3 (IMGT) | VRGAGLGFPFGPDGFDI |
| | Ab50 | |
| 1841 | VLCDR1 (IMGT) | RSLLHSDGKTY |
| | Ab50 | |
| 1842 | VLCDR2 (IMGT) | ELS |
| | Ab50 | |
| 1843 | VLCDR3 (IMGT) | MQYIEAPLT |
| | Ab50 | |
| 1844 | VHCDR1 (IMGT) | GFTFSSYA |
| | Ab51 | |
| 1845 | VHCDR2 (IMGT) | ISYDGSNK |
| | Ab51 | |
| 1846 | VHCDR3 (IMGT) | VRGAGRGFPYGPDGFDI |
| | Ab51 | |
| 1847 | VLCDR1 (IMGT) | RSLLHSDGKTY |
| | Ab51 | |
| 1848 | VLCDR2 (IMGT) | ELS |
| | Ab51 | |
| 1849 | VLCDR3 (IMGT) | MQYIEAPLT |
| | Ab51 | |
| 1850 | VHCDR1 (IMGT) | GFTFSSYA |
| | Ab52 | |
| 1851 | VHCDR2 (IMGT) | ISYDGSNK |
| | Ab52 | |
| 1852 | VHCDR3 (IMGT) | VRGAGLGYPNGPDGFDI |
| | Ab52 | |
| 1853 | VLCDR1 (IMGT) | RSLLHSDGKTY |
| | Ab52 | |
| 1854 | VLCDR2 (IMGT) | ELS |
| | Ab52 | |
| 1855 | VLCDR3 (IMGT) | MQYIEAPLT |
| | Ab52 | |
| 1856 | VHCDR1 (IMGT) | GFTFSSYA |
| | Ab53 | |
| 1857 | VHCDR2 (IMGT) | ISYDGSNK |
| | Ab53 | |
| 1858 | VHCDR3 (IMGT) | VRGAGQGFPYGPDGFDI |
| | Ab53 | |
| 1859 | VLCDR1 (IMGT) | RSLLHSDGKTY |
| | Ab53 | |
| 1860 | VLCDR2 (IMGT) | ELS |
| | Ab53 | |
| 1861 | VLCDR3 (IMGT) | MQYIEAPLT |
| | Ab53 | |
| 1862 | VHCDR1 (IMGT) | GFTFSSYA |
| | Ab54 | |
| 1863 | VHCDR2 (IMGT) | ISYDGSNK |
| | Ab54 | |
| 1864 | VHCDR3 (IMGT) | VRGAGQGYPYGPDGFDI |
| | Ab54 | |
| 1865 | VLCDR1 (IMGT) | RSLLHSDGKTY |
| | Ab54 | |
| 1866 | VLCDR2 (IMGT) | ELS |
| | Ab54 | |
| 1867 | VLCDR3 (IMGT) | MQYIEAPLT |
| | Ab54 | |
| 1868 | VHCDR1 (IMGT) | GFTFSSYA |
| | Ab55 | |
| 1869 | VHCDR2 (IMGT) | ISYDGSNK |
| | Ab55 | |
| 1870 | VHCDR3 (IMGT) | VRGAGTGFTYGPDGFDI |
| | Ab55 | |
| 1871 | VLCDR1 (IMGT) | RSLLHSDGKTY |
| | Ab55 | |
| 1872 | VLCDR2 (IMGT) | ELS |
| | Ab55 | |
| 1873 | VLCDR3 (IMGT) | MQYIEAPLT |
| | Ab55 | |
| 1874 | VHCDR1 (IMGT) | GFTFSSYA |
| | Ab56 | |
| 1875 | VHCDR2 (IMGT) | ISYDGSNK |
| | Ab56 | |
| 1876 | VHCDR3 (IMGT) | VRGAGMGLTYGPDGFDI |
| | Ab56 | |
| 1877 | VLCDR1 (IMGT) | RSLLHSDGKTY |
| | Ab56 | |
| 1878 | VLCDR2 (IMGT) | ELS |
| | Ab56 | |
| 1879 | VLCDR3 (IMGT) | MQYIEAPLT |
| | Ab56 | |
| 1880 | VHCDR1 (IMGT) | GFTFSSYA |
| | Ab57 | |
| 1881 | VHCDR2 (IMGT) | ISYDGSNK |
| | Ab57 | |
| 1882 | VHCDR3 (IMGT) | VRGAGLGFPYGPDGFDI |
| | Ab57 | |
| 1883 | VLCDR1 (IMGT) | RSLLHSDGKTY |
| | Ab57 | |
| 1884 | VLCDR2 (IMGT) | ELS |
| | Ab57 | |
| 1885 | VLCDR3 (IMGT) | MQYIEAPLT |
| | Ab57 | |
| 1886 | VHCDR1 (IMGT) | GFTFSSYA |
| | Ab58 | |
| 1887 | VHCDR2 (IMGT) | ISYDGSNK |
| | Ab58 | |
| 1888 | VHCDR3 (IMGT) | VRGAGLGWAYGPDGFDI |
| | Ab58 | |
| 1889 | VLCDR1 (IMGT) | RSLLHSDGKTY |
| | Ab58 | |
| 1890 | VLCDR2 (IMGT) | ELS |
| | Ab58 | |
| 1891 | VLCDR3 (IMGT) | MQYIEAPLT |
| | Ab58 | |
| 1892 | VHCDR1 (IMGT) | GFTFSSYA |
| | Ab59 | |
| 1893 | VHCDR2 (IMGT) | ISYDGSNK |
| | Ab59 | |
| 1894 | VHCDR3 (IMGT) | VRGAGRGYPYGPDGFDI |
| | Ab59 | |
| 1895 | VLCDR1 (IMGT) | RSLLHSDGKTY |
| | Ab59 | |
| 1896 | VLCDR2 (IMGT) | ELS |
| | Ab59 | |
| 1897 | VLCDR3 (IMGT) | MQYIEAPLT |
| | Ab59 | |
| 1898 | VHCDR1 (IMGT) | GFDFAGYA |
| | Ab60 | |
| 1899 | VHCDR2 (IMGT) | ISYDGSNK |
| | Ab60 | |
| 1900 | VHCDR3 (IMGT) | VRGAGRGLTYGPDGFDI |
| | Ab60 | |
| 1901 | VLCDR1 (IMGT) | RSLLHSDGKTY |
| | Ab60 | |
| 1902 | VLCDR2 (IMGT) | ELS |
| | Ab60 | |
| 1903 | VLCDR3 (IMGT) | MQYIEAPLT |
| | Ab60 | |
| 1904 | VHCDR1 (IMGT) | GFTFSSYA |
| | Ab61 | |
| 1905 | VHCDR2 (IMGT) | ISYDGSNK |
| | Ab61 | |
| 1906 | VHCDR3 (IMGT) | VRGAGLGLTYGPDGFDI |
| | Ab61 | |
| 1907 | VLCDR1 (IMGT) | RSLLHSDGKTY |
| | Ab61 | |
| 1908 | VLCDR2 (IMGT) | ELS |
| | Ab61 | |
| 1909 | VLCDR3 (IMGT) | MQYIEAPLT |
| | Ab61 | |
| 1910 | VHCDR1 (IMGT) | GFTFSSYA |
| | Ab62 | |
| 1911 | VHCDR2 (IMGT) | ISYDGSNK |
| | Ab62 | |
| 1912 | VHCDR3 (IMGT) | VRGAGLGYPYGPDGFDI |
| | Ab62 | |
| 1913 | VLCDR1 (IMGT) | RSLLHSDGKTY |
| | Ab62 | |
| 1914 | VLCDR2 (IMGT) | ELS |
| | Ab62 | |
| 1915 | VLCDR3 (IMGT) | MQYIEAPLT |
| | Ab62 | |
| 1916 | VHCDR1 (IMGT) | GFTFSSYA |
| | Ab63 | |
| 1917 | VHCDR2 (IMGT) | ISYDGSNK |
| | Ab63 | |
| 1918 | VHCDR3 (IMGT) | VRGAGRGYPHGPDGFDI |
| | Ab63 | |
| 1919 | VLCDR1 (IMGT) | RSLLHSDGKTY |
| | Ab63 | |
| 1920 | VLCDR2 (IMGT) | ELS |
| | Ab63 | |
| 1921 | VLCDR3 (IMGT) | MQYIEAPLT |
| | Ab63 | |
| 1922 | VHCDR1 (IMGT) | GFTFSSYA |
| | Ab64 | |
| 1923 | VHCDR2 (IMGT) | ISYDGSNK |
| | Ab64 | |
| 1924 | VHCDR3 (IMGT) | VRGAGIGYPHGPDGFDI |
| | Ab64 | |
| 1925 | VLCDR1 (IMGT) | RSLLHSDGKTY |
| | Ab64 | |
| 1926 | VLCDR2 (IMGT) | ELS |
| | Ab64 | |
| 1927 | VLCDR3 (IMGT) | MQYIEAPLT |
| | Ab64 | |
| 1928 | VHCDR1 (IMGT) | GFTFSSYA |
| | Ab65 | |
| 1929 | VHCDR2 (IMGT) | ISYDGSNK |
| | Ab65 | |
| 1930 | VHCDR3 (IMGT) | VRGAGRGYPYGPDGFDI |
| | Ab65 | |
| 1931 | VLCDR1 (IMGT) | RSLLHSDGKTY |
| | Ab65 | |
| 1932 | VLCDR2 (IMGT) | ELS |
| | Ab65 | |
| 1933 | VLCDR3 (IMGT) | MQYIEAPLT |
| | Ab65 | |
| 1934 | VHCDR1 (IMGT) | GFTFSSYA |
| | Ab66 | |
| 1935 | VHCDR2 (IMGT) | ISYDGSNK |
| | Ab66 | |
| 1936 | VHCDR3 (IMGT) | VRGAGLGFPFGPDGFDI |
| | Ab66 | |
| 1937 | VLCDR1 (IMGT) | RSLLHSDGKTY |
| | Ab66 | |
| 1938 | VLCDR2 (IMGT) | ELS |
| | Ab66 | |
| 1939 | VLCDR3 (IMGT) | MQYIEAPLT |
| | Ab66 | |
| 1940 | VHCDR1 (IMGT) | GFTFSSYA |
| | Ab67 | |
| 1941 | VHCDR2 (IMGT) | ISYDGSNK |
| | Ab67 | |
| 1942 | VHCDR3 (IMGT) | VRGAGRGFPYGPDGFDI |
| | Ab67 | |
| 1943 | VLCDR1 (IMGT) | RSLLHSDGKTY |
| | Ab67 | |
| 1944 | VLCDR2 (IMGT) | ELS |
| | Ab67 | |
| 1945 | VLCDR3 (IMGT) | MQYIEAPLT |
| | Ab67 | |
| 1946 | VHCDR1 (IMGT) | GFTFSSYA |
| | Ab68 | |
| 1947 | VHCDR2 (IMGT) | ISYDGSNK |
| | Ab68 | |
| 1948 | VHCDR3 (IMGT) | VRGAGLGYPNGPDGFDI |
| | Ab68 | |
| 1949 | VLCDR1 (IMGT) | RSLLHSDGKTY |
| | Ab68 | |
| 1950 | VLCDR2 (IMGT) | ELS |
| | Ab68 | |
| 1951 | VLCDR3 (IMGT) | MQYIEAPLT |
| | Ab68 | |
| 1952 | VHCDR1 (IMGT) | GFTFSSYA |
| | Ab69 | |
| 1953 | VHCDR2 (IMGT) | ISYDGSNK |
| | Ab69 | |
| 1954 | VHCDR3 (IMGT) | VRGAGQGFPYGPDGFDI |
| | Ab69 | |
| 1955 | VLCDR1 (IMGT) | RSLLHSDGKTY |
| | Ab69 | |
| 1956 | VLCDR2 (IMGT) | ELS |
| | Ab69 | |
| 1957 | VLCDR3 (IMGT) | MQYIEAPLT |
| | Ab69 | |
| 1958 | VHCDR1 (IMGT) | GFTFSSYA |
| | Ab70 | |
| 1959 | VHCDR2 (IMGT) | ISYDGSNK |
| | Ab70 | |
| 1960 | VHCDR3 (IMGT) | VRGAGQGYPYGPDGFDI |
| | Ab70 | |
| 1961 | VLCDR1 (IMGT) | RSLLHSDGKTY |
| | Ab70 | |
| 1962 | VLCDR2 (IMGT) | ELS |
| | Ab70 | |
| 1963 | VLCDR3 (IMGT) | MQYIEAPLT |
| | Ab70 | |
| 1964 | VHCDR1 (IMGT) | GFTFSSYA |
| | Ab71 | |
| 1965 | VHCDR2 (IMGT) | ISYDGSNK |
| | Ab71 | |
| 1966 | VHCDR3 (IMGT) | VRGAGTGFTYGPDGFDI |
| | Ab71 | |
| 1967 | VLCDR1 (IMGT) | RSLLHSDGKTY |
| | Ab71 | |
| 1968 | VLCDR2 (IMGT) | ELS |
| | Ab71 | |
| 1969 | VLCDR3 (IMGT) | MQYIEAPLT |
| | Ab71 | |
| 1970 | VHCDR1 (IMGT) | GFTFSSYA |
| | Ab72 | |
| 1971 | VHCDR2 (IMGT) | ISYDGSNK |
| | Ab72 | |
| 1972 | VHCDR3 (IMGT) | VRGAGMGLTYGPDGFDI |
| | Ab72 | |
| 1973 | VLCDR1 (IMGT) | RSLLHSDGKTY |
| | Ab72 | |
| 1974 | VLCDR2 (IMGT) | ELS |
| | Ab72 | |
| 1975 | VLCDR3 (IMGT) | MQYIEAPLT |
| | Ab72 | |
| 1976 | VHCDR1 (IMGT) | GFTFSSYA |
| | Ab73 | |
| 1977 | VHCDR2 (IMGT) | ISYDGSNK |
| | Ab73 | |
| 1978 | VHCDR3 (IMGT) | VRGAGLGFPYGPDGFDI |
| | Ab73 | |
| 1979 | VLCDR1 (IMGT) | RSLLHSDGKTY |
| | Ab73 | |
| 1980 | VLCDR2 (IMGT) | ELS |
| | Ab73 | |
| 1981 | VLCDR3 (IMGT) | MQYIEAPLT |
| | Ab73 | |
| 1982 | VHCDR1 (IMGT) | GFTFSSYA |
| | Ab74 | |
| 1983 | VHCDR2 (IMGT) | ISYDGSNK |
| | Ab74 | |
| 1984 | VHCDR3 (IMGT) | VRGAGLGWAYGPDGFDI |
| | Ab74 | |
| 1985 | VLCDR1 (IMGT) | RSLLHSDGKTY |
| | Ab74 | |
| 1986 | VLCDR2 (IMGT) | ELS |
| | Ab74 | |
| 1987 | VLCDR3 (IMGT) | MQYIEAPLT |
| | Ab74 | |
| 1988 | VHCDR1 (IMGT) | GFTFSSYA |
| | Ab75 | |
| 1989 | VHCDR2 (IMGT) | ISYDGSNK |
| | Ab75 | |
| 1990 | VHCDR3 (IMGT) | VRGAGRGYPYGPDGFDI |
| | Ab75 | |
| 1991 | VLCDR1 (IMGT) | RSLLHSDGKTY |
| | Ab75 | |
| 1992 | VLCDR2 (IMGT) | ELS |
| | Ab75 | |
| 1993 | VLCDR3 (IMGT) | MQYIEAPLT |
| | Ab75 | |
| 1994 | VHCDR1 (IMGT) | GFDFAGYA |
| | Ab76 | |
| 1995 | VHCDR2 (IMGT) | ISYDGSNK |
| | Ab76 | |
| 1996 | VHCDR3 (IMGT) | VRGAGRGLTYGPDGFDI |
| | Ab76 | |
| 1997 | VLCDR1 (IMGT) | RSLLHSDGKTY |
| | Ab76 | |
| 1998 | VLCDR2 (IMGT) | ELS |
| | Ab76 | |
| 1999 | VLCDR3 (IMGT) | MQYIEAPLT |
| | Ab76 | |
| 2000 | VHCDR1 (IMGT) | GFTFSSYA |
| | Ab77 | |
| 2001 | VHCDR2 (IMGT) | ISYDGSNK |
| | Ab77 | |
| 2002 | VHCDR3 (IMGT) | VRGAGLGLTYGPDGFDI |
| | Ab77 | |
| 2003 | VLCDR1 (IMGT) | RSLLHSDGKTY |
| | Ab77 | |
| 2004 | VLCDR2 (IMGT) | ELS |
| | Ab77 | |
| 2005 | VLCDR3 (IMGT) | MQYIEAPLT |
| | Ab77 | |
| 2006 | VHCDR1 (IMGT) | GFTFSSYA |
| | Ab78 | |
| 2007 | VHCDR2 (IMGT) | ISYDGSNK |
| | Ab78 | |
| 2008 | VHCDR3 (IMGT) | VRGAGLGYPYGPDGFDI |
| | Ab78 | |
| 2009 | VLCDR1 (IMGT) | RSLLHSDGKTY |
| | Ab78 | |
| 2010 | VLCDR2 (IMGT) | ELS |
| | Ab78 | |
| 2011 | VLCDR3 (IMGT) | MQYIEAPLT |
| | Ab78 | |
| 2012 | VHCDR1 (IMGT) | GFTFSSYA |
| | Ab79 | |
| 2013 | VHCDR2 (IMGT) | ISYDGSNK |
| | Ab79 | |
| 2014 | VHCDR3 (IMGT) | VRGAGRGYPHGPDGFDI |
| | Ab79 | |
| 2015 | VLCDR1 (IMGT) | RSLLHSDGKTY |
| | Ab79 | |
| 2016 | VLCDR2 (IMGT) | ELS |
| | Ab79 | |
| 2017 | VLCDR3 (IMGT) | MQYIEAPLT |
| | Ab79 | |
| 2018 | VHCDR1 (IMGT) | GFTFSSYA |
| | Ab80 | |
| 2019 | VHCDR2 (IMGT) | ISYDGSNK |
| | Ab80 | |
| 2020 | VHCDR3 (IMGT) | VRGAGIGYPHGPDGFDI |
| | Ab80 | |
| 2021 | VLCDR1 (IMGT) | RSLLHSDGKTY |
| | Ab80 | |
| 2022 | VLCDR2 (IMGT) | ELS |
| | Ab80 | |
| 2023 | VLCDR3 (IMGT) | MQYIEAPLT |
| | Ab80 | |
| 2024 | VHCDR1 (IMGT) | GFTFSSYA |
| | Ab81 | |
| 2025 | VHCDR2 (IMGT) | ISYDGSNK |
| | Ab81 | |
| 2026 | VHCDR3 (IMGT) | VRGAGRGYPYGPDGFDI |
| | Ab81 | |
| 2027 | VLCDR1 (IMGT) | RSLLHSDGKTY |
| | Ab81 | |
| 2028 | VLCDR2 (IMGT) | ELS |
| | Ab81 | |
| 2029 | VLCDR3 (IMGT) | MQYIEAPLT |
| | Ab81 | |
| 2030 | VHCDR1 (IMGT) | GFTFSSYA |
| | Ab82 | |
| 2031 | VHCDR2 (IMGT) | ISYDGSNK |
| | Ab82 | |
| 2032 | VHCDR3 (IMGT) | VRGAGLGFPFGPDGFDI |
| | Ab82 | |
| 2033 | VLCDR1 (IMGT) | RSLLHSDGKTY |
| | Ab82 | |
| 2034 | VLCDR2 (IMGT) | ELS |
| | Ab82 | |
| 2035 | VLCDR3 (IMGT) | MQYIEAPLT |
| | Ab82 | |
| 2036 | VHCDR1 (IMGT) | GFTFSSYA |
| | Ab83 | |
| 2037 | VHCDR2 (IMGT) | ISYDGSNK |
| | Ab83 | |
| 2038 | VHCDR3 (IMGT) | VRGAGRGFPYGPDGFDI |
| | Ab83 | |
| 2039 | VLCDR1 (IMGT) | RSLLHSDGKTY |
| | Ab83 | |
| 2040 | VLCDR2 (IMGT) | ELS |
| | Ab83 | |
| 2041 | VLCDR3 (IMGT) | MQYIEAPLT |
| | Ab83 | |
| 2042 | VHCDR1 (IMGT) | GFTFSSYA |
| | Ab84 | |
| 2043 | VHCDR2 (IMGT) | ISYDGSNK |
| | Ab84 | |
| 2044 | VHCDR3 (IMGT) | VRGAGLGYPNGPDGFDI |
| | Ab84 | |
| 2045 | VLCDR1 (IMGT) | RSLLHSDGKTY |
| | Ab84 | |
| 2046 | VLCDR2 (IMGT) | ELS |
| | Ab84 | |
| 2047 | VLCDR3 (IMGT) | MQYIEAPLT |
| | Ab84 | |
| 2048 | VHCDR1 (IMGT) | GFTFSSYA |
| | Ab85 | |
| 2049 | VHCDR2 (IMGT) | ISYDGSNK |
| | Ab85 | |
| 2050 | VHCDR3 (IMGT) | VRGAGQGFPYGPDGFDI |
| | Ab85 | |
| 2051 | VLCDR1 (IMGT) | RSLLHSDGKTY |
| | Ab85 | |
| 2052 | VLCDR2 (IMGT) | ELS |
| | Ab85 | |
| 2053 | VLCDR3 (IMGT) | MQYIEAPLT |
| | Ab85 | |
| 2054 | VHCDR1 (IMGT) | GFTFSSYA |
| | Ab86 | |
| 2055 | VHCDR2 (IMGT) | ISYDGSNK |
| | Ab86 | |
| 2056 | VHCDR3 (IMGT) | VRGAGRGLTYGPDGFDI |
| | Ab86 | |
| 2057 | VLCDR1 (IMGT) | RSLLWSDGKTY |
| | Ab86 | |
| 2058 | VLCDR2 (IMGT) | ELS |
| | Ab86 | |
| 2059 | VLCDR3 (IMGT) | MQYIEAPLT |
| | Ab86 | |
| 2060 | VHCDR1 (IMGT) | GFTFSSYA |
| | Ab87 | |
| 2061 | VHCDR2 (IMGT) | ISYDGSNK |
| | Ab87 | |
| 2062 | VHCDR3 (IMGT) | VRGAGRGFTWGPDGFDI |
| | Ab87 | |
| 2063 | VLCDR1 (IMGT) | RSLLHSDGKTY |
| | Ab87 | |
| 2064 | VLCDR2 (IMGT) | ELS |
| | Ab87 | |
| 2065 | VLCDR3 (IMGT) | MQYVEAPLT |
| | Ab87 | |
| 2066 | VHCDR1 (IMGT) | GFTFSSYA |
| | Ab88 | |
| 2067 | VHCDR2 (IMGT) | ISYDGSNK |
| | Ab88 | |
| 2068 | VHCDR3 (IMGT) | VRGAGRGLTYGPDGFDI |
| | Ab88 | |
| 2069 | VLCDR1 (IMGT) | RSLLHSDGKTY |
| | Ab88 | |
| 2070 | VLCDR2 (IMGT) | ELS |
| | Ab88 | |
| 2071 | VLCDR3 (IMGT) | MQYIEAPLT |
| | Ab88 | |
| 2072 | VHCDR1 (IMGT) | GFTFSSYA |
| | Ab89 | |
| 2073 | VHCDR2 (IMGT) | ISYDGSNK |
| | Ab89 | |
| 2074 | VHCDR3 (IMGT) | VRGAGRGFPYGPDGFDI |
| | Ab89 | |
| 2075 | VLCDR1 (IMGT) | RSLLHSDGKTY |
| | Ab89 | |
| 2076 | VLCDR2 (IMGT) | ELS |
| | Ab89 | |
| 2077 | VLCDR3 (IMGT) | MQYIEVPLT |
| | Ab89 | |
| 2078 | VHCDR1 (IMGT) | GFTFSSYA |
| | Ab90 | |
| 2079 | VHCDR2 (IMGT) | ISYDGSNK |
| | Ab90 | |
| 2080 | VHCDR3 (IMGT) | VRGAGRGYPYGPDGFDI |
| | Ab90 | |
| 2081 | VLCDR1 (IMGT) | RSLLHSDGKTY |
| | Ab90 | |
| 2082 | VLCDR2 (IMGT) | ELS |
| | Ab90 | |
| 2083 | VLCDR3 (IMGT) | MQYVEVPLT |
| | Ab90 | |
| 2084 | VHCDR1 (IMGT) | GFTFSSYA |
| | Ab91 | |
| 2085 | VHCDR2 (IMGT) | ISYDGSNK |
| | Ab91 | |
| 2086 | VHCDR3 (IMGT) | VRGAGQGYMHGPDGFDI |
| | Ab91 | |
| 2087 | VLCDR1 (IMGT) | RSLLWSDGKTY |
| | Ab91 | |
| 2088 | VLCDR2 (IMGT) | ELS |
| | Ab91 | |
| 2089 | VLCDR3 (IMGT) | MQYIEAPLT |
| | Ab91 | |
| 2090 | VHCDR1 (IMGT) | GFTFSSYA |
| | Ab92 | |
| 2091 | VHCDR2 (IMGT) | ISYDGSNK |
| | Ab92 | |
| 2092 | VHCDR3 (IMGT) | VRGAGHGFPTGPDGFDI |
| | Ab92 | |
| 2093 | VLCDR1 (IMGT) | RSLLHSDGKTY |
| | Ab92 | |
| 2094 | VLCDR2 (IMGT) | ELS |
| | Ab92 | |
| 2095 | VLCDR3 (IMGT) | MQYVEAPLT |
| | Ab92 | |
| 2096 | VHCDR1 (IMGT) | GFTFSSYA |
| | Ab93 | |
| 2097 | VHCDR2 (IMGT) | ISYDGSNK |
| | Ab93 | |
| 2098 | VHCDR3 (IMGT) | VRGAGLGFPYGPDGFDI |
| | Ab93 | |
| 2099 | VLCDR1 (IMGT) | RSLLHSDGKTY |
| | Ab93 | |
| 2100 | VLCDR2 (IMGT) | ELS |
| | Ab93 | |
| 2101 | VLCDR3 (IMGT) | MQYIEAPLT |
| | Ab93 | |
| 2102 | VHCDR1 (IMGT) | GFTFSSYA |
| | Ab94 | |
| 2103 | VHCDR2 (IMGT) | ISYDGSNK |
| | Ab94 | |
| 2104 | VHCDR3 (IMGT) | VRGAGLGWPYGPDGFDI |
| | Ab94 | |
| 2105 | VLCDR1 (IMGT) | RSLLHSDGKTY |
| | Ab94 | |
| 2106 | VLCDR2 (IMGT) | ELS |
| | Ab94 | |
| 2107 | VLCDR3 (IMGT) | MQYIEAPLT |
| | Ab94 | |
| 2108 | VHCDR1 (IMGT) | GFTFSSYA |
| | Ab95 | |
| 2109 | VHCDR2 (IMGT) | ISYDGSNK |
| | Ab95 | |
| 2110 | VHCDR3 (IMGT) | VRGAGLGWPYGPDGFDI |
| | Ab95 | |
| 2111 | VLCDR1 (IMGT) | RSLLHSDGKTY |
| | Ab95 | |
| 2112 | VLCDR2 (IMGT) | ELS |
| | Ab95 | |
| 2113 | VLCDR3 (IMGT) | MQYIEAPLT |
| | Ab95 | |
| 2114 | VHCDR1 (IMGT) | GFTFSSYA |
| | Ab96 | |
| 2115 | VHCDR2 (IMGT) | ISYDGSNK |
| | Ab96 | |
| 2116 | VHCDR3 (IMGT) | VRGAGLGWPYGPDGFDI |
| | Ab96 | |
| 2117 | VLCDR1 (IMGT) | RSLLHSDGKTY |
| | Ab96 | |
| 2118 | VLCDR2 (IMGT) | ELS |
| | Ab96 | |
| 2119 | VLCDR3 (IMGT) | MQYIEAPLT |
| | Ab96 | |
| 2120 | VHCDR1 (IMGT) | GFTFSSYA |
| | Ab97 | |
| 2121 | VHCDR2 (IMGT) | ISYDGSNK |
| | Ab97 | |
| 2122 | VHCDR3 (IMGT) | VRGAGLGWPYGPDGFDI |
| | Ab97 | |
| 2123 | VLCDR1 (IMGT) | RSLLHSDGKTY |
| | Ab97 | |
| 2124 | VLCDR2 (IMGT) | ELS |
| | Ab97 | |
| 2125 | VLCDR3 (IMGT) | MQYVEAPLT |
| | Ab97 | |
| 2126 | VHCDR1 (IMGT) | GFTFSSYA |
| | Ab98 | |
| 2127 | VHCDR2 (IMGT) | ISYDGSNK |
| | Ab98 | |
| 2128 | VHCDR3 (IMGT) | VRGAGLGWPYGPDGFDI |
| | Ab98 | |
| 2129 | VLCDR1 (IMGT) | RSLLHSDGKTY |
| | Ab98 | |
| 2130 | VLCDR2 (IMGT) | ELS |
| | Ab98 | |
| 2131 | VLCDR3 (IMGT) | MQYVEVPLT |
| | Ab98 | |
| 2132 | VHCDR1 (IMGT) | GFTFSSYA |
| | Ab99 | |
| 2133 | VHCDR2 (IMGT) | ISYDGSNK |
| | Ab99 | |
| 2134 | VHCDR3 (IMGT) | VRGAGLGWPYGPDGFDI |
| | Ab99 | |
| 2135 | VLCDR1 (IMGT) | RSLLWSDGKTY |
| | Ab99 | |
| 2136 | VLCDR2 (IMGT) | ELS |
| | Ab99 | |
| 2137 | VLCDR3 (IMGT) | MQYIEAPLT |
| | Ab99 | |
| 2138 | VHCDR1 (IMGT) | GFTFSSYA |
| | Ab100 | |
| 2139 | VHCDR2 (IMGT) | ISYDGSNK |
| | Ab100 | |
| 2140 | VHCDR3 (IMGT) | VRGAGLGWPYGPDGFDI |
| | Ab100 | |
| 2141 | VLCDR1 (IMGT) | RSLLWSDGKTY |
| | Ab100 | |
| 2142 | VLCDR2 (IMGT) | ELS |
| | Ab100 | |
| 2143 | VLCDR3 (IMGT) | MQYVEAPLT |
| | Ab100 | |
| 2144 | VHCDR1 (IMGT) | GFTFSSYA |
| | Ab101 | |
| 2145 | VHCDR2 (IMGT) | ISYDGSNK |
| | Ab101 | |
| 2146 | VHCDR3 (IMGT) | VRGAGRGFTLGPDGFDI |
| | Ab101 | |
| 2147 | VLCDR1 (IMGT) | RSLLHSDGKTY |
| | Ab101 | |
| 2148 | VLCDR2 (IMGT) | ELS |
| | Ab101 | |
| 2149 | VLCDR3 (IMGT) | MQYIEAPLT |
| | Ab101 | |
| 2150 | VHCDR1 (IMGT) | GFTFSSYA |
| | Ab102 | |
| 2151 | VHCDR2 (IMGT) | ISYDGSNK |
| | Ab102 | |
| 2152 | VHCDR3 (IMGT) | VRGAGRGFTLGPDGFDI |
| | Ab102 | |
| 2153 | VLCDR1 (IMGT) | RSLLHSDGKTY |
| | Ab102 | |
| 2154 | VLCDR2 (IMGT) | ELS |
| | Ab102 | |
| 2155 | VLCDR3 (IMGT) | MQYIEAPLT |
| | Ab102 | |
| 2156 | VHCDR1 (IMGT) | GFTFSSYA |
| | Ab103 | |
| 2157 | VHCDR2 (IMGT) | ISYDGSNK |
| | Ab103 | |
| 2158 | VHCDR3 (IMGT) | VRGAGRGFTLGPDGFDI |
| | Ab103 | |
| 2159 | VLCDR1 (IMGT) | RSLLHSDGKTY |
| | Ab103 | |
| 2160 | VLCDR2 (IMGT) | ELS |
| | Ab103 | |
| 2161 | VLCDR3 (IMGT) | MQYIEAPLT |
| | Ab103 | |
| 2162 | VHCDR1 (IMGT) | GFTFSSYA |
| | Ab104 | |
| 2163 | VHCDR2 (IMGT) | ISYDGSNK |
| | Ab104 | |
| 2164 | VHCDR3 (IMGT) | VRGAGRGFTLGPDGFDI |
| | Ab104 | |
| 2165 | VLCDR1 (IMGT) | RSLLHSDGKTY |
| | Ab104 | |
| 2166 | VLCDR2 (IMGT) | ELS |
| | Ab104 | |
| 2167 | VLCDR3 (IMGT) | MQYVEAPLT |
| | Ab104 | |
| 2168 | VHCDR1 (IMGT) | GFTFSSYA |
| | Ab105 | |
| 2169 | VHCDR2 (IMGT) | ISYDGSNK |
| | Ab105 | |
| 2170 | VHCDR3 (IMGT) | VRGAGRGFTLGPDGFDI |
| | Ab105 | |
| 2171 | VLCDR1 (IMGT) | RSLLHSDGKTY |
| | Ab105 | |
| 2172 | VLCDR2 (IMGT) | ELS |
| | Ab105 | |
| 2173 | VLCDR3 (IMGT) | MQYVEVPLT |
| | Ab105 | |
| 2174 | VHCDR1 (IMGT) | GFTFSSYA |
| | Ab106 | |
| 2175 | VHCDR2 (IMGT) | ISYDGSNK |
| | Ab106 | |
| 2176 | VHCDR3 (IMGT) | VRGAGRGFTLGPDGFDI |
| | Ab106 | |
| 2177 | VLCDR1 (IMGT) | RSLLWSDGKTY |
| | Ab106 | |
| 2178 | VLCDR2 (IMGT) | ELS |
| | Ab106 | |
| 2179 | VLCDR3 (IMGT) | MQYIEAPLT |
| | Ab106 | |
| 2180 | VHCDR1 (IMGT) | GFTFSSYA |
| | Ab107 | |
| 2181 | VHCDR2 (IMGT) | ISYDGSNK |
| | Ab107 | |
| 2182 | VHCDR3 (IMGT) | VRGAGRGFTLGPDGFDI |
| | Ab107 | |
| 2183 | VLCDR1 (IMGT) | RSLLWSDGKTY |
| | Ab107 | |
| 2184 | VLCDR2 (IMGT) | ELS |
| | Ab107 | |
| 2185 | VLCDR3 (IMGT) | MQYVEAPLT |
| | Ab107 | |
| 2186 | VHCDR1 (IMGT) | GFTFSSYA |
| | Vh1 | |
| 2187 | VHCDR2 (IMGT) | ISYDGSNK |
| | Vh1 | |
| 2188 | VHCDR3 (IMGT) | VRGAGRGYTYGPDGFDI |
| | Vh1 | |
| 2189 | VHCDR1 (IMGT) | GFTFSSYA |
| | Vh2 | |
| 2190 | VHCDR2 (IMGT) | ISYDGSNK |
| | Vh2 | |
| 2191 | VHCDR3 (IMGT) | ARGAGRGYTYGPDGFDI |
| | Vh2 | |
| 2192 | VLCDR1 (IMGT) | RSLLHSDGKTY |
| | Vk1 | |
| 2193 | VLCDR2 (IMGT) | ELS |
| | Vk1 | |
| 2194 | VLCDR3 (IMGT) | MQYIEAPLT |
| | Vk1 | |
| 2195 | VLCDR1 (IMGT) | RSLLHSDGKTY |
| | Vk2 | |
| 2196 | VLCDR2 (IMGT) | ELS |
| | Vk2 | |
| 2197 | VLCDR3 (IMGT) | MQYIEAPLT |
| | Vk2 | |
| 2198 | VLCDR1 (IMGT) | RSLLHSDGKTY |
| | Vk3 | |
| 2199 | VLCDR2 (IMGT) | ELS |
| | Vk3 | |
| 2200 | VLCDR3 (IMGT) | MQYIEAPLT |
| | Vk3 | |
| 2201 | VLCDR1 (IMGT) | RSLLHSDGKTY |
| | Vk4 | |
| 2202 | VLCDR2 (IMGT) | ELS |
| | Vk4 | |
| 2203 | VLCDR3 (IMGT) | MQYIEAPLT |
| | Vk4 | |
| 2204 | VLCDR1 (IMGT) | RSLLHSDGKTY |
| | Vk5 | |
| 2205 | VLCDR2 (IMGT) | ELS |
| | Vk5 | |
| 2206 | VLCDR3 (IMGT) | MQYIEAPLT |
| | Vk5 | |
| 2207 | VLCDR1 (IMGT) | QSLLHSDGKTY |
| | Vk6 | |
| 2208 | VLCDR2 (IMGT) | ELS |
| | Vk6 | |
| 2209 | VLCDR3 (IMGT) | MQYIEAPLT |
| | Vk6 | |
| 2210 | VLCDR1 (IMGT) | QSLLHSDGKTY |
| | Vk7 | |
| 2211 | VLCDR2 (IMGT) | ELS |
| | Vk7 | |
| 2212 | VLCDR3 (IMGT) | MQYIEAPLT |
| | Vk7 | |
| 2213 | VLCDR1 (IMGT) | RSLLHSDGKTY |
| | Vk8 | |
| 2214 | VLCDR2 (IMGT) | YLG |
| | Vk8 | |
| 2215 | VLCDR3 (IMGT) | MQYIEAPLT |
| | Vk8 | |
| 2216 | VLCDR1 (IMGT) | QSLLHSDGKTY |
| | Vk9 | |
| 2217 | VLCDR2 (IMGT) | ELS |
| | Vk9 | |
| 2218 | VLCDR3 (IMGT) | MQYIEAPLT |
| | Vk9 | |
| 2219 | VLCDR1 (IMGT) | RSLLHSDGKTY |
| | Vk10 | |
| 2220 | VLCDR2 (IMGT) | ELS |
| | Vk10 | |
| 2221 | VLCDR3 (IMGT) | MQYIEAPLT |
| | Vk10 | |
| 2222 | VLCDR1 (IMGT) | RSLLHSDGKTY |
| | Vk11 | |
| 2223 | VLCDR2 (IMGT) | ELS |
| | Vk11 | |
| 2224 | VLCDR3 (IMGT) | MQYIEAPLT |
| | Vk11 | |
| 2225 | VLCDR1 (IMGT) | RSLLHSDGKTY |
| | Vk12 | |
| 2226 | VLCDR2 (IMGT) | ELS |
| | Vk12 | |
| 2227 | VLCDR3 (IMGT) | MQYIEAPLT |
| | Vk12 | |
| 2228 | VLCDR1 (IMGT) | RSLLHSDGKTY |
| | Vk13 | |
| 2229 | VLCDR2 (IMGT) | ELS |
| | Vk13 | |
| 2230 | VLCDR3 (IMGT) | MQYIEAPLT |
| | Vk13 | |
| 2231 | Consensus #1 | [SGHT][YH]A[MI]H |
| | VHCDR1 | |
| 2232 | Consensus #1 | [VL]ISYDGS[NE]KYYADS[VA]KG |
| | VHCDR2 | |
| 2233 | Consensus #1 | GAG[RTLQHMI]G[YLFW][TPASIM][YNFHLWRK]GPDGDFI |
| | VHCDR3 | |
| 2234 | Consensus #1 | [TRK]SS[RQE]SL[LVI][HWYF][SRGT]DG[KNI]TY[VL][YSN] |
| | VLCDR1 | |
| 2235 | Consensus #1 | [EKQ][LVI]S[NS]RFS |
| | VLCDR2 | |
| 2236 | Consensus #1 | MQ[YA][IVTK][EQNR][AFL]P[LW]T |
| | VLCDR3 | |
| 2237 | Consensus #2 | [SG]YA[MI]H |
| | VHCDR1 | |
| 2238 | Consensus #2 | [VL]ISYDGSNKYYADS[VA]KG |
| | VHCDR2 | |
| 2239 | Consensus #2 | GAG[RTLQHMI]G[YLFW][TPASIM][YNFHLWRK]GPDGDFI |
| | VHCDR3 | |
| 2240 | Consensus #2 | [TRK]SS[RQE]SL[LV][HWY][SR]DG[KN]TY[VL][YS] |
| | VLCDR1 | |
| 2241 | Consensus #2 | [EK][LV]SNRFS |
| | VLCDR2 | |
| 2242 | Consensus #2 | MQ[YA][IVT][EQ][AF]P[LW]T |
| | VLCDR3 | |
| 2243 | Consensus #3 | [SG]YAMH |
| | VHCDR1 | |
| 2244 | Consensus #3 | VISYDGSNKYYADSVKG |
| | VHCDR2 | |
| 2245 | Consensus #3 | GAG[RTLQHMI]G[YLFW][TPASIM][YNFHLWRK]GPDGDFI |
| | VHCDR3 | |
| 2246 | Consensus #3 | [TRK]SS[RQ]SLL[HW]SDGKTY[VL]Y |
| | VLCDR1 | |
| 2247 | Consensus #3 | ELSNRFS |
| | VLCDR2 | |
| 2248 | Consensus #3 | MQY[IV]EAPLT |
| | VLCDR3 | |
| 2249 | Consensus #1 | GF[TSD]F[SGTA][SGHT][YH]A |
| | VHCDR1 (IMGT) | |
| 2250 | Consensus #1 | ISYDGS[NE]K |
| | VHCDR2 (IMGT) | |
| 2251 | Consensus #1 | VRGAG[RTLQHMI]G[YLFW][TPASIM][YNFHLWRK]GPDGDFI |
| | VHCDR3 (IMGT) | |
| 2252 | Consensus #1 | [RQE]SL[LVI][HWYF][SRGT]DG[KNI]TY |
| | VLCDR1 (IMGT) | |
| 2253 | Consensus #1 | [EKQ][LVI]S |
| | VLCDR2 (IMGT) | |
| 2254 | Consensus #1 | MQ[YA][IVTK][EQNR][AFL]P[LW]T |
| | VLCDR3 (IMGT) | |
| 2255 | Consensus #2 | GF[TD]F[SA][SG]YA |
| | VHCDR1 (IMGT) | |
| 2256 | Consensus #2 | ISYDGSNK |
| | VHCDR2 (IMGT) | |
| 2257 | Consensus #2 | VRGAG[RTLQHMI]G[YLFW][TPASIM][YNFHLWRK]GPDGDFI |
| | VHCDR3 (IMGT) | |
| 2258 | Consensus #2 | [RQE]SL[LV][HWY][SR]DG[KN]TY |
| | VLCDR1 (IMGT) | |
| 2259 | Consensus #2 | [EK][LV]S |
| | VLCDR2 (IMGT) | |
| 2260 | Consensus #2 | MQ[YA][IVT][EQ][AF]P[LW]T |
| | VLCDR3 (IMGT) | |
| 2261 | Consensus #3 | GF[TD]F[SA][SG]YA |
| | VHCDR1 (IMGT) | |
| 2262 | Consensus #3 | ISYDGSNK |
| | VHCDR2 (IMGT) | |
| 2263 | Consensus #3 | VRGAG[RTLQHMI]G[YLFW][TPASIM][YNFHLWRK]GPDGDFI |
| | VHCDR3 (IMGT) | |
| 2264 | Consensus #3 | [RQ]SLL[HW]SDGKTY |
| | VLCDR1 (IMGT) | |
| 2265 | Consensus #3 | ELS |
| | VLCDR2 (IMGT) | |
| 2266 | Consensus #3 | MQY[IV]EAPLT |
| | VLCDR3 (IMGT) | |

### EQUIVALENTS:

Those skilled in the art will recognize, or be able to ascertain using no more than routine experimentation, many equivalents of the specific embodiments disclosed herein. Such equivalents are intended to be encompassed by the following claims.

1. An antibody, or antigen-binding portion thereof, which binds to human FGFR1c, FGFR2c, FGFR3c, and/or FGFR4.

2. The antibody, or antigen-binding portion thereof, of claim 1, which does not bind to FGFR1b, FGFR2b, and/or FGFR3b.

3. The antibody, or antigen-binding portion thereof, of claim 1, wherein the antibody inhibits the binding of FGF1 and/or FGF2 to FGFR1c, FGFR2c, FGFR3c, and/or FGFR4.

4-11. (canceled)

12. An isolated monoclonal antibody, or antigen-binding portion thereof, which specifically binds to FGFR1c, FGFR2c, FGFR3c, and/or FGFR4, and comprises the three heavy chain CDRs and the three light chain CDRs that are in the heavy and light chain variable region pairs selected from the group consisting of: SEQ ID NOs: 29 and 30; 40 and 41; 51 and 52; 62 and 63; 73 and 74; 84 and 85; 95 and 96; 106 and 107; 117 and 118; 128 and 129; 139 and 140;
150 and 151; 161 and 162; 172 and 173; 183 and 184; 194 and 195; 204 and 205; 214 and 215;
224 and 225; 234 and 235; 244 and 245; 254 and 255; 264 and 265; 274 and 275; 284 and 285;
294 and 295; 304 and 305; 314 and 315; 324 and 325; 334 and 335; 344 and 345; 354 and 355;
364 and 365; 374 and 375; 384 and 385; 394 and 395; 404 and 405; 414 and 415; 424 and 425;
434 and 435; 444 and 445; 454 and 455; 464 and 465; 474 and 475; 484 and 485; 494 and 495;
504 and 505; 514 and 515; 524 and 525; 534 and 535; 544 and 545; 554 and 555; 564 and 565;
574 and 575; 584 and 585; 594 and 595; 604 and 605; 614 and 615; 624 and 625; 634 and 635;
644 and 645; 654 and 655; 664 and 665; 674 and 675; 684 and 685; 694 and 695; 704 and 705;
714 and 715; 724 and 725; 734 and 735; 744 and 745; 754 and 755; 764 and 765; 774 and 775;
784 and 785; 794 and 795; 804 and 805; 814 and 815; 824 and 825; 834 and 835; 844 and 845; 854 and 855; 864 and 865; 874 and 875; 884 and 885; 894 and 895; 904 and 905; 914 and 915; 924 and 925; 934 and 935; 944 and 945; 954 and 955; 964 and 965; 974 and 975; 984 and 985; 994 and 995; 1004 and 1005; 1014 and 1015; 1024 and 1025; 1034 and 1035; 1044 and 1045; 1054 and 1055; 1064 and 1065; 1074 and 1075; 1084 and 1085; 1094 and 1095; and 1104 and 1105.

13-36. (canceled)

37. An isolated monoclonal antibody, or antigen binding portion thereof, which binds to FGFR1c, FGFR2c, FGFR3c, and/or FGFR4, and comprises heavy and light chain variable region sequences which are at least 90%, 95%, 98%, 99%, or 100% identical to the amino acid sequences selected from the group consisting of: SEQ ID NOs: 29 and 30; 40 and 41; 51 and 52; 62 and 63; 73 and 74; 84 and 85; 95 and 96; 106 and 107; 117 and 118; 128 and 129; 139 and 140; 150 and 151; 161 and 162; 172 and 173; 183 and 184; 194 and 195; 204 and 205; 214 and 215; 224 and 225; 234 and 235; 244 and 245; 254 and 255; 264 and 265; 274 and 275; 284 and 285; 294 and 295; 304 and 305; 314 and 315; 324 and 325; 334 and 335; 344 and 345; 354 and 355; 364 and 365; 374 and 375; 384 and 385; 394 and 395; 404 and 405; 414 and 415; 424 and 425; 434 and 435; 444 and 445; 454 and 455; 464 and 465; 474 and 475; 484 and 485; 494 and 495; 504 and 505; 514 and 515; 524 and 525; 534 and 535; 544 and 545; 554 and 555; 564 and 565; 574 and 575; 584 and 585; 594 and 595; 604 and 605; 614 and 615; 624 and 625; 634 and 635; 644 and 645; 654 and 655; 664 and 665; 674 and 675; 684 and 685; 694 and 695; 704 and 705; 714 and 715; 724 and 725; 734 and 735; 744 and 745; 754 and 755; 764 and 765; 774 and 775; 784 and 785; 794 and 795; 804 and 805; 814 and 815; 824 and 825; 834 and 835; 844 and 845; 854 and 855; 864 and 865; 874 and 875; 884 and 885; 894 and 895; 904 and 905; 914 and 915; 924 and 925; 934 and 935; 944 and 945; 954 and 955; 964 and 965; 974 and 975; 984 and 985; 994 and 995; 1004 and 1005; 1014 and 1015; 1024 and 1025; 1034 and 1035; 1044 and 1045; 1054 and 1055; 1064 and 1065; 1074 and 1075; 1084 and 1085; 1094 and 1095; and 1104 and 1105.

38. (canceled)

39. An isolated monoclonal antibody, or antigen binding portion thereof, which binds to FGFR1c, FGFR2c, FGFR3c, and/or FGFR4, and comprises heavy and light chain sequences which are at least 90%, 95%, 98%, 99%, or 100% identical to the amino acid sequences selected from the group consisting of: SEQ ID NOs: 32 and 33; 43 and 44; 54 and 55; 65 and 66; 76 and 77; 87 and 88; 98 and 99; 109 and 110; 120 and 121; 131 and 132; 142 and 143; 153 and 154; 164 and 165; 175 and 176; 186 and 187; 196 and 197; 206 and 207; 216 and 217; 226 and 227; 236 and 237; 246 and 247; 256 and 257; 266 and 267; 276 and 277; 286 and 287; 296 and 297; 306 and 307; 316 and 317; 326 and 327; 336 and 337; 346 and 347; 356 and 357; 366 and 367; 376 and 377; 386 and 387; 396 and 397; 406 and 407; 416 and 417; 426 and 427; 436 and 437; 446 and 447; 456 and 457; 466 and 467; 476 and 477; 486 and 487; 496 and 497; 506 and 507; 516 and 517; 526 and 527; 536 and 537; 546 and 547; 556 and 557; 566 and 567; 576 and 577; 586 and 587; 596 and 597; 606 and 607; 616 and 617; 626 and 627; 636 and 637; 646 and 647; 656 and 657; 666 and 667; 676 and 677; 686 and 687; 696 and 697; 706 and 707; 716 and 717; 726 and 727; 736 and 737; 746 and 747; 756 and 757; 766 and 767; 776 and 777; 786 and 787; 796 and 797; 806 and 807; 816 and 817; 826 and 827; 836 and 837; 846 and 847; 856 and 857; 866 and 867; 876 and 877; 886 and 887; 896 and 897; 906 and 907; 916 and 917; 926 and 927; 936 and 937; 946 and 947; 956 and 957; 966 and 967; 976 and 977; 986 and 987; 996 and 997; 1006 and 1007; 1016 and 1017; 1026 and 1027; 1036 and 1037; 1046 and 1047; 1056 and 1057; 1066 and 1067; 1076 and 1077; 1086 and 1087; 1096 and 1097; and 1106 and 1107.

40-42. (canceled)

43. An isolated monoclonal antibody, or antigen-binding portion thereof, which specifically binds to FGFR1c, FGFR2c, FGFR3c, and/or FGFR4, and comprises heavy chain CDR1, CDR2, and CDR3 sequences [SGHT][YH]A[MI]H (SEQ ID NO: 2231), [VL]ISYDGS[NE]KYYADS[VA]KG (SEQ ID NO: 2232), and GAG[RTLQHMI]G[YLFW][TPASIM][YNFHLWRK]GPDGDFI (SEQ ID NO: 2233), respectively, and light chain CDR1, CDR2, and CDR3 sequences [TRK]SS[RQE]SL[LVI][HWYF][SRGT]DG[KNI]TY[VL][YSN] (SEQ ID NO: 2234), [EKQ][LVI]S[NS]RFS (SEQ ID NO: 2235), and MQ[YA][IVTK][EQNR][AFL]P[LW]T (SEQ ID NO: 2236), respectively.

44-45. (canceled)

46. An isolated monoclonal antibody, or antigen-binding portion thereof, which specifically binds to FGFR1c, FGFR2c, FGFR3c, and/or FGFR4, and comprises IMGT heavy chain CDR1, CDR2, and CDR3 sequences GF[TSD]F[SGTA][SGHT][YH]A (SEQ ID NO: 2249), ISYDGS[NE]K (SEQ ID NO: 2250), and VRGAG[RTLQHMI]G[YLFW][TPASIM][YNFHLWRK]GPDGDFI (SEQ ID NO: 2251), respectively, and IMGT light chain CDR1, CDR2, and CDR3 sequences [RQE]SL[LVI][HWYF][SRGT]DG[KNI]TY (SEQ ID NO: 2252), [EKQ][LVI]S (SEQ ID NO: 2253), and MQ[YA][IVTK][EQNR][AFL]P[LW]T (SEQ ID NO: 2254), respectively.

47-49. (canceled)

50. The antibody, or antigen-binding portion thereof, of claim 1, wherein the antibody is an IgG1, an IgG2, an IgG3, an IgG4, or a variant thereof.

51-53. (canceled)

54. The antibody of claim 50, wherein the antibody comprises an Fc region with reduced or no effector function.

55. The antibody of claim 1, wherein the antibody is a human antibody.

56. (canceled)

57. A modified antibody that binds to FGFR1c, wherein the antibody exhibits increased tolerability as compared to an antibody comprising identical heavy and light chain variable region sequences and an IgG1 constant region when administered to a mammal.

58. A modified antibody that binds to FGFR1c, wherein administration of the antibody to a mammal does not result in significant weight loss.

59-65. (canceled)

66. A multispecific molecule comprising the antibody of claim 1 linked to a molecule having a further binding specificity for a target molecule which is not a FGF receptor.

67. A nucleic acid encoding the heavy and/or light chain variable region of the antibody, or antigen-binding portion thereof, of claim 1.

68. An expression vector comprising the nucleic acid molecule of claim 67.

69. A cell transformed with an expression vector of claim 68.

70. An immunoconjugate comprising the antibody, or antigen-binding portion thereof, of claim 1, linked to a binding moiety, a labeling moiety, a biologically active moiety, or a therapeutic agent.

71. A composition comprising the antibody, or antigen-binding portion thereof, of claim 1.

72. (canceled)

73. A kit comprising the antibody, or antigen-binding portion thereof, of claim 1, and instructions for use.

74. A method of preparing an anti-FGFR antibody, or antigen binding portion thereof, comprising expressing the antibody, or antigen binding portion thereof, in the cell of claim 69 and isolating the antibody, or antigen binding portion thereof, from the cell.

75. A method of blocking FGF1 or FGF2 binding to FGFR1c, FGFR2c, FGFR3c, and/or FGFR4 in a cell comprising contacting the cell with an effective amount of the antibody, or antigen-binding portion thereof, of claim 1.

76-77. (canceled)

78. A method of inhibiting FGF-mediated signaling in a cell comprising contacting the cell with an effective amount of the antibody, or antigen-binding portion thereof, of claim 1.

79-80. (canceled)

81. A method of inhibiting the growth of tumor cells comprising administering to a subject -with a tumor a therapeutically effective amount of an antibody, or antigen-binding portion, of claim 1.

82-83. (canceled)

84. A method of treating cancer comprising administering to a subject in need thereof a therapeutically effective amount of the antibody, or antigen-binding portion, of claim 1.

85-97. (canceled)

98. A method of detecting the presence of FGFR (e.g., FGFR1c, FGFR2c, FGFR3c, and/or FGFR4) in a sample comprising contacting the sample with the anti-FGFR antibody of claim 1 under conditions that allow for formation of a complex between the antibody and FGFR protein, and detecting the formation of a complex.
